SEQUENCE LISTING (89 sequences)

SEQ ID NO: 1 - DNA, 1998 bp, Pseudomonas meridiana
  CDS (1)..(1995)
  sig_peptide (1)..(60)
  mat_peptide (61)..(1995)
atgactgtcctcagccgttgcttgttggtcctgggtgtattgctggccggcgcctgcgcccagcaacccgcgccttatactccccccgccgagcgcccgacgccgacgcgtgaagcgccgtggccgaaaaaccacttcctgggcattgcctaccacgacattgaagaccgcgaccccgaccaagccgtagtggcggtgcgcaccgagcgtctgatcgaccaattggcctggctgcgcgagaacggttaccagccggtcagcgttgatcagatcctggccgcccgcagcggtgggccggagctgccgcccaaagccgtcatgctcagcttcgatgacggctactccagtttctatacccgtgtaatgccaatcctgcgtgcctataactggcctgcaattctggccccggtgggctattggatcgacacgcccttgaacaaaccggtggattttgccggcacgccgagagcgcgttccgacttcctcacctgggagcaggtgcgcgagatttccaagtcgccgctggtggaaatcgccgcccacaccgacgcgagccactccggcatcctggccaacccccaaggtaacctggagcccgccgccgcgacccgacgctatgacgccgccaccggcaactatgaaaccgaagctcagttccaggcgcggatgcgggctgacgtaaccgccatttccaacaaactgcgcgccgttaccggcaaagcaccgcgcgtgtgggtgtggccttatggcgctgccgatggcacctcgttggccgtcgtgggcgagcagggttaccagatggccctgacactggacgacggcctcgacgacctcgccaacctcaagagcagcccgcgctttctggtggcttcggacccggatggcgagcattacgccaacgccattgtcggcacgcaggccaaaccctcgatgcgcgtgctgcacgtggacctagacaacgtttacgacccggacccggcccaagaggcgcgtaacctcgaccagttgatccagcgtgtggtggacatgggcgccagcaccgtattcctgcaagccttcgctgaccctaaaggtgacggcctggtgcactcgctgtacttccctaaccgccatctgccggtgcgtcaggacctgttcaaccgcgtgacctggcaactgcgtactcgtggccatgtggcgatttacgcgtggatgccggtgctcagttttgccctggacccggcgttgccgcgcgtcacccgctgggacccgaaaaccggccaggtcggcatcgacccggaccaatacaagcgtctgtcgccgtttgacccgaaggtgcgtcagcagatcggtgagatctacgaggacctggcgcgcaacacggccatcgacggcgtgctgttccacgacgacgcgctcttttcagatttcgaagacgccagccccgcagcgctcaaggcctatgccgcgaacggcctgccgagcagcattgccgccttgcgcgccgaccccgccacgatgcaacgctggacgcgcttcaagagccgttacctgatcgacttcacccaggaactgatcggcaaggttcgcgccattcgtgggccgcaggtgcagaccgcgcgcaacatcttcgccgagccgatgctcaacccggccagcgaaacctggttcgcgcagaaccttgatgacttccttcagacctacgactggaccgcgccgatggccatgccgctgatggaaggcgaaacattgaaaaactccaatgcctggctggaaaaactcgtggccacggtcaaggcgcgccccggcgccttgcagcgcacggtgtttgaactgcaagccaaagactggcggacctcagacgcgcccaacattgatggcgcgcagatggctgaatggatgggggtgcttaaacgccagggggtcaggagcttcggctactacccggacaacttcctggaaaattcgccggacctgaagacggtacgtccggccctgtccaaccaatggaacccttga

SEQ ID NO: 2 - protein, 665 aa, Pseudomonas meridiana
MTVLSRCLLVLGVLLAGACAQQPAPYTPPAERPTPTREAPWPKNHFLGIAYHDIEDRDPDQAVVAVRTERLIDQLAWLRENGYQPVSVDQILAARSGGPELPPKAVMLSFDDGYSSFYTRVMPILRAYNWPAILAPVGYWIDTPLNKPVDFAGTPRARSDFLTWEQVREISKSPLVEIAAHTDASHSGILANPQGNLEPAAATRRYDAATGNYETEAQFQARMRADVTAISNKLRAVTGKAPRVWVWPYGAADGTSLAVVGEQGYQMALTLDDGLDDLANLKSSPRFLVASDPDGEHYANAIVGTQAKPSMRVLHVDLDNVYDPDPAQEARNLDQLIQRVVDMGASTVFLQAFADPKGDGLVHSLYFPNRHLPVRQDLFNRVTWQLRTRGHVAIYAWMPVLSFALDPALPRVTRWDPKTGQVGIDPDQYKRLSPFDPKVRQQIGEIYEDLARNTAIDGVLFHDDALFSDFEDASPAALKAYAANGLPSSIAALRADPATMQRWTRFKSRYLIDFTQELIGKVRAIRGPQVQTARNIFAEPMLNPASETWFAQNLDDFLQTYDWTAPMAMPLMEGETLKNSNAWLEKLVATVKARPGALQRTVFELQAKDWRTSDAPNIDGAQMAEWMGVLKRQGVRSFGYYPDNFLENSPDLKTVRPALSNQWNP

SEQ ID NO: 3 - protein, 645 aa, Pseudomonas meridiana
QQPAPYTPPAERPTPTREAPWPKNHFLGIAYHDIEDRDPDQAVVAVRTERLIDQLAWLRENGYQPVSVDQILAARSGGPELPPKAVMLSFDDGYSSFYTRVMPILRAYNWPAILAPVGYWIDTPLNKPVDFAGTPRARSDFLTWEQVREISKSPLVEIAAHTDASHSGILANPQGNLEPAAATRRYDAATGNYETEAQFQARMRADVTAISNKLRAVTGKAPRVWVWPYGAADGTSLAVVGEQGYQMALTLDDGLDDLANLKSSPRFLVASDPDGEHYANAIVGTQAKPSMRVLHVDLDNVYDPDPAQEARNLDQLIQRVVDMGASTVFLQAFADPKGDGLVHSLYFPNRHLPVRQDLFNRVTWQLRTRGHVAIYAWMPVLSFALDPALPRVTRWDPKTGQVGIDPDQYKRLSPFDPKVRQQIGEIYEDLARNTAIDGVLFHDDALFSDFEDASPAALKAYAANGLPSSIAALRADPATMQRWTRFKSRYLIDFTQELIGKVRAIRGPQVQTARNIFAEPMLNPASETWFAQNLDDFLQTYDWTAPMAMPLMEGETLKNSNAWLEKLVATVKARPGALQRTVFELQAKDWRTSDAPNIDGAQMAEWMGVLKRQGVRSFGYYPDNFLENSPDLKTVRPALSNQWNP

SEQ ID NO: 4 - DNA, 1962 bp, Halomonas sp-62262
  CDS (1)..(1959)
  sig_peptide (1)..(72)
  mat_peptide (73)..(1959)
atgacactctggcgcgtcattttattgggtgctttcttgctagtggtcgttaatattcagcaagcccaggccgcccgctcaccaaatgattacgtggtgattagctaccacgatattgtggattccagcgtcacgccggatatggatatctattcacaaaccattacacgcagtcgattaatcgaacattttaaccttattgacgctgggggttatcagcctgtcagtttgcagcaaatcattgatgcaaaagcgggaggcttaccgctgccagaaaaagcagtgctgctgaccttcgacgatggctatcgcagtttttacgatatcgtctttccgctactccagctgtataacttccccgccgttcaagccgttgtcggtagctggttggatgtgcctgctggcgggcaagtgccctatggcagtattacgctgcctcgtgagcgttttttatcgtgggcacaagtcaaaacgttagacgcgtcgccgctggttgaaattgcttctcactcttacaacctgcattacggtgtggtcggcaaccccatgggtaacgaacaggcagcagcagtgaccagtatttggagcgcctatggtgggtacgaaagtgaggagcagtaccttgcgcggttgagacaagacatggcgcaaacacagcagcgcttccaagagcaggtgggccgtagcccacgcatcatcgtgtggccctacggcgcctatagtgaagcaacgcttaacattgccgccgaatatggcatggactatacattcagcttgcttagcgccccgaaccagttaaacgactcaatgcgtacgatgaaccgctacctcatcgaccaggaaaccagcctgcaaaccattgatgaaatgctttccaaccgcgtgtgggagcctgaagatctgcgcattgtgcatgtcgatttagactatgtgtatgaccctgacccgattcaacaggagcaaaatcttgaccgcctgattgagcgcatttctcgctacggcgtaagtaccgtttaccttcaggcctatgcagaccctgacggtgacggcgtagccgatgcgctttacttccctaaccgccacctgccggtaagagctgacctgttcaatcgcgtggcctggcagttaaagaaacgcgctagcgtaaaggtatacgcctggatgccggtgctgtcgtttgacctgggagcaggctaccaatatgtcaccgatgtccgaaccggcgcggaagcacctgaccactaccgtcggctctcaccttacgtggaagagaaccgccgtatcatccgcgaaatctatcaggacctcggccgcctgactaagttcgatgggctgctgttccacgatgacgccttcttcaccgactttgaagatgcaacccctgacgccattgctgcctatgaagatgcctcattgccaagcgatattaatacaatacgtaacgacgatagcttaatgaccacctggacgcgcttcaaaacggcgtatctaactgacttcacctacgagttggagcaggccgcaaactactatcgtcaggctgataacaaagtatttaccacatcgcgcaacctgtatgccgtcacggtgatggagccacgcagccagcgatggtttgctcaggatatccaaagcttcgccgccggctacgactttgtggccgttatggccatgccgtatatggaagaagcagaaaatcccgacgaatggcttcgagcacttgcccagcgctcgctcgcgcaggtaagcgctgaacaactggtgtttgagctacaaacgcagaattggcacacccaaaccccgattcccagcgaagagatagctcagtgggtacgtatcctgcgcgaagaaggcatcaagaacattggctattacccggatgattttcttcaaaatcacccagacgctaatgtcatgaggccggtcttttctatcggacgtcggtttagggcaacgccatga

SEQ ID NO: 5 - protein, 653 aa, Halomonas sp-62262
MTLWRVILLGAFLLVVVNIQQAQAARSPNDYVVISYHDIVDSSVTPDMDIYSQTITRSRLIEHFNLIDAGGYQPVSLQQIIDAKAGGLPLPEKAVLLTFDDGYRSFYDIVFPLLQLYNFPAVQAVVGSWLDVPAGGQVPYGSITLPRERFLSWAQVKTLDASPLVEIASHSYNLHYGVVGNPMGNEQAAAVTSIWSAYGGYESEEQYLARLRQDMAQTQQRFQEQVGRSPRIIVWPYGAYSEATLNIAAEYGMDYTFSLLSAPNQLNDSMRTMNRYLIDQETSLQTIDEMLSNRVWEPEDLRIVHVDLDYVYDPDPIQQEQNLDRLIERISRYGVSTVYLQAYADPDGDGVADALYFPNRHLPVRADLFNRVAWQLKKRASVKVYAWMPVLSFDLGAGYQYVTDVRTGAEAPDHYRRLSPYVEENRRIIREIYQDLGRLTKFDGLLFHDDAFFTDFEDATPDAIAAYEDASLPSDINTIRNDDSLMTTWTRFKTAYLTDFTYELEQAANYYRQADNKVFTTSRNLYAVTVMEPRSQRWFAQDIQSFAAGYDFVAVMAMPYMEEAENPDEWLRALAQRSLAQVSAEQLVFELQTQNWHTQTPIPSEEIAQWVRILREEGIKNIGYYPDDFLQNHPDANVMRPVFSIGRRFRATP

SEQ ID NO: 6 - protein, 629 aa, Halomonas sp-62262
ARSPNDYVVISYHDIVDSSVTPDMDIYSQTITRSRLIEHFNLIDAGGYQPVSLQQIIDAKAGGLPLPEKAVLLTFDDGYRSFYDIVFPLLQLYNFPAVQAVVGSWLDVPAGGQVPYGSITLPRERFLSWAQVKTLDASPLVEIASHSYNLHYGVVGNPMGNEQAAAVTSIWSAYGGYESEEQYLARLRQDMAQTQQRFQEQVGRSPRIIVWPYGAYSEATLNIAAEYGMDYTFSLLSAPNQLNDSMRTMNRYLIDQETSLQTIDEMLSNRVWEPEDLRIVHVDLDYVYDPDPIQQEQNLDRLIERISRYGVSTVYLQAYADPDGDGVADALYFPNRHLPVRADLFNRVAWQLKKRASVKVYAWMPVLSFDLGAGYQYVTDVRTGAEAPDHYRRLSPYVEENRRIIREIYQDLGRLTKFDGLLFHDDAFFTDFEDATPDAIAAYEDASLPSDINTIRNDDSLMTTWTRFKTAYLTDFTYELEQAANYYRQADNKVFTTSRNLYAVTVMEPRSQRWFAQDIQSFAAGYDFVAVMAMPYMEEAENPDEWLRALAQRSLAQVSAEQLVFELQTQNWHTQTPIPSEEIAQWVRILREEGIKNIGYYPDDFLQNHPDANVMRPVFSIGRRFRATP

SEQ ID NO: 7 - DNA, 1998 bp, Pseudomonas migulae
  CDS (1)..(1995)
  sig_peptide (1)..(60)
  mat_peptide (61)..(1995)
atgaccgtcctttgccgttgcctgttggtcctgggtgtaatgctggccagtgcctgcgcccagcaacccgcgcccttcactccacctgccgagcggccgatactggccaacgaagcgccgtggccgaaaaaccatttcctgggcatcgcctaccacgacgtcgaggatcgcgaccccgaccaggcggtggtggcggtgcgcaccgaacgcttgatcgaacagctggcgtggttgcgtgagaacggctaccaggcggtcagcgtcgaccagatcctggcggcacgcaacggcggccccgagttgccacccaaggccatcatgctcagcttcgacgacggctattcgagcttttacacccgcgtgatgccgattctgcgcgcctatcgctggccggccttgctggcgccggtggggtattggatcgacacgccgctcaatcaaccggtggacttcgccggctcgccgcgcccacgtggggaattcctcacctggcaacagattcgcgaagtgtcgcaatcgggcctggtggaaatcgccgcgcacaccgacaacaaccacaaagggattttggccaacccccagggcaacctggagccggcggcgaccagcctgcgctttgacccggccaccgggcgttatgaagactcggccaaattcgatgcgcgcatgcgggctgatgtcgcggcgatcaccaacaagatccgcagcgtaaccggcaaggcgcctcgtgtatgggtatggccttacggcgccgccaagggcacctcgctggcgattgtcggcgagcagggctaccagatggcgctgaccctggaagacggcctcgacagcctcggcaacctgatgaacagcccgcgctttttggtggcctcggacccggatggcgaacacttcgccaacagcatggtggccgtgcaaacccaggcaccgctgcgcgtgctgcacgtggacctcgacaacgtttacgaccccgacccggcccagcaagcgcgcaacctcgaccagttggtacagcgtgtggtcgacatgggcgcgggcaccgtgttcctgcaagcttttgccgaccccaaaggcgatggcctggtgcactcgctgtacttccccaaccgccatttgccggtgcgcgccgacctgttcaaccgcgtctcctggcagttgcacacccgtgcccatgcggcggtttacgcctggatgccggtgctgagtttcgcccttgaccccaagttgcagcgtgtgacccgctgggacccgcaaaccggccaggtcgccaccgaccctgaccaatacaagcgcttgtcgccgttcgacccgcaggtgcgcaagatcatcggcgagatctatgaagacctggcccgcaacaatgccatcgacggtgtgctgtaccacgacgacgccgtgctttccgacttcgaagacgccagccccgctgcgctcaaggcttatgccgccaacggcctgccgggcagcattgctgcactgcgcgcagacccggcagtgatgcaacgctggacgcgtttcaagagccgctacctgatcgacttcaccaacgagctgaccgccaaggtccgcgccatcggtggcccgcaggtgcagaccgcacgcaatatcttcgccgagccgatgctcaacccggccagcgaagcctggtttgcacagaacctcgatgacttcctccaggcctacgactggaccgcgcccatggccatgccgttgatggaaggccaggagttgaaaacctccaacgcctggctggaaaaactcgttgccaccgtcaaggcgcgccccggcgcgctggaaaaaaccgtgttcgagctgcaagccaaagactggcggaccaaagcggcgccggatatcaatgctgtgcaaatggccgaatggatgggcgtgctcaaacgccagggggtcaagagctttggctactacccggataacttcctggaaaactcgccggacctgaagacagttcgtccggccctttccaaccagtggaacccttga

SEQ ID NO: 8 - protein, 665 aa, Pseudomonas migulae
MTVLCRCLLVLGVMLASACAQQPAPFTPPAERPILANEAPWPKNHFLGIAYHDVEDRDPDQAVVAVRTERLIEQLAWLRENGYQAVSVDQILAARNGGPELPPKAIMLSFDDGYSSFYTRVMPILRAYRWPALLAPVGYWIDTPLNQPVDFAGSPRPRGEFLTWQQIREVSQSGLVEIAAHTDNNHKGILANPQGNLEPAATSLRFDPATGRYEDSAKFDARMRADVAAITNKIRSVTGKAPRVWVWPYGAAKGTSLAIVGEQGYQMALTLEDGLDSLGNLMNSPRFLVASDPDGEHFANSMVAVQTQAPLRVLHVDLDNVYDPDPAQQARNLDQLVQRVVDMGAGTVFLQAFADPKGDGLVHSLYFPNRHLPVRADLFNRVSWQLHTRAHAAVYAWMPVLSFALDPKLQRVTRWDPQTGQVATDPDQYKRLSPFDPQVRKIIGEIYEDLARNNAIDGVLYHDDAVLSDFEDASPAALKAYAANGLPGSIAALRADPAVMQRWTRFKSRYLIDFTNELTAKVRAIGGPQVQTARNIFAEPMLNPASEAWFAQNLDDFLQAYDWTAPMAMPLMEGQELKTSNAWLEKLVATVKARPGALEKTVFELQAKDWRTKAAPDINAVQMAEWMGVLKRQGVKSFGYYPDNFLENSPDLKTVRPALSNQWNP

SEQ ID NO: 9 - protein, 645 aa, Pseudomonas migulae
QQPAPFTPPAERPILANEAPWPKNHFLGIAYHDVEDRDPDQAVVAVRTERLIEQLAWLRENGYQAVSVDQILAARNGGPELPPKAIMLSFDDGYSSFYTRVMPILRAYRWPALLAPVGYWIDTPLNQPVDFAGSPRPRGEFLTWQQIREVSQSGLVEIAAHTDNNHKGILANPQGNLEPAATSLRFDPATGRYEDSAKFDARMRADVAAITNKIRSVTGKAPRVWVWPYGAAKGTSLAIVGEQGYQMALTLEDGLDSLGNLMNSPRFLVASDPDGEHFANSMVAVQTQAPLRVLHVDLDNVYDPDPAQQARNLDQLVQRVVDMGAGTVFLQAFADPKGDGLVHSLYFPNRHLPVRADLFNRVSWQLHTRAHAAVYAWMPVLSFALDPKLQRVTRWDPQTGQVATDPDQYKRLSPFDPQVRKIIGEIYEDLARNNAIDGVLYHDDAVLSDFEDASPAALKAYAANGLPGSIAALRADPAVMQRWTRFKSRYLIDFTNELTAKVRAIGGPQVQTARNIFAEPMLNPASEAWFAQNLDDFLQAYDWTAPMAMPLMEGQELKTSNAWLEKLVATVKARPGALEKTVFELQAKDWRTKAAPDINAVQMAEWMGVLKRQGVKSFGYYPDNFLENSPDLKTVRPALSNQWNP

SEQ ID NO: 10 - DNA, 2001 bp, Pseudomonas sp-62331
  CDS (1)..(1998)
  sig_peptide (1)..(60)
  mat_peptide (61)..(1998)
atgtctggaatcacacgctgcatcctgctgctgggcgtcatgctgctcaccgcttgcgcccaacaagctccagccttcacgccgccgtcacagcgaccgataccggccaatgaaaagccctggccgaaaaaccatgtgctgggcattgcctaccacgatgtcgaagatcgcgaccccgatcaggccgttgttgccgtgcgcaccgagcggctgatcgagcaactcgcctggctgcgcgagaacggctacacagcagtcaccgtcgaccagatcatggcggcgcgcaacggcggcccggagctgccgcccaaggccattctgctgagtttcgacgacggctattcgagcttctacacccgtgtcatgccggtgttgcgggcctacaactggcacgctttgctggcgccggtcggggtctggatcgatacgccgctggataaacccgtggacttcgcgggcaccccgcgccagcgttcggacttcctgacctgggcgcagatcaccgaggtctcgaagtccggcctggtggaaatcgccgctcacaccgacgccagtcacaagggcatcctggccaacccgcaaggtaacctgcaacctgcggcggcgacccgtggttatgacgcggcaaccgggcgctacgagtcggaagccgatttccaggcgcgtctgcgcaaggacgtcgtcaccatcactgaaaaaatccgcaaggccaccggctataagccgcgtgtttgggtctggccttatggtacggcggatggcaccgcgttgcaggtcatcggttccgagggctaccagatggccctgaccctcgacgacggcctcgattcactgaataacctgatgagcagtccgcgtttcctggtggcctccgatccggatggcgaacacttcgccaacagcatcgttgcggtgcaatccgacgcgccgatgcgcgtggtgcacgttgacctggataacgtctacgacccggacccggttcagcaggacgccaacctcggcaagctgatccagcgcatggccgacatgggcgccaacacagtgttcctgcaagccttcgccgaccctaaaggcgacgggctggtgcattcgctgtatttccccaaccgccatttgccggtgcgcgccgacatcttcgaccgcgtcgcctggcagctgcgcactcgcgcccacgtcaaagtcttcgcctggatgccggtgttgagtttcgccctggatccggccttgccccgcgtgacgcgctggaacccggacattggccagacctcggttgatcccggccagtaccggcgcctgtcgccgttcgacccgaacgtgcggcggatcatcggtgagatctatgaggacgtggcgcgtctgacctcggtcgacggcatcctctaccacgacgatgcgctgctctcggatttcgaagacgccagcccgcaggcgctgaaggtttacgccgcgaacggcttgcctgactcgatggccgccttgcgcgccgatccggcaacgctgcaacgctggagccgtttcaagagccgctacctgatcgacttcaccaacgaactcaccgccaaagtccgcgctattcgcgggccgcaggtgcagaccgctcgcaacctgttcgctgaaccgatgctcaacccgtccagcgaagcctggttcgcgcagaacctcgacgacttcctcggcgcctacgactggaccgcgccgatggccatgccgctgatggaaggcaagagctgcgagcaatccggcgcctggttggaagcgctggtggcgaccgttcgatcgcaccccggcgcgctggatcgcacggtgttcgaactgcaggcccgcgactggacccgcaaggacgccgacgcagaactgaacggcgagcaactggcggactggatggggcgcctcaaacgtcagggcgcgaccagtttcggctactacccggacaacttcctcgagaaccagccggacctgaaaaccgtgcgtcccgcactctccaacaagtggaatccttaa

SEQ ID NO: 11 - protein, 666 aa, Pseudomonas sp-62331
MSGITRCILLLGVMLLTACAQQAPAFTPPSQRPIPANEKPWPKNHVLGIAYHDVEDRDPDQAVVAVRTERLIEQLAWLRENGYTAVTVDQIMAARNGGPELPPKAILLSFDDGYSSFYTRVMPVLRAYNWHALLAPVGVWIDTPLDKPVDFAGTPRQRSDFLTWAQITEVSKSGLVEIAAHTDASHKGILANPQGNLQPAAATRGYDAATGRYESEADFQARLRKDVVTITEKIRKATGYKPRVWVWPYGTADGTALQVIGSEGYQMALTLDDGLDSLNNLMSSPRFLVASDPDGEHFANSIVAVQSDAPMRVVHVDLDNVYDPDPVQQDANLGKLIQRMADMGANTVFLQAFADPKGDGLVHSLYFPNRHLPVRADIFDRVAWQLRTRAHVKVFAWMPVLSFALDPALPRVTRWNPDIGQTSVDPGQYRRLSPFDPNVRRIIGEIYEDVARLTSVDGILYHDDALLSDFEDASPQALKVYAANGLPDSMAALRADPATLQRWSRFKSRYLIDFTNELTAKVRAIRGPQVQTARNLFAEPMLNPSSEAWFAQNLDDFLGAYDWTAPMAMPLMEGKSCEQSGAWLEALVATVRSHPGALDRTVFELQARDWTRKDADAELNGEQLADWMGRLKRQGATSFGYYPDNFLENQPDLKTVRPALSNKWNP

SEQ ID NO: 12 - protein, 646 aa, Pseudomonas sp-62331
QQAPAFTPPSQRPIPANEKPWPKNHVLGIAYHDVEDRDPDQAVVAVRTERLIEQLAWLRENGYTAVTVDQIMAARNGGPELPPKAILLSFDDGYSSFYTRVMPVLRAYNWHALLAPVGVWIDTPLDKPVDFAGTPRQRSDFLTWAQITEVSKSGLVEIAAHTDASHKGILANPQGNLQPAAATRGYDAATGRYESEADFQARLRKDVVTITEKIRKATGYKPRVWVWPYGTADGTALQVIGSEGYQMALTLDDGLDSLNNLMSSPRFLVASDPDGEHFANSIVAVQSDAPMRVVHVDLDNVYDPDPVQQDANLGKLIQRMADMGANTVFLQAFADPKGDGLVHSLYFPNRHLPVRADIFDRVAWQLRTRAHVKVFAWMPVLSFALDPALPRVTRWNPDIGQTSVDPGQYRRLSPFDPNVRRIIGEIYEDVARLTSVDGILYHDDALLSDFEDASPQALKVYAANGLPDSMAALRADPATLQRWSRFKSRYLIDFTNELTAKVRAIRGPQVQTARNLFAEPMLNPSSEAWFAQNLDDFLGAYDWTAPMAMPLMEGKSCEQSGAWLEALVATVRSHPGALDRTVFELQARDWTRKDADAELNGEQLADWMGRLKRQGATSFGYYPDNFLENQPDLKTVRPALSNKWNP

SEQ ID NO: 13 - DNA, 1998 bp, Pseudomonas jessenii
  CDS (1)..(1995)
  sig_peptide (1)..(81)
  mat_peptide (82)..(1995)
atgcctttgatttcgcgtttcatccttctgctgggagcgctgctggtcagcgcctgcgcccagcaagctccggcctttgcgccgccatccgagcgtccgctggcggccagcgaaaaaccgtggccgaaaaatcacgtgctcggcattgcctatcacgacgttgaagaccgcgatcccgatcaggccgtggtggcggtgcgcaccgagcgcatgatcgagcagctcgcgtggttgcgcgagaacggctacaaaccggtcaccgtcgaccagatcatggccgcgcgcaagggcggcccggagctgccggcgaaagcgatcctgctcagcttcgacgacggctattcgagcttttacacccgcgtactgccggtgctgcgcgcctataactggaacgcgctgctggcgccggtcggcagctggatcgacacgccgctcaatcaaccggtggattttgccggtgcgccgcgtgcgcgttccgacttcctgacctgggatcagattcgcgaaatctcgcaatccggtctggtggaaattgccgcgcacaccgacgccaaccacaaagggatcctggccaacccgcagggcaacctgcagcctgctgccgcgacccggcgctatgatccgctgaccaaacgttatgagtccgaggccgaattccaagcacggattcgcaccgacgtgaacaacatctcggaaaaaatccgcaaggtcaccggcaagaaaccacgcgtctgggtctggccgtatggcgcggcggacggcacatcgctgacggtggttggcgaggagggctacgaaatggccctgaccctcgaagacggtctcgacgccctcgacaacctgatgagcagcccgcgctttctcgttgcttcggatcctgacagcgagcgtttcgccaacagtatcgtcgacgtgcagtcggatttcgtcatgcgcgttgtgcatgttgatctggacaacgtttacgacccggatccggcgcagcaggacatcaacctcggaaaactggtgcagcgcatcgctgacttgggcgccaacacggtgttcctgcaagccttcgccgatcccaagggcgatggcctggtgcattcgctgtacttccccaaccgtcacttgccggtgcgtgcggatatttttaaccgcgtcgcctggcaactacgtacccgcgctaacgtgaaggtcttcgcgtggatgccggtgctgagttttggtctcgactcgaaactgccgcgcgtgacccgttgggacccgaaaaccggcatcacctcggtggatccggatcagtaccaacgcttgtcgccgttcgatcctgaagtgcgacggatcatcggtgaaatctacgaagacgtggcgcgcctgacctcggtcaacggcattctttaccacgacgacgcggtactgtcggatttcgaagatgccggccctgaagccctgaaagcctacgctgcccacggcttgccgggttcgattgcagcattgcgcgacgatccggcagcgatgcagcgctggacgcgcttcaagagccaatacctcattgacttcaccaacgagctgaccgccaaagtccgtgcgatccgcggcccgcaggtgctgaccgcacgcaatattttcgccgagccaatgctcaatcccgagagcgaagcatggtacgcacagaacctcgatgacttcttgcagacctacgactggaccgcgccgatggccatgccgctgatggaaaaacagacccaggcagagtccggcccgtggctggaagcgttggtggctacagtcaggaaacgccccggcgcgctggatcgtacggtgttcgaattgcaggcacgcgactggacgaaaaaggatcaggccgacattgacggtgcccttctggctgactggatgggtcgcctcaagcgtcagggcgcgaccagtttcggttactacccggacaacttcctcgacaacctgccggacctgaaaaccgtaaggcctgcactctccaacaaatggaatccataa

SEQ ID NO: 14 - protein, 665 aa, Pseudomonas jessenii
MPLISRFILLLGALLVSACAQQAPAFAPPSERPLAASEKPWPKNHVLGIAYHDVEDRDPDQAVVAVRTERMIEQLAWLRENGYKPVTVDQIMAARKGGPELPAKAILLSFDDGYSSFYTRVLPVLRAYNWNALLAPVGSWIDTPLNQPVDFAGAPRARSDFLTWDQIREISQSGLVEIAAHTDANHKGILANPQGNLQPAAATRRYDPLTKRYESEAEFQARIRTDVNNISEKIRKVTGKKPRVWVWPYGAADGTSLTVVGEEGYEMALTLEDGLDALDNLMSSPRFLVASDPDSERFANSIVDVQSDFVMRVVHVDLDNVYDPDPAQQDINLGKLVQRIADLGANTVFLQAFADPKGDGLVHSLYFPNRHLPVRADIFNRVAWQLRTRANVKVFAWMPVLSFGLDSKLPRVTRWDPKTGITSVDPDQYQRLSPFDPEVRRIIGEIYEDVARLTSVNGILYHDDAVLSDFEDAGPEALKAYAAHGLPGSIAALRDDPAAMQRWTRFKSQYLIDFTNELTAKVRAIRGPQVLTARNIFAEPMLNPESEAWYAQNLDDFLQTYDWTAPMAMPLMEKQTQAESGPWLEALVATVRKRPGALDRTVFELQARDWTKKDQADIDGALLADWMGRLKRQGATSFGYYPDNFLDNLPDLKTVRPALSNKWNP

SEQ ID NO: 15 - protein, 638 aa, Pseudomonas jessenii
PPSERPLAASEKPWPKNHVLGIAYHDVEDRDPDQAVVAVRTERMIEQLAWLRENGYKPVTVDQIMAARKGGPELPAKAILLSFDDGYSSFYTRVLPVLRAYNWNALLAPVGSWIDTPLNQPVDFAGAPRARSDFLTWDQIREISQSGLVEIAAHTDANHKGILANPQGNLQPAAATRRYDPLTKRYESEAEFQARIRTDVNNISEKIRKVTGKKPRVWVWPYGAADGTSLTVVGEEGYEMALTLEDGLDALDNLMSSPRFLVASDPDSERFANSIVDVQSDFVMRVVHVDLDNVYDPDPAQQDINLGKLVQRIADLGANTVFLQAFADPKGDGLVHSLYFPNRHLPVRADIFNRVAWQLRTRANVKVFAWMPVLSFGLDSKLPRVTRWDPKTGITSVDPDQYQRLSPFDPEVRRIIGEIYEDVARLTSVNGILYHDDAVLSDFEDAGPEALKAYAAHGLPGSIAALRDDPAAMQRWTRFKSQYLIDFTNELTAKVRAIRGPQVLTARNIFAEPMLNPESEAWYAQNLDDFLQTYDWTAPMAMPLMEKQTQAESGPWLEALVATVRKRPGALDRTVFELQARDWTKKDQADIDGALLADWMGRLKRQGATSFGYYPDNFLDNLPDLKTVRPALSNKWNP

SEQ ID NO: 16 - DNA, 1998 bp, Pseudomonas koreensis
  CDS (1)..(1995)
  sig_peptide (1)..(81)
  mat_peptide (82)..(1995)
atgcctttgattacgcgtttcatccttttgctgggagtgctgctggtcagcgcctgcgcccagcaagctccggccttcgcgccgccgtcgcagcgtccggtagcggccaatgaaaagccgtggccgaaaaaccatgtgctgggtatcgcctatcacgacgtcgaagaccgtgaccccgatcaggccgtggtggcggtgcgcaccgagcgcatgatcgagcaactcgcgtggctgcgcgagaacggctacaaaccggtcaccgtcgaccagatcatggccgcgcgcaaaggcggcccggaactgccaccgaaggcgatcctgctcagcttcgacgacggttactcaagcttctatacccgcgtgctgccgctgctgcgcgcctacaactggcacgccttgctggcgccggtcggcgtgtggatcgacacgccgctcaatcaaccggtggatttcgccggtgcgccacgggcacgaaccgacttcctgacctgggatcaggtgcgggaaatctcgcagtcggggctggtggaaatcgccgcgcacacggacgccagtcacaaagggatcctcgccaacccgcagggcaacctgcagccggcggcggcgacccgccgctacgatccggtcaccaagcgctacgagtccgaagaggatttccgtgcacggatccgcaacgacgtcgcgaccatttcggaaaagatccgcaaggtcaccggcaagaaaccgcgcatctgggtctggccgtacggcgcggcggacggcacttcgctggcggtggtcggcgagcagggctacgaaatggccctgactctggacgacggtctcgacgccctcgacaacctgatgagcagcccgcgcttcctggtggcctccgatcctgacggcgagcatttcgccaacagcatcgtcgcggcgcaggcggatttcaccatgcgcgtggtccacgtcgatctggacaatgtctatgacccggacccggcgcagcaggaaatcaaccttggcaagctgatccagcgcatggctgacatgggcgccaacaccgtgttcctgcaggccttcgctgatccgaagggtgacggtctggtgcactcgctgtacttccccaaccgtcacctgccagtacgcgccgacatcttcgaccgcgtcgcctggcaactgcgcacccgcgcccatgtgaaggtgttcgcctggatgccggtgctcagttttggcctcgatgcgaaactgccgcgtgtgactcgctgggatccgaagactggcaccactggggtcgatccggatcagtatcagcgcctgtcgccgttcgatccggaagtacggcggatcatcggtgaaatctacgaagacgtggcgcgcctgacctcggtcgacggcattctctaccacgacgacgcggtgctctcggacttcgaagacgccagcccccaggccttgaaagcctatgccgcgcagggtttgccggggtcgattgccgcgcttcgcgatgatccggcagcgatgcagcgctggacgcgcttcaagagccgctacctgatcgatttcaccaatgaactgaccgccaaggtccgggcgattcgtggcccgcaggtggagaccgcacgcaacatctttgccgaaccgatcctcaaccccgacagcgaagcctggtttgcacagaacctcgacgacttcctcgtgacctacgactggacggcgccgatggccatgccgctgatggagaaacagaccctggcgcaatccggcccgtggctcgaagaactggtggcgaagatcaagcagcgccccggtgcactggatcacacggtgttcgaactgcaggcccgcgactggacgaaaaaggaccaggccgacattgacggcgcacgactggccgactggatgggccggctcaagcgccagggcgcgaacagtttcggttactacccggacaacttcctcgacaacctgccggacctgaaaaccgtaaggcctgcgctctccaacaaatggaacccatga

SEQ ID NO: 17 - protein, 665 aa, Pseudomonas koreensis
MPLITRFILLLGVLLVSACAQQAPAFAPPSQRPVAANEKPWPKNHVLGIAYHDVEDRDPDQAVVAVRTERMIEQLAWLRENGYKPVTVDQIMAARKGGPELPPKAILLSFDDGYSSFYTRVLPLLRAYNWHALLAPVGVWIDTPLNQPVDFAGAPRARTDFLTWDQVREISQSGLVEIAAHTDASHKGILANPQGNLQPAAATRRYDPVTKRYESEEDFRARIRNDVATISEKIRKVTGKKPRIWVWPYGAADGTSLAVVGEQGYEMALTLDDGLDALDNLMSSPRFLVASDPDGEHFANSIVAAQADFTMRVVHVDLDNVYDPDPAQQEINLGKLIQRMADMGANTVFLQAFADPKGDGLVHSLYFPNRHLPVRADIFDRVAWQLRTRAHVKVFAWMPVLSFGLDAKLPRVTRWDPKTGTTGVDPDQYQRLSPFDPEVRRIIGEIYEDVARLTSVDGILYHDDAVLSDFEDASPQALKAYAAQGLPGSIAALRDDPAAMQRWTRFKSRYLIDFTNELTAKVRAIRGPQVETARNIFAEPILNPDSEAWFAQNLDDFLVTYDWTAPMAMPLMEKQTLAQSGPWLEELVAKIKQRPGALDHTVFELQARDWTKKDQADIDGARLADWMGRLKRQGANSFGYYPDNFLDNLPDLKTVRPALSNKWNP

SEQ ID NO: 18 - protein, 638 aa, Pseudomonas koreensis
PPSQRPVAANEKPWPKNHVLGIAYHDVEDRDPDQAVVAVRTERMIEQLAWLRENGYKPVTVDQIMAARKGGPELPPKAILLSFDDGYSSFYTRVLPLLRAYNWHALLAPVGVWIDTPLNQPVDFAGAPRARTDFLTWDQVREISQSGLVEIAAHTDASHKGILANPQGNLQPAAATRRYDPVTKRYESEEDFRARIRNDVATISEKIRKVTGKKPRIWVWPYGAADGTSLAVVGEQGYEMALTLDDGLDALDNLMSSPRFLVASDPDGEHFANSIVAAQADFTMRVVHVDLDNVYDPDPAQQEINLGKLIQRMADMGANTVFLQAFADPKGDGLVHSLYFPNRHLPVRADIFDRVAWQLRTRAHVKVFAWMPVLSFGLDAKLPRVTRWDPKTGTTGVDPDQYQRLSPFDPEVRRIIGEIYEDVARLTSVDGILYHDDAVLSDFEDASPQALKAYAAQGLPGSIAALRDDPAAMQRWTRFKSRYLIDFTNELTAKVRAIRGPQVETARNIFAEPILNPDSEAWFAQNLDDFLVTYDWTAPMAMPLMEKQTLAQSGPWLEELVAKIKQRPGALDHTVFELQARDWTKKDQADIDGARLADWMGRLKRQGANSFGYYPDNFLDNLPDLKTVRPALSNKWNP

SEQ ID NO: 19 - DNA, 1890 bp, Stenotrophomonas rhizophila
  CDS (1)..(1887)
  sig_peptide (1)..(69)
  mat_peptide (70)..(1887)
atggacttgaagcgcttcacctcctggctgctgctggccctgctggcggtctgcggcggcgtgcaggccgcaccgccggtgctggactcggccgacaacgggctgctggtgctcagctaccacgatatccgcgacgatgtggcgaccaagggcgaccccgacgcgtattcggtaagcacgcagaacttcgccgcgcacctggactggctgtcggcgcatggctaccacccggtgtcgctgtcgcagctgatcaccgcctcgcagggcggcacgccgctgccgcccaagccggtgctgctgaccttcgatgacggcctgcgcagcgtgtacaccaaggtgttcccgctgctgcgcgcctacaactacccggcgctggtggcggtgatcaccgactacgtggacatgccggccggccgccagatcgactacggctaccgccccttcacccacgacgacttcctgacctgggaacagatccgcgagatgcagcgcagcggcctggtcgagctggccagccacaccgacaacctgcaccacggcgtgcagtccaacccgcagggcaactcgaccccggcggtgatcacgcgcacctacgacccggccacgcagcagtacgaaaccgccgcacagtacgaagcccgcctgcgtgccgacctgggcctgagcgtgaagcgcatccaggacaatgtgggggtcagcccgaaggcgatcgtgtggccgtacgcggcctacaacgcactcagcaacaggatcgccgaagacctgggcatgccggtgtcgttcgacctggaaggccgcagcaccccggtcacccgcgacctgcatgggctggcccgcctgttggtgaccggcaacccaccggtgacccagctggcctacgaactgcgccgcgacatcgaacgcgatggcatgcgcgccctgcagatcgacctggacgcggtgtacgacaccaacccggcacagcaggcgaagaacctggacgcgctgatcgaccgggtcaagaagatcggcccgacccacgtgttcctgcaggccttcgccgatccggacggcaacggttcggccgatgcgctgtacttccccaaccgccacctgccgatgcgcgcggacctgttcaaccgcgtggcctggcagctcaagacccgcgccggcgtgaaggtgtacgcgtggctgccggtgctgggctatgagctcaaggacccggcgatccgtgcggcgctggcgatcgagagcccggagaaggacggcatcttccggctcgacttcaccaacccgaaggtgcggcagatcatcgacgacgtctacgaagacctggccatcaactcgtatttcgagggcctgctgttccatgacgatgcctacctgcgcgataccgagctggccaacctgcggcaggagggcgacgacggcgcgcgtacccaggcgctgatcgacttcaccctggagctgcgcaccgccgcgcagcgctggcgcccgaagctggccaccgtgcgcaacctgtacgcgcagccggtgctgcagccgcagagcgccagctggttcgcccagcgcctggacctgttcaacaaagcctacgaccacaccgcgttgatggcgatgccgtggatggaaggcagccgcaatccggagaagtggctggaccggctggtagtggccgtgcgcgagcacgacccgcagttgtcgcagacgatgttcgagctgcagacggtggactggcgcacgcagacgccgatttccggcgaacgcctgcgcgcgcagatccggcgcctgcaggcgcagggcgtgcgtcatttcgcctggtacccggacgatttcatcgccgggaaaccgtccacctacgatgcgcgcgcggcgatgtcggcacgcaccttcccttacgaggaaaagtga

SEQ ID NO: 20 - protein, 629 aa, Stenotrophomonas rhizophila
MDLKRFTSWLLLALLAVCGGVQAAPPVLDSADNGLLVLSYHDIRDDVATKGDPDAYSVSTQNFAAHLDWLSAHGYHPVSLSQLITASQGGTPLPPKPVLLTFDDGLRSVYTKVFPLLRAYNYPALVAVITDYVDMPAGRQIDYGYRPFTHDDFLTWEQIREMQRSGLVELASHTDNLHHGVQSNPQGNSTPAVITRTYDPATQQYETAAQYEARLRADLGLSVKRIQDNVGVSPKAIVWPYAAYNALSNRIAEDLGMPVSFDLEGRSTPVTRDLHGLARLLVTGNPPVTQLAYELRRDIERDGMRALQIDLDAVYDTNPAQQAKNLDALIDRVKKIGPTHVFLQAFADPDGNGSADALYFPNRHLPMRADLFNRVAWQLKTRAGVKVYAWLPVLGYELKDPAIRAALAIESPEKDGIFRLDFTNPKVRQIIDDVYEDLAINSYFEGLLFHDDAYLRDTELANLRQEGDDGARTQALIDFTLELRTAAQRWRPKLATVRNLYAQPVLQPQSASWFAQRLDLFNKAYDHTALMAMPWMEGSRNPEKWLDRLVVAVREHDPQLSQTMFELQTVDWRTQTPISGERLRAQIRRLQAQGVRHFAWYPDDFIAGKPSTYDARAAMSARTFPYEEK

SEQ ID NO: 21 - protein, 606 aa, Stenotrophomonas rhizophila
APPVLDSADNGLLVLSYHDIRDDVATKGDPDAYSVSTQNFAAHLDWLSAHGYHPVSLSQLITASQGGTPLPPKPVLLTFDDGLRSVYTKVFPLLRAYNYPALVAVITDYVDMPAGRQIDYGYRPFTHDDFLTWEQIREMQRSGLVELASHTDNLHHGVQSNPQGNSTPAVITRTYDPATQQYETAAQYEARLRADLGLSVKRIQDNVGVSPKAIVWPYAAYNALSNRIAEDLGMPVSFDLEGRSTPVTRDLHGLARLLVTGNPPVTQLAYELRRDIERDGMRALQIDLDAVYDTNPAQQAKNLDALIDRVKKIGPTHVFLQAFADPDGNGSADALYFPNRHLPMRADLFNRVAWQLKTRAGVKVYAWLPVLGYELKDPAIRAALAIESPEKDGIFRLDFTNPKVRQIIDDVYEDLAINSYFEGLLFHDDAYLRDTELANLRQEGDDGARTQALIDFTLELRTAAQRWRPKLATVRNLYAQPVLQPQSASWFAQRLDLFNKAYDHTALMAMPWMEGSRNPEKWLDRLVVAVREHDPQLSQTMFELQTVDWRTQTPISGERLRAQIRRLQAQGVRHFAWYPDDFIAGKPSTYDARAAMSARTFPYEEK

SEQ ID NO: 22 - DNA, 1998 bp, Pseudomonas sp-62498
  CDS (1)..(1995)
  sig_peptide (1)..(75)
  mat_peptide (76)..(1995)
atgcctttgatttcgcgtttcatccttctgctgggagcgctgctggtcagcgcctgcgcccagcaagccccggccttcgtgccgccgtccgagcgaccggtggcagccaatgaaaagccctggccgaaaaaccacgtgctgggtattgcctaccacgacgtcgaagatcgtgaccccgatcaggcggtggtggcggtgcgcaccgagcgcctgctcgaacagctcgcctggctgcgggaaaacaactacaaaccggtgacggtcgaccagatcatggccgcgcgtaaaggcgggccggaactgccgcccaaggcaatcctgctcagtttcgacgacggttacgcaagcttctacacccgtgtgctgccggtattgcgcgcctataactggcatgcgctgctggcgccggtcggcacctggatcgatacgccgctgaaccagccggtggatttcgccggtacgccgcgtccgcgctcggacttcctgacctgggctcaggtgcgggaaatctcgcaatcgggcctggtggaaatcgccgcccacaccgacgccagccacaaaggcatcctcgccaacccgcaaggcaacatgcagccggcggccgcgacccgacgctacgatccggtgagcaaacgctacgaatccgaggccgatttccaggcgcggatccgcgctgacgtgcaggcgatttcggagaagatccgcaagaacaccggtaaaaaaccgcgtatctgggtctggccttacggaacggcggacggcacgtcgctgaccgtggtcagcgaggaaggctatgaaatggccctgaccctcgatgacggtctcgatgctctcgacaacctgatgagcgggccgcgtttcctggtcgcctccgaccctgacggcgagcacttcgccaacagcgtggtcggcgtgcaatcggacttcaccatgcgcgtcgtgcacgtggatctggacaacgtctacgacccggacccggcgcagcaggaaatcaacctcggcaaactgatccagcgcatggcggacatgggcgccaacaccgtgttcctgcaagccttcgccgatccgaagggcgatggcctggtgcactcgctgtacttccccaaccgtcatctgccgatgcgcgccgacctgttcgaccgcgtcgcgtggcaactgcgtacccgagctcacgtgaaggtctttgcgtggatgccggtgctgagtttcgaactggattcgaagctgccacgcgtaacgcgctgggatccgaagaccggcacgaccgctgtcgacccggatcaatacaaacgcctgtcgccgttcgatcccgaagtgcggcggatcatcggcgagatctacgaagacgtggcacgcctgacttcggtcgacggcattctctatcacgacgacgcggtgctttcggacttcgaagacgccggccctcaggcgctgaaggcctatgccgcccacggcttgccgggctcgatcgccgccctgcgtgacgatccggcggcgatgcagcgctggacgcgattcaagagccgttacctgatcgacttcaccaatgagctgactgccaaagtgcgcgcgattcgtggcccgcaagtgctcaccgcacggaacatcttcgccgagccggtgctcaatccgcacagcgaagcgtggttcgcgcagaacctcgacgacttcctcgtgagctacgactggacggcgccgatggccatgccgctcatggaaaaacagacccaagcgcaatccggtccgtggcttgaagagctggtggcgaagatcaaacagcgccccggtgcgctggatcgcacggtgttcgaattgcaggcccgggactggacgaaaaaggaacaggccgacatcgacggcgcgcaactggccgactggatgagccgcctcaagcgtcagggcgccaccagtttcggctactacccggacaacttcctcgagaacctgccggacctgaaaaccgtgcggcctgcgctctccaacaaatggaacccatga

SEQ ID NO: 23 - protein, 665 aa, Pseudomonas sp-62498
MPLISRFILLLGALLVSACAQQAPAFVPPSERPVAANEKPWPKNHVLGIAYHDVEDRDPDQAVVAVRTERLLEQLAWLRENNYKPVTVDQIMAARKGGPELPPKAILLSFDDGYASFYTRVLPVLRAYNWHALLAPVGTWIDTPLNQPVDFAGTPRPRSDFLTWAQVREISQSGLVEIAAHTDASHKGILANPQGNMQPAAATRRYDPVSKRYESEADFQARIRADVQAISEKIRKNTGKKPRIWVWPYGTADGTSLTVVSEEGYEMALTLDDGLDALDNLMSGPRFLVASDPDGEHFANSVVGVQSDFTMRVVHVDLDNVYDPDPAQQEINLGKLIQRMADMGANTVFLQAFADPKGDGLVHSLYFPNRHLPMRADLFDRVAWQLRTRAHVKVFAWMPVLSFELDSKLPRVTRWDPKTGTTAVDPDQYKRLSPFDPEVRRIIGEIYEDVARLTSVDGILYHDDAVLSDFEDAGPQALKAYAAHGLPGSIAALRDDPAAMQRWTRFKSRYLIDFTNELTAKVRAIRGPQVLTARNIFAEPVLNPHSEAWFAQNLDDFLVSYDWTAPMAMPLMEKQTQAQSGPWLEELVAKIKQRPGALDRTVFELQARDWTKKEQADIDGAQLADWMSRLKRQGATSFGYYPDNFLENLPDLKTVRPALSNKWNP

SEQ ID NO: 24 - protein, 640 aa, Pseudomonas sp-62498
FVPPSERPVAANEKPWPKNHVLGIAYHDVEDRDPDQAVVAVRTERLLEQLAWLRENNYKPVTVDQIMAARKGGPELPPKAILLSFDDGYASFYTRVLPVLRAYNWHALLAPVGTWIDTPLNQPVDFAGTPRPRSDFLTWAQVREISQSGLVEIAAHTDASHKGILANPQGNMQPAAATRRYDPVSKRYESEADFQARIRADVQAISEKIRKNTGKKPRIWVWPYGTADGTSLTVVSEEGYEMALTLDDGLDALDNLMSGPRFLVASDPDGEHFANSVVGVQSDFTMRVVHVDLDNVYDPDPAQQEINLGKLIQRMADMGANTVFLQAFADPKGDGLVHSLYFPNRHLPMRADLFDRVAWQLRTRAHVKVFAWMPVLSFELDSKLPRVTRWDPKTGTTAVDPDQYKRLSPFDPEVRRIIGEIYEDVARLTSVDGILYHDDAVLSDFEDAGPQALKAYAAHGLPGSIAALRDDPAAMQRWTRFKSRYLIDFTNELTAKVRAIRGPQVLTARNIFAEPVLNPHSEAWFAQNLDDFLVSYDWTAPMAMPLMEKQTQAQSGPWLEELVAKIKQRPGALDRTVFELQARDWTKKEQADIDGAQLADWMSRLKRQGATSFGYYPDNFLENLPDLKTVRPALSNKWNP

SEQ ID NO: 25 - DNA, 1209 bp, Acinetobacter bouvetii
  CDS (1)..(1206)
  sig_peptide (1)..(66)
  mat_peptide (67)..(1206)
atgcagtcgaagctgctgagcatattggcctgcatgggtttagcaattgcaggaacctgcaatgccgaaactgtgcaaattgcgcagcatcccattcaagacttagtgaagcgctatgcagcgtctgcgcctgcgcggcaaaaaacagctgaactgcggactgctcagcagcagaacatgcgcatcatgcatattgatttagactacgtttatgatgccgacccaatacagcaaaagcgcaatcttcgggaacttatccgccgcattcaaaccattcagcccaatactgtttttttgcaggcctttgccgatcctgatgccaatggttcagccaatgccgtttattttcaaaacaggcacatcccgctgcgcgaagatttatttcaccgcgtgctgctggagatccgccagcatacgcaagtccaggcggtatatggctggctgccattaatcgcgtgggaattccctaaaaagtatcaattgcagtacgtgcagaatagcggcggaaagaaaggctacatccgcgtttcgccttttgatgcaaaaaatctgcagtataccgctgaaatatttttagacttcattcagcagaaccctgttgacggcattctgtatcatgatgatattaccctcagcgattttgaagatgacagcgcgccggcccatgccgcctatcaaaaatggggctttaacagcgcatccttgattgagcagccggaacatccgcatcagttaaaattttcccgctataaaaccgcctatttagatcaattcgccgcaggcatcagcgaaatattgaaacagcgcgagccgaatctacgctttgcccgcaacatgtatgcagaagccgtactgaatccagacagtgaaaaatggttcagccaatccaatgccggcacctatcaacactatgactataatgccattatggcgatgccctatatggaaaaagctgacgatcaccgtcaattttatttagacttgattcagcgggcaaaaaaatacgacccaaatttgagccgtaccatttttgaattgcaggccactgactggaatcatcaaaatcaaatagctgatgaagagttaatcgacaccatacaattgctggagcagaatggcgttcagcatataggctattatcctgatgattttgtgcaagggcatccggatgccaagcagctgaaagatgcatttgcaaaagctgaataa

SEQ ID NO: 26 - protein, 402 aa, Acinetobacter bouvetii
MQSKLLSILACMGLAIAGTCNAETVQIAQHPIQDLVKRYAASAPARQKTAELRTAQQQNMRIMHIDLDYVYDADPIQQKRNLRELIRRIQTIQPNTVFLQAFADPDANGSANAVYFQNRHIPLREDLFHRVLLEIRQHTQVQAVYGWLPLIAWEFPKKYQLQYVQNSGGKKGYIRVSPFDAKNLQYTAEIFLDFIQQNPVDGILYHDDITLSDFEDDSAPAHAAYQKWGFNSASLIEQPEHPHQLKFSRYKTAYLDQFAAGISEILKQREPNLRFARNMYAEAVLNPDSEKWFSQSNAGTYQHYDYNAIMAMPYMEKADDHRQFYLDLIQRAKKYDPNLSRTIFELQATDWNHQNQIADEELIDTIQLLEQNGVQHIGYYPDDFVQGHPDAKQLKDAFAKAE

SEQ ID NO: 27 - protein, 380 aa, Acinetobacter bouvetii
ETVQIAQHPIQDLVKRYAASAPARQKTAELRTAQQQNMRIMHIDLDYVYDADPIQQKRNLRELIRRIQTIQPNTVFLQAFADPDANGSANAVYFQNRHIPLREDLFHRVLLEIRQHTQVQAVYGWLPLIAWEFPKKYQLQYVQNSGGKKGYIRVSPFDAKNLQYTAEIFLDFIQQNPVDGILYHDDITLSDFEDDSAPAHAAYQKWGFNSASLIEQPEHPHQLKFSRYKTAYLDQFAAGISEILKQREPNLRFARNMYAEAVLNPDSEKWFSQSNAGTYQHYDYNAIMAMPYMEKADDHRQFYLDLIQRAKKYDPNLSRTIFELQATDWNHQNQIADEELIDTIQLLEQNGVQHIGYYPDDFVQGHPDAKQLKDAFAKAE

SEQ ID NO: 28 - DNA, 1998 bp, Pseudomonas panacis
  CDS (1)..(1995)
  sig_peptide (1)..(60)
  mat_peptide (61)..(1995)
atgaccgtcctttgccgttgcctgttggtcctgggtgtaatgctggccagtgcctgcgcccagcaacccgcgcccttcactccacccgccgaacggccgatagcggccaacgaagcgccgtggccgaaaaatcatttcctcggcattgcctaccacgacgtcgaggatcgcgaccccgaccaggcggtggtggcagtgcgcaccgagcgcttgatcgaacagctggcgtggttgcgtgagaacggctaccaggcggtcagcgtcgaccagattctggcggcacgcaacggcggccccgagttgccgcccaaagccatcatgctcagcttcgatgacggctattcgagcttctacacccgcgtgatgccgattctgcgtgcctatcactggccggccttgctggcgccggtggggtattggatcgatacgccgctcaatcaaccggtggacttcgccggctcgccgcgcccacgtggagaattcctcacctggcagcagattcgtgaagtgtcgcagtcgggcctggtggaaatcgccgcgcacaccgacaacaaccacaaagggatcttggccaacccccagggcaacctggaaccggcggcgaccagcctgcgctttgacccggccaccgggcgttatgaagaccaggccaaattcgatacgcgcatgcgggctgatgtcgcggcgatcaccaacaagatccgcagcgtgaccggcaaggcgcctcgggtgtgggtgtggccgtatggcgccgccaaaggcacctcgctggcgattgtcggcgagcagggctaccagatggcgctgaccctggaagacggcctcgacagcctcggcaacttgatgaacagcccgcgctttttggtggcttcggacccggacggcgaacacttcgccaacagcatggtggccgtgcaaacccaggcaccgctgcgcgtgctgcacgtggacctggacaacgtttacgaccccgacccggcccaacaagcgcgcaacctcgaccagcttgtacaacgtgtggtcgacatgggcgcgggcaccgtgttcctgcaagccttcgccgaccccaagggcgatggcctggtgcattcgctgtacttccccaaccgccacttgccggtgcgcgccgacctgttcaaccgcgttgcctggcagttgcgcacccgtgcccatgccagtgtgtatgcctggatgccggtgctgagtttcgccctcgaccccaagctgccgcgcgtgactcgctgggacccgcaaaccggccaggtcgccaccgacccggaccaatacaagcgtttgtcgccgttcgacccgcaggtgcgcaagatcatcggcgagatctatgaagacctggcccgcaacaatgctatcgacggtgtgctgtaccacgacgacgccgtgctttccgacttcgaagacgccagccccgctgcgctcaaggcctatgccgccaacggcctgccgggtagcattgccgcgctgcgtgcagacccagcagtgatgcaacgctggacgcgcttcaagagccgctacctgatcgacttcaccaacgagctcaccgccaaggtccgcgccatcggtggcccgcaggtgcagaccgcacgcaatatctttgccgagccgatgctcaacccggccagcgaagcctggtttgcacagaacctcgatgacttccttcaggcctacgactggaccgcgcccatggccatgccgttgatggaaggccaagagctgaaaacctccaacgcctggctggaaaaactcgtcgccaccgttaaggcacgccccggcgcgctggaaaaaaccgtgttcgagctgcaagccaaagactggcggacccaagcagcgccggatatcaatggcgcgcaaatggccgaatggatgggcgtgctcaaacgccagggggtcaagagctttggctactacccggataacttcctggaaaactcgccggacttgaagacagttcgaccggccctttccaaccagtggaacccttga

SEQ ID NO: 29 - protein, 665 aa, Pseudomonas panacis
MTVLCRCLLVLGVMLASACAQQPAPFTPPAERPIAANEAPWPKNHFLGIAYHDVEDRDPDQAVVAVRTERLIEQLAWLRENGYQAVSVDQILAARNGGPELPPKAIMLSFDDGYSSFYTRVMPILRAYHWPALLAPVGYWIDTPLNQPVDFAGSPRPRGEFLTWQQIREVSQSGLVEIAAHTDNNHKGILANPQGNLEPAATSLRFDPATGRYEDQAKFDTRMRADVAAITNKIRSVTGKAPRVWVWPYGAAKGTSLAIVGEQGYQMALTLEDGLDSLGNLMNSPRFLVASDPDGEHFANSMVAVQTQAPLRVLHVDLDNVYDPDPAQQARNLDQLVQRVVDMGAGTVFLQAFADPKGDGLVHSLYFPNRHLPVRADLFNRVAWQLRTRAHASVYAWMPVLSFALDPKLPRVTRWDPQTGQVATDPDQYKRLSPFDPQVRKIIGEIYEDLARNNAIDGVLYHDDAVLSDFEDASPAALKAYAANGLPGSIAALRADPAVMQRWTRFKSRYLIDFTNELTAKVRAIGGPQVQTARNIFAEPMLNPASEAWFAQNLDDFLQAYDWTAPMAMPLMEGQELKTSNAWLEKLVATVKARPGALEKTVFELQAKDWRTQAAPDINGAQMAEWMGVLKRQGVKSFGYYPDNFLENSPDLKTVRPALSNQWNP

SEQ ID NO: 30 - protein, 645 aa, Pseudomonas panacis
QQPAPFTPPAERPIAANEAPWPKNHFLGIAYHDVEDRDPDQAVVAVRTERLIEQLAWLRENGYQAVSVDQILAARNGGPELPPKAIMLSFDDGYSSFYTRVMPILRAYHWPALLAPVGYWIDTPLNQPVDFAGSPRPRGEFLTWQQIREVSQSGLVEIAAHTDNNHKGILANPQGNLEPAATSLRFDPATGRYEDQAKFDTRMRADVAAITNKIRSVTGKAPRVWVWPYGAAKGTSLAIVGEQGYQMALTLEDGLDSLGNLMNSPRFLVASDPDGEHFANSMVAVQTQAPLRVLHVDLDNVYDPDPAQQARNLDQLVQRVVDMGAGTVFLQAFADPKGDGLVHSLYFPNRHLPVRADLFNRVAWQLRTRAHASVYAWMPVLSFALDPKLPRVTRWDPQTGQVATDPDQYKRLSPFDPQVRKIIGEIYEDLARNNAIDGVLYHDDAVLSDFEDASPAALKAYAANGLPGSIAALRADPAVMQRWTRFKSRYLIDFTNELTAKVRAIGGPQVQTARNIFAEPMLNPASEAWFAQNLDDFLQAYDWTAPMAMPLMEGQELKTSNAWLEKLVATVKARPGALEKTVFELQAKDWRTQAAPDINGAQMAEWMGVLKRQGVKSFGYYPDNFLENSPDLKTVRPALSNQWNP

SEQ ID NO: 31 - DNA, 1962 bp, Enviromental bacterial community L
  CDS (1)..(1959)
  sig_peptide (1)..(72)
  mat_peptide (73)..(1959)
atgacaccctggcgcgtcattgttatgagcgtggttttgctggtgattgttaatattcagcaggcccaagccgcacgctcagttgatgactatgtggtcatcagctaccacgacattgtggacactaccgtcacgcctgatctgccgatctattcgcagaccattacccgtagtcggttaatcgagcatttcaaccttatctccgtagggggctaccatccggttagcttgcaacaaatcatcgacgctaaagcgggcggccaaccactgccagataaagcggtgttattaaccttcgatgacggctatcgaagcttctacgaaatagtctttccgctgcttaaactgtatggctttcccgctgttcaagcggttgttggtagctggttagacgttcctgccggtggacgagtgccctatggcagcaccaccctgccccgcgaacgctttctaagctgggagcaggtcaaaacgctagaagaatcgccactcgttgaaatcgcctctcactcttatagcctgcattatggtgttagcggcaacccaatgggtaacgagcaagcggctgccgtcaccagcgtctggaacgcacagactggctacgaaagcgaagcggcctaccttgagcggatacgccaggacatggcgcaaacccaacggcggtttcaagagcaggtaggccaatccccgcgcatcatggtatggccctacggcgcgtatagcgaagcaacgttagatatcgccgctgaatatggcatgacttacaccttcagcctactgagtgcgcctaaccagcttcgagatcccatgcgggtcatgaatcgctacctcattgatcaggaaaccagtcttcaaaccatcgacgagattctctccaaccgtgtgtgggaaacggaagatctgcgtatcgtccatgttgacctggactatgtgtttgaccctgatcccatccagcaggaacaaaatctcgacaagctgattgaacgcatcttccgcttcggcgttagcaccgtttacttacaagcttacgccgacccagatggtgacggtgtcgctgatgagctctatttccccaaccgccacctccccgtaagagctgaccttttcaatcgtgttgcatggcagttgaagaaacgcgccggcgtaaaggtttacgcatggatgccggtgatgtcatttgatcttggtgatggtcatcgttatgtaaccgatgtgcgtaccggcgaagagtcacccgaccaataccgccgcctctcaccttatgtggaagataaccggcgcattattcgggaaatctatgaagaccttggccgcctgaccaagttcgatggcctgctgtttcacgacgatgctttctttaccgactttgaagatgccaaccccgaagcattaaccgcctatcagagcgcttctttgccaagggatatcaataccattcgcaatgatgacgcgatgatggcgaactgggtgcgctttaaaacagcgtatctcacggactttaccaacgagcttgagcgcgcagccaactactaccgacaagctgacaataagcaatttaccacctcgcgcaatatctatgccgtgacgatcatgaacccaagtagccagcgctggttctcgcaaagtctcgaaaacttcgccactaactatgactatgtcgccgtcatggcaatgccctacatggaagaagccgaaaaccctgacgaatggctgcgctcacttgctcaacgctcattggcgcaggttagcgcaagtcaactggtcttcgagctacaaacacaggactggcgtacccaaacgcctatccccagcgaagaaatcgctcagtgggtacgcatactgcgcgaagaaggcatcaagaacattggctactatccagatgatttccaccaaaatcatccagacgtgaatgttatgaggccggtcttctctatcggacgtcgtttcagggcaacgccatga

SEQ ID NO: 32 - protein, 653 aa, Enviromental bacterial community L
MTPWRVIVMSVVLLVIVNIQQAQAARSVDDYVVISYHDIVDTTVTPDLPIYSQTITRSRLIEHFNLISVGGYHPVSLQQIIDAKAGGQPLPDKAVLLTFDDGYRSFYEIVFPLLKLYGFPAVQAVVGSWLDVPAGGRVPYGSTTLPRERFLSWEQVKTLEESPLVEIASHSYSLHYGVSGNPMGNEQAAAVTSVWNAQTGYESEAAYLERIRQDMAQTQRRFQEQVGQSPRIMVWPYGAYSEATLDIAAEYGMTYTFSLLSAPNQLRDPMRVMNRYLIDQETSLQTIDEILSNRVWETEDLRIVHVDLDYVFDPDPIQQEQNLDKLIERIFRFGVSTVYLQAYADPDGDGVADELYFPNRHLPVRADLFNRVAWQLKKRAGVKVYAWMPVMSFDLGDGHRYVTDVRTGEESPDQYRRLSPYVEDNRRIIREIYEDLGRLTKFDGLLFHDDAFFTDFEDANPEALTAYQSASLPRDINTIRNDDAMMANWVRFKTAYLTDFTNELERAANYYRQADNKQFTTSRNIYAVTIMNPSSQRWFSQSLENFATNYDYVAVMAMPYMEEAENPDEWLRSLAQRSLAQVSASQLVFELQTQDWRTQTPIPSEEIAQWVRILREEGIKNIGYYPDDFHQNHPDVNVMRPVFSIGRRFRATP

SEQ ID NO: 33 - protein, 629 aa, Enviromental bacterial community L
ARSVDDYVVISYHDIVDTTVTPDLPIYSQTITRSRLIEHFNLISVGGYHPVSLQQIIDAKAGGQPLPDKAVLLTFDDGYRSFYEIVFPLLKLYGFPAVQAVVGSWLDVPAGGRVPYGSTTLPRERFLSWEQVKTLEESPLVEIASHSYSLHYGVSGNPMGNEQAAAVTSVWNAQTGYESEAAYLERIRQDMAQTQRRFQEQVGQSPRIMVWPYGAYSEATLDIAAEYGMTYTFSLLSAPNQLRDPMRVMNRYLIDQETSLQTIDEILSNRVWETEDLRIVHVDLDYVFDPDPIQQEQNLDKLIERIFRFGVSTVYLQAYADPDGDGVADELYFPNRHLPVRADLFNRVAWQLKKRAGVKVYAWMPVMSFDLGDGHRYVTDVRTGEESPDQYRRLSPYVEDNRRIIREIYEDLGRLTKFDGLLFHDDAFFTDFEDANPEALTAYQSASLPRDINTIRNDDAMMANWVRFKTAYLTDFTNELERAANYYRQADNKQFTTSRNIYAVTIMNPSSQRWFSQSLENFATNYDYVAVMAMPYMEEAENPDEWLRSLAQRSLAQVSASQLVFELQTQDWRTQTPIPSEEIAQWVRILREEGIKNIGYYPDDFHQNHPDVNVMRPVFSIGRRFRATP

SEQ ID NO: 34 - DNA, 1962 bp, Halomonas zhanjiangensis DSM 21076
  CDS (1)..(1959)
  sig_peptide (1)..(72)
  mat_peptide (73)..(1959)
atgaaattttggcgcgtctttcttgttaacgctgccctgctggtcatgctggggctacaacaagcccatgcagaacgtacccctggtgattatgtagtgatcagctatcacgacattgttgataccactgtcacaccggacttacccatttactcgcaaaccatcactcgtgcccggttaatagaacacttcaatctactttcggcaggtggttatcagccggttagcttgcagcaaattatcgatgccaaagcaggcgggcagcctctgcccgacaaagccgtgctgctcaccttcgatgatggctatcgcagcttctacgacattgtcttcccgctgctaaagctgtacgggtttcctgccgttcaagccgtcgtcggcagttggctggatgtccctgaaggcggccgggtaccttacggcaatattaccctaccccgggagcggtttttaacctgggagcaggtgcagattctggacgaatcgccgctggtcgaaatcgcttctcatacctacaacctgcattacggggttaaaggcaacccgatgggcaacgagcaggcagccgcggtaactagcatctggaacccggaaaccggctacgaaagcgaggccgcttatgtagaacgaattcgcaacgatatggcacgaacccaacagcagtttcagacacacatgggtcgctcgccccgcatgatggtatggccttacggcgcatctagtcaggcaacgctggatatcgccgctgaatacggcatgatctatacctttagcctgttaagcgcacctaaccagctgcatagctccatgcgctctatcaaccgctacctgatcgaccaggaaaccggccttcagacgattgaagaaattctttccgaccgcgtctgggaacccgaagacctgcgcattgttcacgtggatatagattatgtttacgacgacgaccccattcagcaagaacagaatctcgacaggctgatcgagcgtatctacaactacggtgttaccaccgtttatctgcaggcgtttgcagatcctgatggtgatggcgtggccgatgaactctacttccccaaccgccatttgccggttagagccgacctgtttaaccgggtagcctggcagttaaaaaaacgcgcgggcgtcaaggtatatgcgtggatgccggtactctcgttcgatcttggtgacgactaccgctatgtcatcgactcacaaacaggccttgaagccccagaccgctaccgtcgcctttcaccctatgtggaagacaaccggcgcatcattcgcgaaatttatcaggaccttggacgcctgacgaaattcgatggcgtgatatttcacgatgacgcttttttaaacgattttgaggatgccaaccccgaagcactggctgcctacgctcaggcctccttacctcgcgatattaccgctattaagaatgacgcaagccttatggcggcctggacacagcataaaacccaatttttaaccgacttcacccgagagctggagcaggcagccaactactatcgccaggcagacaataaagaattcactactgcgcgtaacctgtatgcccgcacggtgatgaatccagaaagccagcaatggttctcgcaggatattcaaaacttcgcatcgggttacgattttgttgccgtaatggccatgccctacatggaagaggccgaaaatccggatgagtggttgcgggaattagccagacgttcgcttgctcaggtgagttccaagcagttggtctttgaactgcaaacccaggattggcgcactcaaacacccgtttccagcgaagagttagcacgatgggtacgcctgctgcgcgaagaaggcattcagcatatcggctactacccggatgattttctccagaaccatccaaatcttagtgtgctacgaccagctttttccaatggccgccgctttagggcaacaccatga

SEQ ID NO: 35 - protein, 653 aa, Halomonas zhanjiangensis DSM 21076
MKFWRVFLVNAALLVMLGLQQAHAERTPGDYVVISYHDIVDTTVTPDLPIYSQTITRARLIEHFNLLSAGGYQPVSLQQIIDAKAGGQPLPDKAVLLTFDDGYRSFYDIVFPLLKLYGFPAVQAVVGSWLDVPEGGRVPYGNITLPRERFLTWEQVQILDESPLVEIASHTYNLHYGVKGNPMGNEQAAAVTSIWNPETGYESEAAYVERIRNDMARTQQQFQTHMGRSPRMMVWPYGASSQATLDIAAEYGMIYTFSLLSAPNQLHSSMRSINRYLIDQETGLQTIEEILSDRVWEPEDLRIVHVDIDYVYDDDPIQQEQNLDRLIERIYNYGVTTVYLQAFADPDGDGVADELYFPNRHLPVRADLFNRVAWQLKKRAGVKVYAWMPVLSFDLGDDYRYVIDSQTGLEAPDRYRRLSPYVEDNRRIIREIYQDLGRLTKFDGVIFHDDAFLNDFEDANPEALAAYAQASLPRDITAIKNDASLMAAWTQHKTQFLTDFTRELEQAANYYRQADNKEFTTARNLYARTVMNPESQQWFSQDIQNFASGYDFVAVMAMPYMEEAENPDEWLRELARRSLAQVSSKQLVFELQTQDWRTQTPVSSEELARWVRLLREEGIQHIGYYPDDFLQNHPNLSVLRPAFSNGRRFRATP

SEQ ID NO: 36 - protein, 629 aa, Halomonas zhanjiangensis DSM 21076
ERTPGDYVVISYHDIVDTTVTPDLPIYSQTITRARLIEHFNLLSAGGYQPVSLQQIIDAKAGGQPLPDKAVLLTFDDGYRSFYDIVFPLLKLYGFPAVQAVVGSWLDVPEGGRVPYGNITLPRERFLTWEQVQILDESPLVEIASHTYNLHYGVKGNPMGNEQAAAVTSIWNPETGYESEAAYVERIRNDMARTQQQFQTHMGRSPRMMVWPYGASSQATLDIAAEYGMIYTFSLLSAPNQLHSSMRSINRYLIDQETGLQTIEEILSDRVWEPEDLRIVHVDIDYVYDDDPIQQEQNLDRLIERIYNYGVTTVYLQAFADPDGDGVADELYFPNRHLPVRADLFNRVAWQLKKRAGVKVYAWMPVLSFDLGDDYRYVIDSQTGLEAPDRYRRLSPYVEDNRRIIREIYQDLGRLTKFDGVIFHDDAFLNDFEDANPEALAAYAQASLPRDITAIKNDASLMAAWTQHKTQFLTDFTRELEQAANYYRQADNKEFTTARNLYARTVMNPESQQWFSQDIQNFASGYDFVAVMAMPYMEEAENPDEWLRELARRSLAQVSSKQLVFELQTQDWRTQTPVSSEELARWVRLLREEGIQHIGYYPDDFLQNHPNLSVLRPAFSNGRRFRATP

SEQ ID NO: 37 - DNA, 1962 bp, Halomonas sp-63456
  CDS (1)..(1959)
  sig_peptide (1)..(72)
  mat_peptide (73)..(1959)
gtgacacattggcgcatcgcgatcttgggcattgccctgctgctcatcctagggtttcaacaggctcatgcggcacgttcaaccggcgactacgtcgtcatcagctaccatgatgtggtggacatcaatgtcacacccgaccagcctctctatccacagaccatcacacggactcggctgatagaacatttcaaccttatcgccgagggtggttaccagcccgtcagcctgcagcagataatcgatgccagagcgggtcggcagccgctgccacacaaggctgtgctgctcacgttcgatgatggctatcgtagcttctacgatattgtctttcctctgctgaaattgtatgacttccccgccattcaagcggtggtgggcagttggatggatattcctgccggcggacaagtgccctatggcgatatctatctaccccgcgatcgcttccttacctggcagcaagtcgaaacgctgcatgagtcggttctcgtggagatcgcctcccatacctacgacctccaccatggggttatcggcaaccccatgggtaatgagcaggctgccgccgtcaccagcatatgggaccctcgaaccggctatgaaagtgaagagcgctatgtcgaacgcgtgcagcaggacatggcgcgaacggtggaacagtttcaggagcggcttggccaaggccccagagtcatgatctggccctatggcgcctatagtgaagccaccctggatatggccgccgagtatggcatggcctataccttcagcctgctaagtgcgcccaaccggcttcacgatgagatgcgtgtcatgaatcgctatctcatcgatcaggaaaccagcctgcagaccatcgacgagatcctttccaaccgtgtatgggaacccgagaacctccgtatcgttcatgtcgacctggattatgtctatgaccccgacccacagcagcaggagagaaacctggacaagctggtcgagcgcatcgccgggtatggcgtcaacaccgtctatctgcaggcctatgccgacccgaacgggaatggcgttgccgaggagctctactttcccaatcgccaccttcccgtcaggggcgatctcttcaaccgcgttgcatggcagctaaagaagcgtgccgatgtcaaggtctatgcatggatgccagtgcttgcctttgaccttggtgccggtcatcgctacgtcaccgatgtgagaacaggaactgagtcgccagaacactaccgccggctctcgccctatgtggaagagaaccgcaggatcattcgcgagatctacgaagatctcggaaggtttaccaaattcgatggcctgctgttccacgatgacgctttcctcactgactttgaagatgcaaacctcgaagcactagcgcaatatagtaacgccttcctgccagaggatatcgatgcgattagatccaatgatggtctgatgaactcatgggccaggcacaagacagaatttctcacggatttcaccctggatctcaagcaatcggccaactattatcgtcagtctgataacaaagtatttacaacatcacgaaatctctatgccgttacggtaatgaatcccaacagtcggctgtggttctcccaggatattgaaaattttacttctgcctacgattatgtcgcggtgatggccatgccctatatggaggaagcggataatccaaaggagtggttaacggaactcgcacagcgatctcttgaaaaggtgggcgaagacaagctggtcttcgagctacagacgcaggactggcgtacccagaccccgattcccagtgaagagatatcgggatgggtgcggtttttgcgcaacgagggaatcaagcatatcggctactatccggatgattttcataacaaccatcctgatgtcaatgttatgaagccccatttttccatcggtcgtcgttttagagaatcgccatga

SEQ ID NO: 38 - protein, 653 aa, Halomonas sp-63456
VTHWRIAILGIALLLILGFQQAHAARSTGDYVVISYHDVVDINVTPDQPLYPQTITRTRLIEHFNLIAEGGYQPVSLQQIIDARAGRQPLPHKAVLLTFDDGYRSFYDIVFPLLKLYDFPAIQAVVGSWMDIPAGGQVPYGDIYLPRDRFLTWQQVETLHESVLVEIASHTYDLHHGVIGNPMGNEQAAAVTSIWDPRTGYESEERYVERVQQDMARTVEQFQERLGQGPRVMIWPYGAYSEATLDMAAEYGMAYTFSLLSAPNRLHDEMRVMNRYLIDQETSLQTIDEILSNRVWEPENLRIVHVDLDYVYDPDPQQQERNLDKLVERIAGYGVNTVYLQAYADPNGNGVAEELYFPNRHLPVRGDLFNRVAWQLKKRADVKVYAWMPVLAFDLGAGHRYVTDVRTGTESPEHYRRLSPYVEENRRIIREIYEDLGRFTKFDGLLFHDDAFLTDFEDANLEALAQYSNAFLPEDIDAIRSNDGLMNSWARHKTEFLTDFTLDLKQSANYYRQSDNKVFTTSRNLYAVTVMNPNSRLWFSQDIENFTSAYDYVAVMAMPYMEEADNPKEWLTELAQRSLEKVGEDKLVFELQTQDWRTQTPIPSEEISGWVRFLRNEGIKHIGYYPDDFHNNHPDVNVMKPHFSIGRRFRESP

SEQ ID NO: 39 - protein, 629 aa, Halomonas sp-63456
ARSTGDYVVISYHDVVDINVTPDQPLYPQTITRTRLIEHFNLIAEGGYQPVSLQQIIDARAGRQPLPHKAVLLTFDDGYRSFYDIVFPLLKLYDFPAIQAVVGSWMDIPAGGQVPYGDIYLPRDRFLTWQQVETLHESVLVEIASHTYDLHHGVIGNPMGNEQAAAVTSIWDPRTGYESEERYVERVQQDMARTVEQFQERLGQGPRVMIWPYGAYSEATLDMAAEYGMAYTFSLLSAPNRLHDEMRVMNRYLIDQETSLQTIDEILSNRVWEPENLRIVHVDLDYVYDPDPQQQERNLDKLVERIAGYGVNTVYLQAYADPNGNGVAEELYFPNRHLPVRGDLFNRVAWQLKKRADVKVYAWMPVLAFDLGAGHRYVTDVRTGTESPEHYRRLSPYVEENRRIIREIYEDLGRFTKFDGLLFHDDAFLTDFEDANLEALAQYSNAFLPEDIDAIRSNDGLMNSWARHKTEFLTDFTLDLKQSANYYRQSDNKVFTTSRNLYAVTVMNPNSRLWFSQDIENFTSAYDYVAVMAMPYMEEADNPKEWLTELAQRSLEKVGEDKLVFELQTQDWRTQTPIPSEEISGWVRFLRNEGIKHIGYYPDDFHNNHPDVNVMKPHFSIGRRFRESP

SEQ ID NO: 40 - DNA, 1875 bp, Luteibacter rhizovicinus
  CDS (1)..(1872)
  sig_peptide (1)..(57)
  mat_peptide (58)..(1872)
atgcgttcgctgttgttcctgctagcccttctcgggctcgcgcttcccgcatacgcgcaggccgtcgctccggcacggccctcgctgatcgtgctcgcctatcacgatgtccgcgacgacgtcggcctgcaggccgatcgcgatccggatgcgaccagtaccgaccacctcatctcccactttgactggctcaaggccaacggatacagcatcgtcagcctgcagcaggtggtcgatgccagccatggcggcgccgcgctgccgccgaatgccgtgctgctcaccttcgacgatggcctcgaaagcttctacacccgcgtctacccgctgctgcgcgcctacaactatcccgccgtggctgccatcgtcggctcgtggatggacatgcccgccggcaagcgcatgccttacaacggctccgattgcgaccgcgactgcttcctcacctgggaccaggtgacggaaatgcacaagagcgggctggtggaattcgcgtcgcactcctggggcatgcacgagggcaccaacggcaacccgcagggtaacctgctgcccgccgccgcctcgctgcgctacgacgacaaggctcaggcctatgaaagcgaagcccagtaccgcgcgcgcgtcggtgacgacctgcagcacagtgccgacgagatcgccgagcacacgggcgtgcgtccgcgtgccgtcgcctggccctatggtgcctatacgcgcgtgggacgcgaggtggcggcccagcacggcatgccggtgaccttcagcctcggcgaccgcccgcctaccctggaagccggcaagacgattccgcgcctgcttgtcagcggcaacatcgtcgcgaaccggcttggatggctcatgcgccacctggaccgcgtcgagccgacgcgcgccgtgcaggtggacctggactacgtctacgatccggacccggcgcagcaggaccgcaacctttcgaagctgctcgaccgcatcaagcgcctgcaccccagccaggtatggctgcaggcctatgccgacccggacggcgatggcgtggccgatgcggtgtatttcccgaaccggcacctgccgatgcgcgcggacctgttttcccgtgtgtcatggcaattgcgcacccgcgccggcgttcgcgtgtacgcgtggatgccggtgctcgccttccgcttcccggacggcaaggacctgccctccctcgccggcgaacccaaacccggtggcgaccacttccggctggcaccgtacgacccgaaggtccgcgccatgatcggcgatgtctatgaagacctcgccatgcacgccgacactgccggcgtccttttctcggacgacgcctatatccgcgacaccgataacctcggcccgtgggcgaagaacacgccggcgcagaacacccaggcgctgatcgacttcacgctggagctctccgagcgcatgcagaagtggcgaccgcagttgcgcacggcacgtaacatgttcgccctgcccgtcctgcagccgaatgcggaagcctggacggcgcaaagcctgccggccttcatcaaggcctatgacatgacggccgtgatggcgatgccgcagctcgaccagcagtcggatcgcctgggctggtacaagcacctggctgcccaggtcgcgacgatacccaacggcttcgagcgcacgctgttcgagttcgccacccagaactggcgcacgcatgccccgatcgacgaccgcgacatggccctgcgcatgcgtaccatgcaggccgccggcgcatggcatatcggttactacccggatgacttcctgcacgatcatccggacctggaaaccatccggccgtacatctcggcgtccgactacccctatgcggagcccacgcgatga

SEQ ID NO: 41 - protein, 624 aa, Luteibacter rhizovicinus
MRSLLFLLALLGLALPAYAQAVAPARPSLIVLAYHDVRDDVGLQADRDPDATSTDHLISHFDWLKANGYSIVSLQQVVDASHGGAALPPNAVLLTFDDGLESFYTRVYPLLRAYNYPAVAAIVGSWMDMPAGKRMPYNGSDCDRDCFLTWDQVTEMHKSGLVEFASHSWGMHEGTNGNPQGNLLPAAASLRYDDKAQAYESEAQYRARVGDDLQHSADEIAEHTGVRPRAVAWPYGAYTRVGREVAAQHGMPVTFSLGDRPPTLEAGKTIPRLLVSGNIVANRLGWLMRHLDRVEPTRAVQVDLDYVYDPDPAQQDRNLSKLLDRIKRLHPSQVWLQAYADPDGDGVADAVYFPNRHLPMRADLFSRVSWQLRTRAGVRVYAWMPVLAFRFPDGKDLPSLAGEPKPGGDHFRLAPYDPKVRAMIGDVYEDLAMHADTAGVLFSDDAYIRDTDNLGPWAKNTPAQNTQALIDFTLELSERMQKWRPQLRTARNMFALPVLQPNAEAWTAQSLPAFIKAYDMTAVMAMPQLDQQSDRLGWYKHLAAQVATIPNGFERTLFEFATQNWRTHAPIDDRDMALRMRTMQAAGAWHIGYYPDDFLHDHPDLETIRPYISASDYPYAEPTR

SEQ ID NO: 42 - protein, 605 aa, Luteibacter rhizovicinus
QAVAPARPSLIVLAYHDVRDDVGLQADRDPDATSTDHLISHFDWLKANGYSIVSLQQVVDASHGGAALPPNAVLLTFDDGLESFYTRVYPLLRAYNYPAVAAIVGSWMDMPAGKRMPYNGSDCDRDCFLTWDQVTEMHKSGLVEFASHSWGMHEGTNGNPQGNLLPAAASLRYDDKAQAYESEAQYRARVGDDLQHSADEIAEHTGVRPRAVAWPYGAYTRVGREVAAQHGMPVTFSLGDRPPTLEAGKTIPRLLVSGNIVANRLGWLMRHLDRVEPTRAVQVDLDYVYDPDPAQQDRNLSKLLDRIKRLHPSQVWLQAYADPDGDGVADAVYFPNRHLPMRADLFSRVSWQLRTRAGVRVYAWMPVLAFRFPDGKDLPSLAGEPKPGGDHFRLAPYDPKVRAMIGDVYEDLAMHADTAGVLFSDDAYIRDTDNLGPWAKNTPAQNTQALIDFTLELSERMQKWRPQLRTARNMFALPVLQPNAEAWTAQSLPAFIKAYDMTAVMAMPQLDQQSDRLGWYKHLAAQVATIPNGFERTLFEFATQNWRTHAPIDDRDMALRMRTMQAAGAWHIGYYPDDFLHDHPDLETIRPYISASDYPYAEPTR

SEQ ID NO: 43 - DNA, 1962 bp, Enviromental bacterial community R
  CDS (1)..(1959)
  sig_peptide (1)..(69)
  mat_peptide (70)..(1959)
atgagcgccagattggccattgttttcggcctcgttatcctcagcagcctcgtacagccagccaaagccgaacgggagagcggcgacttcatcgtgatcagttaccacgacgtggtcgacatggcagcaacgacaaccgggggttcactcttaccccagacgatcacccgaactcgcctcatcgagcacttcaacttgatccgttcgcgaggcttccagcccgtttcgtttcagcagattctcgatgccgagtccggcatggcttcgctacccgacaaggcgatcctgctcgtcttcgacgatggttatcgaagtttctacgacaccgtctacccactgctcaagctgtatggctaccccgcgataacggctgtcgtgggtagctggatcgatgtgcctccgggagggcgggcgccttacggcgatactttcctcgaccgcgatcgcttcatgacccgtgcgcagctacaggagctgcacgacgacccactcgtcgaaatcgcctctcatacctatgaccttcactacggtttgccaggcaacccccaaggcaatcagcaggccgctgccgtcacgagccgttggcagccaacgggttatgaaagtgaaagcgattatctgcaacgcatacgtcgcgatatgcagcgggcgagcacccacctggaagagattaccggaacaccaccacgcatcatggtatggccttatggcgcctacagtcgggccacgctggatatcgccgctgaatacggcatgccatacagtttcagcctgctcagccaacccaaccgagtcgataacggcgcacgagtcatgggccggtatctcatcgaccaggaaaccagcctgcaaaccttcgaggaaatactgagcaaccgcgtgtgggaaagggatgaccttaggatcgtccatgtcgatctcgattacgtctacgatcccgaccccatgcagcaggaggccaacctggacctgctaatagaccggatctaccgttttggcatcagtaccgtctacttgcaggctttcgccgacccgaacggcaatggcgtagcggaggctctgtactttcccaaccggcacctccccatgcgcgcggacctgttcaatcgcgtcgcctggcagctgaaaaagcgcgccaatgtaaaggtctatgcctggatgccggtactcgccttcgacctgggcccgggtcatgaatacgtcacggatgcgcgaaccggacaggtcgccaacgaacactacctgcgactgtcgccctattctgcaaccaacagacgcatcattggcgagatctaccaggatctcggcaggctgaccaagtttgacggcctgctgttccatgacgatgcctatctcaccgattacgaagacgccggtgaggacgccttgggaacctatgaaacggcgtggaacctgccccgcgacatcgaaaagatacgccgggacgaaacgctgatggctgattggacgcgtaggaaaaccgctttcctgaccgatttcaccctcgaactggcacagtcggccgactactatcgccaggcggacaacaagagattcacgacgtcacgcaacatttacgccctgccaatcatcgaaccacagagccaggcgtggttcgcccaatccgcagaaaatttcgccgctgcctacgattacgtggccgtcatggcgatgccgtatatggaacaggcggacgatccccggcgctggctgcagaagctggctcaggacgccctacgggacgtgccggccgatcagcttgttttcgaactgcagacgagagactggcgagacgagagtccgatacccaacgagaccctgacgagctggatcgacaccatcaagcatgccggtatacggcatatcggctactacccggacgacttccataacaaccatcccgacatgggtgtcttgcgtccgcatttttccataagtcaaaggtttggggtgagtaaatga

SEQ ID NO: 44 - protein, 653 aa, Enviromental bacterial community R
MSARLAIVFGLVILSSLVQPAKAERESGDFIVISYHDVVDMAATTTGGSLLPQTITRTRLIEHFNLIRSRGFQPVSFQQILDAESGMASLPDKAILLVFDDGYRSFYDTVYPLLKLYGYPAITAVVGSWIDVPPGGRAPYGDTFLDRDRFMTRAQLQELHDDPLVEIASHTYDLHYGLPGNPQGNQQAAAVTSRWQPTGYESESDYLQRIRRDMQRASTHLEEITGTPPRIMVWPYGAYSRATLDIAAEYGMPYSFSLLSQPNRVDNGARVMGRYLIDQETSLQTFEEILSNRVWERDDLRIVHVDLDYVYDPDPMQQEANLDLLIDRIYRFGISTVYLQAFADPNGNGVAEALYFPNRHLPMRADLFNRVAWQLKKRANVKVYAWMPVLAFDLGPGHEYVTDARTGQVANEHYLRLSPYSATNRRIIGEIYQDLGRLTKFDGLLFHDDAYLTDYEDAGEDALGTYETAWNLPRDIEKIRRDETLMADWTRRKTAFLTDFTLELAQSADYYRQADNKRFTTSRNIYALPIIEPQSQAWFAQSAENFAAAYDYVAVMAMPYMEQADDPRRWLQKLAQDALRDVPADQLVFELQTRDWRDESPIPNETLTSWIDTIKHAGIRHIGYYPDDFHNNHPDMGVLRPHFSISQRFGVSK

SEQ ID NO: 45 - protein, 630 aa, Enviromental bacterial community R
ERESGDFIVISYHDVVDMAATTTGGSLLPQTITRTRLIEHFNLIRSRGFQPVSFQQILDAESGMASLPDKAILLVFDDGYRSFYDTVYPLLKLYGYPAITAVVGSWIDVPPGGRAPYGDTFLDRDRFMTRAQLQELHDDPLVEIASHTYDLHYGLPGNPQGNQQAAAVTSRWQPTGYESESDYLQRIRRDMQRASTHLEEITGTPPRIMVWPYGAYSRATLDIAAEYGMPYSFSLLSQPNRVDNGARVMGRYLIDQETSLQTFEEILSNRVWERDDLRIVHVDLDYVYDPDPMQQEANLDLLIDRIYRFGISTVYLQAFADPNGNGVAEALYFPNRHLPMRADLFNRVAWQLKKRANVKVYAWMPVLAFDLGPGHEYVTDARTGQVANEHYLRLSPYSATNRRIIGEIYQDLGRLTKFDGLLFHDDAYLTDYEDAGEDALGTYETAWNLPRDIEKIRRDETLMADWTRRKTAFLTDFTLELAQSADYYRQADNKRFTTSRNIYALPIIEPQSQAWFAQSAENFAAAYDYVAVMAMPYMEQADDPRRWLQKLAQDALRDVPADQLVFELQTRDWRDESPIPNETLTSWIDTIKHAGIRHIGYYPDDFHNNHPDMGVLRPHFSISQRFGVSK

SEQ ID NO: 46 - DNA, 2100 bp, Enviromental bacterial community H
  CDS (1)..(2097)
  sig_peptide (1)..(63)
  mat_peptide (64)..(2097)
atgcaaaccctgttcaagcttcttacgatgtcattgctttggttctgcatggcagcccaggccgcatccccggcgggaggatattcgctggaattcctgccgccgcctgacccgaacgatggtctgacataccgggtgctctgcctgcacgacgtgcgggacaacctgcgcgccagctttgacaccaatccggatccgtatgctatcgacacccgtacgcttgtcgacatcttcgagtggttgagggcgaaggaatttcacccggttagtatgtcccagatcatcgatgctcggaatggtggaaaacccctacctcccaagcccgtgttgctgactttcgatgacggactggtcagcggctacaccaaggtgttcccgctgctcaagcagttcaactatcctgcggtattcgccctggtcacccgctgggtcgacatgcccgatacggataccgtaccgatcagcgccaaggtggcgctcaccggcaaggacttcctgaactgggatcaggtgcgcgaaatgtcggagtccaatctggtcgagttcgcctcgcactcgcacgacatgcaccgcggcatcatcgccaacccccagaacaacgaaaagccggccgcctcattccggatctatgacaaggttacccagacatacgaaaccttcgaggagttccgggcccggctgcgcgcagacctggctgaaagcattcgcctgatcgagcagcacacgggcaagcgtccgcgcatcatggcatggccctacggcgcgcaaaaccgtcacagcgacgacgtggcgcgtgagctcggcctggacgtcatgctgacgctgcgcccggggcccaacacccctgatgtccccctggaccaggtgcggcggatcctgctgaactatgaggtcacggtaggccagctgacgagcgaaatgcgccagcccatgacgaatgcaggcatcctgcgccccgtgcaacgcgtcgtccaggttgacctggactacgtgtacgaccctgatcccgcccagcaggaacgcaacctttcccggttgatcgaccgcatcaaggacttgaagcccagtacggtgtacctgcaggccttcgccgacgagaaaggtacaggcgaagtcacctccgtgtacttccctaaccggcacatgcccatgcgggccgacctgttcggtcgcgtcgcctggcaattgcgtacccgcgccgaggtgcaggtttttgcatggatgccagtactgacgttttcgcttcccaaggagcatcccgcatcgggacgtgtcgtccagtcgtcgggacgcgcgccgggtgagcgtggcctgggcagcccgacacggctgagcccatacgacccgactgtgcgccaggtgatcgcggagatctacgaggatctggccgccacgacccgctttgaaggcgttctctttcatgatgacgccgtactggattcaaccgaggatgccagtccacaggctctgcaaacctatgcatcctgggggctgccgccagacctggctgcaatcaggcagtctcccgaactgaaccagcgctggatacgtggcaagacgcgccacctgatcgacttcacgcatgagctgacgaacactgtcaatgcttaccagcatggagaaaccctgctcaccgttcgtaacctgtatgcccgccctgtccttgagccgcaagccgaaggcgacttttcgcaaaacctgacggacttcctcgcgaactacgactacgttgcggtcatggccatgccttacatggagaacgcaagcgaccctgaaggctggctgaaagacctggtcaaggccgtcgatgcgcaaaaaggcctgccccgcaccatattcgagctccaggcaatggactggcgtacagcaaaaccggtcgactcgaatgaaatgcttggacatatgcaaacccttcgcagcatgggcgccgtccactatgggtactaccccgatgacttcatctccaaccaccccaatgtcgaggtgctgcgcgaagccatgtcgctgaagcagtctttggaagtcaggaagctgcctcccctccaggaagccacgcgccagcttgtacttggcatttcaaacgacactcaggtcaggtga

SEQ ID NO: 47 - protein, 699 aa, Enviromental bacterial community H
MQTLFKLLTMSLLWFCMAAQAASPAGGYSLEFLPPPDPNDGLTYRVLCLHDVRDNLRASFDTNPDPYAIDTRTLVDIFEWLRAKEFHPVSMSQIIDARNGGKPLPPKPVLLTFDDGLVSGYTKVFPLLKQFNYPAVFALVTRWVDMPDTDTVPISAKVALTGKDFLNWDQVREMSESNLVEFASHSHDMHRGIIANPQNNEKPAASFRIYDKVTQTYETFEEFRARLRADLAESIRLIEQHTGKRPRIMAWPYGAQNRHSDDVARELGLDVMLTLRPGPNTPDVPLDQVRRILLNYEVTVGQLTSEMRQPMTNAGILRPVQRVVQVDLDYVYDPDPAQQERNLSRLIDRIKDLKPSTVYLQAFADEKGTGEVTSVYFPNRHMPMRADLFGRVAWQLRTRAEVQVFAWMPVLTFSLPKEHPASGRVVQSSGRAPGERGLGSPTRLSPYDPTVRQVIAEIYEDLAATTRFEGVLFHDDAVLDSTEDASPQALQTYASWGLPPDLAAIRQSPELNQRWIRGKTRHLIDFTHELTNTVNAYQHGETLLTVRNLYARPVLEPQAEGDFSQNLTDFLANYDYVAVMAMPYMENASDPEGWLKDLVKAVDAQKGLPRTIFELQAMDWRTAKPVDSNEMLGHMQTLRSMGAVHYGYYPDDFISNHPNVEVLREAMSLKQSLEVRKLPPLQEATRQLVLGISNDTQVR

SEQ ID NO: 48 - protein, 678 aa, Enviromental bacterial community H
ASPAGGYSLEFLPPPDPNDGLTYRVLCLHDVRDNLRASFDTNPDPYAIDTRTLVDIFEWLRAKEFHPVSMSQIIDARNGGKPLPPKPVLLTFDDGLVSGYTKVFPLLKQFNYPAVFALVTRWVDMPDTDTVPISAKVALTGKDFLNWDQVREMSESNLVEFASHSHDMHRGIIANPQNNEKPAASFRIYDKVTQTYETFEEFRARLRADLAESIRLIEQHTGKRPRIMAWPYGAQNRHSDDVARELGLDVMLTLRPGPNTPDVPLDQVRRILLNYEVTVGQLTSEMRQPMTNAGILRPVQRVVQVDLDYVYDPDPAQQERNLSRLIDRIKDLKPSTVYLQAFADEKGTGEVTSVYFPNRHMPMRADLFGRVAWQLRTRAEVQVFAWMPVLTFSLPKEHPASGRVVQSSGRAPGERGLGSPTRLSPYDPTVRQVIAEIYEDLAATTRFEGVLFHDDAVLDSTEDASPQALQTYASWGLPPDLAAIRQSPELNQRWIRGKTRHLIDFTHELTNTVNAYQHGETLLTVRNLYARPVLEPQAEGDFSQNLTDFLANYDYVAVMAMPYMENASDPEGWLKDLVKAVDAQKGLPRTIFELQAMDWRTAKPVDSNEMLGHMQTLRSMGAVHYGYYPDDFISNHPNVEVLREAMSLKQSLEVRKLPPLQEATRQLVLGISNDTQVR

SEQ ID NO: 49 - protein, 352 aa, Halomonas sp-62262
RIVHVDLDYVYDPDPIQQEQNLDRLIERISRYGVSTVYLQAYADPDGDGVADALYFPNRHLPVRADLFNRVAWQLKKRASVKVYAWMPVLSFDLGAGYQYVTDVRTGAEAPDHYRRLSPYVEENRRIIREIYQDLGRLTKFDGLLFHDDAFFTDFEDATPDAIAAYEDASLPSDINTIRNDDSLMTTWTRFKTAYLTDFTYELEQAANYYRQADNKVFTTSRNLYAVTVMEPRSQRWFAQDIQSFAAGYDFVAVMAMPYMEEAENPDEWLRALAQRSLAQVSAEQLVFELQTQNWHTQTPIPSEEIAQWVRILREEGIKNIGYYPDDFLQNHPDANVMRPVFSIGRRFRATP

SEQ ID NO: 50 - protein, 357 aa, Pseudomonas migulae
APLRVLHVDLDNVYDPDPAQQARNLDQLVQRVVDMGAGTVFLQAFADPKGDGLVHSLYFPNRHLPVRADLFNRVSWQLHTRAHAAVYAWMPVLSFALDPKLQRVTRWDPQTGQVATDPDQYKRLSPFDPQVRKIIGEIYEDLARNNAIDGVLYHDDAVLSDFEDASPAALKAYAANGLPGSIAALRADPAVMQRWTRFKSRYLIDFTNELTAKVRAIGGPQVQTARNIFAEPMLNPASEAWFAQNLDDFLQAYDWTAPMAMPLMEGQELKTSNAWLEKLVATVKARPGALEKTVFELQAKDWRTKAAPDINAVQMAEWMGVLKRQGVKSFGYYPDNFLENSPDLKTVRPALSNQWNP

SEQ ID NO: 51 - protein, 358 aa, Pseudomonas sp-62331
APMRVVHVDLDNVYDPDPVQQDANLGKLIQRMADMGANTVFLQAFADPKGDGLVHSLYFPNRHLPVRADIFDRVAWQLRTRAHVKVFAWMPVLSFALDPALPRVTRWNPDIGQTSVDPGQYRRLSPFDPNVRRIIGEIYEDVARLTSVDGILYHDDALLSDFEDASPQALKVYAANGLPDSMAALRADPATLQRWSRFKSRYLIDFTNELTAKVRAIRGPQVQTARNLFAEPMLNPSSEAWFAQNLDDFLGAYDWTAPMAMPLMEGKSCEQSGAWLEALVATVRSHPGALDRTVFELQARDWTRKDADAELNGEQLADWMGRLKRQGATSFGYYPDNFLENQPDLKTVRPALSNKWNP

SEQ ID NO: 52 - protein, 354 aa, Pseudomonas jessenii
RVVHVDLDNVYDPDPAQQDINLGKLVQRIADLGANTVFLQAFADPKGDGLVHSLYFPNRHLPVRADIFNRVAWQLRTRANVKVFAWMPVLSFGLDSKLPRVTRWDPKTGITSVDPDQYQRLSPFDPEVRRIIGEIYEDVARLTSVNGILYHDDAVLSDFEDAGPEALKAYAAHGLPGSIAALRDDPAAMQRWTRFKSQYLIDFTNELTAKVRAIRGPQVLTARNIFAEPMLNPESEAWYAQNLDDFLQTYDWTAPMAMPLMEKQTQAESGPWLEALVATVRKRPGALDRTVFELQARDWTKKDQADIDGALLADWMGRLKRQGATSFGYYPDNFLDNLPDLKTVRPALSNKWNP

SEQ ID NO: 53 - protein, 357 aa, Pseudomonas panacis
APLRVLHVDLDNVYDPDPAQQARNLDQLVQRVVDMGAGTVFLQAFADPKGDGLVHSLYFPNRHLPVRADLFNRVAWQLRTRAHASVYAWMPVLSFALDPKLPRVTRWDPQTGQVATDPDQYKRLSPFDPQVRKIIGEIYEDLARNNAIDGVLYHDDAVLSDFEDASPAALKAYAANGLPGSIAALRADPAVMQRWTRFKSRYLIDFTNELTAKVRAIGGPQVQTARNIFAEPMLNPASEAWFAQNLDDFLQAYDWTAPMAMPLMEGQELKTSNAWLEKLVATVKARPGALEKTVFELQAKDWRTQAAPDINGAQMAEWMGVLKRQGVKSFGYYPDNFLENSPDLKTVRPALSNQWNP

SEQ ID NO: 54 - protein, 354 aa, Pseudomonas koreensis
RVVHVDLDNVYDPDPAQQEINLGKLIQRMADMGANTVFLQAFADPKGDGLVHSLYFPNRHLPVRADIFDRVAWQLRTRAHVKVFAWMPVLSFGLDAKLPRVTRWDPKTGTTGVDPDQYQRLSPFDPEVRRIIGEIYEDVARLTSVDGILYHDDAVLSDFEDASPQALKAYAAQGLPGSIAALRDDPAAMQRWTRFKSRYLIDFTNELTAKVRAIRGPQVETARNIFAEPILNPDSEAWFAQNLDDFLVTYDWTAPMAMPLMEKQTLAQSGPWLEELVAKIKQRPGALDHTVFELQARDWTKKDQADIDGARLADWMGRLKRQGANSFGYYPDNFLDNLPDLKTVRPALSNKWNP

SEQ ID NO: 55 - protein, 354 aa, Pseudomonas sp-62498
RVVHVDLDNVYDPDPAQQEINLGKLIQRMADMGANTVFLQAFADPKGDGLVHSLYFPNRHLPMRADLFDRVAWQLRTRAHVKVFAWMPVLSFELDSKLPRVTRWDPKTGTTAVDPDQYKRLSPFDPEVRRIIGEIYEDVARLTSVDGILYHDDAVLSDFEDAGPQALKAYAAHGLPGSIAALRDDPAAMQRWTRFKSRYLIDFTNELTAKVRAIRGPQVLTARNIFAEPVLNPHSEAWFAQNLDDFLVSYDWTAPMAMPLMEKQTQAQSGPWLEELVAKIKQRPGALDRTVFELQARDWTKKEQADIDGAQLADWMSRLKRQGATSFGYYPDNFLENLPDLKTVRPALSNKWNP

SEQ ID NO: 56 - protein, 343 aa, Halomonas zhanjiangensis DSM 21076
RIVHVDIDYVYDDDPIQQEQNLDRLIERIYNYGVTTVYLQAFADPDGDGVADELYFPNRHLPVRADLFNRVAWQLKKRAGVKVYAWMPVLSFDLGDDYRYVIDSQTGLEAPDRYRRLSPYVEDNRRIIREIYQDLGRLTKFDGVIFHDDAFLNDFEDANPEALAAYAQASLPRDITAIKNDASLMAAWTQHKTQFLTDFTRELEQAANYYRQADNKEFTTARNLYARTVMNPESQQWFSQDIQNFASGYDFVAVMAMPYMEEAENPDEWLRELARRSLAQVSSKQLVFELQTQDWRTQTPVSSEELARWVRLLREEGIQHIGYYPDDFLQNHPNLSVLRPAFS

SEQ ID NO: 57 - protein, 344 aa, Halomonas sp-63456
RIVHVDLDYVYDPDPQQQERNLDKLVERIAGYGVNTVYLQAYADPNGNGVAEELYFPNRHLPVRGDLFNRVAWQLKKRADVKVYAWMPVLAFDLGAGHRYVTDVRTGTESPEHYRRLSPYVEENRRIIREIYEDLGRFTKFDGLLFHDDAFLTDFEDANLEALAQYSNAFLPEDIDAIRSNDGLMNSWARHKTEFLTDFTLDLKQSANYYRQSDNKVFTTSRNLYAVTVMNPNSRLWFSQDIENFTSAYDYVAVMAMPYMEEADNPKEWLTELAQRSLEKVGEDKLVFELQTQDWRTQTPIPSEEISGWVRFLRNEGIKHIGYYPDDFHNNHPDVNVMKPHFSI

SEQ ID NO: 58 - protein, 345 aa, Enviromental bacterial community U
RIVHVDLDYVYDPDPIQQEANLDLLVDRIYRFGVTTVYLQAFADPNGNGVAEALYFPNRHLPMRADLFNRVAWQLKKRANVKVYAWMPVLAFDLGPGHEYVTDARTGVVASEHYLRLSPYAATNRRIISEIYRDLGRLAKFDGLLFHDDAFLTDFEDAGDDALSLHETTWNLPRDIEAIRREEALMDNWTRRKTAFLTDFTLELADSANYYRQSDNKRFTTSRNIYALPIIEPESQAWFAQAAENFASAYDYVAVMAMPYMEEADDPQRWLEELARKSLAEIPAEKLVFELQTRDWRDESLVRNEVLTSWIDTIKHAGIRHIGYYPDDFHNNHPDMSALRPYFSI

SEQ ID NO: 59 - protein, 5 aa, Artificial
  Motif
  MISC_FEATURE (1)..(1): Xaa = Y (Tyr) or W (Trp)
  MISC_FEATURE (3)..(3): Xaa = any natural amino acid
  MISC_FEATURE (4)..(4): Xaa = D (Asp) or N (Asn)
XPXXF

SEQ ID NO: 60 - protein, 4 aa, Artificial
  Motif
  MISC_FEATURE (1)..(1): Xaa = M (Met) or E (Glu) or Y (Tyr) or F (Phe)
  MISC_FEATURE (4)..(4): Xaa = P (Pro) or G (Gly)
XAMX

SEQ ID NO: 61 - protein, 27 aa, Artificial
  Signal peptide
MKKPLGKIVASTALLISVAFSSSIASA

SEQ ID NO: 62 - protein, 8 aa, Artificial
  His tag
HHHHHHPR

SEQ ID NO: 63 - DNA, 2067 bp, Vibrio proteolyticus
  CDS (1)..(2064)
  sig_peptide (1)..(63)
  mat_peptide (64)..(2064)
atgaatatgtcacaaatcatcattctgatcatcgccgtactgtctgttgccagtactaaggcggccgattccagcatcacaccatcgattgcaaacatgacagtgagtgaactggacaatacccgtactattcatcacttcaaccaactggtgccccgtcctttgcgcagtgcgccaagggatgacttatcgtacgttgcgttgtgttatcacgatttcattgatctacgcttaacgccggatgccaagatttttccggacacgctgacccgcgaccgattggtggagcactttaactggcttaaacaacagggatatcacccggtcagcttccaacaaatcattgatgctcaaactggtaaagccccgttacctgacaaagctgtactgctgacttttgatgatggctacgccagcttctactacaccgtgtatccgcttctcaagttgtacaacttcccggcggtgatggcgatagtcggtcactggctggaaccttctgcgaatagcatggtgccgtatggcaagctgatgatctcacaaaagcgctttctcaactgggaacagatcaaagagatgcaggactccggactgattgagattgcttcgcatacctacaattcgcattacggtatttacgctaactcattcgggaatgaagagccggcagtgacctcaccagcttatgatcccaagcaaggccgctacgaaactctgacccagtacaaacagcgtttgctgagcgacttcgagcagagccgggaccaaatggtgaaacacggtatccgtccaccccgaatcatggtctggccgtatggtgcgtacaatctggcggcgctggaagtggcggataaagtcggtatgacatacgccttgtcattggatgaaggtgtgaattcggtgcatgagtctggccgcaatgtgaaacgttatctgatcgagcaagagattaacctggaccgactggatgaaattctctccggagagccccaatttgaaggctccgaacgcattgtgcatgtggatcttgattacgtctattcagacgatccggccgagatgaatcgcaatctggatatcctgattgagcggatcaaaggggcggggattaatacggtctacttgcaggcatttgcggatccggatggtgacggcgtggcggatgcactctactttcagaacagttatctgccagtcagagcggatctgtttaaccgggtcgcctggcagttgaaaacccgtgctggcgtcaaagtgtttgcctggatgccagtacttggctttgatctgggaccaaattaccagtacgtcaccgatgtgcgcttcggtgcgccagataaagggcgttatctgcgtttatcgcctttctcagtgaaaaatcgtaatgcgattcggcagatataccgtgaactgggggcatacggcaagtttgctggcatcttgttccatgatgatgctttcctgacggatttcgaagatgcgtctccggccgctttgcaacagtatcgccgttgggggttaaaccctaatgtgactgcaatccgagaggatggcgagcaaatggccagttggactcagcacaagacacaatacctgattgattttacgctggatttagcagaaaacgcccgtcagtacgcgtctggcagcggttcccctatgatgctggcgcgcaatatctacgcagaagcagtgcttaacccagccagtgaagcgtggtttgcgcaaagtatgtctgcgtttggccaggcgtacgattacaccgcagtaatggcgatgccctatatggaacaggcagagcagccgatggcctggctgcgcaatattgcaaagctgtcattgcaaagtgttccggccgacaagctggtgtttgagcttcaggcccgcaactggcgcactggtaaaccgctgtcgtcagaagagctggcacagcagatgaaaatcatcgcgcaagaaggcattcaacactacgggtactatccggatgatttcattagcgcacagccggatctcaaggtgttgcgtccggtattctccaactcgggcaagatggagggggcgaaatga

SEQ ID NO: 64 - protein, 688 aa, Vibrio proteolyticus
MNMSQIIILIIAVLSVASTKAADSSITPSIANMTVSELDNTRTIHHFNQLVPRPLRSAPRDDLSYVALCYHDFIDLRLTPDAKIFPDTLTRDRLVEHFNWLKQQGYHPVSFQQIIDAQTGKAPLPDKAVLLTFDDGYASFYYTVYPLLKLYNFPAVMAIVGHWLEPSANSMVPYGKLMISQKRFLNWEQIKEMQDSGLIEIASHTYNSHYGIYANSFGNEEPAVTSPAYDPKQGRYETLTQYKQRLLSDFEQSRDQMVKHGIRPPRIMVWPYGAYNLAALEVADKVGMTYALSLDEGVNSVHESGRNVKRYLIEQEINLDRLDEILSGEPQFEGSERIVHVDLDYVYSDDPAEMNRNLDILIERIKGAGINTVYLQAFADPDGDGVADALYFQNSYLPVRADLFNRVAWQLKTRAGVKVFAWMPVLGFDLGPNYQYVTDVRFGAPDKGRYLRLSPFSVKNRNAIRQIYRELGAYGKFAGILFHDDAFLTDFEDASPAALQQYRRWGLNPNVTAIREDGEQMASWTQHKTQYLIDFTLDLAENARQYASGSGSPMMLARNIYAEAVLNPASEAWFAQSMSAFGQAYDYTAVMAMPYMEQAEQPMAWLRNIAKLSLQSVPADKLVFELQARNWRTGKPLSSEELAQQMKIIAQEGIQHYGYYPDDFISAQPDLKVLRPVFSNSGKMEGAK

SEQ ID NO: 65 - protein, 667 aa, Vibrio proteolyticus
ADSSITPSIANMTVSELDNTRTIHHFNQLVPRPLRSAPRDDLSYVALCYHDFIDLRLTPDAKIFPDTLTRDRLVEHFNWLKQQGYHPVSFQQIIDAQTGKAPLPDKAVLLTFDDGYASFYYTVYPLLKLYNFPAVMAIVGHWLEPSANSMVPYGKLMISQKRFLNWEQIKEMQDSGLIEIASHTYNSHYGIYANSFGNEEPAVTSPAYDPKQGRYETLTQYKQRLLSDFEQSRDQMVKHGIRPPRIMVWPYGAYNLAALEVADKVGMTYALSLDEGVNSVHESGRNVKRYLIEQEINLDRLDEILSGEPQFEGSERIVHVDLDYVYSDDPAEMNRNLDILIERIKGAGINTVYLQAFADPDGDGVADALYFQNSYLPVRADLFNRVAWQLKTRAGVKVFAWMPVLGFDLGPNYQYVTDVRFGAPDKGRYLRLSPFSVKNRNAIRQIYRELGAYGKFAGILFHDDAFLTDFEDASPAALQQYRRWGLNPNVTAIREDGEQMASWTQHKTQYLIDFTLDLAENARQYASGSGSPMMLARNIYAEAVLNPASEAWFAQSMSAFGQAYDYTAVMAMPYMEQAEQPMAWLRNIAKLSLQSVPADKLVFELQARNWRTGKPLSSEELAQQMKIIAQEGIQHYGYYPDDFISAQPDLKVLRPVFSNSGKMEGAK

SEQ ID NO: 66 - DNA, 1962 bp, Aquitalea magnusonii
  CDS (1)..(1959)
  sig_peptide (1)..(63)
  mat_peptide (64)..(1959)
atgaaaacgaaacaatggtttttgctgatgtgcttggccctgtgcgcactgttggcgctggctgcaccggtgcgggctgactcccgcttgatcattctctgttaccacgaagtagggcagccggatgcgcgcagtgacgaccccttcgccgtggatgcacgcagcctggtgcggcagatggcatggatgcgcgggcagggttatcagtttgtcagcgtggaggatgtgctggccgaccgtgccggcaagaagccgctgccggacaaggcggtattgctgaccttcgatgatggctaccgcagcgtctacacccaggtttatccggtattgcgcagctttcatgcgccggcgctgattgcgctggtgggaagctggctggaggctgcggatggcgagcaagtgcgttatggcgacggcctggtgccgcgcagcacctttctgagctgggcgcagatccgcgaaatgcaggccagcggcctggtggaggtggccagccacagctatgccgagcattttggccaacgggccaatccgcagggcaatagcgagccggccctcacctcgctggcctggcaggcgggcagctatgaaaccccggaagcctaccgggcgcgcatccgtgccgatctgctgcgcaacagcacactgctcaaggccaggctgggccaggcgccgcgggtgatggtctggccctatggcagctatacccgcgaaacggcggccatcgccggcgagctgggcatgccgctgaccatgagcctggacgagggcatcaacacccgccagaccccgctggcggggctgcgccggctattgctggacgccaatatgagcctggccgacctggcctggcagttccagcagctggaaacctggccggacggcatacgccccgagcccagccgcatcatgcatgtcgatctcgactatatctacgaccccgatccggcgcggcaggaggcaaacctgggccggctgctggagcgggtcaaggccatgggcgccagtacggtctatctgcaggcctttgccaaccccgaaggcgatggcgtggcgcgggcgctgtatttccccaatcgccacctgccgatgcgggccgacctgttcaaccgcgccgtctggcaattgcgcacccgtgtcggcgtgcgtgtctacgcctggatgccactgctggctttcgacctgccgcgcgacaacccgctgcgccagcaaagagtgctggcactggggcggcaagggctgccggagcagcagggctatatccggctgacgccgtattcggccgaggcgcgccagaccatccgcgagatttaccaggacctggcgcgttcggtgcagttcgacggcctgctgttccatgacgacgccaccctgtccgacgtggaagatgtcagcggccccgccatggcggccaaccgcgccctgggcctgccggattccttaccggccctgcgccaggacgaagcggccctgcagcgctggaacgacagcaagatcaagctgctggacgatttcacgctggagctggcgcaagtggtgcgtacctggcagcccacgctgaaaaccgcccgcaatctctacgccggggtggtgatgaacccggccacggaaagctggtttgcccagtcttatgccacggcgctgcagcactacgaccgggtggccatcatggccatgccctatatggagaaggccgcgcagccgcggcaatggctgcgccagctgtttgacaaggtaaaggccgtacccggcgcgctcgatcgcacggtgttcgaattgcaggcgcgcgactggcgcaacggccagccgattccgacccgggaaatggccgaggtggtggatgagctgcatacccttggtgcacgccacattgcctattacccggacgacctgttccaggaccagccacgcctggccgatttcaaggcggccttttccatgcgcagccaccccgaacaataa

SEQ ID NO: 67 - protein, 653 aa, Aquitalea magnusonii
MKTKQWFLLMCLALCALLALAAPVRADSRLIILCYHEVGQPDARSDDPFAVDARSLVRQMAWMRGQGYQFVSVEDVLADRAGKKPLPDKAVLLTFDDGYRSVYTQVYPVLRSFHAPALIALVGSWLEAADGEQVRYGDGLVPRSTFLSWAQIREMQASGLVEVASHSYAEHFGQRANPQGNSEPALTSLAWQAGSYETPEAYRARIRADLLRNSTLLKARLGQAPRVMVWPYGSYTRETAAIAGELGMPLTMSLDEGINTRQTPLAGLRRLLLDANMSLADLAWQFQQLETWPDGIRPEPSRIMHVDLDYIYDPDPARQEANLGRLLERVKAMGASTVYLQAFANPEGDGVARALYFPNRHLPMRADLFNRAVWQLRTRVGVRVYAWMPLLAFDLPRDNPLRQQRVLALGRQGLPEQQGYIRLTPYSAEARQTIREIYQDLARSVQFDGLLFHDDATLSDVEDVSGPAMAANRALGLPDSLPALRQDEAALQRWNDSKIKLLDDFTLELAQVVRTWQPTLKTARNLYAGVVMNPATESWFAQSYATALQHYDRVAIMAMPYMEKAAQPRQWLRQLFDKVKAVPGALDRTVFELQARDWRNGQPIPTREMAEVVDELHTLGARHIAYYPDDLFQDQPRLADFKAAFSMRSHPEQ

SEQ ID NO: 68 - protein, 632 aa, Aquitalea magnusonii
APVRADSRLIILCYHEVGQPDARSDDPFAVDARSLVRQMAWMRGQGYQFVSVEDVLADRAGKKPLPDKAVLLTFDDGYRSVYTQVYPVLRSFHAPALIALVGSWLEAADGEQVRYGDGLVPRSTFLSWAQIREMQASGLVEVASHSYAEHFGQRANPQGNSEPALTSLAWQAGSYETPEAYRARIRADLLRNSTLLKARLGQAPRVMVWPYGSYTRETAAIAGELGMPLTMSLDEGINTRQTPLAGLRRLLLDANMSLADLAWQFQQLETWPDGIRPEPSRIMHVDLDYIYDPDPARQEANLGRLLERVKAMGASTVYLQAFANPEGDGVARALYFPNRHLPMRADLFNRAVWQLRTRVGVRVYAWMPLLAFDLPRDNPLRQQRVLALGRQGLPEQQGYIRLTPYSAEARQTIREIYQDLARSVQFDGLLFHDDATLSDVEDVSGPAMAANRALGLPDSLPALRQDEAALQRWNDSKIKLLDDFTLELAQVVRTWQPTLKTARNLYAGVVMNPATESWFAQSYATALQHYDRVAIMAMPYMEKAAQPRQWLRQLFDKVKAVPGALDRTVFELQARDWRNGQPIPTREMAEVVDELHTLGARHIAYYPDDLFQDQPRLADFKAAFSMRSHPEQ

SEQ ID NO: 69 - DNA, 1947 bp, Halomonas ilicicola
  CDS (1)..(1944)
  sig_peptide (1)..(57)
  mat_peptide (58)..(1944)
gtgagcaaatggattgccgccctgctggcctgcgtgctggcgatgcctgccctcgctggcgagggcccggccgaatacagtgtcatcagctaccacgacatcatcgacgtcgcccagacccccgacatgaaggtctacccgcagaccatcacccgggacatgctggtcaggcacttcaacctgatcgacgacctgggctacgagccggtgagcttccagcaggtgctcgacgccaaggccggcaaatcgccgttgccggacaaggcggtactgctgaccttcgacgatggctaccgcagcgtccatgacatcgtctttcccttgctcaagctctatgactttcccgcggtcgtggccccggtgggcagctggatggaggtccccgagggaggcgaggtgccctacggcaacatcatgctgccccgggaacgcttcatgagctgggagcaactgcgcgagctgaacgactcgccgctggtcgagatcgcctcgcatacccatgacctgcactacggcatcattggcaacccccagggcaacgagcagccagcggcaaccacgccgaaatggaccccggccggctacgagagcgagcaggaatacctcgagcgggtgcgcaacgatctcacccgggcgcgagaccagctcgaacgcgagctgggcgaggccccgcggatcatcatctggccctatggcgcctacagcgaagcggtgctggatatcgccggcgaggtgggcatgccgcacaccttcagtctgttggcgaagatcaacgacaccgacgagagcacccgggccatgggccgctacctggtcgaccaagaaacctccctggaactcttcgaggagtatctcgccgggcgcacctggaagcgcaaggccgagcgggtcgtgcatgtcgatctcgactacgtcttcgatcccgacaaggcccagcaggggcgcaacctcgacaagctgctcgacgagatcaaggcccagggcatcagcacggtctatctccaggcctacgccgatgtggacggcgacggcgtggccgaaggactctactttcccaacgcacacctgccggttaaggccgacctgttcaatcgcgtggcctggcagctcaagaagcgcgccgaggtcaaggtgtatgcctggatgccggtgatggccttcgatctgggtcggggccatgactacgtcagcgacgtgcgactcggcaagcccaacccggaacactacaagcggctttcgccctacgtcgagcgcaaccgcgagatcatcaaggatatctatcgcgacctggggctttataccaagttcgacggcgtgctgttccacgacgacgcctttctgagcgacttcgaggacgccagcctcgccgcccggaactggtatcgccaggaatggggccttccggatgccgtcaccgagattcgccgggacgatgccctgatgacgcgctggacccggcgcaagacccgcttcctgatcgacttcacccacgccctgggcgagcaggccaaccactatcgcatgctcgacaacgacccgttcgccctggcgcgcaacatttacgcacggcccatcctcgagcccgagagtcagcgctggttcgcccaggacctcgaggccttcgccgaagcctacgactataccgccgtgatggccatgccctacatggaaggggccgacgatcccgatgcctggctcaggaagctggctcgcgtatcgctcgacaaggtgcccgccgacaggctggtgttcgaattgcaggcccaggactggcgcgatcagacgcccatcccgagcgagcagcttgcccactggatgcgggtgatccgccaggcaggcatcgagaactacggctactacccccacgacttcctcaacgatcatccggacaccagcatattgcgccgtgacttctcgctttcctcgaccctggaggcttctcgatga

SEQ ID NO: 70 - protein, 648 aa, Halomonas ilicicola
VSKWIAALLACVLAMPALAGEGPAEYSVISYHDIIDVAQTPDMKVYPQTITRDMLVRHFNLIDDLGYEPVSFQQVLDAKAGKSPLPDKAVLLTFDDGYRSVHDIVFPLLKLYDFPAVVAPVGSWMEVPEGGEVPYGNIMLPRERFMSWEQLRELNDSPLVEIASHTHDLHYGIIGNPQGNEQPAATTPKWTPAGYESEQEYLERVRNDLTRARDQLERELGEAPRIIIWPYGAYSEAVLDIAGEVGMPHTFSLLAKINDTDESTRAMGRYLVDQETSLELFEEYLAGRTWKRKAERVVHVDLDYVFDPDKAQQGRNLDKLLDEIKAQGISTVYLQAYADVDGDGVAEGLYFPNAHLPVKADLFNRVAWQLKKRAEVKVYAWMPVMAFDLGRGHDYVSDVRLGKPNPEHYKRLSPYVERNREIIKDIYRDLGLYTKFDGVLFHDDAFLSDFEDASLAARNWYRQEWGLPDAVTEIRRDDALMTRWTRRKTRFLIDFTHALGEQANHYRMLDNDPFALARNIYARPILEPESQRWFAQDLEAFAEAYDYTAVMAMPYMEGADDPDAWLRKLARVSLDKVPADRLVFELQAQDWRDQTPIPSEQLAHWMRVIRQAGIENYGYYPHDFLNDHPDTSILRRDFSLSSTLEASR

SEQ ID NO: 71 - protein, 629 aa, Halomonas ilicicola
GEGPAEYSVISYHDIIDVAQTPDMKVYPQTITRDMLVRHFNLIDDLGYEPVSFQQVLDAKAGKSPLPDKAVLLTFDDGYRSVHDIVFPLLKLYDFPAVVAPVGSWMEVPEGGEVPYGNIMLPRERFMSWEQLRELNDSPLVEIASHTHDLHYGIIGNPQGNEQPAATTPKWTPAGYESEQEYLERVRNDLTRARDQLERELGEAPRIIIWPYGAYSEAVLDIAGEVGMPHTFSLLAKINDTDESTRAMGRYLVDQETSLELFEEYLAGRTWKRKAERVVHVDLDYVFDPDKAQQGRNLDKLLDEIKAQGISTVYLQAYADVDGDGVAEGLYFPNAHLPVKADLFNRVAWQLKKRAEVKVYAWMPVMAFDLGRGHDYVSDVRLGKPNPEHYKRLSPYVERNREIIKDIYRDLGLYTKFDGVLFHDDAFLSDFEDASLAARNWYRQEWGLPDAVTEIRRDDALMTRWTRRKTRFLIDFTHALGEQANHYRMLDNDPFALARNIYARPILEPESQRWFAQDLEAFAEAYDYTAVMAMPYMEGADDPDAWLRKLARVSLDKVPADRLVFELQAQDWRDQTPIPSEQLAHWMRVIRQAGIENYGYYPHDFLNDHPDTSILRRDFSLSSTLEASR

SEQ ID NO: 72 - DNA, 2010 bp, Alkanindiges illinoisensis
  CDS (1)..(2007)
  sig_peptide (1)..(66)
  mat_peptide (67)..(2007)
atgacgcggcttttatcttttttgtcggtggtgctgtgtattgcttttattgtgcccggctatgccaaacccatcgcttcaaaccagacttccaataaatcatctgttgccatgcctgccgaactgaccattatcggctaccatgaaattgcagccccgcagcaagccctgattcccgattatgctgttactccgcaacagtttgaacaacaggtgatctggctaaaaaaccatggtttccagtttgttagcatggatgatgtgctggcagcgcgtgcgggtaaaaagccacttccggccaaggctgtgctgctgagctttgatgacggctatgcgtcgttttatcagcatgcctatcccctgatcaaacactacaaaattccggttgtcctggcactagttggcaactggctattgccaccagaaaatcaaccggtggattttgatggaaagaccattagccgcaacagcatgtttagctgggcccaattgcgcgaaatgagtgatagcggtctggtggaaattgccagccacacttatgacctgcacgatgggattctggccaatccacagggcaatatgcagcctgccgttactgcgcgcctgtatgacccggctaccagaacctatgaaccggatcaggcttaccggatgcgcctgtacaatgattttaagaccaacaatgatttattcattaaacatggcttgaaaaaaccacggattatggtctggccatatggtcgctataataatgatgcgctggacttagccagccagttgggtatgcctattacctttacactggatgacgggccaaatctgccgtccaccccgctctcggtgttgcgccgcattctggtagacagcagcatgacgcctgctgatctgggccatgaaatcagcattcgcgagcagcagctcacggacaacaaccgtgcccagaaaatcatgcatgtggatctggactatatttatgatccaggcccggagcaacaggaacgcaatctgggccatttgctggaccggattgccgccatgcatgtcaatacggtttacctgcaagcgttttctgacccagatgccaatggctcagccgatatggtgtattttcccaaccggcatttgcctatgcgggcagacctgtttaaccgggtagcgtggcaaatccgtacccgcacacaagtaaaacgcgtgtatgcgtggatgcctttgctggcatgggaactgccggacagtgacccagcctcaaaagatattgtggtcacggaacaaggcaaaaacagccagcatttgaatatgggctatcatcgcctgtcgcccttttcggcactggcccggcaaaccattagcgagatttatcaggatctggccaagtctgtcccgtttgatggcatcctgtttcatgatgatgtcaccatgtcggactatgaagatgccagtcccaaggccaaagcacagtaccaagcctggggcctgcctgatgacttgagtgccattcggggtaatgatgccctgctgcaaaaatggaccgcgctcaaaacccagacactggatgaatttgccctgtcactggcggcagaggtgcgcaagcagcaaccctgcctgctgacggcacgcaacctgtatgcacaggtggccttaaaagcctatgcagaaaactggtatgcacaatcactggaaaactccctgcgcgattatgactttactgccattatggccatgccttatatggaacaggtcaaagaccctgatgcgttttatagcagtatcgtcaagcgggtcaaaagctatccgcaaggcctgaaaaaaactgtatttgaacttcaggctactgactggcgtagcaatacacctgtgtccacggacgaaatcgccaacaccattagcaagctgtatttacaaggggtacgccacgttggctattatccggatgacccgatcaaggatcacccggacccagccattctgcgccgggtatttgacagtaaaaccagtgcagccctgccctaa

SEQ ID NO: 73 - protein, 669 aa, Alkanindiges illinoisensis
MTRLLSFLSVVLCIAFIVPGYAKPIASNQTSNKSSVAMPAELTIIGYHEIAAPQQALIPDYAVTPQQFEQQVIWLKNHGFQFVSMDDVLAARAGKKPLPAKAVLLSFDDGYASFYQHAYPLIKHYKIPVVLALVGNWLLPPENQPVDFDGKTISRNSMFSWAQLREMSDSGLVEIASHTYDLHDGILANPQGNMQPAVTARLYDPATRTYEPDQAYRMRLYNDFKTNNDLFIKHGLKKPRIMVWPYGRYNNDALDLASQLGMPITFTLDDGPNLPSTPLSVLRRILVDSSMTPADLGHEISIREQQLTDNNRAQKIMHVDLDYIYDPGPEQQERNLGHLLDRIAAMHVNTVYLQAFSDPDANGSADMVYFPNRHLPMRADLFNRVAWQIRTRTQVKRVYAWMPLLAWELPDSDPASKDIVVTEQGKNSQHLNMGYHRLSPFSALARQTISEIYQDLAKSVPFDGILFHDDVTMSDYEDASPKAKAQYQAWGLPDDLSAIRGNDALLQKWTALKTQTLDEFALSLAAEVRKQQPCLLTARNLYAQVALKAYAENWYAQSLENSLRDYDFTAIMAMPYMEQVKDPDAFYSSIVKRVKSYPQGLKKTVFELQATDWRSNTPVSTDEIANTISKLYLQGVRHVGYYPDDPIKDHPDPAILRRVFDSKTSAALP

SEQ ID NO: 74 - protein, 647 aa, Alkanindiges illinoisensis
KPIASNQTSNKSSVAMPAELTIIGYHEIAAPQQALIPDYAVTPQQFEQQVIWLKNHGFQFVSMDDVLAARAGKKPLPAKAVLLSFDDGYASFYQHAYPLIKHYKIPVVLALVGNWLLPPENQPVDFDGKTISRNSMFSWAQLREMSDSGLVEIASHTYDLHDGILANPQGNMQPAVTARLYDPATRTYEPDQAYRMRLYNDFKTNNDLFIKHGLKKPRIMVWPYGRYNNDALDLASQLGMPITFTLDDGPNLPSTPLSVLRRILVDSSMTPADLGHEISIREQQLTDNNRAQKIMHVDLDYIYDPGPEQQERNLGHLLDRIAAMHVNTVYLQAFSDPDANGSADMVYFPNRHLPMRADLFNRVAWQIRTRTQVKRVYAWMPLLAWELPDSDPASKDIVVTEQGKNSQHLNMGYHRLSPFSALARQTISEIYQDLAKSVPFDGILFHDDVTMSDYEDASPKAKAQYQAWGLPDDLSAIRGNDALLQKWTALKTQTLDEFALSLAAEVRKQQPCLLTARNLYAQVALKAYAENWYAQSLENSLRDYDFTAIMAMPYMEQVKDPDAFYSSIVKRVKSYPQGLKKTVFELQATDWRSNTPVSTDEIANTISKLYLQGVRHVGYYPDDPIKDHPDPAILRRVFDSKTSAALP

SEQ ID NO: 75 - DNA, 1962 bp, Halomonas sp.
  CDS (1)..(1959)
  sig_peptide (1)..(72)
  mat_peptide (73)..(1959)
atgacactctggcgcatcattgtgatgggcgcagttatgatagcaatggtgggtattcaacaagcccaggcagcccggacgccgaatgattacgtggtaatcagttaccacgatattgtcgataccagcgttacccccaaccttgatatctatccccagaccattacccgcaaccgattggtcgagcacttcaatttaattgatgtcggcggctataacccggtgagtctgcaacagatcatcgatgccaaagcaggtaagcaacctctgccggaaaaagccgtgctgctgacgttcgatgacggatatcgcagcttttacgacatcgtctttccattactcaaactatatggttttcccgccgtgcaggcagtggtcggcagttggctggatgtacctgaaggtggccgggtgccctacggcaataccaccctgccccgtgagcgttttttgtcatgggaacaggttaaaaccctggacgaatcaccgctggtggaaatagcctcgcattcctacgaccttcactacggggttgtcggtaaccccatgggtaacgagcaagccgccgccgttaccagcacttggaacgcacgcaacgggtatgaaagtgaaacggcctatatcgagcgggtacgcagcgatatggcacggacacagcagcgttttcaggaacagatggggcgaagccctaggatcatggtttggccctacggtgcctatagccaggcaacgctcgatatcgccgcggaatacggcatggactacaccttcagtctactcagcgcacccaaccgtctcagcgactcgatgcgtaccatgaaccgctatctcatcgatcaggaaaccagcctgcaaaccatcgaagagattctctccaaccgaatatgggagccagaaaagctgcgcattgttcacgttgacctggactacgtgtatgacccggaccctacccagcaggcacaaaatctcgacaggctgatcgagcgcattgctgagtacggcgttagcaccgtttatcttcaggcctttgcagaccctgatggagacggcgttgccgatgccctctactttcctaaccgccatttaccggtcagggcagaccttttcaatcgcgttgcgtggcagttgaagaaacgtgccaatgtaaaagtgtacgcctggatgccggtgctgtcctttgacctggggagcggccaccggtatgtcaccgatgtaacaaccgggcgcgagtcccccgatcactacttgcgcctatcgccttatgtagaaagcaaccgtcgtataatccgtgaaatttatcaggatctcggtcggctgaccaaattcgacggcctgctcttccacgatgacgccttctttactgactttgaagacgccaacccagaagccctagccgcctatgagcgcgcttcgttaccgggggatatcaacgcaatacgcaacgacgatgacttgatgtcgacctgggctcgcttcaagacagagtatctcaccgatttcacgctggagctggagcaggccgctaactactatcgccaagccgacaacgtggtctttaccaccgctcgcaacctctatgccaagacggtgatggagccgagtagccagcaatggtttgctcaagatcccagggacttcgccaccggctacgactatgtggccgtcatggccatgccttatatggaagaagccgagaaccccgatgcgtggttaagaagccttgcccagcgctcgctttcgcaggtaagcgccgaccagctggtcttcgagcttcaggcgcagaactggcacacccagacacccattcccagcgaggagatagcgcaatgggttcgcgtactccgcgaagaaggcatcaagcacattggttactaccctgatgattttcaccagaatcatcctgatatcaataccatgagacccgtcttttccataggacgccggttcagggccatccaatga

SEQ ID NO: 76 - protein, 653 aa, Halomonas sp.
MTLWRIIVMGAVMIAMVGIQQAQAARTPNDYVVISYHDIVDTSVTPNLDIYPQTITRNRLVEHFNLIDVGGYNPVSLQQIIDAKAGKQPLPEKAVLLTFDDGYRSFYDIVFPLLKLYGFPAVQAVVGSWLDVPEGGRVPYGNTTLPRERFLSWEQVKTLDESPLVEIASHSYDLHYGVVGNPMGNEQAAAVTSTWNARNGYESETAYIERVRSDMARTQQRFQEQMGRSPRIMVWPYGAYSQATLDIAAEYGMDYTFSLLSAPNRLSDSMRTMNRYLIDQETSLQTIEEILSNRIWEPEKLRIVHVDLDYVYDPDPTQQAQNLDRLIERIAEYGVSTVYLQAFADPDGDGVADALYFPNRHLPVRADLFNRVAWQLKKRANVKVYAWMPVLSFDLGSGHRYVTDVTTGRESPDHYLRLSPYVESNRRIIREIYQDLGRLTKFDGLLFHDDAFFTDFEDANPEALAAYERASLPGDINAIRNDDDLMSTWARFKTEYLTDFTLELEQAANYYRQADNVVFTTARNLYAKTVMEPSSQQWFAQDPRDFATGYDYVAVMAMPYMEEAENPDAWLRSLAQRSLSQVSADQLVFELQAQNWHTQTPIPSEEIAQWVRVLREEGIKHIGYYPDDFHQNHPDINTMRPVFSIGRRFRAIQ

SEQ ID NO: 77 - protein, 629 aa, Halomonas sp.
ARTPNDYVVISYHDIVDTSVTPNLDIYPQTITRNRLVEHFNLIDVGGYNPVSLQQIIDAKAGKQPLPEKAVLLTFDDGYRSFYDIVFPLLKLYGFPAVQAVVGSWLDVPEGGRVPYGNTTLPRERFLSWEQVKTLDESPLVEIASHSYDLHYGVVGNPMGNEQAAAVTSTWNARNGYESETAYIERVRSDMARTQQRFQEQMGRSPRIMVWPYGAYSQATLDIAAEYGMDYTFSLLSAPNRLSDSMRTMNRYLIDQETSLQTIEEILSNRIWEPEKLRIVHVDLDYVYDPDPTQQAQNLDRLIERIAEYGVSTVYLQAFADPDGDGVADALYFPNRHLPVRADLFNRVAWQLKKRANVKVYAWMPVLSFDLGSGHRYVTDVTTGRESPDHYLRLSPYVESNRRIIREIYQDLGRLTKFDGLLFHDDAFFTDFEDANPEALAAYERASLPGDINAIRNDDDLMSTWARFKTEYLTDFTLELEQAANYYRQADNVVFTTARNLYAKTVMEPSSQQWFAQDPRDFATGYDYVAVMAMPYMEEAENPDAWLRSLAQRSLSQVSADQLVFELQAQNWHTQTPIPSEEIAQWVRVLREEGIKHIGYYPDDFHQNHPDINTMRPVFSIGRRFRAIQ

SEQ ID NO: 78 - DNA, 1962 bp, Halomonas sp
  CDS (1)..(1959)
  sig_peptide (1)..(72)
  mat_peptide (73)..(1959)
gtgacacatcggcgcatcacgatcttgggcattgccctgctagtcatcctcggtcttcaacaggctcatgcggcacgctcaacggatgactacgtcgtcatcagctaccacgacgtggtggataccaacgtcacacccgatcagaccctttacccgcagaccattacacgcacgcgattgatagagcatttcaatcttatcgccgagggtggttatcagcccgtcagcctgcagcagatcatcgatgccagggcgggtcgacagccgctgccggacaaggccgtgctgctcacgttcgatgacggttatcgtagcttctacgatattgtctttcccctgctcaaactgtacgactttcctgctattcaagcggtggtgggcagctggatggatgtccctgccggtggacaggtttcctatggcgacatctacctgccccgcgaacgcttgctcacgtggcagcaagtcaagacgctgcatgagtcgcctctcgtggagatcgcctcccatacctatgaccttcaccatggagtcatcggcaatcccatgggcaaccagcaggctgccgccgtcaccagcatatgggaccctagaaccggctatgaaagtgaagtgcaatatgtcgaacgcattcggcaggatatggcacggacggtggagcagtttgaagggcgccttggccaaggccccagagccatgatctggccctatggcgcctatagtgaagcgatccttgatattgcagccgagtatggcatgacctataccttcggcctgctgggcgctcctaaccagcttcacgatgagatgcaggtaatcaatcgctatctgatcgatcaggaaaccagtctgcagaccttggacgaattcctttccaaccgcgtatgggagcgcgaagacctccgtatcgttcatgtcgacctggattatgtctacgatcccgatccgcagcagcaggagagaaatctggacaagctggtcgagcgcattgcgggttatggcgtcaacaccgtttatctgcaggcctatgccgacccgaatgggaatggcgttgccgaggagctctacttccccaatcgtcacctccccgtcagggccgacctcttcaaccgggtagcatggcagctaaagacacgtgccagtgtcaaggtctatgcatggatgccggtgcttgcctttgacctcggtgccggtcaccgctacgtcaccgatgtgagaacggggactgagtcgccggaacattaccgtcgactctcgccctatttggaagaaaaccggaggatcattcgcgagatctacgaagacctcggaagactgaccaaattcgacggtctgctttttcatgatgacgccttcatgacggactttgaagatgcaaatcccgaagcattgagacagtatggctcggcctccctgccggaggatatcgatgccatacgtgccaatgatgggctgatggcatcatgggccaggtacaagacagaatttctcacggatttcacccaggaactcaaacaatcggccaactattaccgtcaggcagattacagggtgttagccacatcacgtaatctatatgccattactgtaatggatccacgcagtcgactatggttctcccagaatatcgaaaattttgcttctgcctacgattatgtcgctgtcatggccatgccttatatggaggaagcgaataatccgactacgtggttgacggaactcgcacaacgatccctagaaaaggtaggtgccgagcagctggtattcgagctgcagacacaggactggcgtacccagaccccgattcctagccaagagatatcaggatgggtgcaattattacgcaatgagggaatcaagcatatcggatactatccggatgattttcataataaccatcctgatctcgatatcatgaagccacatttttccatcggtcgtcgctttagagaatcaccatga

SEQ ID NO: 79 - protein, 653 aa, Halomonas sp
VTHRRITILGIALLVILGLQQAHAARSTDDYVVISYHDVVDTNVTPDQTLYPQTITRTRLIEHFNLIAEGGYQPVSLQQIIDARAGRQPLPDKAVLLTFDDGYRSFYDIVFPLLKLYDFPAIQAVVGSWMDVPAGGQVSYGDIYLPRERLLTWQQVKTLHESPLVEIASHTYDLHHGVIGNPMGNQQAAAVTSIWDPRTGYESEVQYVERIRQDMARTVEQFEGRLGQGPRAMIWPYGAYSEAILDIAAEYGMTYTFGLLGAPNQLHDEMQVINRYLIDQETSLQTLDEFLSNRVWEREDLRIVHVDLDYVYDPDPQQQERNLDKLVERIAGYGVNTVYLQAYADPNGNGVAEELYFPNRHLPVRADLFNRVAWQLKTRASVKVYAWMPVLAFDLGAGHRYVTDVRTGTESPEHYRRLSPYLEENRRIIREIYEDLGRLTKFDGLLFHDDAFMTDFEDANPEALRQYGSASLPEDIDAIRANDGLMASWARYKTEFLTDFTQELKQSANYYRQADYRVLATSRNLYAITVMDPRSRLWFSQNIENFASAYDYVAVMAMPYMEEANNPTTWLTELAQRSLEKVGAEQLVFELQTQDWRTQTPIPSQEISGWVQLLRNEGIKHIGYYPDDFHNNHPDLDIMKPHFSIGRRFRESP

SEQ ID NO: 80 - protein, 629 aa, Halomonas sp
ARSTDDYVVISYHDVVDTNVTPDQTLYPQTITRTRLIEHFNLIAEGGYQPVSLQQIIDARAGRQPLPDKAVLLTFDDGYRSFYDIVFPLLKLYDFPAIQAVVGSWMDVPAGGQVSYGDIYLPRERLLTWQQVKTLHESPLVEIASHTYDLHHGVIGNPMGNQQAAAVTSIWDPRTGYESEVQYVERIRQDMARTVEQFEGRLGQGPRAMIWPYGAYSEAILDIAAEYGMTYTFGLLGAPNQLHDEMQVINRYLIDQETSLQTLDEFLSNRVWEREDLRIVHVDLDYVYDPDPQQQERNLDKLVERIAGYGVNTVYLQAYADPNGNGVAEELYFPNRHLPVRADLFNRVAWQLKTRASVKVYAWMPVLAFDLGAGHRYVTDVRTGTESPEHYRRLSPYLEENRRIIREIYEDLGRLTKFDGLLFHDDAFMTDFEDANPEALRQYGSASLPEDIDAIRANDGLMASWARYKTEFLTDFTQELKQSANYYRQADYRVLATSRNLYAITVMDPRSRLWFSQNIENFASAYDYVAVMAMPYMEEANNPTTWLTELAQRSLEKVGAEQLVFELQTQDWRTQTPIPSQEISGWVQLLRNEGIKHIGYYPDDFHNNHPDLDIMKPHFSIGRRFRESP

SEQ ID NO: 81 - DNA, 1875 bp, Luteibacter sp
  CDS (1)..(1872)
  sig_peptide (1)..(57)
  mat_peptide (58)..(1872)
atgcgtccgctgttgtttctgctgatcctgctcggggtggccctgcccgccgccgcgcagccggtcgctgccccgcaccctaccctcatcgtcctggcctaccacgacgtgcgcgacgacgtcggcctccaggccgatcacgatccggatgcgaccagcaccgatcacctcatcgctcacttcgactggctgcgcgccaacggctacaacatcgtcagcctgtcgcaggtggtggacgccagcaacggcggcaagcccttgccgaaggacgccgtgctgctgaccttcgacgatggcctcgagagcttctacacccgcgtgtatccgctgctgcgtgcctataactaccctgcgctgcaggcggtggtcggctcatggatggacatggccgacggcaagcacatgccgtacaacggcggtgactgcacgcgttcgtgtttcctcacgtgggaccaggtcgccgagatgcagaagtccggcctcgtcgagttcggctcgcacacctgggagatgcatcagggccagaacggcaatccgcagggcaaccagatgcccatggcggcctcgatcgcctacgacagcaccacgaagcattacgagaccgaagccgaatacgccgcacgcgtccgcaaggatctttcgcacagcgcggatgagatcgctgcgcataccggtgtgcgtccgcgcgcggcggtctggccttacggcgcctacacccgggtgggtcgcgaggtagccgccgatctgggcatgagcgtgtcgctcagcctgggcgacagcgtgccgacgctggtcccgggcaagacgatccctcgcctgctggtcagcggcaatgtcaccgccaaccgcctcggctggctgatgcgtcatcaggcccgtgtcgacgccacccgcgccgtgcaggtcgacctcgactacatctacgacccggatcccgcacagcaggatcgcaacctgtccaaactgctggatcgcatcaagcggatgcaccccagccaggtctggctgcaggcgtatgccgacccggatggcgacggcgtggccgatgccgtttacttccccaaccgtcatctgccggtgcgttccgacctgttttcgcgcgtctcgtggcagctgcgcacccgtgccggcgtgcgcgtctacgcgtggatgcccgtgctcgccttccgtttcccggacggcaaagacctgccctcgctggcaggtgagccgaagcccggcggcgatcatttccgtctggcaccgtacgacccgcgcgttcgccagatgatcggcgacgtgtacgaggacctcgcgatgcacgccgacgaggcaggcatcctgttctccgacgacgcttacattcgcgataccgacaaccttggcccgtgggcgaagaatacgccggcacagaatacgcaggcgctcatcgacttcacgaaggaactgaccgagcgcgtgcgccgctggcgtccgcaggtactgacggcccgcaatctctacgcgcgcccggtgctgcagccgcaggcggaagcctggatggcccagagcctgcccgccttcgtcgccgcttacgacatgaccgcgatcatggcgatgccacagctcgatcagcagtcggaccgcctcggctggtatcgcaagctgaccgcacaagtggccgccgtacccggtgccatggagcgcacgttgttcgaattcgccacgatgaactggcgcacgaagaagccgattcccgatctcgaccttgccgcccgcatgcgcgaagtgcaggctcagggtgcgcgccacatcggttactaccccgacgacttcctgcacaatcatcccgacctggagacgatccgcccgttcatctccgcgtccgactatccctatccggagccggcacgatga

SEQ ID NO: 82 - protein, 624 aa, Luteibacter sp
MRPLLFLLILLGVALPAAAQPVAAPHPTLIVLAYHDVRDDVGLQADHDPDATSTDHLIAHFDWLRANGYNIVSLSQVVDASNGGKPLPKDAVLLTFDDGLESFYTRVYPLLRAYNYPALQAVVGSWMDMADGKHMPYNGGDCTRSCFLTWDQVAEMQKSGLVEFGSHTWEMHQGQNGNPQGNQMPMAASIAYDSTTKHYETEAEYAARVRKDLSHSADEIAAHTGVRPRAAVWPYGAYTRVGREVAADLGMSVSLSLGDSVPTLVPGKTIPRLLVSGNVTANRLGWLMRHQARVDATRAVQVDLDYIYDPDPAQQDRNLSKLLDRIKRMHPSQVWLQAYADPDGDGVADAVYFPNRHLPVRSDLFSRVSWQLRTRAGVRVYAWMPVLAFRFPDGKDLPSLAGEPKPGGDHFRLAPYDPRVRQMIGDVYEDLAMHADEAGILFSDDAYIRDTDNLGPWAKNTPAQNTQALIDFTKELTERVRRWRPQVLTARNLYARPVLQPQAEAWMAQSLPAFVAAYDMTAIMAMPQLDQQSDRLGWYRKLTAQVAAVPGAMERTLFEFATMNWRTKKPIPDLDLAARMREVQAQGARHIGYYPDDFLHNHPDLETIRPFISASDYPYPEPAR

SEQ ID NO: 83 - protein, 605 aa, Luteibacter sp
QPVAAPHPTLIVLAYHDVRDDVGLQADHDPDATSTDHLIAHFDWLRANGYNIVSLSQVVDASNGGKPLPKDAVLLTFDDGLESFYTRVYPLLRAYNYPALQAVVGSWMDMADGKHMPYNGGDCTRSCFLTWDQVAEMQKSGLVEFGSHTWEMHQGQNGNPQGNQMPMAASIAYDSTTKHYETEAEYAARVRKDLSHSADEIAAHTGVRPRAAVWPYGAYTRVGREVAADLGMSVSLSLGDSVPTLVPGKTIPRLLVSGNVTANRLGWLMRHQARVDATRAVQVDLDYIYDPDPAQQDRNLSKLLDRIKRMHPSQVWLQAYADPDGDGVADAVYFPNRHLPVRSDLFSRVSWQLRTRAGVRVYAWMPVLAFRFPDGKDLPSLAGEPKPGGDHFRLAPYDPRVRQMIGDVYEDLAMHADEAGILFSDDAYIRDTDNLGPWAKNTPAQNTQALIDFTKELTERVRRWRPQVLTARNLYARPVLQPQAEAWMAQSLPAFVAAYDMTAIMAMPQLDQQSDRLGWYRKLTAQVAAVPGAMERTLFEFATMNWRTKKPIPDLDLAARMREVQAQGARHIGYYPDDFLHNHPDLETIRPFISASDYPYPEPAR

SEQ ID NO: 84 - DNA, 2076 bp, Variovorax boronicumulans
  CDS (1)..(2073)
  sig_peptide (1)..(87)
  mat_peptide (88)..(2073)
atgcgaagaacgacgaagaaccctttcaccttcctgcgcgcagccctgccatggctgctgctggcgctctgcctgccggccctcgcacagccgctgccgagcgccgaccccgacgacggcctgagctttcgcgtgatctccttccacgacgtgcgcaccaacgtgcgtgcgagcttcgagacctcgcccgacgagactgccgtggacgagcgcaccttcgccgaagtgttcgcctggctgcaatacagcggctaccacccggtgagcctgcagcagatcgtcgacgcgcgcgccggcggcaagccgctgccgcccaggcccgtgctgctgaccttcgacgacggctaccgcagcgcctacaccaaggtgtttccgctgctcaagcgctataactaccccgcgctgatggcgctggtgaccagctggctcgaagtgccccaaggccagcaggtgcactggggcgacaagcccgcgccgcgcgaagacttcctgctgtggagcgaagccgccgagatggcgcgttcgggcctcgtcgagttcgcgagtcataccgacgcaatgcacaccggcatcctcgccaacccgcagggcaacatgctgccggccgcggccacgcaccgctacgacccgaagaccgaccgctacgaggacgacgccgcctacacccggcgcatcgagaccgacctgcgccgcagccgcgagctcatcgagaaacgcaccggcgcgaaggtgcgcgccatggtgtggccctacggcgcctacaacgacgcggcgctgaaagcctccgcgcgcgcgggcatgcccatcaccttcacgctggacgacggctcgaacacgcccaccgtgccgctcacccgaatccgccgagcgctcgcggcctacgacaacgaggcgcccgaatacgcgcagctgctgcgcagccccgtgggcggcgaagtgcggcccgtcaaccgcgtgatgcacgtcgacctcgactacgtgtacgaccccgacccggcccagcaggagcgcaacctctcggcgctgatcgaccgcgtggccgccgtgcgcccgcgcgcggtgttcctgcaggcgtacgccgaccccgacggcgacggcgtggccgacgcgctgtactttcccaaccgccacctgccggtgcgcgccgacctgttcggccgcgctgcgtggcaactgcgcagccgcaccggcgtgaaggtctacgcgtggatgccggtaacggccttccggctgccggcctcgaacccgctcgccacgcacacggtggcggcgcagggcggcgcgatgccggccgaccgctaccaccggctcacgcccttcgaccccgcggtgcgcgcgctggtcggcgacatctacgaagacctgggcagccacgccttcttcgaaggcctgctgttccacgacgacgcgaccctgtcggacgacgaagacgcgagccccgccgcgctggccacctatgcgcaatggggcctgccggccgacgtggccgcgatccgcgcgaaccccgagctgatggcgcgctggaccgccgccaagacgcgctacctgatcgcgttcacgcaggagctcgccgcgcgcgtgggagcctggcggcccgcgctggaaactgcgcgcaacctctacgcgcgcccggtgctggagccccaggccgagcagtggttcgcgcagaactacgaagcctcgctcgccgcctacgactacgtcgcgttgatggcgatgccgcgcatggagcgcgagaccgatgcggacgcgtggctgggccggctcgcgcgtcgcgtggctgacacgccgcgcggcctcgacggcacggtgttcgagctgcaggcgcgcgactggcgcaccggcaagcccgtgcccgacgcggagctcgcgcgccagtggacgctgctgcagcgcgccggcgtgcgccacctgggctactaccccgacgacttcctcgataaccagccatcgctggagaccgtgcgccgcgcgatctcggtgcgcacgctgctgccgcgtgcattgcgcacgccttccgccgcggaaactcccgcacagcccgcgcaagccgcgccaggagaacggtcgccatga

SEQ ID NO: 85 - protein, 691 aa, Variovorax boronicumulans
MRRTTKNPFTFLRAALPWLLLALCLPALAQPLPSADPDDGLSFRVISFHDVRTNVRASFETSPDETAVDERTFAEVFAWLQYSGYHPVSLQQIVDARAGGKPLPPRPVLLTFDDGYRSAYTKVFPLLKRYNYPALMALVTSWLEVPQGQQVHWGDKPAPREDFLLWSEAAEMARSGLVEFASHTDAMHTGILANPQGNMLPAAATHRYDPKTDRYEDDAAYTRRIETDLRRSRELIEKRTGAKVRAMVWPYGAYNDAALKASARAGMPITFTLDDGSNTPTVPLTRIRRALAAYDNEAPEYAQLLRSPVGGEVRPVNRVMHVDLDYVYDPDPAQQERNLSALIDRVAAVRPRAVFLQAYADPDGDGVADALYFPNRHLPVRADLFGRAAWQLRSRTGVKVYAWMPVTAFRLPASNPLATHTVAAQGGAMPADRYHRLTPFDPAVRALVGDIYEDLGSHAFFEGLLFHDDATLSDDEDASPAALATYAQWGLPADVAAIRANPELMARWTAAKTRYLIAFTQELAARVGAWRPALETARNLYARPVLEPQAEQWFAQNYEASLAAYDYVALMAMPRMERETDADAWLGRLARRVADTPRGLDGTVFELQARDWRTGKPVPDAELARQWTLLQRAGVRHLGYYPDDFLDNQPSLETVRRAISVRTLLPRALRTPSAAETPAQPAQAAPGERSP

SEQ ID NO: 86 - protein, 662 aa, Variovorax boronicumulans
QPLPSADPDDGLSFRVISFHDVRTNVRASFETSPDETAVDERTFAEVFAWLQYSGYHPVSLQQIVDARAGGKPLPPRPVLLTFDDGYRSAYTKVFPLLKRYNYPALMALVTSWLEVPQGQQVHWGDKPAPREDFLLWSEAAEMARSGLVEFASHTDAMHTGILANPQGNMLPAAATHRYDPKTDRYEDDAAYTRRIETDLRRSRELIEKRTGAKVRAMVWPYGAYNDAALKASARAGMPITFTLDDGSNTPTVPLTRIRRALAAYDNEAPEYAQLLRSPVGGEVRPVNRVMHVDLDYVYDPDPAQQERNLSALIDRVAAVRPRAVFLQAYADPDGDGVADALYFPNRHLPVRADLFGRAAWQLRSRTGVKVYAWMPVTAFRLPASNPLATHTVAAQGGAMPADRYHRLTPFDPAVRALVGDIYEDLGSHAFFEGLLFHDDATLSDDEDASPAALATYAQWGLPADVAAIRANPELMARWTAAKTRYLIAFTQELAARVGAWRPALETARNLYARPVLEPQAEQWFAQNYEASLAAYDYVALMAMPRMERETDADAWLGRLARRVADTPRGLDGTVFELQARDWRTGKPVPDAELARQWTLLQRAGVRHLGYYPDDFLDNQPSLETVRRAISVRTLLPRALRTPSAAETPAQPAQAAPGERSP

SEQ ID NO: 87 - DNA, 1974 bp, Silvimonas terrae
  CDS (1)..(1971)
  sig_peptide (1)..(81)
  mat_peptide (82)..(1971)
atgcccaatgtgcataccttgctccggcactttgcagcactggccctgacggcactggtgctggccctgccggcccgcgccgataccgccacctggccggccgacagctttgaagtgctgtgctaccacgaagtgcgtgacgacgtgtgggtgctgccagacccctacgccatcagcacgcgccgactagcgcagcattttgagtggctgcgggagaacggttaccacgtgatcagcgtggatgacattctggacgcccgcgctggcaagaaaccattgccacccaaagccatcttgctgacttttgatgatggctatcgtagcttctacaccaacgtgttcccgctgctcaaagcctttggttaccacgccgtacaaggcatcgtcggccactggatcgacgccactgaaggcgcagagatcactgctgaatcccgcaattacagtcgggatgacatcatgacctgggcgcagatcaaggagttgtctgactccggtctggtggaaattgcctcgcacagcttcaacgagcaccacggcagccaggccaacccccaaggcaaccagttgccggcgctgattacgcacgtgtacgaccccagcacagggatgtacgagagtgacgatgagtatcaatcccgcatccggtctgacttgcaacagaactcggatgtgattgccaggcacatcggcaaggccccgcgcgtcatggtgtggccgtatggccgctacaaccaggccaccatcaacgccgccaaagccgccggcatgcctattaccctgacgctggatgatggcaccaacagcccggacgtggggctggaccggctgcgccgcgtactgatcacgcataacgatggcgagagtaccaaagcgctggatgccgccatgcgtgcgtcctccgtacgcccgatccgcgtgatgcatgtggatctggactatgtatacgacccggaccccaagcagcaggaaatcaacctgggcaagttgctggaccgcgtcaaagccagcggcgccaccaccgtgtttctgcaagcgtatgccgacccatctggcgcaggtgccgcgacggcgctgtatttccctaatcgccacttgcccatgcgcgccgatttgttcagccgcgccgcctggcaactggaaacccgtgccggcgtaaaggtgtacgcctggatgccgctgatggcctacgtgctgccggagaaagacccactggccagccatgtggtgacggcgagcgaccccaagggtgccaatgcctatcatcgcctcagcccgtttgatccggccgcgcgggcgttgattggcgatctgtatgaggacctggccacccacgccatctttcagggcgtgctgttccatgacgacgccaccctgaccgattttgaagatgacagcccggccgctcgtaccgtgtatcaaagctggggtctgccgggaaccgtggcagacatccgcaaagacccggcactgatgagtcgctggagcacgctcaagacggcgtatctgaccgacttctccatgcaactggctgagcgcgtgcgccagtggcaacccaatctgaaaaccgcccgcaatatctacgcccgcgtggtattgcagccagaagcgcaagcctggtttgcgcaatccctgccagattttctcaaccattacgactacaccgccatcatggcgatgccgtatatggaaggcgccaaagaaccgctgccgtggctgaagaacctggcgcaaatcgtggggcagattccgggcgcgctggataaaagcgtgtttgaactgcaagccacagactggaaaaccagcatgccgatcagcggccatgaactggccacgcaaatggacgtgctgattgatgctggcgcccgtaactacggctactacccggatgactttgtgcgcgaccagcccaagctggacgaattgattgagcgcttctcgcagcgcagctttccgtaccggagcggcaagtga

SEQ ID NO: 88 - protein, 657 aa, Silvimonas terrae
MPNVHTLLRHFAALALTALVLALPARADTATWPADSFEVLCYHEVRDDVWVLPDPYAISTRRLAQHFEWLRENGYHVISVDDILDARAGKKPLPPKAILLTFDDGYRSFYTNVFPLLKAFGYHAVQGIVGHWIDATEGAEITAESRNYSRDDIMTWAQIKELSDSGLVEIASHSFNEHHGSQANPQGNQLPALITHVYDPSTGMYESDDEYQSRIRSDLQQNSDVIARHIGKAPRVMVWPYGRYNQATINAAKAAGMPITLTLDDGTNSPDVGLDRLRRVLITHNDGESTKALDAAMRASSVRPIRVMHVDLDYVYDPDPKQQEINLGKLLDRVKASGATTVFLQAYADPSGAGAATALYFPNRHLPMRADLFSRAAWQLETRAGVKVYAWMPLMAYVLPEKDPLASHVVTASDPKGANAYHRLSPFDPAARALIGDLYEDLATHAIFQGVLFHDDATLTDFEDDSPAARTVYQSWGLPGTVADIRKDPALMSRWSTLKTAYLTDFSMQLAERVRQWQPNLKTARNIYARVVLQPEAQAWFAQSLPDFLNHYDYTAIMAMPYMEGAKEPLPWLKNLAQIVGQIPGALDKSVFELQATDWKTSMPISGHELATQMDVLIDAGARNYGYYPDDFVRDQPKLDELIERFSQRSFPYRSGK

SEQ ID NO: 89 - protein, 630 aa, Silvimonas terrae
DTATWPADSFEVLCYHEVRDDVWVLPDPYAISTRRLAQHFEWLRENGYHVISVDDILDARAGKKPLPPKAILLTFDDGYRSFYTNVFPLLKAFGYHAVQGIVGHWIDATEGAEITAESRNYSRDDIMTWAQIKELSDSGLVEIASHSFNEHHGSQANPQGNQLPALITHVYDPSTGMYESDDEYQSRIRSDLQQNSDVIARHIGKAPRVMVWPYGRYNQATINAAKAAGMPITLTLDDGTNSPDVGLDRLRRVLITHNDGESTKALDAAMRASSVRPIRVMHVDLDYVYDPDPKQQEINLGKLLDRVKASGATTVFLQAYADPSGAGAATALYFPNRHLPMRADLFSRAAWQLETRAGVKVYAWMPLMAYVLPEKDPLASHVVTASDPKGANAYHRLSPFDPAARALIGDLYEDLATHAIFQGVLFHDDATLTDFEDDSPAARTVYQSWGLPGTVADIRKDPALMSRWSTLKTAYLTDFSMQLAERVRQWQPNLKTARNIYARVVLQPEAQAWFAQSLPDFLNHYDYTAIMAMPYMEGAKEPLPWLKNLAQIVGQIPGALDKSVFELQATDWKTSMPISGHELATQMDVLIDAGARNYGYYPDDFVRDQPKLDELIERFSQRSFPYRSGK

(19) United States
(12) Patent Application Publication
(10) Pub. No.: US 2020/0109352 A1
(43) Pub. Date: Apr. 9, 2020

(54) POLYPEPTIDE COMPOSITIONS AND USES THEREOF

(71) Applicant: Novozymes A/S, Bagsvaerd (DK)

(72) Inventors: Rebecca Munk Vejborg, Allerod (DK); Christian Berg Oehlenschlaeger, Valby (DK); Dorotea Raventos Segura, Rungsted (DK); Jesper Salomon, Holte (DK); Henrik Marcus Geertz-Hansen, Copenhagen (DK); Christian Bech Rosen, Kobenhavn (DK)

(73) Assignee: Novozymes A/S, Bagsvaerd (DK)

(21) Appl. No.: 16/499,876

(22) PCT Filed: Apr. 4, 2018
(86) PCT No.: PCT/EP2018/058589

§ 371 Date: Oct. 1, 2019

(30) Foreign Application Priority Data
Apr. 4, 2017 (EP) 17164863.7

(51) Int. Cl. C11D 3/386; C12N 9/28; C11D 11/00

(52) U.S. Cl. CPC C11D 3/386; C11D 11/0017; C12N 9/2417

(57) ABSTRACT

The present invention relates to polypeptides, and polynucleotides encoding the polypeptides. The invention also relates to nucleic acid constructs, vectors, and host cells comprising the polynucleotides as well as methods of producing and using the polypeptides.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to polypeptides comprising a GHL13 pFam domain (PF14883) and polynucleotides encoding the polypeptides. The invention further relates to compositions comprising such polypeptides such as cleaning compositions, use of polypeptides comprising the GHL13 domain in cleaning processes and/or use of polypeptides comprising the GHL13 domain for cleaning e.g. deep cleaning of biofilm and components hereof, methods for removal or reduction of biofilm related soiling. The invention further relates to nucleic acid constructs, vectors, and host cells comprising the polynucleotides as well as methods of producing and using the polypeptides.

### Description of the Related Art

Enzymes have been used in detergents for decades. Usually a cocktail of various enzymes is added to detergent compositions. The enzyme cocktail often comprises various enzymes, wherein each enzyme targets its specific substrate e.g. amylases are active towards starch stains, proteases on protein stains and so forth. Textiles and surfaces such as laundry and dishes become soiled with many different types of soiling. The soiling may compose of proteins, grease, starch etc. One type of soiling comes from organic matter such as biofilm; the presence of biofilm provides several disadvantages. Biofilm comprises an extracellular polymeric matrix, composed of polysaccharides, extracellular DNA (eDNA), and proteins. The extracellular polymeric matrix may be sticky or glueing, which when present on textile, give rise to redeposition or backstaining of soil resulting in a greying of the textile. Another drawback is that malodor may be trapped within the organic structure. Organic matter such as biofilm is therefore not desirable in textiles and surfaces associated with cleaning such as washing machines etc. As organic soiling is a complex mixture of polysaccharides, proteins, DNA etc. there is a need for enzymes which effectively prevent, remove or reduce components of such soiling e.g. polysaccharides of components hereof on items such of fabrics.

### SUMMARY OF THE INVENTION

The invention relates to polypeptides and to compositions e.g. a cleaning composition e.g. a detergent composition, an ADW composition, a laundry composition, comprising a polypeptide according to the invention. In a first aspect, the invention relates to a composition comprising at least 0.01 mg of active polypeptide per gram of composition, wherein the polypeptide comprises a GHL13 domain and at least one cleaning component, preferably the polypeptide further comprises a CE4 domain. In a second aspect the composition according to the invention, comprises a polypeptide which is of the YPDDF clade, comprising one or more of the motif(s) [Y/W]PX[D/N]F (SEQ ID NO 59), [M/E/Y/F]AM[P/G] (SEQ ID NO 60) or WPY. One aspect of the invention relates to a polypeptide having hydrolytic and/or deacetyl activity, wherein the polypeptide is of the YPDDF clade, comprising one or more of the motif(s) [Y/W]PX[D/N]F (SEQ ID NO 59), [M/E/Y/F]AM[P/G] (SEQ ID NO 60) or WPY and wherein the polypeptide is selected from the group consisting of:

(a) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 3;

(b) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 6;

(c) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 9;

(d) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 12;

(e) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 15;

(f) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 18;

(g) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 21;

(h) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 24;

(i) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO 27;

(j) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 30;

(k) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 33;

(l) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 36;

(m) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 39;

(n) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 42;

(o) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 45;

(p) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 48;

(q) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 65;

(r) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 68;

(s) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 71;

(t) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 74;

(u) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 77;

(v) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 80;

(x) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 83;

(y) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 86;

(z) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 89;

(aa) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO 65, SEQ ID NO 68, SEQ ID NO 71, SEQ ID NO 74, SEQ ID NO 77, SEQ ID NO 80, SEQ ID NO 83, SEQ ID NO 86 and SEQ ID NO 89, wherein the variant has hydrolytic and/or deacetylase activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions;

(bb) a polypeptide comprising the polypeptide of (a) to (aa) and a N-terminal and/or C-terminal His-tag and/or HQ-tag;

(cc) a polypeptide comprising the polypeptide of (a) to (aa) and a N-terminal and/or C-terminal extension of between 1 and 10 amino acids; and

(dd) a fragment of the polypeptide of (a) to (aa) having hydrolytic and/or deacetylase activity and having at least 90% of the length of the mature polypeptide.

The present invention also relates to cleaning methods using the polypeptides of the present invention and to the use in cleaning processes. In aspects relates to the use of a polypeptide comprising a GHL13 domain, preferably a polypeptide comprising a GHL13 domain and a CE4 domain in a cleaning process, such as laundry and/or dish wash. Antoher aspect relates to the use of a polypeptide comprising a GHL13 domain, preferably a polypeptide comprising a GHL13 domain and a CE4 domain
a) for preventing, reducing or removing stickiness of the item, or
b) for preventing, reducing or removing a PNAG comprising stain from an item
c) for preventing, reducing or removing redeposition of soil from an item during a wash cycle, wherein the item is a textile.

The invention further relates to use of a polypeptide according to the invention for deep cleaning of an item, such as textile e.g. fabric. The invention further relates to the use of a polypeptide according to the invention,
(i) for preventing, reducing or removing stickiness of the item;
(ii) for pretreating stains on the item;
(iii) for preventing, reducing or removing redeposition of soil during a wash cycle;
(iv) for preventing, reducing or removing adherence of soil to the item;
(v) for maintaining or improving whiteness of the item;
(vi) for preventing, reducing or removal malodor from the item, wherein the item is a textile.

The invention also relates to a method for reducing or removing staining associated with organic matters e.g. extracellular polymeric substances (EPS) from a fabric, wherein the method comprises the steps of
a. contacting the fabric with a composition comprising a polypeptide having a GHL13 and/or a CE4 domain, and optionally
b. rinse the fabric.

The invention further relates to a cleaning or laundering method for cleaning or laundering an item comprising the steps of:

a. Exposing an item to a wash liquor comprising a polypeptide or a detergent composition comprising the polypeptides, wherein the polypeptide comprises a GHL13 and/or a CE4 domain and has hydrolytic and/or deacetylase activity, wherein the polypeptide is selected from the group consisting of the polypeptides shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77, SEQ ID NO 80, SEQ ID NO 83, SEQ ID NO 86 and SEQ ID NO 89 or a polypeptide having at least 60%, at least 65%, at least 70%, at least 75%, at least 80%, at least 85%, at least 90% at least 95%, at least 96%, at least 97%, at least 98%, at least 99% or 100% sequence identity hereto;

b. Completing at least one wash cycle; and

c. Optionally rinsing the item,

wherein the item is a textile.

An additional aspect relates to the use of a polypeptide comprising a GHL13 and/or CE4 domain wherein the polypeptide is selected from the group consisting of the polypeptides shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77, SEQ ID NO 80, SEQ ID NO 83 and SEQ ID NO 86 or a polypeptide having at least 60%, at least 65%, at least 70%, at least 75%, at least 80%, at least 85%, at least 90% at least 95%, at least 96%, at least 97%, at least 98%, at least 99% or 100%

identity for cleaning e.g. deep cleaning of an item.

The invention further relates to a polynucleotide encoding the polypeptide of the invention. A nucleic acid construct or expression vector comprising a polynucleotide encoding a polypeptide of the invention, which is operably linked to one or more control sequences that direct the production of the polypeptide in an expression host. The invention further relates to a recombinant host cell comprising a polynucleotide encoding a polypeptide of the invention, which is operably linked to one or more control sequences that direct the production of the polypeptide, wherein the method may further comprise cultivating a cell, which in its wild-type form produces the polypeptide, under conditions conducive for production of the polypeptide and optionally recovering the polypeptide.

### Overview of Sequences

SEQ ID NO 1 DNA sequence obtained from Pseudomonas meridiana
SEQ ID NO 2 is the polypeptide sequence derived from SEQ ID NO 1
SEQ ID NO 3 is mature polypeptide obtained from Pseudomonas meridiana
SEQ ID NO 4 DNA sequence obtained from Halomonas sp-62262
SEQ ID NO 5 is the polypeptide sequence derived from SEQ ID NO 3
SEQ ID NO 6 mature polypeptide obtained from Halomonas sp-62262
SEQ ID NO 7 DNA sequence obtained from Pseudomonas migulae
SEQ ID NO 8 is the polypeptide sequence derived from SEQ ID NO 7
SEQ ID NO 9 mature polypeptide obtained from Pseudomonas migulae
SEQ ID NO 10 DNA sequence obtained from of Pseudomonas sp-62331
SEQ ID NO 11 is the polypeptide sequence derived from SEQ ID NO 10
SEQ ID NO 12 mature polypeptide obtained from Pseudomonas sp-62331
SEQ ID NO 13 DNA sequence obtained from of Pseudomonas jessenii
SEQ ID NO 14 is the polypeptide sequence derived from SEQ ID NO 13
SEQ ID NO 15 mature polypeptide obtained from Pseudomonas jessenii
SEQ ID NO 16 DNA sequence obtained from Pseudomonas koreensis
SEQ ID NO 17 is the polypeptide sequence derived from SEQ ID NO 16
SEQ ID NO 18 mature polypeptide obtained from Pseudomonas koreensis
SEQ ID NO 19 DNA sequence obtained from Stenotrophomonas rhizophila
SEQ ID NO 20 is the polypeptide sequence derived from SEQ ID NO 19
SEQ ID NO 21 mature polypeptide obtained from Stenotrophomonas rhizophila
SEQ ID NO 22 DNA sequence obtained from Pseudomonas sp-62498
SEQ ID NO 23 is the polypeptide sequence derived from SEQ ID NO 22
SEQ ID NO 24 mature polypeptide obtained from Pseudomonas sp-62498
SEQ ID NO 25 DNA sequence obtained from Acinetobacter bouvetii
SEQ ID NO 26 is the polypeptide sequence derived from SEQ ID NO 25
SEQ ID NO 27 mature polypeptide obtained from Acinetobacter bouvetii
SEQ ID NO 28 DNA sequence obtained from of Pseudomonas panacis
SEQ ID NO 29 is the polypeptide sequence derived from SEQ ID NO 28
SEQ ID NO 30 mature polypeptide obtained from Pseudomonas panacis
SEQ ID NO 31 DNA sequence obtained from Enviromental bacterial community L
SEQ ID NO 32 is the polypeptide sequence derived from SEQ ID NO 31
SEQ ID NO 33 mature polypeptide obtained from Enviromental bacterial community L
SEQ ID NO 34 DNA sequence obtained from Halomonas zhanjiangensis DSM 21076
SEQ ID NO 35 is the polypeptide sequence derived from SEQ ID NO 34
SEQ ID NO 36 mature polypeptide obtained from Halomonas zhanjiangensis DSM 21076
SEQ ID NO 37 DNA sequence obtained from Halomonas sp-63456
SEQ ID NO 38 is the polypeptide sequence derived from SEQ ID NO 37
SEQ ID NO 39 mature polypeptide obtained from Halomonas sp-63456
SEQ ID NO 40 DNA sequence obtained from Luteibacter rhizovicinus
SEQ ID NO 41 is the polypeptide sequence derived from SEQ ID NO 40
SEQ ID NO 42 mature polypeptide obtained from Luteibacter rhizovicinus
SEQ ID NO 43 DNA sequence obtained from Enviromental bacterial community R
SEQ ID NO 44 is the polypeptide sequence derived from SEQ ID NO 43
SEQ ID NO 45 mature polypeptide obtained from Enviromental bacterial community R
SEQ ID NO 46 DNA sequence obtained from Enviromental bacterial community H
SEQ ID NO 47 is the polypeptide sequence derived from SEQ ID NO 46
SEQ ID NO 48 mature polypeptide obtained from Enviromental bacterial community H
SEQ ID NO 49 mature polypeptide obtained from Halomonas sp-62262
SEQ ID NO 50 mature polypeptide obtained from Pseudomonas migulae
SEQ ID NO 51 mature polypeptide obtained from Pseudomonas sp-62331
SEQ ID NO 52 mature polypeptide obtained from Pseudomonas jessenii
SEQ ID NO 53 mature polypeptide obtained from Pseudomonas panacis
SEQ ID NO 54 mature polypeptide obtained from Pseudomonas koreensis
SEQ ID NO 55 mature polypeptide obtained from Pseudomonas sp-62498
SEQ ID NO 56 mature polypeptide obtained from Halomonas zhanjiangensis 21076
SEQ ID NO 57 mature polypeptide obtained from Halomonas sp-63456
SEQ ID NO 58 mature polypeptide obtained from Enviromental bacterial community U
SEQ ID NO 59 motif [YW]PX[DN]F
SEQ ID NO 60 motif [MEYF]AM[PG]
SEQ ID NO 61 signal peptide MKKPLGKIVASTALLISVAFSSSIASA
SEQ ID NO 62 HHHHHHPR
SEQ ID NO 63 DNA sequence obtained from Vibrio proteolyticus
SEQ ID NO 64 is the polypeptide sequence derived from SEQ ID NO 63
SEQ ID NO 65 is mature polypeptide obtained from Vibrio proteolyticus
SEQ ID NO 66 DNA sequence obtained from Aquitalea magnusonii
SEQ ID NO 67 is the polypeptide sequence derived from SEQ ID NO 66
SEQ ID NO 68 is mature polypeptide obtained from Aquitalea magnusonii
SEQ ID NO 69 DNA sequence obtained from Halomonas ilicicola
SEQ ID NO 70 is the polypeptide sequence derived from SEQ ID NO 69
SEQ ID NO 71 is mature polypeptide obtained from Halomonas ilicicola
SEQ ID NO 72 DNA sequence obtained from Alkanindiges illinoisensis
SEQ ID NO 73 is the polypeptide sequence derived from SEQ ID NO 72
SEQ ID NO 74 is mature polypeptide obtained from Alkanindiges illinoisensis
SEQ ID NO 75 DNA sequence obtained from Halomonas sp.
SEQ ID NO 76 is the polypeptide sequence derived from SEQ ID NO 75
SEQ ID NO 77 is mature polypeptide obtained from Halomonas sp.
SEQ ID NO 78 DNA sequence obtained from Halomonas sp.
SEQ ID NO 79 is the polypeptide sequence derived from SEQ ID NO 78
SEQ ID NO 80 is mature polypeptide obtained from Halomonas sp.
SEQ ID NO 81 DNA sequence obtained from Luteibacter sp.
SEQ ID NO 82 is the polypeptide sequence derived from SEQ ID NO 81
SEQ ID NO 83 is mature polypeptide obtained from Luteibacter sp.
SEQ ID NO 84 DNA sequence obtained from Variovorax boronicumulans
SEQ ID NO 85 is the polypeptide sequence derived from SEQ ID NO 84
SEQ ID NO 86 is mature polypeptide obtained from Variovorax boronicumulans
SEQ ID NO 87 DNA sequence obtained from Silvimonas terrae
SEQ ID NO 88 is the polypeptide sequence derived from SEQ ID NO 87
SEQ ID NO 89 is mature polypeptide obtained from Silvimonas terrae

### DETAILED DESCRIPTION OF THE INVENTION

Various enzymes are applied in cleaning processes each targeting specific types of soiling such as protein, starch and grease soiling. Very effective often modified enzymes are standard ingredients in detergents for laundry and dish wash. The effectiveness of these commercial enzymes provides detergents which removes much of the soiling. However, organic matters such as EPS (extracellular polymeric substance) comprised in much biofilm constitute a challenging type of soiling due to the complex nature of such organic matters. None of the commercially available detergents effectively remove or reduce EPS related soiling. Biofilm is a group of microorganisms in which cells stick to each other or stick to a surface, such as a textile, dishware or hard surface or another kind of surface. These adherent cells are frequently embedded within a self-produced matrix of extracellular polymeric substance (EPS), which constitute 50% to 90% of the biofilm's total organic matter. EPS is mostly composed of polysaccharides (exopolysaccharides) and proteins, but include other macro-molecules such as DNA, lipids and human substances. EPS is the construction material of bacterial settlements and either remain attached to the cell's outer surface, or is secreted into its growth medium. EPS is required for the development and integrity of biofilms produced by a wide variety of bacteria. An important exopolysaccharide found in many bacterial biofilms is the linear poly-β-(1,6)-N-acetyl-D-glucosamine (PNAG). The exopolymer is found in biofilms of both Gram-positive bacteria, e.g. Staphylococcus species where it is referred to as polysaccharide intercellular adhesion (PIA), and Gram negative bacteria, e.g. Eschericia coli where it is refered to as PGA and Bordetella species where it is referred to as Bps. Both PIA and PGA are partially deacetylated. The production of PGA is in many bacteria controlled by proteins encoded by a cluster of four tightly linked genes in the pgaABCD operon. Among these four proteins, E. coli PgaB (EcPgaB) has been demonstrated experimentally to be a de-N-acetylase in vitro (Little et al., 2012, J Biol Chem 287: 31126-31137). Similaly, the PNAG polymer Bps is de-N-acetylated by the BpsB enzyme (Little et al., 2015 J Biol Chem. 2015 Sep. 11; 290(37):22827-40). The de-N-acetylation of PGA is essential for productive PGA-dependent biofilm formation. In Escherichia coli, partial de-N-acetylation of PGA by the periplasmic protein PgaB is required for polysaccharide-mediated biofilm formation. PGA molecules are de-N-acetylated by PgaB during export. A wide variety of biofilm-forming bacteria produce partially de-N-acetylated poly-β-1,6-N-acetyl-d-glucosamine (dPNAG) exopolysaccharides. The C-terminal domain of PgaB has structural similarity to many glycoside hydrolases and based on amino acid sequence identity, the PFAM database (Pfam version 31.0 Finn (2016). Nucleic Acids Research, Database Issue 44: D279-D285) recently categorized both BpsB and PgaB C-terminal domains as members of the GHL13 family (PFAM domain id PF14883). PgaB enzyme is further classified as a member of the family 4 carbohydrate esterases (CE4) enzymes as defined by the CAZY database [http://www.cazy.org/ (Coutinho & Henrissat, 1999)]. The polypeptides of the invention are BpsB and PgaB homologs comprising a GHL13 domain and show activity towards PNAG substrate. Some polypeptides of the invention also comprise the CE4 domain. Provided here are PgaA/BpsB homologs comprising a C-terminus glycosyl hydrolase domain (GHL13) and optionally a N-terminus deacetylase domain (CE4). The polypeptides of the invention were cloned and expressed with both domains (CE4 and GHL13) and some were truncated expressing the GHL13 domain alone without the deacetylase CE4 domain (the polypeptides shown in SEQ ID NO 49, SEQ ID NO 50, SEQ ID NO 51, SEQ ID NO 52, SEQ ID NO 53, SEQ ID NO 54, SEQ ID NO 55, SEQ ID NO 56, SEQ ID NO 57, SEQ ID NO 58). The inventors have shown that polypeptides comprising the GHL13 domain alone without the deacetylase CE4 domain have hydrolytic activity to PNAG and thus having the potential to reduce or remove components of EPS and related soiling of e.g. textiles.

One embodiment of the invention relates to polypeptides comprising the amino acids sequence shown in SEQ ID NO 49, SEQ ID NO 50, SEQ ID NO 51, SEQ ID NO 52, SEQ ID NO 53, SEQ ID NO 54, SEQ ID NO 55, SEQ ID NO 56, SEQ ID NO 57 or SEQ ID NO 58, wherein the polypeptide has hydrolytic activity. One embodiment of the invention relates to polypeptides comprising the amino acids sequence shown in SEQ ID NO 49, SEQ ID NO 50, SEQ ID NO 51, SEQ ID NO 52, SEQ ID NO 53, SEQ ID NO 54, SEQ ID NO 55, SEQ ID NO 56, SEQ ID NO 57, SEQ ID NO 58 or a polypeptide having at least 60%, at least 65%, at least 70%, at least 75%, at least 80%, at least 85%, at least 90% at least 95%, at least 96%, at least 97%, at least 98%, at least 99% or 100% sequence identity hereto, wherein the polypeptide has hydrolytic activity. One embodiment relates to a cleaning composition comprising a polypeptide selected from the group consisting of the polypeptides shown in SEQ ID NO 49, SEQ ID NO 50, SEQ ID NO 51, SEQ ID NO 52, SEQ ID NO 53, SEQ ID NO 54, SEQ ID NO 55, SEQ ID NO 56, SEQ ID NO 57, SEQ ID NO 58 and polypeptides having at least 80% sequence identity hereto, wherein the polypeptide has hydrolytic activity. One embodiment relates to a polypeptide comprising amino acid 4 to 324 of SEQ ID NO 49, one embodiment relates to a polypeptide comprising amino acid 7 to 333 of SEQ ID NO 50, one embodiment relates to a polypeptide comprising amino acid 7 to 334 of SEQ ID NO 51, one embodiment relates to a polypeptide comprising amino acid 4 to 330 of SEQ ID NO 52, one embodiment relates to a polypeptide comprising amino acid 7 to 333 of SEQ ID NO 53, one embodiment relates to a polypeptide comprising amino acid 4 to 330 of SEQ ID NO 54, one embodiment relates to a polypeptide comprising amino acid 4 to 330 of SEQ ID NO 55, one embodiment relates to a polypeptide comprising amino acid 4 to 324 of SEQ ID NO 56, one embodiment relates to a polypeptide comprising amino acid 4 to 324 of SEQ ID NO 57 and one embodiment relates to a polypeptide comprising amino acid 4 to 325 of SEQ ID NO 58.

The present invention provides polypeptides with hydrolase activity, wherein the polypeptides comprise the Pfam database domain GHL13 (PFAM domain id PF14883, Pfam version 31.0 Finn (2016). Nucleic Acids Research, Database Issue 44:D279-D285). The domain is a functional domain providing hydrolytic activity to the polypeptide. The polypeptides of the invention preferably in addition to the GHL13 domain comprises the CE4 domain (CE, CAZY database http://www.cazy.org/ (Coutinho & Henrissat, 1999) and have deacetylase activity. The invention further provides detergent compositions comprising polypeptides comprising the GHL13 domain and the use of such polypeptides for cleaning e.g. deep cleaning in cleaning processes. The polypeptides of the present invention comprising the GHL13 domain have beneficial properties such as cleaning e.g. deep cleaning properties in cleaning processes. Cleaning processes include laundry and dish wash. The polypeptides of the present invention belong to the glycoside hydrolases and preferably in addition to having hydrolytic activity preferably have deacetylase activity. Accordingly, the present invention relates to polypeptides comprising a GHL13 domain and preferably a CE4 domain, wherein the polypeptides is selected from the group consisting of:

(a) a polypeptide having at least 60%, at least 65%, at least 70%, at least 75%, at least 80%, at least 85%, at least 90% at least 95%, at least 96%, at least 97%, at least 98%, at least 99% or 100% sequence identity to the mature polypeptide shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86 or SEQ ID NO 89;

(b) a variant of the mature polypeptide shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86 or SEQ ID NO 89 comprising a substitution, deletion, and/or insertion at one or more (e.g., several) positions; and

(c) a fragment of the polypeptide of (a) or (b) that has hydrolytic and/or deacetylase activity.

The present invention relates to polypeptides comprising a CE4 domain, wherein the polypeptide is selected from the group of polypeptides; a polypeptide having at least 60%, at least 70%, at least 80%, at least 90%, at least 95% or 100% sequence identity to the mature polypeptide of SEQ ID NO 2, wherein the polypeptide comprises a CE4 domain defined as amino acid 71 to amino acid 300 of SEQ ID NO 2, a polypeptide having at least 60%, at least 70%, at least 80%, at least 90%, at least 95% or 100% sequence identity to the mature polypeptide of SEQ ID NO 5, wherein the polypeptide comprises a CE4 domain defined as amino acid 41 to amino acid 286 of SEQ ID NO 5, a polypeptide having at least 60%, at least 70%, at least 80%, at least 90%, at least 95% or 100% sequence identity to the mature polypeptide of SEQ ID NO 8, wherein the polypeptide comprises a CE4 domain defined as amino acid 71 to amino acid 300 of SEQ ID NO 8, a polypeptide having at least 60%, at least 70%, at least 80%, at least 90%, at least 95% or 100% sequence identity to the mature polypeptide of SEQ ID NO 11, wherein the polypeptide comprises a CE4 domain defined as amino acid 71 to amino acid 300 of SEQ ID NO 11, a polypeptide having at least 60%, at least 70%, at least 80%, at least 90%, at least 95% or 100% sequence identity to the mature polypeptide of SEQ ID NO 14, wherein the polypeptide comprises a CE4 domain defined as amino acid 64 to amino acid 293 of SEQ ID NO 14, a polypeptide having at least 60%, at least 70%, at least 80%, at least 90%, at least 95% or 100% sequence identity to the mature polypeptide of SEQ ID NO 17, wherein the polypeptide comprises a CE4 domain defined as amino acid 64 to amino acid 293 of SEQ ID NO 17, a polypeptide having at least 60%, at least 70%, at least 80%, at least 90%, at least 95% or 100% sequence identity to the mature polypeptide of SEQ ID NO 20, wherein the polypeptide comprises a CE4 domain defined as amino acid 45 to amino acid 290 of SEQ ID NO 20, a polypeptide having at least 60%, at least 70%, at least 80%, at least 90%, at least 95% or 100% sequence identity to the mature polypeptide of SEQ ID NO 23, wherein the polypeptide comprises a CE4 domain defined as amino acid 66 to amino acid 295 of SEQ ID NO 23, a polypeptide having at least 60%, at least 70%, at least 80%, at least 90%, at least 95% or 100% sequence identity to the mature polypeptide of SEQ ID NO 26, wherein the polypeptide comprises a CE4 domain defined as amino acid 45 to amino acid 368 of SEQ ID NO 26, a polypeptide having at least 60%, at least 70%, at least 80%, at least 90%, at least 95% or 100% sequence identity to the mature polypeptide of SEQ ID NO 29, wherein the polypeptide comprises a CE4 domain defined as amino acid 71 to amino acid 300 of SEQ ID NO 29, a polypeptide having at least 60%, at least 70%, at least 80%, at least 90%, at least 95% or 100% sequence identity to the mature polypeptide of SEQ ID NO 32, wherein the polypeptide comprises a CE4 domain defined as amino acid 47 to amino acid 286 of SEQ ID NO 32, a polypeptide having at least 60%, at least 70%, at least 80%, at least 90%, at least 95% or 100% sequence identity to the mature polypeptide of SEQ ID NO 35, wherein the polypeptide comprises a CE4 domain defined as amino acid 41 to amino acid 286 of SEQ ID NO 35, a polypeptide having at least 60%, at least 70%, at least 80%, at least 90%, at least 95% or 100% sequence identity to the mature polypeptide of SEQ ID NO 38, wherein the polypeptide comprises a CE4 domain defined as amino acid 41 to amino acid 286 of SEQ ID NO 38, a polypeptide having at least 60%, at least 70%, at least 80%, at least 90%, at least 95% or 100% sequence identity to the mature polypeptide of SEQ ID NO 41, wherein the polypeptide comprises a CE4 domain defined as amino acid 43 to amino acid 286 of SEQ ID NO 41, a polypeptide having at least 60%, at least 70%, at least 80%, at least 90%, at least 95% or 100% sequence identity to the mature polypeptide of SEQ ID NO 44, wherein the polypeptide comprises a CE4 domain defined as amino acid 42 to amino acid 286 of SEQ ID NO 44, a polypeptide having at least 60%, at least 70%, at least 80%, at least 90%, at least 95% or 100% sequence identity to the mature polypeptide of SEQ ID NO 47, wherein the polypeptide comprises a CE4 domain defined as amino acid 73 to amino acid 309 of SEQ ID NO 47, a polypeptide having at least 60%, at least 70%, at least 80%, at least 90%, at least 95% or 100% sequence identity to the mature polypeptide of SEQ ID NO 64, wherein the polypeptide comprises a CE4 domain defined as amino acid 78 to amino acid 324 of SEQ ID NO 64, a polypeptide having at least 60%, at least 70%, at least 80%, at least 90%, at least 95% or 100% sequence identity to the mature polypeptide of SEQ ID NO 67, wherein the polypeptide comprises a CE4 domain defined as amino acid 7 to amino acid 288 of SEQ ID NO 67, a polypeptide having at least 60%, at least 70%, at least 80%, at least 90%, at least 95% or 100% sequence identity to the mature polypeptide of SEQ ID NO 70, wherein the polypeptide comprises a CE4 domain defined as amino acid 47 to amino acid 285 of SEQ ID NO 70, a polypeptide having at least 60%, at least 70%, at least 80%, at least 90%, at least 95% or 100% sequence identity to the mature polypeptide of SEQ ID NO 73, wherein the polypeptide comprises a CE4 domain defined as amino acid 52 to amino acid 301 of SEQ ID NO 73, a polypeptide having at least 60%, at least 70%, at least 80%, at least 90%, at least 95% or 100% sequence identity to the mature polypeptide of SEQ ID NO 76, wherein the polypeptide comprises a CE4 domain defined as amino acid 64 to amino acid 231 of SEQ ID NO 76, a polypeptide having at least 60%, at least 70%, at least 80%, at least 90%, at least 95% or 100% sequence identity to the mature polypeptide of SEQ ID NO 79, wherein the polypeptide comprises a CE4 domain defined as amino acid 41 to amino acid 286 of SEQ ID NO 79, a polypeptide having at least 60%, at least 70%, at least 80%, at least 90%, at least 95% or 100% sequence identity to the mature polypeptide of SEQ ID NO 82, wherein the polypeptide comprises a CE4 domain defined as amino acid 43 to amino acid 286 of SEQ ID NO 82, a polypeptide having at least 60%, at least 70%, at least 80%, at least 90%, at least 95% or 100% sequence identity to the mature polypeptide of SEQ ID NO 85, wherein the polypeptide comprises a CE4 domain defined as amino acid 65 to amino acid 297 of SEQ ID NO 85 and a polypeptide having at least 60%, at least 70%, at least 80%, at least 90%, at least 95% or 100% sequence identity to the mature polypeptide of SEQ ID NO 88, wherein the polypeptide comprises a CE4 domain defined as amino acid 65 to amino acid 297 of SEQ ID NO 88.

The present invention also relates to polypeptides comprising a GHL13 domain, wherein the polypeptide is selected from the group of polypeptides; a polypeptide having at least 60%, at least 70%, at least 80%, at least 90%, at least 95% or 100% sequence identity to the mature polypeptide of SEQ ID NO 2, wherein the polypeptide comprises a GHL13 domain defined as amino acid 295 to amino acid 621 of SEQ ID NO 2, a polypeptide having at least 60%, at least 70%, at least 80%, at least 90%, at least 95% or 100% sequence identity to the mature polypeptide of SEQ ID NO 5, wherein the polypeptide comprises a GHL13 domain defined as amino acid 281 to amino acid 601 of SEQ ID NO 5, a polypeptide having at least 60%, at least 70%, at least 80%, at least 90%, at least 95% or 100% sequence identity to the mature polypeptide of SEQ ID NO 8, wherein the polypeptide comprises a GHL13 domain defined as amino acid 295 to amino acid 621 of SEQ ID NO 8, a polypeptide having at least 60%, at least 70%, at least 80%, at least 90%, at least 95% or 100% sequence identity to the mature polypeptide of SEQ ID NO 11, wherein the polypeptide comprises a GHL13 domain defined as amino acid 295 to amino acid 622 of SEQ ID NO 11, a polypeptide having at least 60%, at least 70%, at least 80%, at least 90%, at least 95% or 100% sequence identity to the mature polypeptide of SEQ ID NO 14, wherein the polypeptide comprises a GHL13 domain defined as amino acid 288 to amino acid 614 of SEQ ID NO 14, a polypeptide having at least 60%, at least 70%, at least 80%, at least 90%, at least 95% or 100% sequence identity to the mature polypeptide of SEQ ID NO 17, wherein the polypeptide comprises a GHL13 domain defined as amino acid 288 to amino acid 614 of SEQ ID NO 17, a polypeptide having at least 60%, at least 70%, at least 80%, at least 90%, at least 95% or 100% sequence identity to the mature polypeptide of SEQ ID NO 20, wherein the polypeptide comprises a GHL13 domain defined as amino acid 285 to amino acid 578 of SEQ ID NO 20, a polypeptide having at least 60%, at least 70%, at least 80%, at least 90%, at least 95% or 100% sequence identity to the mature polypeptide of SEQ ID NO 23, wherein the polypeptide comprises a GHL13 domain defined as amino acid 290 to amino acid 616 of SEQ ID NO 23, a polypeptide having at least 60%, at least 70%, at least 80%, at least 90%, at least 95% or 100% sequence identity to the mature polypeptide of SEQ ID NO 26, wherein the polypeptide comprises a GHL13 domain defined as amino acid 42 to amino acid 358 of SEQ ID NO 26, a polypeptide having at least 60%, at least 70%, at least 80%, at least 90%, at least 95% or 100% sequence identity to the mature polypeptide of SEQ ID NO 29, wherein the polypeptide comprises a GHL13 domain defined as amino acid 295 to amino acid 621 of SEQ ID NO 29, a polypeptide having at least 60%, at least 70%, at least 80%, at least 90%, at least 95% or 100% sequence identity to the mature polypeptide of SEQ ID NO 32, wherein the polypeptide comprises a GHL13 domain defined as amino acid 281 to amino acid 601 of SEQ ID NO 32, a polypeptide having at least 60%, at least 70%, at least 80%, at least 90%, at least 95% or 100% sequence identity to the mature polypeptide of SEQ ID NO 35, wherein the polypeptide comprises a GHL13 domain defined as amino acid 281 to amino acid 601 of SEQ ID NO 35, a polypeptide having at least 60%, at least 70%, at least 80%, at least 90%, at least 95% or 100% sequence identity to the mature polypeptide of SEQ ID NO 38, wherein the polypeptide comprises a GHL13 domain defined as amino acid 281 to amino acid 601 of SEQ ID NO 38, a polypeptide having at least 60%, at least 70%, at least 80%, at least 90%, at least 95% or 100% sequence identity to the mature polypeptide of SEQ ID NO 41, wherein the polypeptide comprises a GHL13 domain defined as amino acid 442 to amino acid 575 of SEQ ID NO 41, a polypeptide having at least 60%, at least 70%, at least 80%, at least 90%, at least 95% or 100% sequence identity to the mature polypeptide of SEQ ID NO 44, wherein the polypeptide comprises a GHL13 domain defined as amino acid 281 to amino acid 602 of SEQ ID NO 44, a polypeptide having at least 60%, at least 70%, at least 80%, at least 90%, at least 95% or 100% sequence identity to the mature polypeptide of SEQ ID NO 47, wherein the polypeptide comprises a GHL13 domain defined as amino acid 304 to amino acid 628 of SEQ ID NO 47, a polypeptide having at least 60%, at least 70%, at least 80%, at least 90%, at least 95% or 100% sequence identity to the mature polypeptide of SEQ ID NO 64, wherein the polypeptide comprises a GHL13 domain defined as amino acid 314 to amino acid 658 of SEQ ID NO 64, a polypeptide having at least 60%, at least 70%, at least 80%, at least 90%, at least 95% or 100% sequence identity to the mature polypeptide of SEQ ID NO 67, wherein the polypeptide comprises a GHL13 domain defined as amino acid 284 to amino acid 606 of SEQ ID NO 67, a polypeptide having at least 60%, at least 70%, at least 80%, at least 90%, at least 95% or 100% sequence identity to the mature polypeptide of SEQ ID NO 70, wherein the polypeptide comprises a GHL13 domain defined as amino acid 280 to amino acid 601 of SEQ ID NO 70, a polypeptide having at least 60%, at least 70%, at least 80%, at least 90%, at least 95% or 100% sequence identity to the mature polypeptide of SEQ ID NO 73, wherein the polypeptide comprises a GHL13 domain defined as amino acid 296 to amino acid 620 of SEQ ID NO 73, a polypeptide having at least 60%, at least 70%, at least 80%, at least 90%, at least 95% or 100% sequence identity to the mature polypeptide of SEQ ID NO 76, wherein the polypeptide comprises a GHL13 domain defined as amino acid 281 to amino acid 601 of SEQ ID NO 76, a polypeptide having at least 60%, at least 70%, at least 80%, at least 90%, at least 95% or 100% sequence identity to the mature polypeptide of SEQ ID NO 79, wherein the polypeptide comprises a GHL13 domain defined as amino acid 281 to amino acid 601 of SEQ ID NO 79, a polypeptide having at least 60%, at least 70%, at least 80%, at least 90%, at least 95% or 100% sequence identity to the mature polypeptide of SEQ ID NO 82, wherein the polypeptide comprises a GHL13 domain defined as amino acid 282 to amino acid 440 and amino acid 442 to amino acid 575 of SEQ ID NO 82, a polypeptide having at least 60%, at least 70%, at least 80%, at least 90%, at least 95% or 100% sequence identity to the mature polypeptide of SEQ ID NO 85, wherein the polypeptide comprises a GHL13 domain defined as amino acid 292 to amino acid 612 of SEQ ID NO 85 and a polypeptide having at least 60%, at least 70%, at least 80%, at least 90%, at least 95% or 100% sequence identity to the mature polypeptide of SEQ ID NO 88, wherein the polypeptide comprises a GHL13 domain defined as amino acid 277 to amino acid 615 of SEQ ID NO 88.

The invention also relates to polypeptides comprising the CE4 and the GHL13 domain wherein the polypeptide is selected from the group of polypeptides comprising; a polypeptide having at least 60%, at least 70%, at least 80%, at least 90%, at least 95% or 100% sequence identity to the mature polypeptide of SEQ ID NO 2, wherein the polypeptide comprises a CE4 domain defined as amino acid 71 to amino acid 300 of SEQ ID NO 2 and wherein the polypeptide comprises a GHL13 domain defined as amino acid 295 to amino acid 621 of SEQ ID NO 2, a polypeptide having at least 60%, at least 70%, at least 80%, at least 90%, at least 95% or 100% sequence identity to the mature polypeptide of SEQ ID NO 5, wherein the polypeptide comprises a CE4 domain defined as amino acid 41 to amino acid 286 of SEQ ID NO 5 and wherein the polypeptide comprises a GHL13 domain defined as amino acid 281 to amino acid 601 of SEQ ID NO 5, a polypeptide having at least 60%, at least 70%, at least 80%, at least 90%, at least 95% or 100% sequence identity to the mature polypeptide of SEQ ID NO 8, wherein the polypeptide comprises a CE4 domain defined as amino acid 71 to amino acid 300 of SEQ ID NO 8 and wherein the polypeptide comprises a GHL13 domain defined as amino acid 295 to amino acid 621 of SEQ ID NO 8, a polypeptide having at least 60%, at least 70%, at least 80%, at least 90%, at least 95% or 100% sequence identity to the mature polypeptide of SEQ ID NO 11, wherein the polypeptide comprises a CE4 domain defined as amino acid 71 to amino acid 300 of SEQ ID NO 11 and wherein the polypeptide comprises a GHL13 domain defined as amino acid 295 to amino acid 622 of SEQ ID NO 11, a polypeptide having at least 60%, at least 70%, at least 80%, at least 90%, at least 95% or 100% sequence identity to the mature polypeptide of SEQ ID NO 14, wherein the polypeptide comprises a CE4 domain defined as amino acid 64 to amino acid 293 of SEQ ID NO 14 and wherein the polypeptide comprises a GHL13 domain defined as amino acid 288 to amino acid 614 of SEQ ID NO 14, a polypeptide having at least 60%, at least 70%, at least 80%, at least 90%, at least 95% or 100% sequence identity to the mature polypeptide of SEQ ID NO 17, wherein the polypeptide comprises a CE4 domain defined as amino acid 64 to amino acid 293 of SEQ ID NO 17 and wherein the polypeptide comprises a GHL13 domain defined as amino acid 288 to amino acid 614 of SEQ ID NO 17, a polypeptide having at least 60%, at least 70%, at least 80%, at least 90%, at least 95% or 100% sequence identity to the mature polypeptide of SEQ ID NO 20, wherein the polypeptide comprises a CE4 domain defined as amino acid 45 to amino acid 290 of SEQ ID NO 20 and wherein the polypeptide comprises a GHL13 domain defined as amino acid 285 to amino acid 578 of SEQ ID NO 20, a polypeptide having at least 60%, at least 70%, at least 80%, at least 90%, at least 95% or 100% sequence identity to the mature polypeptide of SEQ ID NO 23, wherein the polypeptide comprises a CE4 domain defined as amino acid 66 to amino acid 295 of SEQ ID NO 23 and wherein the polypeptide comprises a GHL13 domain defined as amino acid 290 to amino acid 616 of SEQ ID NO 23, a polypeptide having at least 60%, at least 70%, at least 80%, at least 90%, at least 95% or 100% sequence identity to the mature polypeptide of SEQ ID NO 26, wherein the polypeptide comprises a CE4 domain defined as amino acid 45 to amino acid 368 of SEQ ID NO 26 and wherein the polypeptide comprises a GHL13 domain defined as amino acid 42 to amino acid 358 of SEQ ID NO 26, a polypeptide having at least 60%, at least 70%, at least 80%, at least 90%, at least 95% or 100% sequence identity to the mature polypeptide of SEQ ID NO 29, wherein the polypeptide comprises a CE4 domain defined as amino acid 71 to amino acid 300 of SEQ ID NO 29 and wherein the polypeptide comprises a GHL13 domain defined as amino acid 295 to amino acid 621 of SEQ ID NO 29, a polypeptide having at least 60%, at least 70%, at least 80%, at least 90%, at least 95% or 100% sequence identity to the mature polypeptide of SEQ ID NO 32, wherein the polypeptide comprises a CE4 domain defined as amino acid 47 to amino acid 286 of SEQ ID NO 32 and wherein the polypeptide comprises a GHL13 domain defined as amino acid 281 to amino acid 601 of SEQ ID NO 32, a polypeptide having at least 60%, at least 70%, at least 80%, at least 90%, at least 95% or 100% sequence identity to the mature polypeptide of SEQ ID NO 35, wherein the polypeptide comprises a CE4 domain defined as amino acid 41 to amino acid 286 of SEQ ID NO 35 and wherein the polypeptide comprises a GHL13 domain defined as amino acid 281 to amino acid 601 of SEQ ID NO 35, a polypeptide having at least 60%, at least 70%, at least 80%, at least 90%, at least 95% or 100% sequence identity to the mature polypeptide of SEQ ID NO 38, wherein the polypeptide comprises a CE4 domain defined as amino acid 41 to amino acid 286 of SEQ ID NO 38 and wherein the polypeptide comprises a GHL13 domain defined as amino acid 281 to amino acid 601 of SEQ ID NO 38, a polypeptide having at least 60%, at least 70%, at least 80%, at least 90%, at least 95% or 100% sequence identity to the mature polypeptide of SEQ ID NO 41, wherein the polypeptide comprises a CE4 domain defined as amino acid 43 to amino acid 286 of SEQ ID NO 41 and wherein the polypeptide comprises a GHL13 domain defined as amino acid 442 to amino acid 575 of SEQ ID NO 41, a polypeptide having at least 60%, at least 70%, at least 80%, at least 90%, at least 95% or 100% sequence identity to the mature polypeptide of SEQ ID NO 44, wherein the polypeptide comprises a CE4 domain defined as amino acid 42 to amino acid 286 of SEQ ID NO 44 and wherein the polypeptide comprises a GHL13 domain defined as amino acid 281 to amino acid 602 of SEQ ID NO 44,a polypeptide having at least 60%, at least 70%, at least 80%, at least 90%, at least 95% or 100% sequence identity to the mature polypeptide of SEQ ID NO 47, wherein the polypeptide comprises a CE4 domain defined as amino acid 73 to amino acid 309 of SEQ ID NO 47 and wherein the polypeptide comprises a GHL13 domain defined as amino acid 304 to amino acid 628 of SEQ ID NO 47, a polypeptide having at least 60%, at least 70%, at least 80%, at least 90%, at least 95% or 100% sequence identity to the mature polypeptide of SEQ ID NO 64, wherein the polypeptide comprises a CE4 domain defined as amino acid 78 to amino acid 324 of SEQ ID NO 64 and wherein the polypeptide comprises a GHL13 domain defined as amino acid 315 to amino acid 658 of SEQ ID NO 64, a polypeptide having at least 60%, at least 70%, at least 80%, at least 90%, at least 95% or 100% sequence identity to the mature polypeptide of SEQ ID NO 67, wherein the polypeptide comprises a CE4 domain defined as amino acid 7 to amino acid 288 of SEQ ID NO 67 and wherein the polypeptide comprises a GHL13 domain defined as amino acid 284 to amino acid 606 of SEQ ID NO 67, a polypeptide having at least 60%, at least 70%, at least 80%, at least 90%, at least 95% or 100% sequence identity to the mature polypeptide of SEQ ID NO 70, wherein the polypeptide comprises a CE4 domain defined as amino acid 47 to amino acid 285 of SEQ ID NO 70 and wherein the polypeptide comprises a GHL13 domain defined as amino acid 280 to amino acid 601 of SEQ ID NO 70, a polypeptide having at least 60%, at least 70%, at least 80%, at least 90%, at least 95% or 100% sequence identity to the mature polypeptide of SEQ ID NO 73, wherein the polypeptide comprises a CE4 domain defined as amino acid 52 to amino acid 301 of SEQ ID NO 73 and wherein the polypeptide comprises a GHL13 domain defined as amino acid 296 to amino acid 620 of SEQ ID NO 73, a polypeptide having at least 60%, at least 70%, at least 80%, at least 90%, at least 95% or 100% sequence identity to the mature polypeptide of SEQ ID NO 76, wherein the polypeptide comprises a CE4 domain defined as amino acid 64 to amino acid 231 of SEQ ID NO 76 and wherein the polypeptide comprises a GHL13 domain defined as amino acid 281 to amino acid 601 of SEQ ID NO 76, a polypeptide having at least 60%, at least 70%, at least 80%, at least 90%, at least 95% or 100% sequence identity to the mature polypeptide of SEQ ID NO 79, wherein the polypeptide comprises a CE4 domain defined as amino acid 41 to amino acid 286 of SEQ ID NO 79 and wherein the polypeptide comprises a GHL13 domain defined as amino acid 281 to amino acid 601 of SEQ ID NO 79, a polypeptide having at least 60%, at least 70%, at least 80%, at least 90%, at least 95% or 100% sequence identity to the mature polypeptide of SEQ ID NO 82, wherein the polypeptide comprises a CE4 domain defined as amino acid 43 to amino acid 286 of SEQ ID NO 82 and wherein the polypeptide comprises a GHL13 domain defined as amino acid 282 to amino acid 440 and amino acid 442 to amino acid 575 of SEQ ID NO 82, a polypeptide having at least 60%, at least 70%, at least 80%, at least 90%, at least 95% or 100% sequence identity to the mature polypeptide of SEQ ID NO 85, wherein the polypeptide comprises a CE4 domain defined as amino acid 65 to amino acid 297 of SEQ ID NO 85 and wherein the polypeptide comprises a GHL13 domain defined as amino acid 292 to amino acid 612 of SEQ ID NO 85 and a polypeptide having at least 60%, at least 70%, at least 80%, at least 90%, at least 95% or 100% sequence identity to the mature polypeptide of SEQ ID NO 88, wherein the polypeptide comprises a CE4 domain defined as amino acid 57 to amino acid 287 of SEQ ID NO 88 and wherein the polypeptide comprises a GHL13 domain defined as amino acid 277 to amino acid 615 of SEQ ID NO 88.

The invention also relates to a polypeptide comprising amino acids 71 to 621 of SEQ ID NO 2, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 71 to 621 of SEQ ID NO 2.

The invention also relates to a polypeptide comprising amino acids 41 to 601 of SEQ ID NO 5, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 41 to 601 of SEQ ID NO 5.

The invention also relates to a polypeptide comprising amino acids 71 to 621 of SEQ ID NO 8, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 71 to 621 of SEQ ID NO 8.

The invention also relates to a polypeptide comprising amino acids 71 to 622 of SEQ ID NO 11, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 71 to 622 of SEQ ID NO 11.

The invention also relates to a polypeptide comprising amino acids 64 to 614 of SEQ ID NO 14, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 64 to 614 of SEQ ID NO 14.

The invention also relates to a polypeptide comprising amino acids 64 to 614 of SEQ ID NO 17, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 64 to 614 of SEQ ID NO 17.

The invention also relates to a polypeptide comprising amino acids 45 to 578 of SEQ ID NO 20, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 45 to 578 of SEQ ID NO 20.

The invention also relates to a polypeptide comprising amino acids 66 to 616 of SEQ ID NO 23, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 66 to 616 of SEQ ID NO 23.

The invention also relates to a polypeptide comprising amino acids 1 to 358 of SEQ ID NO 26, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 1 to 358 of SEQ ID NO 26.

The invention also relates to a polypeptide comprising amino acids 71 to 621 of SEQ ID NO 29, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 71 to 621 of SEQ ID NO 29.

The invention also relates to a polypeptide comprising amino acids 47 to 601 of SEQ ID NO 32, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 47 to 601 of SEQ ID NO 32.

The invention also relates to a polypeptide comprising amino acids 41 to 601 of SEQ ID NO 35, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 66 to 601 of SEQ ID NO 35.

The invention also relates to a polypeptide comprising amino acids 41 to 601 of SEQ ID NO 38, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 66 to 601 of SEQ ID NO 38.

The invention also relates to a polypeptide comprising amino acids 43 to 575 of SEQ ID NO 41, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 66 to 575 of SEQ ID NO 41.

The invention also relates to a polypeptide comprising amino acids 42 to 602 of SEQ ID NO 44, wherein the polypeptide has at least 80 at least 60%, at least 65%, at least 70%, at least 75%, at least 80%, at least 85%, at least 90% at least 95%, at least 96%, at least 97%, at least 98%, at least 99% or 100% sequence identity to amino acids 66 to 602 of SEQ ID NO 44.

The invention also relates to a polypeptide comprising amino acids 73 to 628 of SEQ ID NO 47, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 73 to 628 of SEQ ID NO 47.

The invention also relates to a polypeptide comprising amino acids 78 to 658 of SEQ ID NO 64, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 78 to 658 of SEQ ID NO 64.

The invention also relates to a polypeptide comprising amino acids 7 to 606 of SEQ ID NO 67, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 7 to 606 of SEQ ID NO 67.

The invention also relates to a polypeptide comprising amino acids 47 to 601 of SEQ ID NO 70, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 47 to 601 of SEQ ID NO 70.

The invention also relates to a polypeptide comprising amino acids 52 to 620 of SEQ ID NO 73, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 52 to 620 of SEQ ID NO 73.

The invention also relates to a polypeptide comprising amino acids 64 to 601 of SEQ ID NO 76, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 64 to 601 of SEQ ID NO 76.

The invention also relates to a polypeptide comprising amino acids 41 to 601 of SEQ ID NO 79, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 41 to 601 of SEQ ID NO 79.

The invention also relates to a polypeptide comprising amino acids 43 to 575 of SEQ ID NO 82, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 43 to 575 of SEQ ID NO 82.

The invention also relates to a polypeptide comprising amino acids 65 to 612 of SEQ ID NO 85, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 65 to 612 of SEQ ID NO 85.

The invention also relates to a polypeptide comprising amino acids 65 to 612 of SEQ ID NO 85, wherein the polypeptide has at least 60%, at least 65%, at least 70%, at least 75%, at least 80%, at least 85%, at least 90% at least 95%, at least 96%, at least 97%, at least 98%, at least 99% or 100% sequence identity to amino acids 57 to 615 of SEQ ID NO 88.

One aspect of the invention relates to a polypeptide having hydrolytic and/or deacetyl activity, wherein the polypeptide selected from the group consisting of:

(a) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 3;

(b) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 6;

(c) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 9;

(d) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 12;

(e) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 15;

(f) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 18;

(g) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 21;

(h) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 24;

(i) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO 27;

(j) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 30;

(k) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 33;

(l) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 36;

(m) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 39;

(n) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 42;

(o) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 45;

(p) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 48;

(q) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 65;

(r) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 68;

(s) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 71;

(t) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 74;

(u) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 77;

(v) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 80;

(x) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 83;

(y) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 86; and

(z) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 89.

As mentioned above the polypeptides of the invention comprises one or two of the domains GHL13 and/or CE4. The polypeptides and the domains comprises characteristic domains which are conserved and specific for the polypeptides of the invention and/or for the specific domains.

One embodiment relates to polypeptides belonging to the YPDDF-clade, wherein the polypeptides comprises the GHL13 domain with one or more of the motifs [YW]PX[DN]F (SEQ ID NO 59) or [MEYF]AM[PG] (SEQ ID NO 60) and/or a CE 4 domain. The polypeptides with the CE4 domain comprise the conserved WPY motif, corresponding to amino acids 211 to 213 of SEQ ID NO 36. One embodiment relates to a GHL13 glycosyl hydrolase comprising one or more, or all of the motif(s) [YW]PX[DN]F (SEQ ID NO 59) or [MEYF]AM[PG] (SEQ ID NO 60) and/or a CE 4 domain. One embodiment relates to a GHL13 glycosyl hydrolase comprising two or more, or all of the motif(s) [YW]PX[DN]F (SEQ ID NO 59) or [MEYF]AM[PG] (SEQ ID NO 60) and/or a CE 4 domain. One embodiment relates to a GHL13 glycosyl hydrolase comprising all three motif(s) [YW]PX[DN]F (SEQ ID NO 59) or [MEYF]AM[PG] (SEQ ID NO 60) and/or a CE 4 domain.

One aspect of the invention relates to a polypeptide having hydrolytic and/or deacetyl activity, wherein the polypeptide is of the YPDDF clade, comprising one or more of the motif(s) [Y/W]PX[D/N]F (SEQ ID NO 59), [M/E/Y/F]AM[P/G] (SEQ ID NO 60) or WPY and wherein the polypeptide is selected from the group consisting of:

(a) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 3;

(b) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 6;

(c) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 9;

(d) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 12;

(e) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 15;

(f) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 18;

(g) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 21;

(h) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 24;

(i) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO 27;

(j) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 30;

(k) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 33;

(l) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 36;

(m) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 39;

(n) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 42;

(o) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 45;

(p) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 48;

(q) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 65;

(r) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 68;

(s) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 71;

(t) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 74;

(u) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 77;

(v) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 80;

(x) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 83;

(y) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 86; and

(z) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 89.

One aspect of the invention relates to a polypeptide having hydrolytic and/or deacetyl activity, wherein the polypeptide is of the YPDDF clade, comprising two or all three motif(s) [Y/W]PX[D/N]F (SEQ ID NO 59), [M/E/Y/F]AM[P/G] (SEQ ID NO 60) or WPY and wherein the polypeptide is selected from the group consisting of:

(a) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 3;

(b) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 6;

(c) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 9;

(d) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 12;

(e) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 15;

(f) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 18;

(g) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 21;

(h) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 24;

(i) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO 27;

(j) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 30;

(k) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 33;

(l) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 36;

(m) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 39;

(n) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 42;

(o) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 45;

(p) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 48;

(q) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 65;

(r) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 68;

(s) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 71;

(t) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 74;

(u) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 77;

(v) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 80;

(x) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 83;

(y) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 86; and

(z) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 89.

The PgaB/BpsB homolog polypeptide of the invention belongs to the YPDDF clade and comprises the GHL13 catalytic domain. In some embodiment, the polypeptides additionally comprise the CE4 domain. The polypeptides have PNAG (poly-N-acetyl glucosamine)-hydrolyzing activity in detergent under laundry relevant conditions and may be used for detergent and cleaning processes for cleaning e.g. deep cleaning stains e.g. PNAG or other polysaccharides of surfaces such as textiles.

One embodiment relates to a GHL13 polypeptide, having hydrolytic activity e.g. to poly-N-acetyl glucosamine, wherein the polypeptide is selected from the group consisting of:
(a) a polypeptide having at least 97%, at least 98%, at least 99% or 100% sequence identity to the polypeptide of SEQ ID NO: 3;
(b) a polypeptide having at least 90% at least 95%, at least 96%, at least 97%, at least 98%, at least 99% or 100% sequence identity to the polypeptide of SEQ ID NO: 6;
(c) a polypeptide having at least 99% or 100% sequence identity to the polypeptide of SEQ ID NO: 9;
(d) a polypeptide having at least 97%, at least 98%, at least 99% or 100% sequence identity to the polypeptide of SEQ ID NO: 12;
(e) a polypeptide having at least 99% or 100% sequence sequence identity to the polypeptide of SEQ ID NO: 15;
(f) a polypeptide having at least 97%, at least 98%, at least 99% or 100% sequence identity to the polypeptide of SEQ ID NO: 18;
(g) a polypeptide having at least 96%, at least 97%, at least 98%, at least 99% or 100% sequence identity to the polypeptide of SEQ ID NO: 21;
(h) a polypeptide having at least 99% or 100% sequence identity to the polypeptide of SEQ ID NO: 24;
(i) a polypeptide having 100% sequence identity to the polypeptide of SEQ ID NO 27;
(j) a polypeptide having at least 99% or 100% sequence identity to the polypeptide of SEQ ID NO: 30;
(k) a polypeptide having at least 90% at least 95%, at least 96%, at least 97%, at least 98%, at least 99% or 100% sequence identity to the polypeptide of SEQ ID NO: 33;
(l) a polypeptide having 100% sequence identity to the polypeptide of SEQ ID NO: 36;
(m) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 39;
(n) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 42;
(o) a polypeptide having at least 80%, at least 85%, at least 90% at least 95%, at least 96%, at least 97%, at least 98%, at least 99% or 100% sequence identity to the polypeptide of SEQ ID NO: 45;
(p) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 48;
(q) a polypeptide having 100% sequence identity to the polypeptide of SEQ ID NO: 65;
(r) a polypeptide having at least 70%, at least 75%, at least 80%, at least 85%, at least 90% at least 95%, at least 96%, at least 97%, at least 98%, at least 99% or 100% sequence identity to the polypeptide of SEQ ID NO: 68;
(s) a polypeptide having 100% sequence identity to the polypeptide of SEQ ID NO: 71;
(t) a polypeptide having 100% sequence identity to the polypeptide of SEQ ID NO: 74;
(u) a polypeptide 100% sequence identity to the polypeptide of SEQ ID NO: 77;
(v) a polypeptide having 100% sequence identity to the polypeptide of SEQ ID NO: 80;
(x) a polypeptide having at least 98%, at least 99% or 100% sequence identity to the polypeptide of SEQ ID NO: 83;
(y) a polypeptide having 100% sequence identity to the polypeptide of SEQ ID NO: 86; and
(z) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 89.

One embodiment relates to a GHL13 polypeptide, having hydrolytic activity e.g. to poly-N-acetyl glucosamine, wherein the polypeptide is selected from the group consisting of:
(a) a polypeptide having at least 97%, at least 98%, at least 99% or 100% sequence identity to the polypeptide of SEQ ID NO: 3;
(b) a polypeptide having at least 90% at least 95%, at least 96%, at least 97%, at least 98%, at least 99% or 100% sequence identity to the polypeptide of SEQ ID NO: 6;
(c) a polypeptide having at least 97%, at least 98%, at least 99% or 100% sequence identity to the polypeptide of SEQ ID NO: 12;
(d) a polypeptide having at least 97%, at least 98%, at least 99% or 100% sequence identity to the polypeptide of SEQ ID NO: 18;
(e) a polypeptide having at least 96%, at least 97%, at least 98%, at least 99% or 100% sequence identity to the polypeptide of SEQ ID NO: 21;
(f) a polypeptide having at least 90% at least 95%, at least 96%, at least 97%, at least 98%, at least 99% or 100% sequence identity to the polypeptide of SEQ ID NO: 33;
(g) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 39;
(h) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 42;
(i) a polypeptide having at least 80%, at least 85%, at least 90% at least 95%, at least 96%, at least 97%, at least 98%, at least 99% or 100% sequence identity to the polypeptide of SEQ ID NO: 45;
(j) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 48;
(k) a polypeptide having at least 70%, at least 75%, at least 80%, at least 85%, at least 90% at least 95%, at least 96%, at least 97%, at least 98%, at least 99% or 100% sequence identity to the polypeptide of SEQ ID NO: 68;
(l) a polypeptide having at least 98%, at least 99% or 100% sequence identity to the polypeptide of SEQ ID NO: 83; and
(m) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 89.

The present disclosure also provides a method for preventing, reduction or removal of a PNAG containing organic soiling on an item comprising applying at least one polypeptide comprising the GHL13 domain to the item and optionally rinse the item. The item is preferably a textile or a hard surface, such as dish ware.

Organic matters such as EPS or components hereof may have glue-like properties and the presence of biofilm on e.g. textiles may result in items or areas on items which are “sticky”. Soil will in general adhere to the sticky areas and such soil has shown difficult to remove by commercially available detergent compositions. Further, when dirty laundry items are washed together with less dirty laundry items the dirt present in the wash liquor tend to stick to the biofilm or biofilm components. As a result, the laundry item is more “soiled” after wash than before wash. This is effect may also be termed re-deposition.

Some biofilm or EPS components may cause malodor, which is compounds with an unpleasant smell. Unpleasant smells can be sweat or body odor adhered to an item which has been in contact with human or animal, typical smells includes old man, damp and moldy smells often caused by volatile organic compounds such as e.g. certain alcohols and aldehydes. Another example of malodor can be the odor from tobacco smoke or spices, which sticks to items for example curry or other exotic spices which smell strongly. Such smells are not necessarily caused by the biofilm or EPS components but the smells may be “captured” in the organic structure and thus be difficult to remove.

In particular, the polypeptides comprising a GHL13 and/or CE4 domain(s), as defined above are useful in reducing and preventing malodor of items being washed. The inventors have surprisingly found that the polypeptides comprising the GHL13 domain and having PNAG-hydrolyzing activity are useful for reducing or removing laundry associated PNAG.

The polypeptides of the present invention comprise a C-terminus glycosyl hydrolase domain termed GHL13, hypothetical glycoside hydrolases domain 13 (PFAM domain id PF14883, Pfam version 31.0 Finn (2016). Nucleic Acids Research, Database Issue 44:D279-D285). The polypeptides of the invention have activity towards poly-β-1,6-N-acetyl-D-glucosamine (PNAG) but are distantly related to other PNAG active glycosyl hydrolase molecules, such as dispersins. The polypeptides of the invention are related to PgaB, which is a molecule involved in the PNAG biosynthetic pathway of Gram negative bacteria, where it plays a key role in the deacetylation and secretion of PNAG during biosynthesis. The polypeptides of the invention are also related to homologues of BspB from Bordetella bronchiseptica which is an enzyme with similar function to PgaB as described above. PgaB enzyme is further classified as a member of the family 4 carbohydrate esterases (CE4) enzymes as defined by the CAZY database [http://www.cazy.org/ (Coutinho & Henrissat, 1999)]. The polypeptides of the invention may in addition to the GHL13 domain also comprise the CE4 domain. The CE4 deacetylase domain is related to IcaB, a PNAG deacetylase found in Gram positive bacteria.

The GHL13 domain is identified as amino acid 295 to amino acid 621 of SEQ ID NO 2, as amino acid 281 to amino acid 601of SEQ ID NO 5, as amino acid 295 to amino acid 621 of SEQ ID NO 8, as amino acid 295 to amino acid 622 of SEQ ID NO 11, as amino acid 288 to amino acid 614 of SEQ ID NO 14, as amino acid 288 to amino acid 614 of SEQ ID NO 17, as amino acid 285 to amino acid 578 of SEQ ID NO 20, as amino acid 290 to amino acid 616 of SEQ ID NO 23, as amino acid 42 to amino acid 358 of SEQ ID NO 26, as amino acid 295 to amino acid 621 of SEQ ID NO 29, as amino acid 281 to amino acid 601 of SEQ ID NO 32, as amino acid 281 to amino acid 601 of SEQ ID NO 35, as amino acid 281 to amino acid 601 of SEQ ID NO 38, as amino acid 442 to amino acid 575 of SEQ ID NO 41, as amino acid 281 to amino acid 602 of SEQ ID NO 44, as amino acid 304 to amino acid 628 of SEQ ID NO 47, as amino acid 314 to amino acid 658 of SEQ ID NO 64, as amino acid 284 to amino acid 606 of SEQ ID NO 67, as amino acid 280 to amino acid 601 of SEQ ID NO 70, as amino acid 296 to amino acid 620 of SEQ ID NO 73, as amino acid 281 to amino acid 601 of SEQ ID NO 76, as amino acid 281 to amino acid 601 of SEQ ID NO 79, as amino acid 282 to amino acid 440 and as amino acid 442 to amino acid 575 of SEQ ID NO 82, as amino acid 292 to amino acid 612 of SEQ ID NO 85, as amino acid 277 to amino acid 615 of SEQ ID NO 88.

The CE4 domain defined as amino acid 71 to amino acid 300 of SEQ ID NO 2, as amino acid 41 to amino acid 286 of SEQ ID NO 5, as amino acid 71 to amino acid 300 of SEQ ID NO 8, as amino acid 71 to amino acid 300 of SEQ ID NO 11, as amino acid 64 to amino acid 293 of SEQ ID NO 14, as amino acid 64 to amino acid 293 of SEQ ID NO 17, as amino acid 45 to amino acid 290 of SEQ ID NO 20, as amino acid 66 to amino acid 295 of SEQ ID NO 23, as amino acid amino acid 45 to amino acid 368 of SEQ ID NO 26, as amino acid 71 to amino acid 300 of SEQ ID NO 29, as amino acid 47 to amino acid 286 of SEQ ID NO 32, as amino acid 41 to amino acid 286 of SEQ ID NO 35, as amino acid 41 to amino acid 286 of SEQ ID NO 38, as amino acid 43 to amino acid 286 of SEQ ID NO 41, as amino acid 42 to amino acid 286 of SEQ ID NO 44,as amino acid 73 to amino acid 309 of SEQ ID NO 47, as amino acid 292 to amino acid 612 of SEQ ID NO 64, as amino acid 284 to amino acid 606 of SEQ ID NO 67, as amino acid 280 to amino acid 601 of SEQ ID NO 70, as amino acid 296 to amino acid 620 of SEQ ID NO 73, as amino acid 281 to amino acid 601 of SEQ ID NO 76, as amino acid 281 to amino acid 601 of SEQ ID NO 79, as amino acid 282 to amino acid 440 and amino acid 442 to amino acid 575 of SEQ ID NO 82, as amino acid 292 to amino acid 612 of SEQ ID NO 85 and as amino acid 277 to amino acid 615 of SEQ ID NO 88. The polypeptides of the invention show wash activity in Model Aon EPS stained swatches comprising PNAG. Applying the polypeptides of the invention in cleaning processes such as laundry show reduction or removal of PNAG staining. The polypeptides of the invention are therefore useful for reduction and/or removal of PNAG comprising components such as EPS. One embodiment of the invention relates to the use of polypeptides comprising the GHL13 domain and/or CE4 domain in cleaning processes for cleaning e.g. deep cleaning of textiles and hard surfaces, preferably in cleaning processes or detergents limited to non-medical surfaces. Such cleaning processes may be dish wash or laundry.

In one aspect, the polypeptides of the present invention have at least 20%, e.g., at least 40%, at least 50%, at least 60%, at least 70%, at least 80%, at least 90%, at least 95%, or at least 100% of the PNAG-hydrolyzing activity of the mature polypeptide e.g. the polypeptide shown in SEQ ID NO 3.

One embodiment of the invention relates to a polypeptide having hydrolytic and/or deacetylase activity comprising amino acids 1 to 645 of SEQ ID NO 2, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 645 of SEQ ID NO 2 i.e. the mature polypeptide shown in SEQ ID NO 3.

One embodiment of the invention relates to a polypeptide having hydrolytic and/or deacetylase activity comprising amino acids 1 to 629 of SEQ ID NO 5, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 629 of SEQ ID NO 5 i.e. the mature polypeptide shown in SEQ ID NO 6.

One embodiment of the invention relates to a polypeptide having hydrolytic and/or deacetylase activity comprising amino acids 1 to 645 of SEQ ID NO 8, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 645 of SEQ ID NO 8 i.e. the mature polypeptide shown in SEQ ID NO 9.

One embodiment of the invention relates to a polypeptide having hydrolytic and/or deacetylase activity comprising amino acids 1 to 646 of SEQ ID NO 11, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 646 of SEQ ID NO 11 i.e. the mature polypeptide shown in SEQ ID NO 12.

One embodiment of the invention relates to a polypeptide having hydrolytic and/or deacetylase activity comprising amino acids 1 to 638 of SEQ ID NO 14, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 638 of SEQ ID NO 14 i.e. the mature polypeptide shown in SEQ ID NO 15.

One embodiment of the invention relates to a polypeptide having hydrolytic and/or deacetylase activity comprising amino acids 1 to 638 of SEQ ID NO 17, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 638 of SEQ ID NO 17 i.e. the mature polypeptide shown in SEQ ID NO 18.

One embodiment of the invention relates to a polypeptide having hydrolytic and/or deacetylase activity comprising amino acids 1 to 606 of SEQ ID NO 20, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 606 of SEQ ID NO 20 i.e. the mature polypeptide shown in SEQ ID NO 21.

One embodiment of the invention relates to a polypeptide having hydrolytic and/or deacetylase activity comprising amino acids 1 to 640 of SEQ ID NO 23, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 640 of SEQ ID NO 23 i.e. the mature polypeptide shown in SEQ ID NO 24.

One embodiment of the invention relates to a polypeptide having hydrolytic activity comprising amino acids 1 to 380 of SEQ ID NO 26, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 380 of SEQ ID NO 26 i.e. the mature polypeptide shown in SEQ ID NO 27.

One embodiment of the invention relates to a polypeptide having hydrolytic and/or deacetylase activity comprising amino acids 1 to 645 of SEQ ID NO 29, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 645 of SEQ ID NO 29 i.e. the mature polypeptide shown in SEQ ID NO 30.

One embodiment of the invention relates to a polypeptide having hydrolytic and/or deacetylase activity comprising amino acids 1 to 629 of SEQ ID NO 32, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 629 of SEQ ID NO 32 i.e. the mature polypeptide shown in SEQ ID NO 33.

One embodiment of the invention relates to a polypeptide having hydrolytic and/or deacetylase activity comprising amino acids 1 to 629 of SEQ ID NO 35, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 629 of SEQ ID NO 35 i.e. the mature polypeptide shown in SEQ ID NO 36.

One embodiment of the invention relates to a polypeptide having hydrolytic and/or deacetylase activity comprising amino acids 1 to 629 of SEQ ID NO 38, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 629 of SEQ ID NO 38 i.e. the mature polypeptide shown in SEQ ID NO 39.

One embodiment of the invention relates to a polypeptide having hydrolytic and/or deacetylase activity comprising amino acids 1 to 605 of SEQ ID NO 41, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 605 of SEQ ID NO 41 i.e. the mature polypeptide shown in SEQ ID NO 42.

One embodiment of the invention relates to a polypeptide having hydrolytic and/or deacetylase activity comprising amino acids 1 to 630 of SEQ ID NO 44, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 630 of SEQ ID NO 44 i.e. the mature polypeptide shown in SEQ ID NO 45.

One embodiment of the invention relates to a polypeptide having hydrolytic and/or deacetylase activity comprising amino acids 1 to 678 of SEQ ID NO 47, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 678 of SEQ ID NO 47 i.e. the mature polypeptide shown in SEQ ID NO 48.

One embodiment of the invention relates to a polypeptide having hydrolytic and/or deacetylase activity comprising amino acids 1 to 667 of SEQ ID NO 64, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 667 of SEQ ID NO 64 i.e. the mature polypeptide shown in SEQ ID NO 65.

One embodiment of the invention relates to a polypeptide having hydrolytic and/or deacetylase activity comprising amino acids 1 to 632 of SEQ ID NO 67, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 632 of SEQ ID NO 67 i.e. the mature polypeptide shown in SEQ ID NO 68.

One embodiment of the invention relates to a polypeptide having hydrolytic and/or deacetylase activity comprising amino acids 1 to 629 of SEQ ID NO 70, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 629 of SEQ ID NO 70 i.e. the mature polypeptide shown in SEQ ID NO 71.

One embodiment of the invention relates to a polypeptide having hydrolytic and/or deacetylase activity comprising amino acids 1 to 647 of SEQ ID NO 73, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 647 of SEQ ID NO 73 i.e. the mature polypeptide shown in SEQ ID NO 74.

One embodiment of the invention relates to a polypeptide having hydrolytic and/or deacetylase activity comprising amino acids 1 to 629 of SEQ ID NO 76, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 629 of SEQ ID NO 76 i.e. the mature polypeptide shown in SEQ ID NO 77.

One embodiment of the invention relates to a polypeptide having hydrolytic and/or deacetylase activity comprising amino acids 1 to 629 of SEQ ID NO 79, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 629 of SEQ ID NO 79 i.e. the mature polypeptide shown in SEQ ID NO 80.

One embodiment of the invention relates to a polypeptide having hydrolytic and/or deacetylase activity comprising amino acids 1 to 605 of SEQ ID NO 82, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 605 of SEQ ID NO 82 i.e. the mature polypeptide shown in SEQ ID NO 83.

One embodiment of the invention relates to a polypeptide having hydrolytic and/or deacetylase activity comprising amino acids 1 to 662 of SEQ ID NO 85, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 662 of SEQ ID NO 85 i.e. the mature polypeptide shown in SEQ ID NO 86.

One embodiment of the invention relates to a polypeptide having hydrolytic and/or deacetylase activity comprising amino acids 1 to 630 of SEQ ID NO 88, wherein the polypeptide has at least 60%, such as at least 65%, at least 70%, at least 75%, at least 80%, at least 85%, at least 90%, at least 95%, at least 98%, at least 99% or at least 100% sequence identity to amino acids 1 to 630 of SEQ ID NO 88 i.e. the mature polypeptide shown in SEQ ID NO 89.

In some aspect, the invention relates to a polypeptide which comprises or consists of the amino acid sequence shown in SEQ ID NO 3.

In some aspect, the invention relates to a polypeptide which comprises or consists of the amino acid sequence shown in SEQ ID NO 6.

In some aspect, the invention relates to a polypeptide which comprises or consists of the amino acid sequence shown in SEQ ID NO 9.

In some aspect, the invention relates to a polypeptide which comprises or consists of the amino acid sequence shown in SEQ ID NO 12.

In some aspect, the invention relates to a polypeptide which comprises or consists of the amino acid sequence shown in SEQ ID NO 15.

In some aspect, the invention relates to a polypeptide which comprises or consists of the amino acid sequence shown in SEQ ID NO 18.

In some aspect, the invention relates to a polypeptide which comprises or consists of the amino acid sequence shown in SEQ ID NO 21.

In some aspect, the invention relates to a polypeptide which comprises or consists of the amino acid sequence shown in SEQ ID NO 24.

In some aspect, the invention relates to a polypeptide which comprises or consists of the amino acid sequence shown in SEQ ID NO 27.

In some aspect, the invention relates to a polypeptide which comprises or consists of the amino acid sequence shown in SEQ ID NO 30.

In some aspect, the invention relates to a polypeptide which comprises or consists of the amino acid sequence shown in SEQ ID NO 33.

In some aspect, the invention relates to a polypeptide which comprises or consists of the amino acid sequence shown in SEQ ID NO 36.

In some aspect, the invention relates to a polypeptide which comprises or consists of the amino acid sequence shown in SEQ ID NO 39.

In some aspect, the invention relates to a polypeptide which comprises or consists of the amino acid sequence shown in SEQ ID NO 42.

In some aspect, the invention relates to a polypeptide which comprises or consists of the amino acid sequence shown in SEQ ID NO 45.

In some aspect, the invention relates to a polypeptide which comprises or consists of the amino acid sequence shown in SEQ ID NO 48.

In some aspect, the invention relates to a polypeptide which comprises or consists of the amino acid sequence shown in SEQ ID NO 65.

In some aspect, the invention relates to a polypeptide which comprises or consists of the amino acid sequence shown in SEQ ID NO 68.

In some aspect, the invention relates to a polypeptide which comprises or consists of the amino acid sequence shown in SEQ ID NO 71.

In some aspect, the invention relates to a polypeptide which comprises or consists of the amino acid sequence shown in SEQ ID NO 74.

In some aspect, the invention relates to a polypeptide which comprises or consists of the amino acid sequence shown in SEQ ID NO 77.

In some aspect, the invention relates to a polypeptide which comprises or consists of the amino acid sequence shown in SEQ ID NO 80.

In some aspect, the invention relates to a polypeptide which comprises or consists of the amino acid sequence shown in SEQ ID NO 83.

In some aspect, the invention relates to a polypeptide which comprises or consists of the amino acid sequence shown in SEQ ID NO 86.

In some aspect, the invention relates to a polypeptide which comprises or consists of the amino acid sequence shown in SEQ ID NO 89.

The polypeptides of the invention are as described useful for removing and reducing PNAG staining e.g. associated with organic matter such as EPS. In one embodiment, the polypeptides of the invention are useful in laundry processes or dish wash.

In another embodiment, the present invention relates to a polynucleotide having a sequence identity to the mature polypeptide coding sequence of SEQ ID NO 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 4 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 7 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 10 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 13 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 16 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 19 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 22 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 25 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 28 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 31 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 34 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 37 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 40 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 43 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 46 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 63 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 66 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 69 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 72 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 75 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 78 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 81 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 84 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to a polynucleotide having a sequence identity to the mature polypeptide coding sequence of SEQ ID NO 87 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In another embodiment, the present invention relates to variants of any of the mature polypeptides shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86 or SEQ ID NO 89 comprising a substitution, deletion, and/or insertion at one or more (e.g., several) positions. In one embodiment, the number of amino acid substitutions, deletions and/or insertions introduced into the mature polypeptide shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45,SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86 or SEQ ID NO 89 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for activity to PNAG identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage Dsplay (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide. The polypeptide may be a hybrid polypeptide in which a region of one polypeptide is fused at the N-terminus or the C-terminus of a region of another polypeptide. The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48

To be useful in cleaning processes the enzymes need to perform its action in detergents under the conditions of cleaning processes such as laundry, which includes stability in the presence of detergent components such as surfactants, builders and bleach components. The components of a detergent may significantly effect on the performance of the enzymes. The present application surprisingly shows that polypeptides comprising the GHL13 domain and/or CE4 domain and which have hydrolytic and/or deacetylation activity e.g. the polypeptides shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45,SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86, SEQ ID NO 89 or a polypeptide having at least 60%, at least 65%, at least 70%, at least 75%, at least 80%, at least 85%, at least 90% at least 95%, at least 96%, at least 97%, at least 98%, at least 99% or 100% sequence identity hereto are useful for reduction and/or removing of PNAG comprised soiling associated with cleaning e.g. on textiles or washing machines.

The polypeptides of the invention have deep cleaning effect in a broad range of detergents and are useful e.g. in detergents with different surfactant composition such as in detergent comprising anionic, non-ionic, cationic and/or amphoteric surfactants and in different ratios of e.g. anionic and nonionic surfactants.

Thus, some aspects of the invention relate the use in a cleaning process of a polypeptide comprising the amino acids sequence shown SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45 or SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86, SEQ ID NO 89 or a polypeptide having at least 60%, at least 65%, at least 70%, at least 75%, at least 80%, at least 85%, at least 90% at least 95%, at least 96%, at least 97%, at least 98%, at least 99% or 100% sequence identity hereto, wherein the polypeptide comprises the GHL13 and/or CE4 domain(s). Some aspects of the invention relate to detergent compositions comprising a) one or more polypeptide selected from the group consisting of the polypeptide comprising the amino acid sequence shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45,SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86 and SEQ ID NO 89 or a polypeptide having at least 60%, at least 65%, at least 70%, at least 75%, at least 80%, at least 85%, at least 90% at least 95%, at least 96%, at least 97%, at least 98%, at least 99% or 100% sequence identity hereto, wherein the polypeptide comprises a GHL13 and/or CE4 domain(s) and b) at least one surfactant, preferably at least one surfactant selected from the group consisting of anionic, nonionic and/or cationic surfactants. Some aspects of the invention relate to a detergent composition comprising:
a) at least 0.02 ppm of active enzyme polypeptide, wherein the polypeptide comprises a GHL13 domain and/or CE4 catalytic domains, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 3 ora polypeptide comprising the GHL13 domain and/or CE4 catalytic domain of SEQ ID NO 3 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 6 or a polypeptide comprising the GHL13 domain and/or CE4 catalytic domain of SEQ ID NO 6 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 9 ora polypeptide comprising the GHL13 domain and/or CE4 catalytic domain of SEQ ID NO 9 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 12 ora polypeptide comprising the GHL13 domain and/or CE4 catalytic domain of SEQ ID NO 12 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 15 ora polypeptide comprising the GHL13 domain and/or CE4 catalytic domain of SEQ ID NO 15 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 18 ora polypeptide comprising the GHL13 domain and/or CE4 catalytic domain of SEQ ID NO 18 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 21 ora polypeptide comprising the GHL13 domain and/or CE4 catalytic domain of SEQ ID NO 21 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 24 ora polypeptide comprising the GHL13 domain and/or CE4 catalytic domain of SEQ ID NO 24 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 27 or a polypeptide comprising the GHL13 catalytic domain of SEQ ID NO 27 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 30 or a polypeptide comprising the GHL13 catalytic domain of SEQ ID NO 30 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 33 or a polypeptide comprising the GHL13 catalytic domain of SEQ ID NO 33 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 36 or a polypeptide comprising the GHL13 catalytic domain of SEQ ID NO 36 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 39 or a polypeptide comprising the GHL13 catalytic domain of SEQ ID NO 39 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 42 or a polypeptide comprising the GHL13 catalytic domain of SEQ ID NO 42 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 45 or a polypeptide comprising the GHL13 catalytic domain of SEQ ID NO 45 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 48 or a polypeptide comprising the GHL13 catalytic domain of SEQ ID NO 48 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 65 or a polypeptide comprising the GHL13 catalytic domain of SEQ ID NO 65 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 68 or a polypeptide comprising the GHL13 catalytic domain of SEQ ID NO 68 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 71 or a polypeptide comprising the GHL13 catalytic domain of SEQ ID NO 71 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 74 or a polypeptide comprising the GHL13 catalytic domain of SEQ ID NO 74 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 77 or a polypeptide comprising the GHL13 catalytic domain of SEQ ID NO 77 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 80 or a polypeptide comprising the GHL13 catalytic domain of SEQ ID NO 80 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 83 or a polypeptide comprising the GHL13 catalytic domain of SEQ ID NO 83 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 86 or a polypeptide comprising the GHL13 catalytic domain of SEQ ID NO 86 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.
   Some aspects of the invention relate to a detergent composition comprising:
a) at least 0.02 ppm of active enzyme polypeptide, comprising the amino acid sequence shown in SEQ ID NO 89 or a polypeptide comprising the GHL13 catalytic domain of SEQ ID NO 89 and having at least 60%, at least 65%, at least 70%, at least 75%, at least 80%, at least 85%, at least 90% at least 95%, at least 96%, at least 97%, at least 98%, at least 99% or 100% sequence identity hereto, and
b) from about 1 wt % to about 60 wt % surfactant.

The surfactant may be selected among nonionic, anionic and/or amphoteric surfactants as described above, preferably anionic or nonionic surfactants but also amphoteric surfactants may be used. In general, bleach-stable surfactants are preferred. Preferred anionic surfactants are sulphate surfactants and in particular alkyl ether sulphates, especially C9-C15 alcohol ethersulfates, C12-C15 primary alcohol ethoxylate, C8-C16 ester sulphates and C10-C14 ester sulphates, such as mono dodecyl ester sulphates Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

The anionic surfactants are preferably added to the detergent in the form of salts. Suitable cations in these salts are alkali metal ions, such as sodium, potassium and lithium and ammonium salts, for example (2-hydroxyethyl)ammonium, bis(2-hydroxyethyl)ammonium and tris(2-hydroxyethyl)ammonium salts. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof. Commercially available nonionic surfactants includes Plurafac™, Lutensol™ and Pluronic™ range from BASF, Dehypon™ series from Cognis and Genapol™ series from Clariant.

The polypeptides of the invention may be formulated in compositions optionally comprising a builder such as compositions comprising:
a) at least 0.02 ppm of active enzyme polypeptide comprising a GHL13 domain and/or a CE4 catalytic domain,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 3 or comprising the GHL13 domain and/or the CE4 catalytic domain of SEQ ID NO 3 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 6 or comprising the GHL13 domain and/or the CE4 catalytic domain of SEQ ID NO 6 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 9 or comprising the GHL13 domain and/or the CE4 catalytic domain of SEQ ID NO 9 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 12 or comprising the GHL13 domain and/or the CE4 catalytic domain of SEQ ID NO 12 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 15 or comprising the GHL13 domain and/or the CE4 catalytic domain of SEQ ID NO 15 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 18 or comprising the GHL13 domain and/or the CE4 catalytic domain of SEQ ID NO 18 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 21 or comprising the GHL13 domain and/or the CE4 catalytic domain of SEQ ID NO 21 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 24 or comprising the GHL13 domain and/or the CE4 catalytic domain of SEQ ID NO 24 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 27 or comprising the GHL13 domain of SEQ ID NO 27 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 30 or comprising the GHL13 domain of SEQ ID NO 30 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 33 or comprising the GHL13 domain of SEQ ID NO 33 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 36 or comprising the GHL13 domain of SEQ ID NO 36 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 39 or comprising the GHL13 domain of SEQ ID NO 39 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 42 or comprising the GHL13 domain of SEQ ID NO 42 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 45 or comprising the GHL13 domain of SEQ ID NO 45 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 48 or comprising the GHL13 domain of SEQ ID NO 48 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 65 or comprising the GHL13 domain of SEQ ID NO 65 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 68 or comprising the GHL13 domain of SEQ ID NO 68 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 71 or comprising the GHL13 domain of SEQ ID NO 71 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 74 or comprising the GHL13 domain of SEQ ID NO 74 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 77 or comprising the GHL13 domain of SEQ ID NO 77 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 80 or comprising the GHL13 domain of SEQ ID NO 80 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 83 or comprising the GHL13 domain of SEQ ID NO 83 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 86 or comprising the GHL13 domain of SEQ ID NO 86 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.
   Some aspects of the invention relate to a composition comprising:
a) at least 0.02 ppm of active enzyme polypeptide comprising the amino acid sequence shown in SEQ ID NO 89 or comprising the GHL13 domain of SEQ ID NO 89 and having at least 80% sequence identity hereto,
b) from about 2 wt % to about 60 wt % of at least one surfactant, and optionally
c) from about 5 wt % to about 50 wt % of at least one builder such as carbonates, zeolites, phosphate builder, calcium sequestering builders or complexing agents.

The builder is preferably selected among phosphates, sodium citrate builders, sodium carbonate, sodium silicate, sodium aluminosilicate (zeolite). Suitable builders are alkali metal or ammonium phosphates, polyphosphates, phosphonates, polyphosphonates, carbonates, bicarbonates, borates, citrates, and polycarboxylates. Citrate builders, e.g., citric acid and soluble salts thereof (particularly sodium salt), are polycarboxylate builders. Citrates can be used in combination with zeolite, silicates like the BRITESIL types, and/or layered silicate builders. The builder is preferably added in an amount of about 0-65% by weight, such as about 5% to about 50% by weight. In the composition of the invention, the level of builder is typically about 40-65% by weight, particularly about 50-65% by weight, particularly from 20% to 50% by weight. The builder and/or co-builder may particularly be a chelating agent that forms water-soluble complexes with Ca and Mg. Any builder and/or co-builder known in the art for use in cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), and (carboxymethyl)inulin (CMI), and combinations thereof. Further non-limiting examples of builders include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycine-N,N- diacetic acid (MGDA), glutamic acid-N,N-diacetic acid (GLDA), 1-hydroxyethane-1,1-diphosphonic acid, N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(sulfomethyl)aspartic acid (SMAS), N-(2-sulfoethyl)-aspartic acid (SEAS), N-(sulfomethylglutamic acid (SMGL), N-(2-sulfoethyl)-glutamic acid (SEGL), N-methyliminodiacetic acid (MIDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and N′-(2-hydroxyethypethylenediamine-N,N,N′-triacetic acid (HEDTA), diethanolglycine (DEG), and combinations and salts thereof.

Phosphonates suitable for use herein include 1-hydroxyethane-1,1-diphosphonic acid (H EDP), ethylenediaminetetrakis(methylenephosphonic acid) (EDTMPA), diethylenetriaminepentakis(methylenephosphonic acid) (DTMPA or DTPMPA or DTPMP), nitrilotris(methylenephosphonic acid) (ATMP or NTMP), 2-phosphonobutane-1,2,4-tricarboxylic acid (PBTC), hexamethylenediaminetetrakis(methylenephosphonic acid) (HDTMP)

The composition of the invention may also contain 0-50% by weight, such as about 5% to about 30%, of a detergent co-builder.

The composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA) or polyaspartic acid.

Further exemplary builders and/or co-builders are described in, e.g., WO 09/102854, U.S. Pat. No. 5,977,053

In one preferred embodiment, the builder is a non-phosphorus based builder such as citric acid and/or methylglycine-N,N-diacetic acid (MGDA) and/or glutamic-N,N-diacetic acid (GLDA) and/or salts thereof.

The detergent composition may contain 0-30% by weight, such as about 1% to about 20%, of a bleaching system. Any bleaching system comprising components known in the art for use in cleaning detergents may be utilized. Suitable bleaching system components include sources of hydrogen peroxide; sources of peracids; and bleach catalysts or boosters.

Sources of Hydrogen Peroxide

Suitable sources of hydrogen peroxide are inorganic persalts, including alkali metal salts such as sodium percarbonate and sodium perborates (usually mono- or tetrahydrate), and hydrogen peroxide-urea (1/1).

Sources of Peracids

Peracids may be (a) incorporated directly as preformed peracids or (b) formed in situ in the wash liquor from hydrogen peroxide and a bleach activator (perhydrolysis) or (c) formed in situ in the wash liquor from hydrogen peroxide and a perhydrolase and a suitable substrate for the latter, e.g., an ester.

a) Suitable preformed peracids include, but are not limited to, peroxycarboxylic acids such as peroxybenzoic acid and its ring-substituted derivatives, peroxy-α-naphthoic acid, peroxyphthalic acid, peroxylauric acid, peroxystearic acid, ε-phthalimidoperoxycaproic acid [phthalimidoperoxyhexanoic acid (PAP)], and o-carboxybenzamidoperoxycaproic acid; aliphatic and aromatic diperoxydicarboxylic acids such as diperoxydodecanedioic acid, diperoxyazelaic acid, diperoxysebacic acid, diperoxybrassylic acid, 2-decyldiperoxybutanedioic acid, and diperoxyphthalic, -isophthalic and -terephthalic acids; perimidic acids; peroxymonosulfuric acid; peroxydisulfuric acid; peroxyphosphoric acid; peroxysilicic acid; and mixtures of said compounds. It is understood that the peracids mentioned may in some cases be best added as suitable salts, such as alkali metal salts (e.g., Oxone®) or alkaline earth-metal salts.

b) Suitable bleach activators include those belonging to the class of esters, amides, imides, nitriles or anhydrides and, where applicable, salts thereof. Suitable examples are tetraacetylethylenediamine (TAED), sodium 4-[(3,5,5-trimethylhexanoyl)oxy]benzene-1-sulfonate (ISONOBS), sodium 4-(dodecanoyloxy)benzene-1-sulfonate (LOBS), sodium 4-(decanoyloxy)benzene-1-sulfonate, 4-(decanoyloxy)benzoic acid (DOBA), sodium 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that they are environmentally friendly. Furthermore, acetyl triethyl citrate and triacetin have good hydrolytically stability in the product upon storage and are efficient bleach activators. Finally, ATC is multifunctional, as the citrate released in the perhydrolysis reaction may function as a builder.

### Bleach Catalysts and Boosters

The bleaching system may also include a bleach catalyst or booster. Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-κN-methanylylidene)triphenolato-κ3O]manganese(III). The bleach catalysts may also be other metal compounds; such as iron or cobalt complexes.

In some embodiments, where a source of a peracid is included, an organic bleach catalyst or bleach booster may be used having one of the following formulae:

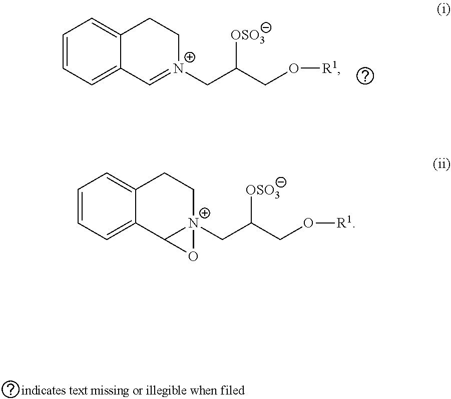

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl.

Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Some aspects of the invention relate to detergent compositions comprising:
a) at least 0.001 ppm of active enzyme polypeptide, comprising a GHL13 domain and/or a CE4 catalytic domain, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 3 or comprising the GHL13 domain and/or the CE4 catalytic domain of SEQ ID NO 3 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 6 or comprising the GHL13 domain and/or the CE4 catalytic domain of SEQ ID NO 6 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 9 or comprising the GHL13 domain and/or the CE4 catalytic domain of SEQ ID NO 9 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 12 or comprising the GHL13 domain and/or the CE4 catalytic domain of SEQ ID NO 12 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 15 or comprising the GHL13 domain and/or the CE4 catalytic domain of SEQ ID NO 15 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 18 or comprising the GHL13 domain and/or the CE4 catalytic domain of SEQ ID NO 18 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 21 or comprising the GHL13 domain and/or the CE4 catalytic domain of SEQ ID NO 21 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 24 or comprising the GHL13 domain and/or the CE4 catalytic domain of SEQ ID NO 24 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 27 or comprising the GHL13 domain of SEQ ID NO 27 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 30 or comprising the GHL13 domain of SEQ ID NO 30 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 33 or comprising the GHL13 domain of SEQ ID NO 33 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 36 or comprising the GHL13 domain of SEQ ID NO 36 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 39 or comprising the GHL13 domain of SEQ ID NO 39 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 42 or comprising the GHL13 domain of SEQ ID NO 42 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 45 or comprising the GHL13 domain of SEQ ID NO 45 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 48 or comprising the GHL13 domain of SEQ ID NO 48 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 65 or comprising the GHL13 domain of SEQ ID NO 65 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
e) at least 0.001 ppm of active enzyme polypeptide, comprising the amino acid sequence shown in SEQ ID NO 68 or comprising the GHL13 domain of SEQ ID NO 68 and having at least 60%, such as at least 70%, such as at least 80% or such as at least 90% sequence identity hereto, and optionally
f) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
g) from about 5 wt % to about 50 wt % surfactant, preferably selected from anionic surfactants such as LAS, AOS, AEOS and/or nonionic surfactants such as AE or AEO, and optionally
h) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 71 or comprising the GHL13 domain of SEQ ID NO 71 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 74 or comprising the GHL13 domain of SEQ ID NO 74 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 77 or comprising the GHL13 domain of SEQ ID NO 77 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 80 or comprising the GHL13 domain of SEQ ID NO 80 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 83 or comprising the GHL13 domain of SEQ ID NO 83 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 86 or comprising the GHL13 domain of SEQ ID NO 86 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).
   Some aspects of the invention relate to detergent compositions comprising:
a) at least 0.001 ppm of active enzyme polypeptide, comprising the amino acid sequence shown in SEQ ID NO 89 or comprising the GHL13 domain of SEQ ID NO 89 and having at least 60%, such as at least 70%, such as at least 80% or such as at least 90% sequence identity hereto, and optionally
b) from about 10 wt % to about 50 wt % builder preferably selected from citric acid, methylglycine-N,N-diacetic acid (MGDA) and/or glutamic acid-N,N-diacetic acid (GLDA) and mixtures thereof, and optionally
c) from about 5 wt % to about 50 wt % surfactant, preferably selected from anionic surfactants such as LAS, AOS, AEOS and/or nonionic surfactants such as AE or AEO, and optionally
d) at least one bleach component, at least one bleach component, wherein the bleach is a peroxide and the bleach catalyst is a manganese compound, wherein, the oxygen bleach is preferably percarbonate and the manganese catalyst preferably 1,4,7-trimethyl-1,4,7-triazacyclononane or manganese (III) acetate tetrahydrate (MnTACN).

Some aspects of the invention relate to the use of a polypeptide comprising a GHL13 domain and/or CE4 catalytic domain for preventing, reducing or removing re-deposition of soil during a wash cycle.

Further, the invention relates the use of a polypeptide comprising a GHL13 domain and/or CE4 catalytic domain for preventing, reducing or removing the adherence of soil to an item. In one embodiment, the item is textile. When the soil does not adhere to the item, the item appears cleaner. Thus, the invention further relates the use of a polypeptide comprising a GHL13 domain and/or CE4 catalytic domain for maintaining or improving the whiteness of the item.

The detergent composition according to the invention may comprise a detergent adjunct; the detergent adjunct ingredient may be surfactants and builders and/or chelators such as those described above. The adjunct ingredients may also be any of the following flocculating aid, dye transfer inhibitors, enzymes, enzyme stabilizers, enzyme inhibitors, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric hueing agents, anti-foaming agents, dispersants, processing aids, and/or pigments.

In one embodiment, the detergent adjunct ingredient is a builder or a clay soil removal/anti-redeposition agent.

In one embodiment, detergent adjunct ingredient is an enzyme. The one or more enzymes may be selected from the group consisting of proteases, lipases, cutinases, amylases, carbohydrases, cellulases, pectinases, mannanases, arabinases, galactanases, xylanases and oxidases.

In addition to the polypeptides comprising a GHL13 domain and/or CE4 catalytic domain e.g. polypeptides comprising the amino acid sequence shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77, SEQ ID NO 80, SEQ ID NO 83, SEQ ID NO 86, SEQ ID NO 89 or a polypeptide comprising a GHL13 domain and/or CE4 catalytic domain and having at least 60%, at least 65%, at least 70%, at least 75%, at least 80%, at least 85%, at least 90% at least 95%, at least 96%, at least 97%, at least 98%, at least 99% or 100% sequence identity to SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77, SEQ ID NO 80, SEQ ID NO 83, SEQ ID NO 86, SEQ ID NO 89 the detergents of the invention may further comprise cellulases. Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having color care benefits. Examples of such cellulases are cellulases described in EP 0 495 257, EP 0 531 372, WO 96/11262, WO 96/29397, WO 98/08940. Other examples are cellulase polypeptides such as those described in WO 94/07998, EP 0 531 315, U.S. Pat. Nos. 5,457,046, 5,686,593, 5,763,254, WO 95/24471, WO 98/12307 and PCT/DK98/00299.

Example of cellulases exhibiting endo-beta-1,4-glucanase activity (EC 3.2.1.4) are those having described in WO02/099091.

Other examples of cellulases include the family 45 cellulases described in WO96/29397, and especially polypeptides thereof having substitution, insertion and/or deletion at one or more of the positions corresponding to the following positions in SEQ ID NO 8 of WO 02/099091: 2, 4, 7, 8, 10, 13, 15, 19, 20, 21, 25, 26, 29, 32, 33, 34, 35, 37, 40, 42, 42a, 43, 44, 48, 53, 54, 55, 58, 59, 63, 64, 65, 66, 67, 70, 72, 76, 79, 80, 82, 84, 86, 88, 90, 91, 93, 95, 95d, 95h, 95j, 97, 100, 101, 102, 103, 113, 114, 117, 119, 121, 133, 136, 137, 138, 139, 140a, 141, 143a, 145, 146, 147, 150e, 150j, 151, 152, 153, 154, 155, 156, 157, 158, 159, 160c, 160e, 160k, 161, 162, 164, 165, 168, 170, 171, 172, 173, 175, 176, 178, 181, 183, 184, 185, 186, 188, 191, 192, 195, 196, 200, and/or 20, preferably selected among P19A, G20K, Q44K, N48E, Q119H or Q146 R.

Commercially available cellulases include Celluzyme™, Celluclean and Carezyme™ (Novozymes A/S), Clazinase™, and Puradax HA™ (Genencor International Inc.), and KAC-500(B)™ (Kao Corporation).

In addition to the polypeptides comprising a GHL13 domain and/or CE4 catalytic domain e.g. polypeptides comprising the amino acid sequence shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77, SEQ ID NO 80, SEQ ID NO 83, SEQ ID NO 86, SEQ ID NO 89 or a polypeptide comprising a GHL13 domain and/or CE4 catalytic domain and having at least 60%, at least 65%, at least 70%, at least 75%, at least 80%, at least 85%, at least 90% at least 95%, at least 96%, at least 97%, at least 98%, at least 99% or 100% sequence identity to SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77, SEQ ID NO 80, SEQ ID NO 83, SEQ ID NO 86, SEQ ID NO 89 the detergents of the invention may further comprise proteases. Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloproteases such as those from M5, M7 or M8 families. The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 24, 27, 42, 55, 59, 60, 66, 74, 85, 96, 97, 98, 99, 100, 101, 102, 104, 116, 118, 121, 126, 127, 128, 154, 156, 157, 158, 161, 164, 176, 179, 182, 185, 188, 189, 193, 198, 199, 200, 203, 206, 211, 212, 216, 218, 226, 229, 230, 239, 246, 255, 256, 268 and 269, wherein the positions correspond to the positions of the Bacillus Lentus protease shown in SEQ ID NO 1 of WO 2016/001449. More preferred the subtilase variants may comprise any of the mutations: S3T, V4I, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V102I, V102Y, V102N, S104A, G116V, G116R, H118D, H118N, A1205, S126L, P127Q, S128A, S154D, A156E, G157D, G157P, S158E, Y161A, R164S, Q176E, N179E, S182E, Q185N, A188P, G189E, V193M, N198D, V199I, Y203W, S206G, L211Q, L211D, N212D, N212S, M216S, A226V, K229L, Q230H, Q239R, N246K, N255W, N255D, N255E, L256E, L256D T268A and R269H. The protease variants are preferably variants of the Bacillus lentus protease (Savinase®) shown in SEQ ID NO 1 of WO 2016/001449, the Bacillus amylolichenifaciens protease (BPN') shown in SEQ ID NO 2 of WO2016/001449. The protease variants preferably have at least 80% sequence identity to SEQ ID NO 1 or SEQ ID NO 2 of WO 2016/001449.

A protease variant comprising a substitution at one or more positions corresponding to positions 171, 173, 175, 179, or 180 of SEQ ID NO 1 of WO2004/067737, wherein said protease variant has a sequence identity of at least 75% but less than 100% to SEQ ID NO 1 of WO2004/067737.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Neutrase®, Everlase® and Esperase® (Novozymes A/S), those sold under the tradename Maxatase®, Maxacal®, Maxapem®, Purafect Ox®, Purafect OxP®, Puramax®, FN2®, FN3®, FN4®, Excellase®, Excellenz P1000™, Excellenz P1250™, Eraser®, Preferenz P100™, Purafect Prime®, Preferenz P110™, Effectenz P1000™, Purafect®™, Effectenz P1050™, Purafect Ox®™, Effectenz P2000™, Purafast®, Properase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

In addition to the polypeptides comprising a GHL13 domain and/or CE4 catalytic domain e.g. polypeptides comprising the amino acid sequence shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77, SEQ ID NO 80, SEQ ID NO 83, SEQ ID NO 86, SEQ ID NO 89 or a polypeptide comprising a GHL13 domain and/or CE4 catalytic domain and having at least 60%, at least 65%, at least 70%, at least 75%, at least 80%, at least 85%, at least 90% at least 95%, at least 96%, at least 97%, at least 98%, at least 99% or 100% sequence identity to SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77, SEQ ID NO 80, SEQ ID NO 83, SEQ ID NO 86, SEQ ID NO 89 the detergents of the invention may further comprise lipases and cutinases which include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase polypeptides such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and polypeptides of the M. smegmatis perhydrolase in particular the S54V variant used in the commercial product Gentle Power Bleach from Huntsman Textile Effects Pte Ltd (WO10/100028).

In addition to the polypeptides comprising a GHL13 domain and/or CE4 catalytic domain e.g. polypeptides comprising the amino acid sequence shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77, SEQ ID NO 80, SEQ ID NO 83, SEQ ID NO 86, SEQ ID NO 89 or a polypeptide comprising a GHL13 domain and/or CE4 catalytic domain and having at least 60%, at least 65%, at least 70%, at least 75%, at least 80%, at least 85%, at least 90% at least 95%, at least 96%, at least 97%, at least 98%, at least 99% or 100% sequence identity to SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77, SEQ ID NO 80, SEQ ID NO 83, SEQ ID NO 86, SEQ ID NO 89 the detergents of the invention may further comprise amylases which can be used together with a polypeptides of the invention. The amylase may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T1651+K178L+T182G+Y305R+G475K wherein the variants are

C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, I203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

E187P+I203Y+G476K

E187P+I203Y+R458N+T459S+D460T+G476K,

wherein the variants optionally further comprise a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of I181 and/or G182. Most preferred amylase variants of SEQ ID NO: 1 are those having the substitutions:

N21D+D97N+V128I, wherein the variants optionally further comprise a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087. Commercially available amylases are Duramyl™, Termamyl™, Fungamyl™, Stainzyme™, Stainzyme™, Natalase™, Liquozyme X and BAN™ (from Novozymes A/S), and Rapidase™, Purastar™/Effectenz™, Powerase, Preferenz S1000, Preferenz S100 and Preferenz S110 (from Genencor International Inc./DuPont).

In addition to the polypeptides comprising a GHL13 domain and/or CE4 catalytic domain e.g. polypeptidescomprising the amino acid sequence shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77, SEQ ID NO 80, SEQ ID NO 83, SEQ ID NO 86, SEQ ID NO 89 or a polypeptide a GHL13 domain and/or CE4 catalytic domain and having at least 60%, at least 65%, at least 70%, at least 75%, at least 80%, at least 85%, at least 90% at least 95%, at least 96%, at least 97%, at least 98%, at least 99% or 100% sequence identity to SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77, SEQ ID NO 80, SEQ ID NO 83, SEQ ID NO 86, SEQ ID NO 89 the detergents of the invention may further comprise peroxidases/oxidases including those of plant, bacterial or fungal origin. Chemically modified or protein engineered mutants are included. Examples of useful peroxidases include peroxidases from Coprinus, e.g., from C. cinereus, and polypeptides thereof as those described in WO 93/24618, WO 95/10602, and WO 98/15257.

Commercially available peroxidases include Guardzyme™ (Novozymes A/S).

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

The detergent compositions of the invention may also contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), (carboxymethyl)inulin (CMI), and polycarboxylates such as PAA, PAA/PMA, polyaspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light if subjected to ultraviolet light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

The detergent compositions of the present invention can also contain dispersants. In particular, powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

The detergent compositions of the present invention may also include one or more dye transfer inhibiting agents. Suitable polymeric dye transfer inhibiting agents include, but are not limited to, polyvinylpyrrolidone polymers, polyamine-N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the laundry composition of the present invention. The most commonly used fluorescent whitening agents are those belonging to the classes of diaminostilbene-sulfonic acid derivatives, diarylpyrazoline derivatives and biphenyl-distyryl derivatives. Examples of the diaminostilbene-sulfonic acid derivative type of fluorescent whitening agents include the sodium salts of: 4,4′-bis[(4-anilino-6-diethanolamino-s-triazin-2-yl)amino]stilbene-2,2′-disulfonate, 4,4′-bis[(4,6-dianilino-s-triazin-2-yl)amino]stilbene-2,2′-disulfonate, 4,4′-bis{4-anilino-6-[methyl(2-hydroxyethyl)amino]-s-triazin-2-ylamino}stilbene-2,2′-disulfonate, 4,4′-bis(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from BASF. Tinopal DMS is the disodium salt of 4,4′-bis[(4-anilino-6-morpholino-s-triazin-2-yl)amino]stilbene-2,2′-disulfonate. Tinopal CBS is the disodium salt of 2,2′-[biphenyl-4,4′-di(2,1-ethenediyl)]dibenzene-1-sulfonate. Also preferred is the commercially available Parawhite KX, supplied by Paramount Minerals and Chemicals, Mumbai, India. Other fluorescers suitable for use in the invention include the 1-3-diarylpyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

The detergent compositions of the present invention may also include one or more soil-release polymers which aid the removal of soils from fabrics such as cotton and polyester-based fabrics, in particular removal of hydrophobic soils from polyester-based fabrics. The soil release polymers may for example be nonionic or anionic terephthalate-based polymers, polyvinylcaprolactam and related copolymers, vinyl graft copolymers or polyester polyamides; see for example Chapter 7 in Powdered Detergents, Surfactant science series volume 71, Marcel Dekker, Inc. Another type of soil release polymers is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil-release polymers. Suitable graft co-polymers are described in more detail in WO 2007/138054, WO 2006/108856 and WO 2006/113314 (hereby incorporated by reference). Other soil-release polymers are substituted polysaccharide structures especially substituted cellulosic structures such as modified cellulose derivatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof.

The detergent compositions of the present invention may also include one or more anti-redeposition agents such as (carboxymethyl) cellulose (CMC), poly(vinyl alcohol) (PVA), homopolymers of acrylic acid, copolymers of acrylic acid and maleic acid, and ethoxylated polyethyleneimines. The cellulose based polymers described under soil-release polymers above may also function as anti-redeposition agents.

### Sources of Polypeptides

A polypeptide of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

In some aspect, the polypeptide is a Pseudomonas polypeptide, e.g., a polypeptide obtained from Pseudomonas meridiana. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 3 and is obtained from Pseudomonas preferably Pseudomonas meridiana.

In some aspect, the polypeptide is a Halomonas polypeptide, e.g., a polypeptide obtained from Halomonas sp-62262. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 6 and is obtained from Halomonas preferably Halomonas sp-62262.

In some aspect, the polypeptide is a Pseudomonas polypeptide, e.g., a polypeptide obtained from Pseudomonas migulae. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 9 and is obtained from Pseudomonas preferably Pseudomonas migulae.

In some aspect, the polypeptide is a Pseudomonas polypeptide, e.g., a polypeptide obtained from Pseudomonas sp-62331. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 12 and is obtained from Pseudomonas preferably, Pseudomonas sp-62331.

In some aspect, the polypeptide is a Pseudomonas polypeptide, e.g., a polypeptide obtained from Pseudomonas jessenii. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 15 and is obtained from Pseudomonas preferably, Pseudomonas jessenii.

In some aspect, the polypeptide is a Pseudomonas polypeptide, e.g., a polypeptide obtained from Pseudomonas koreensis. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 18 and is obtained from Pseudomonas preferably, Pseudomonas koreensis.

In some aspect, the polypeptide is a Stenotrophomonas polypeptide, e.g., a polypeptide obtained from Stenotrophomonas rhizophila. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 21 and is obtained from Stenotrophomonas preferably Stenotrophomonas rhizophila.

In some aspect, the polypeptide is a Pseudomonas polypeptide, e.g., a polypeptide obtained from Pseudomonas sp-62498. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 24 and is obtained from Pseudomonas preferably, Pseudomonas sp-62498.

In some aspect, the polypeptide is an Acinetobacter polypeptide, e.g., a polypeptide obtained from Acinetobacter bouvetii. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 27 and is obtained from Acinetobacter preferably Acinetobacter bouvetii.

In some aspect, the polypeptide is a Pseudomonas polypeptide, e.g., a polypeptide obtained from Pseudomonas panacis. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 30 and is obtained from Pseudomonas preferably Pseudomonas panacis.

In some aspect, the polypeptide is a Bacterial polypeptide, e.g., a polypeptide obtained from Enviromental bacterial community L. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 33 and is obtained from Bacterial preferably Enviromental bacterial community L.

In some aspect, the polypeptide is a Halomonas polypeptide, e.g., a polypeptide obtained from Halomonas zhanjiangensis. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 36 and is obtained from Halomonas preferably Halomonas zhanjiangensis, more preferably Halomonas zhanjiangensis DSM 21076.

In some aspect, the polypeptide is a Halomonas polypeptide, e.g., a polypeptide obtained from Halomonas sp-63456. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 39 and is obtained from Halomonas preferably Halomonas sp-63456.

In some aspect, the polypeptide is a Luteibacter polypeptide, e.g., a polypeptide obtained from Luteibacter rhizovicinus. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 42 and is obtained from Luteibacter preferably Luteibacter rhizovicinus.

In some aspect, the polypeptide is a Bacterial polypeptide, e.g., a polypeptide obtained from Enviromental bacterial community R. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 45 and is obtained from Bacterial preferably Enviromental bacterial community R.

In some aspect, the polypeptide is a Bacterial polypeptide, e.g., a polypeptide obtained from Enviromental bacterial community H. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 48 and is obtained from Bacterial preferably Enviromental bacterial community H.

In some aspect, the polypeptide is a polypeptide obtained from Vibrio proteolyticus. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 65 and is obtained from Vibrio proteolyticus.

In some aspect, the polypeptide is a polypeptide obtained from Aquitalea magnusonii. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 68 and is obtained from Aquitalea magnusonii.

In some aspect, the polypeptide is a polypeptide obtained from Halomonas ilicicola. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 71 and is obtained from Halomonas ilicicola.

In some aspect, the polypeptide is a polypeptide obtained from Alkanindiges illinoisensis. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 74 and is obtained from Alkanindiges illinoisensis.

In some aspect, the polypeptide is a polypeptide obtained from Halomonas sp. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 77 and is obtained from Halomonas sp.

In some aspect, the polypeptide is a polypeptide obtained from Halomonas sp. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 80 and is obtained from Halomonas sp.

In some aspect, the polypeptide is a polypeptide obtained from Luteibacter sp. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 83 and is obtained from Luteibacter sp.

In some aspect, the polypeptide is a polypeptide obtained from Variovorax boronicumulans. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 86 and is obtained from Variovorax boronicumulans.

In some aspect, the polypeptide is a polypeptide obtained from Silvimonas terrae. In a preferred aspect, the polypeptide is a polypeptide having at least 60%, such as at least 65%, such as at least 70%, such as at least 75%, such as at least 80%, such as at least 85%, such as at least 90%, such as at least 95%, such as at least 96%, such as at least 98% or 100% sequence identity to SEQ ID NO 89 and is obtained from Silvimonas terrae.

In one embodiment, the GHL13 glycosyl hydrolase is obtained from Pseudomonas, preferably Pseudomonas meridiana, Pseudomonas migulae, Pseudomonas sp-62331, Pseudomonas jessenii, Pseudomonas koreensis, Pseudomonas sp-62498, Pseudomonas panacis wherein the GHL13 glycosyl hydrolase is selected from the group consisting of:
(a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 3;
(b) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 9;
(d) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 12;
(e) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 15;
(h) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 18;
(j) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 24; and
(l) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 30.
   In one embodiment, the GHL13 glycosyl hydrolase is obtained from Halomonas, preferably Halomonas sp-62262, Halomonas zhanjiangensis DSM 21076, Halomonas sp-63456, Halomonas ilicicola, Halomonas sp, wherein the GHL13 glycosyl hydrolase is selected from the group consisting of:
(a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(b) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 36;
(d) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 39;
(e) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 71;
(h) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 77; and
(j) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 80.

It will be understood that for the species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in several culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenaturn, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianurn, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is a Halomonas cell. In another aspect, the cell is a Halomonas sp-62262, Halomonas sp-63456 or a Halomonas zhanjiangensis DSM 21076 cell.

In one aspect, the cell is a Pseudomonas cell. In another aspect, the cell is a Pseudomonas migulae, Pseudomonas jessenii, Pseudomonas sp-62498, Pseudomonas panacis or a Pseudomonas koreensis cell. In one aspect, the cell is a Stenotrophomonas cell. In another aspect, the cell is a Stenotrophomonas rhizophila cell. In one aspect, the cell is an Luteibacter cell. In another aspect, the cell is a Luteibacter rhizovicinus cell. In one aspect, the cell is a Bacterial cell. In another aspect, the cell is a Enviromental bacterial community R or a Enviromental bacterial community H cell. In another aspect, the cell is a Silvimonas terrae cell.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermenters in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides having activity to PNAG. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Laundry Soap Bars

The polypeptides of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, PgaB, optionally one or more additional enzymes, a protease inhibitor, and a salt of a monovalent cation and an organic anion may be prepared and the mixture is then plodded. The polypeptides of the invention and optional additional enzymes may be added at the same time as the protease inhibitor for example in liquid form. Besides the mixing step and the plodding step, the process may further comprise the steps of milling, extruding, cutting, stamping, cooling and/or wrapping.

### Formulation of Enzyme in Co-Granule

Non-dusting granulates may be produced, e.g. as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216. The polypeptides may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulate for the detergent industry is disclosed in the IP.com disclosure IPCOM000200739D. Another example of formulation of enzymes using co-granulates are disclosed in WO 2013/188331, which relates to a detergent composition comprising (a) a multi-enzyme co- granule; (b) less than 10 wt zeolite (anhydrous basis); and (c) less than 10 wt phosphate salt (anhydrous basis), wherein said enzyme co-granule comprises from 10 to 98 wt % moisture sink components and the composition additionally comprises from 20 to 80 wt % detergent moisture sink components. An embodiment of the invention relates to an enzyme granule/particle comprising the polypeptides of the invention. The granule is composed of a core, and optionally one or more coatings (outer layers) surrounding the core.

Typically, the granule/particle size, measured as equivalent spherical diameter (volume based average particle size), of the granule is 20-2000 μm, particularly 50-1500 μm, 100-1500 μm or 250-1200 μm.

The core may include additional materials such as fillers, fibre materials (cellulose or synthetic fibres), stabilizing agents, solubilising agents, suspension agents, viscosity regulating agents, light spheres, plasticizers, salts, lubricants and fragrances. The core may include binders, such as synthetic polymer, wax, fat, or carbohydrate. The core may comprise a salt of a multivalent cation, a reducing agent, an antioxidant, a peroxide decomposing catalyst and/or an acidic buffer component, typically as a homogenous blend. The core may consist of an inert particle with the enzyme absorbed into it, or applied onto the surface, e.g., by fluid bed coating. The core may have a diameter of 20-2000 μm, particularly 50-1500 μm, 100-1500 μm or 250-1200 μm. The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation. Methods for preparing the core can be found in Handbook of Powder Technology; Particle size enlargement by C. E. Capes; Volume 1; 1980; Elsevier.

The core of the enzyme granule/particle may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA). Examples of enzyme granules with multiple coatings are shown in WO 93/07263 and WO 97/23606. The coating may be applied in an amount of at least 0.1% by weight of the core, e.g., at least 0.5%, 1% or 5%. The amount may be at most 100%, 70%, 50%, 40% or 30%. The coating is preferably at least 0.1 μm thick, particularly at least 0.5 μm, at least 1 μm or at least 5 μm. In a particular embodiment, the thickness of the coating is below 100 μm. In a more particular embodiment the thickness of the coating is below 60 μm. In an even more particular embodiment the total thickness of the coating is below 40 μm. The coating should encapsulate the core unit by forming a substantially continuous layer. A substantially continuous layer is to be understood as a coating having few or no holes, so that the core unit it is encapsulating/enclosing has few or none uncoated areas. The layer or coating should in particular be homogeneous in thickness. The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight w/w of a salt, e.g., at least 65%, at least 70%, at least 75%, at least 80%, at least 85%, at least 90%, at least 95% or at least 99% by weight w/w. The salt may be added from a salt solution where the salt is completely dissolved or from a salt suspension wherein the fine particles is less than 50 μm, such as less than 10 μm or less than 5 μm. The salt coating may comprise a single salt or a mixture of two or more salts. The salt may be water soluble, in particular having a solubility at least 0.1 grams in 100 g of water at 20° C., preferably at least 0.5 g per 100 g water, e.g., at least 1 g per 100 g water, e.g., at least 5 g per 100 g water. The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminium. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used. The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710. Specific examples of suitable salts are NaCI (CH_{20° C.}=76%), Na₂CO₃ (CH_{20° C.}=92%), NaNO₃ (CH_{20° C.}=73%), Na₂HPO₄ (CH_{20° C.}=95%), Na₃PO4 (CH_{25° C.}=92%), NH₄CI (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCI (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{20° C.}=86%). Other examples include NaH₂PO₄, (NH₄)H₂PO₄, CuSO₄, Mg(NO₃)₂ and magnesium acetate. The salt may be in anhydrous form, or it may be a hydrated salt, i.e. a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄.7H₂O), zinc sulfate heptahydrate (ZnSO₄.7H₂O), sodium phosphate dibasic heptahydrate (Na₂HPO₄.7H₂O), magnesium nitrate hexahydrate (Mg(NO₃)₂(6H₂O)), sodium citrate dihydrate and magnesium acetate tetrahydrate. Preferably the salt is applied as a solution of the salt, e.g., using a fluid bed.

One aspect of the invention relates to a granule comprising:

(a) a core comprising a polypeptide according to the invention, and

(b) optionally a coating consisting of one or more layer(s) surrounding the core.

In one aspect, the present invention provides a granule, which comprises:

(a) a core comprising a polypeptide comprising the amino acid sequence shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86 SEQ ID NO 89 or polypeptides having, at least 60%, at least 65%, at least 70%, at least 75%, at least 80%, at least 85%, at least 90%, at least 95%, at least 97%, at least 98%, at least 99% or 100% sequence identity hereto, and

(b) optionally a coating consisting of one or more layer(s) surrounding the core.

### Medical Cleaning

The present invention further relates to methods of cleaning a medical device and to the use of a composition comprising a GHL13 glycosyl hydrolases and at least one adjunct ingredient for cleaning of a medical device. The invention further relates to a method of preventing biofilm formation on a medical device e.g. an indwelling medical device or implant comprising coating the device with at least one GHL13 glycosyl hydrolase.

One embodiment of the invention relates to a method of preventing biofilm formation on a medical device e.g. an indwelling medical device or implant comprising coating the device with at least one GHL13 glycosyl hydrolase.

The polypeptides suitable for use in medical cleaning and in compositions for medical cleaning are described above and include polypeptides which comprises one or more motif(s) [Y/W]PX[D/N]F (SEQ ID NO 59), [M/E/Y/F]AM[P/G] (SEQ ID NO 60) or WPY and/or polypeptide is selected from the group consisting of polypeptides having the amino acid sequence of SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86, SEQ ID NO 89 and polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto.

One aspect of the invention relates to a method of cleaning a medical device, wherein the method comprises
a) contacting the medical device with the composition comprising a GHL13 glycosyl hydrolase, for a period effective to clean the medical device;
b) cleaning, the medical device; and
c) optionally disinfect the medical device.
   One aspect of the invention relates to a method of cleaning a medical device, wherein the method comprises
a) contacting the medical device with the composition comprising a GHL13 glycosyl hydrolase, which comprises one or more motif(s) [Y/W]PX[D/N]F (SEQ ID NO 59), [W/E/Y/F]AM[P/G] (SEQ ID NO 60) or WPY and/or is selected from the group consisting of GHL13 glycosyl hydrolases having the amino acid sequence of SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86, SEQ ID NO 89 and polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto, for a period effective to clean the medical device;
b) cleaning, the medical device; and
c) optionally disinfect the medical device.

One aspect of the invention relates to the use of a GHL13 glycosyl hydrolase of the invention for cleaning a medical device, wherein the the GHL13 glycosyl hydrolase, which optionally comprises one or more motif(s) [Y/W]PX[D/N]F (SEQ ID NO 59), [M/E/Y/F]AM[P/G] (SEQ ID NO 60) or WPY and/or is selected from the group consisting of GHL13 glycosyl hydrolases comprising the amino acid sequence of SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86, SEQ ID NO 89 and polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto.

One embodiment relates to a composition comprising a GHL13 glycosyl hydrolase, which comprises one or more motif(s) [Y/W]PX[D/N]F (SEQ ID NO 59), [W/E/Y/F]AM[P/G] (SEQ ID NO 60) or WPY and/or is selected from the group consisting of GHL13 glycosyl hydrolases having the amino acid sequence of SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86, SEQ ID NO 89 and polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto and preferably an adjunct ingredient. The composition may be an anti-biofouling composition and the composition may be a cleaning or pharmaceutical composition. The adjunct ingredient may be any excipient suitable for e.g. cleaning or pharmaceutical compositions. The adjuncts/excipients are within the choice of the skilled artisan. The adjunct ingredient may be selected from the group consisting of surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers. The compositions may be used for detaching biofilm or preventing biofilm formation on surfaces such as medical devices. The medical device may be characterized in that at least a portion of a patient-contactable surface of said device is coated with composition comprising a GHL13 glycosyl hydrolase of the invention.

The medical device or implant may be any device or implant that is susceptible to biofilm formation. The medical device may be selected from the group consisting of a catheter such as a central venous catheter, intravascular catheter, urinary catheter, Hickman catheter, peritoneal dialysis catheter, endrotracheal catheter, or wherein the device is a mechanical heart valve, a cardiac pacemaker, an arteriovenous shunt, a scleral buckle, a prosthetic joint, a tympanostomy tube, a tracheostomy tube, a voice prosthetic, a penile prosthetic, an artificial urinary sphincter, a synthetic pubovaginal sling, a surgical suture, a bone anchor, a bone screw, an intraocular lens, a contact lens, an intrauterine device, an aortofemoral graft, a vascular graft, a needle, a Luer-Lok connector, a needleless connector and a surgical instrument.

### Uses

The polypeptides of the invention having hydrolytic activity may be used for cleaning e.g. deep cleaning of an item, such as a textile. In a preferred embodiment the polypeptides of the invention comprise one or more of the motif(s) Y/W]PX[D/N]F (SEQ ID NO 59), [M/E/Y/F]AM[P/G] (SEQ ID NO 60) or WPY. In some embodiment of the invention relates to the use of a polypeptide according to the invention for prevention reduction or removal of malodor. Some embodiment of the invention relates to the use of a polypeptide of the invention for prevention or reduction of anti-redeposition and improvement of whiteness of a textile subjected to multiple washes. One embodiment of the invention relates to the use of a polypeptide according to the invention for deep cleaning of an item, wherein item is a textile. One embodiment of the invention relates to the use of a polypeptide according to the invention
(i) for preventing, reducing or removing stickiness of the item;
(ii) for pretreating stains on the item;
(iii) for preventing, reducing or removing redeposition of soil during a wash cycle;
(iv) for preventing, reducing or removing adherence of soil to the item;
(v) for maintaining or improving whiteness of the item;
(vi) for preventing, reducing or removal malodor from the item,
   wherein the item is a textile.
      One embodiment of the invention relates to the use of a polypeptide according to the invention for deep cleaning of an item, wherein item is a textile. One embodiment of the invention relates to the use of a polypeptide,
(i) for preventing, reducing or removing stickiness of the item;
(ii) for pretreating stains on the item;
(iii) for preventing, reducing or removing redeposition of soil during a wash cycle;
(iv) for preventing, reducing or removing adherence of soil to the item;
(v) for maintaining or improving whiteness of the item;
(vi) for preventing, reducing or removal malodor from the item, optionally wherein the item is a textile, wherein the polypeptide is selected from the group consisting of:
   (a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 3;
   (b) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
   (c) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 9;
   (d) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 12;
   (e) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 15;
   (f) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 18;
   (g) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 21;
   (h) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
   (i) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 27;
   (j) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
   (k) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 33;
   (l) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 36;
   (m) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 39;

(n) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 42;
(o) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 45;
(p) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 48;
(q) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 65;
(r) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 68;
(s) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 71;
(t) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 74;

(u) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 77;
(v) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 80;
(x) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 83;
(y) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 86; and
(z) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 89.

The invention is further summarized in the following paragraphs:
1. Use of a polypeptide comprising a GHL13 domain and/or CE4 catalytic domain for deep cleaning of an item, wherein the item is a textile.
2. Use according to paragraph 1 for preventing, reducing or removing stickiness of the item.
3. Use according to any of paragraphs 1 or 2 for pre-treating stains on the item.
4. Use according to any of paragraphs 1-3 for preventing, reducing or removing re-deposition of soil during a wash cycle.
5. Use according to any of paragraphs 1-4 for preventing, reducing or removing adherence of soil to the item.
6. Use according to any of the preceding paragraphs for maintaining or improving the whiteness of the item.
7. Use according to any of the preceding paragraphs, wherein a malodor is reduced or removed from the item.
8. Use according to any of the preceding composition paragraphs, wherein the surface is a textile surface.
9. Use according to any of the preceding composition paragraphs, wherein the textile is made of cotton, Cotton/Polyester, Polyester, Polyamide, Polyacryl and/or silk.
10. Use according to any of the preceding paragraphs, wherein the polypeptide is a polypeptide of paragraphs 47-61
11. A composition comprising a polypeptide comprising a GHL13 domain and/or CE4 catalytic domain and an adjunct ingredient.
12. Composition according to paragraph 11, wherein the polypeptide is the polypeptide of paragraphs 60-92.
13. Composition according to any of the preceding composition paragraphs, wherein the detergent adjunct ingredient is selected from the group consisting of surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and/or pigments.
14. Composition according to any of the preceding composition paragraphs wherein the composition comprises from about 5 wt % to about 50 wt %, from about 5 wt % to about 40 wt %, from about 5 wt % to about 30 wt %, from about 5 wt % to about 20 wt %, from about 5 wt % to about 10 wt % anionic surfactant, preferably selected from linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.
15. Composition according to any of the preceding composition paragraphs wherein the composition comprises from about 10 wt % to about 50 wt % of at least one builder, preferably selected from citric acid, methylglycine-N,N-diacetic acid (MGDA) and/or glutamic acid-N,N-diacetic acid (GLDA) and mixtures thereof.
16. Composition according to any of the preceding paragraphs comprising from about 5 wt % to about 40 wt % nonionic surfactants, and from about 0 wt % to about 5 wt % anionic surfactants.
17. Composition according to paragraph 16, wherein the nonionic surfactant is selected from alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA) and combinations thereof.
18. Composition according to any of the preceding composition paragraphs, wherein the composition further comprises one or more enzymes selected from the group consisting of proteases, lipases, cutinases, amylases, carbohydrases, cellulases, pectinases, mannanases, arabinases, galactanases, xylanases and oxidases.
19. Composition according to any of the preceding composition paragraphs, wherein the composition is a bar, a homogenous tablet, a tablet having two or more layers, a pouch having one or more compartments, a regular or compact powder, a granule, a paste, a gel, or a regular, compact or concentrated liquid.
20. Composition according to any of the preceding composition paragraphs, wherein the composition is a cleaning composition selected from liquid detergent, powder detergent and granule detergent compositions.
21. Composition according to any of the preceding composition paragraphs wherein the polypeptide comprising a GHL13 domain and/or CE4 catalytic domain is selected from the group consisting of polypeptides having the amino acid sequence of SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86 and SEQ ID NO 89 and polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
22. Composition according to any of the preceding composition paragraphs wherein the polypeptide comprising a GHL13 domain and/or CE4 catalytic domain comprises the amino acid sequence shown SEQ ID NO 3 or polypeptides having at least 60%, at least 65%, at least 70%, at least 75%, at least 80%, at least 85%, at least 90% at least 95%, at least 96%, at least 97%, at least 98%, at least 99% or 100 sequence identity hereto.
23. Composition according to any of the preceding composition paragraphs wherein the polypeptide comprising a GHL13 domain and/or CE4 catalytic domain comprises the amino acid sequence shown SEQ ID NO 6 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
24. Composition according to any of the preceding composition paragraphs wherein the polypeptide comprising a GHL13 domain and/or CE4 catalytic domain comprises the amino acid sequence shown SEQ ID NO 9 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
25. Composition according to any of the preceding composition paragraphs wherein the polypeptide comprising a GHL13 domain and/or CE4 catalytic domain comprises the amino acid sequence shown SEQ ID NO 12 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
26. Composition according to any of the preceding composition paragraphs wherein the polypeptide comprising a GHL13 domain and/or CE4 catalytic domain comprises the amino acid sequence shown SEQ ID NO 15 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
27. Composition according to any of the preceding composition paragraphs wherein the polypeptide comprising a GHL13 domain and/or CE4 catalytic domain comprises the amino acid sequence shown SEQ ID NO 18 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
28. Composition according to any of the preceding composition paragraphs wherein the polypeptide comprising a GHL13 domain and/or CE4 catalytic domain comprises the amino acid sequence shown SEQ ID NO 21 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
29. Composition according to any of the preceding composition paragraphs wherein the polypeptide comprising a GHL13 domain and/or CE4 catalytic domain comprises the amino acid sequence shown SEQ ID NO 24 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
30. Composition according to any of the preceding composition paragraphs wherein the polypeptide comprising a GHL13 domain and/or CE4 catalytic domain comprises the amino acid sequence shown SEQ ID NO 27 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
31. Composition according to any of the preceding composition paragraphs wherein the polypeptide comprising a GHL13 domain and/or CE4 catalytic domain comprises the amino acid sequence shown SEQ ID NO 30 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
32. Composition according to any of the preceding composition paragraphs wherein the polypeptide comprising a GHL13 domain and/or CE4 catalytic domain comprises the amino acid sequence shown SEQ ID NO 33 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
33. Composition according to any of the preceding composition paragraphs wherein the polypeptide comprising a GHL13 domain and/or CE4 catalytic domain comprises the amino acid sequence shown SEQ ID NO 36 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
34. Composition according to any of the preceding composition paragraphs wherein the polypeptide comprising a GHL13 domain and/or CE4 catalytic domain comprises the amino acid sequence shown SEQ ID NO 39 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
35. Composition according to any of the preceding composition paragraphs wherein the polypeptide comprising a GHL13 domain and/or CE4 catalytic domain comprises the amino acid sequence shown SEQ ID NO 42 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
36. Composition according to any of the preceding composition paragraphs wherein the polypeptide comprising a GHL13 domain and/or CE4 catalytic domain comprises the amino acid sequence shown SEQ ID NO 45 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
37. Composition according to any of the preceding composition paragraphs wherein the polypeptide comprising a GHL13 domain and/or CE4 catalytic domain comprises the amino acid sequence shown SEQ ID NO 48 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
38. Composition according to any of the preceding composition paragraphs wherein the polypeptide comprising a GHL13 domain and/or CE4 catalytic domain comprises the amino acid sequence shown SEQ ID NO 65 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
39. Composition according to any of the preceding composition paragraphs wherein the polypeptide comprising a GHL13 domain and/or CE4 catalytic domain comprises the amino acid sequence shown SEQ ID NO 68 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
40. Composition according to any of the preceding composition paragraphs wherein the polypeptide comprising a GHL13 domain and/or CE4 catalytic domain comprises the amino acid sequence shown SEQ ID NO 71 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
41. Composition according to any of the preceding composition paragraphs wherein the polypeptide comprising a GHL13 domain and/or CE4 catalytic domain comprises the amino acid sequence shown SEQ ID NO 74 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
42. Composition according to any of the preceding composition paragraphs wherein the polypeptide comprising a GHL13 domain and/or CE4 catalytic domain comprises the amino acid sequence shown SEQ ID NO 77 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
43. Composition according to any of the preceding composition paragraphs wherein the polypeptide comprising a GHL13 domain and/or CE4 catalytic domain comprises the amino acid sequence shown SEQ ID NO 80 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
44. Composition according to any of the preceding composition paragraphs wherein the polypeptide comprising a GHL13 domain and/or CE4 catalytic domain comprises the amino acid sequence shown SEQ ID NO 83 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
45. Composition according to any of the preceding composition paragraphs wherein the polypeptide comprising a GHL13 domain and/or CE4 catalytic domain comprises the amino acid sequence shown SEQ ID NO 86 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
46. Composition according to any of the preceding composition paragraphs wherein the polypeptide comprising a GHL13 domain and/or CE4 catalytic domain comprises the amino acid sequence shown SEQ ID NO 89 or polypeptides having at least 60%, at least 65%, at least 70%, at least 75%, at least 80%, at least 85%, at least 90% at least 95%, at least 96%, at least 97%, at least 98%, at least 99% or 100% sequence identity hereto.
47. A laundering method for laundering an item comprising the steps of:
   a. Exposing an itm to a wash liquor comprising a polypeptide of paragraphs 61-94 or a composition according to any of paragraphs 11-46;
   b. Completing at least one wash cycle; and
   c. Optionally rinsing the item,
   wherein the item is a textile.
48. A method for deep cleaning of an item, wherein the item is preferably a textile, said method comprising the steps of:
   a. Exposing an item to a polypeptide selected from the group consisting of a polypeptide having at least 60%, at least 65%, at least 70%, at least 75%, at least 80%, at least 85%, at least 90% at least 95%, at least 96%, at least 97%, at least 98%, at least 99% or 100% sequence identity to the mature polypeptide shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45,SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86, SEQ ID NO 89 a wash liquor comprising said polypeptide or a detergent composition according to any preceding paragraphs.
49. Method according to any preceding paragraphs, wherein the pH of the wash liquor is in the range of 1 to 11.
50. Method according to any of the preceding method paragraphs, wherein the pH of the wash liquor is in the range 5.5 to 11, such as in the range of 7 to 9, in the range of 7 to 8 or in the range of 7 to 8.5.
51. Method according to any of the preceding method paragraphs, wherein the temperature of the wash liquor is in the range of 5° C. to 95° C., or in the range of 10° C. to 80° C., in the range of 10° C. to 70° C., in the range of 10° C. to 60° C., in the range of 10° C. to 50° C., in the range of 15° C. to 40° C., in the range of 20° C. to 40° C., in the range of 15° C. to 30° C. or in the range of 20° C. to 30° C.
52. Method according to any of the preceding method paragraphs, wherein the temperature of the wash liquor is from about 20° C. to about 40° C.
53. Method according to any of the preceding method paragraphs, wherein the temperature of the wash liquor is from about 15° C. to about 30° C.
54. Method according to any of the preceding method paragraphs, wherein stains present on the item is pre-treated with a polypeptide of paragraphs 61-94 or a composition e.g. detergent composition according to any of paragraphs 11-46.
55. Method according to any of the preceding method paragraphs, wherein stickiness of the item is reduced.
56. Method according to any of the preceding method paragraphs, wherein redeposition of soil is reduced.
57. Method according to any of the preceding method paragraphs, wherein adherence of soil to the item is reduced or removed.
58. Method according to any of the preceding method paragraphs, wherein whiteness of the item is maintained or improved.
59. Method according to any of the preceding method paragraphs, wherein malodor is reduced or removed from the item.
60. Method according to any of the preceding method paragraphs, wherein the concentration of the polypeptide having hydrolytic and/or deacetylase activity in the wash liquor is at least 0.001 mg of polypeptide, per liter of wash liquor. optionally the concentration of polypeptide in the wash liquor is in the range 0.002 mg/L to 2 mg/L, such as 0.02 mg/L to 2 mg/L, such as 0.2 mg/L to 2 mg/L, or in the range of 0.0001 mg/L to 10 mg/L, or in the range of in the range of 0.001 mg/L to 10 mg/L, or in the range of 0.01 mg/L to 10 mg/L, or in in the range of 0.1 mg/L to 10 mg/L per liter of wash liquor, optionally the concentration of the polypeptide of the invention is 0.0001 to 2 wt %. such as 0.001 to 0.1 wt %. such as 0.005 to 0.1 wt %. such as 0.01 to 0.1 wt %. such as 0.01 to 0.5 wt % or most preferred 0.002 to 0.09 wt % in the total detergent concentration.
61. A polypeptide having hydrolytic and/or deacetylase activity selected from the group consisting of:
   a. a polypeptide having at least 60%, at least 65%, at least 70%, at least 75%, at least 80%, at least 85%, at least 90% at least 95%, at least 96%, at least 97%, at least 98%, at least 99% or 100% sequence identity to the mature polypeptide shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45,SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86 or SEQ ID NO 89;
   b. a polypeptide encoded by a polynucleotide that hybridizes under low stringency conditions with
      i. the mature polypeptide coding sequence of SEQ ID NO 1, SEQ ID NO 4, SEQ ID NO 7, SEQ ID NO 10, SEQ ID NO 13, SEQ ID NO 16, SEQ ID NO 19, SEQ ID NO 22, SEQ ID NO 25, SEQ ID NO 28, SEQ ID NO 31, SEQ ID NO 34, SEQ ID NO 37, SEQ ID NO 40, SEQ ID NO 43 or SEQ ID NO 46, SEQ ID NO 63, SEQ ID NO 66, SEQ ID NO 69, SEQ ID NO 72, SEQ ID NO 75. SEQ ID NO 78, SEQ ID NO 81, SEQ ID NO 84 or SEQ ID NO 87;
      ii. the cDNA sequence thereof, or
      iii. the full-length complement of (i) or (ii);
   c. a polypeptide encoded by a polynucleotide having at least 60%, at least 65%, at least 70%, at least 75%, at least 80%, at least 85%, at least 90% at least 95%, at least 96%, at least 97%, at least 98%, at least 99% or 100% sequence identity to the mature polypeptide coding sequence of SEQ ID NO 1, SEQ ID NO 4, SEQ ID NO 7, SEQ ID NO 10, SEQ ID NO 13, SEQ ID NO 16, SEQ ID NO 19, SEQ ID NO 22, SEQ ID NO 25, SEQ ID NO 28, SEQ ID NO 31, SEQ ID NO 34, SEQ ID NO 37, SEQ ID NO 40, SEQ ID NO 43, SEQ ID NO 46, SEQ ID NO 63, SEQ ID NO 66, SEQ ID NO 69, SEQ ID NO 72, SEQ ID NO 75. SEQ ID NO 78, SEQ ID NO 81, SEQ ID NO 84, SEQ ID NO 87 or the cDNA sequence thereof;
   d. a variant of the mature polypeptide shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45,SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86 or SEQ ID NO 89 comprising a substitution, deletion, and/or insertion at one or more positions or a variant of the mature polypeptide shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86 and SEQ ID nO 89 comprising a substitution, deletion, and/or insertion at one or more positions; and
   e. a fragment of the polypeptide of (a), (b), (c) or (d) that comprises a GHL13 and/or CE4 catalytic domain.
62. The polypeptide of paragraph 61, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 2, SEQ ID NO 5, SEQ ID NO 8, SEQ ID NO 11, SEQ ID NO 14, SEQ ID NO 17, SEQ ID NO 20, SEQ ID NO 23, SEQ ID NO 26, SEQ ID NO 29, SEQ ID NO 32, SEQ ID NO 35, SEQ ID NO 38, SEQ ID NO 41, SEQ ID NO 44, SEQ ID NO 47, SEQ ID NO 64, SEQ ID NO 67, SEQ ID NO 70, SEQ ID NO 73, SEQ ID NO 76, SEQ ID NO 79, SEQ ID NO 82, SEQ ID NO 85, SEQ ID NO 88 or to the mature polypeptide shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45,SEQ ID NO 48 SEQ ID NO 65, SEQ ID NO 68, SEQ ID NO 71, SEQ ID NO 74, SEQ ID NO 77. SEQ ID NO 80, SEQ ID NO 83, SEQ ID NO 86 or SEQ ID NO 89.
63. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 2 or to the mature polypeptide shown in SEQ ID NO 3.
64. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 5 or to the mature polypeptide shown in SEQ ID NO 6.
65. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 8 or to the mature polypeptide shown in SEQ ID NO 9.
66. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 11 or to the mature polypeptide shown in SEQ ID NO 12.
67. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 14 or to the mature polypeptide shown in SEQ ID NO 15.
68. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 17 or to the mature polypeptide shown in SEQ ID NO 18.
69. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 20 or to the mature polypeptide shown in SEQ ID NO 21.
70. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 23 or to the mature polypeptide shown in SEQ ID NO 24.
71. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 26 or to the mature polypeptide shown in SEQ ID NO 27.
72. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 29 or to the mature polypeptide shown in SEQ ID NO 30.
73. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 32 or to the mature polypeptide shown in SEQ ID NO 33.
74. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 35 or to the mature polypeptide shown in SEQ ID NO 36.
75. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 38 or to the mature polypeptide shown in SEQ ID NO 39.
76. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 41 or to the mature polypeptide shown in SEQ ID NO 42.
77. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 44 or to the mature polypeptide shown in SEQ ID NO 45.
78. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 47 or to the mature polypeptide shown in SEQ ID NO 48.
79. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 64 or to the mature polypeptide shown in SEQ ID NO 65.
80. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 67 or to the mature polypeptide shown in SEQ ID NO 68.
81. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 70 or to the mature polypeptide shown in SEQ ID NO 71.
82. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 73 or to the mature polypeptide shown in SEQ ID NO 74.
83. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 76 or to the mature polypeptide shown in SEQ ID NO 77.
84. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 79 or to the mature polypeptide shown in SEQ ID NO 80.
85. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 82 or to the mature polypeptide shown in SEQ ID NO 83.
86. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 85 or to the mature polypeptide shown in SEQ ID NO 86.
87. The polypeptide of paragraph 61 or 62, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO 88 or to the mature polypeptide shown in SEQ ID NO 89.
88. The polypeptide according to any of paragraphs 61 to 87, which is encoded by a polynucleotide that hybridizes under low stringency conditions, low-medium stringency conditions, medium stringency conditions, medium-high stringency conditions, high stringency conditions, or very high stringency conditions with
   i. the mature polypeptide coding sequence of SEQ ID NO 1, SEQ ID NO 4, SEQ ID NO 7, SEQ ID NO 10, SEQ ID NO 13, SEQ ID NO 16, SEQ ID NO 19, SEQ ID NO 22, SEQ ID NO 25, SEQ ID NO 28, SEQ ID NO 31, SEQ ID NO 34, SEQ ID NO 37, SEQ ID NO 40, SEQ ID NO 43,SEQ ID NO 46, SEQ ID NO 63, SEQ ID NO 66, SEQ ID NO 69, SEQ ID NO 72, SEQ ID NO 75, SEQ ID NO 78, SEQ ID NO 81, SEQ ID NO 84 and SEQ ID NO 87;
   ii. the cDNA sequence thereof, or
   iii. the full-length complement of (i) or (ii).
89. The polypeptide according to any of paragraphs 61-88, which is encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO 1, SEQ ID NO 4, SEQ ID NO 7, SEQ ID NO 10, SEQ ID NO 13, SEQ ID NO 16, SEQ ID NO 19, SEQ ID NO 22, SEQ ID NO 25, SEQ ID NO 28, SEQ ID NO 31, SEQ ID NO 34, SEQ ID NO 37, SEQ ID NO 40, SEQ ID NO 43,SEQ ID NO 46, SEQ ID NO 63, SEQ ID NO 66, SEQ ID NO 69, SEQ ID NO 72, SEQ ID NO 75, SEQ ID NO 78, SEQ ID NO 81, SEQ ID NO 84 and SEQ ID NO 87 or the cDNA sequence thereof.
90. The polypeptide according to any of paragraphs 61 to 88, comprising or consisting of SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86 and SEQ ID NO 89 or the mature polypeptide of SEQ ID NO 2, SEQ ID NO 5, SEQ ID NO 8, SEQ ID NO 11, SEQ ID NO 14, SEQ ID NO 17, SEQ ID NO 20, SEQ ID NO 23, SEQ ID NO 26, SEQ ID NO 29, SEQ ID NO 32, SEQ ID NO 35, SEQ ID NO 38, SEQ ID NO 41, SEQ ID NO 44,SEQ ID NO 47, SEQ ID NO 64, SEQ ID NO 67, SEQ ID NO 70, SEQ ID NO 73, SEQ ID NO 76, SEQ ID NO 79, SEQ ID NO 82, SEQ ID NO 85 or SEQ ID NO 88.
91. The polypeptide according to any of paragraphs 61 to 88, which is a variant of SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77, SEQ ID NO 80, SEQ ID NO 83, SEQ ID NO 86 and SEQ ID NO 89 comprising a substitution, deletion, and/or insertion at one or more positions.
92. The polypeptide according to any of preceding paragraphs for use as a medicament.
93. The polypeptide according to any of preceding paragraphs for use in treatment or prevention of a bacterial infection, preferably said bacterial infection is an infection caused by Gram-positive or Gram-negative bacteria, further preferably said bacterial infection is selected from a group consisting of: Staphylococcus spp. (e.g., Staphylococcus epidermidis, S. aureus), Enterococcus spp. (e.g., Enterococcus faecalis), Escherichia spp. (e.g., Escherichia coli), Listeria spp. (e.g., Listeria monocytogenes), Pseudomonas spp. (e.g., Pseudomonas aeruginosa), Bacillus spp., Salmonella spp., Coagulase-negative Staphylococci, Klebsiella spp. (e.g., Klebsiella pneumoniae) infections.
94. The polypeptide according to any of preceding paragraphs for use in treatment or prevention of a disease selected from the group consisting of: Cystic fibrosis pneumonia (e.g., caused by Pseudomonas aeruginosa and/or Burkholderia cepacia), Meloidosis (e.g., caused by Pseudomonas pseudomallei), Necrotizing fasciitis (e.g., caused by Group A streptococci), Musculoskeletal infections (e.g., caused by Staphylococci and other Gram-positive cocci), Otitis media (e.g., caused by Haemophilus influenzae), Biliary tract infection (e.g., caused by E. coli and other enteric bacteria), Urinary catheter cystitis (e.g., caused by E. coli and other Gram-negative rods), Bacterial prostatitis (e.g., E. coli and other Gram-negative bacteria), Periodontitis (e.g., caused by Gram negative anaerobic oral bacteria), Dental caries (e.g., caused by Streptococcus spp. and other acidogenic Gram positive cocci).
95. A polynucleotide encoding the polypeptide according to any of paragraphs 61-94.
96. A nucleic acid construct or expression vector comprising the polynucleotide of paragraph 95 operably linked to one or more control sequences that direct the production of the polypeptide in an expression host.
97. A recombinant host cell comprising the polynucleotide of paragraph 95 operably linked to one or more control sequences that direct the production of the polypeptide.
98. A method of producing the polypeptide of any of paragraphs 61-94, comprising cultivating a cell, which in its wild-type form produces the polypeptide, under conditions conducive for production of the polypeptide.
99. The method of paragraph 98, further comprising recovering the polypeptide.
100. A method of producing a polypeptide according to any of paragraphs 61-94, comprising cultivating the host cell of paragraph 97 under conditions conducive for production of the polypeptide.
101. The method of paragraph 100, further comprising recovering the polypeptide.
102. A nucleic acid construct or expression vector comprising a gene encoding a protein operably linked to the polynucleotide of paragraph 95, wherein the gene is foreign to the polynucleotide encoding the signal peptide.
103. A recombinant host cell comprising a gene encoding a protein operably linked to the polynucleotide of paragraph 95, wherein the gene is foreign to the polynucleotide encoding the signal peptide.
104. A method of producing a protein, comprising cultivating a recombinant host cell comprising a gene encoding a protein operably linked to the polynucleotide of paragraph 95, wherein the gene is foreign to the polynucleotide encoding the signal peptide, under conditions conducive for production of the protein.
105. The method of paragraph 104, further comprising recovering the protein.
106. The recombinant host cell of paragraph 97 further comprising a polynucleotide encoding a second polypeptide of interest; preferably an enzyme of interest; more preferably a secreted enzyme of interest; even more preferably a hydrolase, isomerase, ligase, lyase, oxidoreductase, or a transferase; and most preferably the secreted enzyme is an alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucano-transferase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or a xylanase.
107. The recombinant host cell of paragraph 106, wherein the second polypeptide of interest is heterologous or homologous to the host cell.
108. The recombinant host cell of paragraph 103 or 106, which is a fungal host cell; preferably a filamentous fungal host cell; more preferably an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell; most preferably an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonaturn, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulaturn, Fusarium roseum, Fusarium sambucinum, Fusarium sarcochroum, Fusarium sporotrichioides, Fusarium sulphureum, Fusarium torulosum, Fusarium trichothecioides, Fusarium venenaturn, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.
109. The recombinant host cell of paragraph 103 or 106, which is a bacterial host cell; preferably a prokaryotic host cell; more preferably a Gram-positive host cell; even more preferably a Bacillus, Clostridium, Enterococcus, Geobacillus, Lactobacillus, Lactococcus, Oceanobacillus, Staphylococcus, Streptococcus, or Streptomyces host cell; and most preferably a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis host cell.
110. Item laundered according to the method of any of paragraphs 47-60.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Definitions

Activity: The present inventions relates to glycosyl hydrolases (EC 3.2.1.-), which are a widespread group of enzymes that hydrolyse the glycosidic bond between two or more carbohydrates or between a carbohydrate and a non-carbohydrate moiety. A classification of glycoside hydrolases in families based on amino acid sequence similarities has been proposed. The polypeptides of the invention comprise at least one glycosyl hydrolase domain and are in the present context defined as glycosyl hydrolases. Thus, polypeptides of the invention hydrolyse glycosidic bonds and the polypeptides of the invention have hydrolytic activity. The glycosyl hydrolase domain comprised in the polypeptide of the invention may be classified as a GHL13 domain (PF14883) and in particular as belonging to GHL13 subclade and in a preferred embodiment the polypeptides of the invention have hydrolytic (EC 3.2.1.) activity (http://www.cazy.org/). The polypeptides of the invention are PgaBs and/or BpsB. The C-terminal domain of PgaB has structural similarity to many glycoside hydrolases and based on amino acid sequence identity, the PFAM database (Pfam version 31.0 Finn (2016). Nucleic Acids Research, Database Issue 44: D279-D285) recently categorized both BpsB and PgaB C-terminal domains as members of the GHL13 family (PFAM domain id PF14883). The polypeptides of the invention are BpsB and PgaB homologs comprise a GHL13 domain and show activity towards PNAG (poly-N-acetylglucosamine) substrate. PgaB enzyme is further classified as a member of the family 4 carbohydrate esterases (CE4) enzymes as defined by the CAZY database [http://www.cazy.org/ (Coutinho & Henrissat, 1999)]. Some polypeptides of the invention also comprise the CE4 domain. Thus, in one aspect the polypeptides of the invention comprise deacetylase activity.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Biofilm: A biofilm is organic matter produced by any group of microorganisms in which cells stick to each other or stick to a surface, such as a textile, dishware or hard surface or another kind of surface. These adherent cells are frequently embedded within a self-produced matrix of extracellular polymeric substance (EPS). Biofilm EPS is a polymeric conglomeration generally composed of extracellular DNA, proteins, and polysaccharides. Biofilms may form on living or non-living surfaces. The microbial cells growing in a biofilm are physiologically distinct from planktonic cells of the same organism, which, by contrast, are single-cells that may float or swim in a liquid medium. Bacteria living in a biofilm usually have significantly different properties from planktonic bacteria of the same species, as the dense and protected environment of the film allows them to cooperate and interact in various ways. One benefit of this environment for the microorganisms is increased resistance to detergents and antibiotics, as the dense extracellular matrix and the outer layer of cells protect the interior of the community.

On laundry biofilm producing bacteria can be found among the following species: Acinetobacter sp., Aeromicrobium sp., Brevundimonas sp., Microbacterium sp., Micrococcus luteus, Pseudomonas sp., Staphylococcus epidermidis, and Stenotrophomonas sp. On hard surfaces biofilm producing bacteria can be found among the following species: Acinetobacter sp., Aeromicrobium sp., Brevundimonas sp., Microbacterium sp., Micrococcus luteus, Pseudomonas sp., Staphylococcus epidermidis, Staphylococcus aureus and Stenotrophomonas sp. In one aspect, the biofilm producing strain is Brevundimonas sp. In one aspect, the biofilm producing strain is Pseudomonas e.g. Pseudomonas aeruginosa, Pseudomonas alcaliphila or Pseudomonas fluorescens. In one aspect, the biofilm producing strain is Staphylococcus aureus.

In one embodiment, the biofilm is caused by microorganisms or group of microorganisms which produce PNAG. In another embodiment, the biofilm produce a polysaccharide that is degradable by the GHL13 glycosyl hydrolases of the invention. The biofilm that may be formed on the surface e.g. such as textiles may be caused by any microorganism or group of microorganisms that forms PNAG-dependent biofilm including but not limited to; Acinetobacter sp., Aeromicrobium sp., Brevundimonas sp., Microbacterium sp., Micrococcus luteus, Staphylococcus epidermidis, Staphylococcus aureus, Pseudomonas sp., Pseudomonas aeruginosa, Pseudomonas alcaliphila, Pseudomonas fluorescens, Stenotrophomonas sp., Paraburkholderia, Burkolderia sp., Candida sp., Bordetella pertussis Yersinia pestis, Escherichia coli and Aspergillus sp.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Deep cleaning: The term “deep cleaning” means disruption, reduction or removal of organic components such as polysaccharides e.g. PNAG, proteins, DNA, soil or other components present in organic matter such as biofilm.

Cleaning component: e.g. detergent adjunct ingredient. The cleaning component e.g. the detergent adjunct ingredient is different to the polypeptides of this invention. The precise nature of these additional cleaning (adjunct) components, and levels of incorporation thereof, will depend on the physical form of the composition and the nature of the operation for which it is to be used. Suitable cleaning component or adjunct materials include, but are not limited to the components described below such as surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and/or pigments.

Cleaning composition: The term cleaning composition includes “detergent composition” refers to compositions that find use in the removal of undesired compounds from items to be cleaned, such as textiles. The cleaning or detergent composition may be used to e.g.

clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). In addition to containing the enzyme of the invention, the detergent formulation may contain one or more additional enzymes (such as proteases, amylases, lipases, cutinases, cellulases, endoglucanases, xyloglucanases, pectinases, pectin lyases, xanthanases, peroxidases, haloperoxygenases, catalases and mannanases, or any mixture thereof), and/or detergent adjunct ingredients such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Enzyme Detergency benefit: The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and/or cleaning, prevention or reduction of redeposition of soils released in the washing process (an effect that also is termed anti-redeposition), restoring fully or partly the whiteness of textiles which originally were white but after repeated use and wash have obtained a greyish or yellowish appearance (an effect that also is termed whitening). Textile care benefits, which are not directly related to catalytic stain removal or prevention of redeposition of soils, are also important for enzyme detergency benefits. Examples of such textile care benefits are prevention or reduction of dye transfer from one fabric to another fabric or another part of the same fabric (an effect that is also termed dye transfer inhibition or anti-backstaining), removal of protruding or broken fibers from a fabric surface to decrease pilling tendencies or remove already existing pills or fuzz (an effect that also is termed anti-pilling), improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching components such as hydrogen peroxide or other peroxides.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide or a catalytic domain having one or more (e.g., several) amino acids absent from the amino and/or carboxyl terminus of a mature polypeptide or domain; wherein the fragment has activity.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g. a host cell may be genetically modified to express the polypeptide of the invention. The fermentation broth from that host cell will comprise the isolated polypeptide.

Improved wash performance: The term “improved wash performance” is defined herein as an enzyme displaying an increased wash performance in a detergent composition relative to the wash performance of same detergent composition without the enzyme e.g. by increased stain removal or less re-deposition. The term “improved wash performance” includes wash performance in laundry.

Laundering: The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

Malodor: By the term “malodor” is meant an odor which is not desired on clean items. The cleaned item should smell fresh and clean without malodors adhered to the item. One example of malodor is compounds with an unpleasant smell, which may be produced by microorganisms. Another example is unpleasant smells can be sweat or body odor adhered to an item which has been in contact with human or animal. Another example of malodor can be the odor from spices, which sticks to items for example curry or other exotic spices which smells strongly.

Mature polypeptide: The term “mature polypeptide” means a polypeptide in its final form following translation and any post-translational modifications, such as N-terminal processing, C-terminal truncation, glycosylation, phosphorylation, etc. In some aspects, the mature polypeptide is amino acids 1 to 645 of SEQ ID NO 2 and amino acids −20 to −1 of SEQ ID NO 2 is a signal peptide. In some aspects, the mature polypeptide is the amino acid sequence shown in SEQ ID NO 3. In some aspects, the mature polypeptide is amino acids 1 to 629 of SEQ ID NO 5 and amino acids −24 to −1 of SEQ ID NO 5 is a signal peptide. In some aspects, the mature polypeptide is the amino acid sequence shown in SEQ ID NO 6. In some aspects, the mature polypeptide is amino acids 1 to 645 of SEQ ID NO 8 and amino acids −20 to −1 of SEQ ID NO 8 is a signal peptide. In some aspects, the mature polypeptide is the amino acid sequence having SEQ ID NO 9. In some aspects, the mature polypeptide is amino acids 1 to 646 of SEQ ID NO 11 and amino acids −20 to −1 of SEQ ID NO 11 is a signal peptide. In some aspects, the mature polypeptide is the amino acid sequence having SEQ ID NO 12. In some aspects, the mature polypeptide is amino acids 1 to 638 of SEQ ID NO 14 and amino acids −27 to −1 of SEQ ID NO 14 is a signal peptide. In some aspects, the mature polypeptide is the amino acid sequence having SEQ ID NO 15. In some aspects, the mature polypeptide is amino acids 1 to 638 of SEQ ID NO 17 and amino acids −27 to −1 of SEQ ID NO 17 is a signal peptide. In some aspects, the mature polypeptide is the amino acid sequence having SEQ ID NO 18. In some aspects, the mature polypeptide is amino acids 1 to 606 of SEQ ID NO 20 and amino acids −23 to −1 of SEQ ID NO 20 is a signal peptide. In some aspects, the mature polypeptide is the amino acid sequence having SEQ ID NO 21. In some aspects, the mature polypeptide is amino acids 1 to 640 of SEQ ID NO 23 and amino acids −25 to −1 of SEQ ID NO 23 is a signal peptide. In some aspects, the mature polypeptide is the amino acid sequence having SEQ ID NO 24. In some aspects, the mature polypeptide is amino acids 1 to 380 of SEQ ID NO 26 and amino acids −22 to −1 of SEQ ID NO 26 is a signal peptide. In some aspects, the mature polypeptide is the amino acid sequence having SEQ ID NO 27. In some aspects, the mature polypeptide is amino acids 1 to 645 of SEQ ID NO 29 and amino acids −20 to −1 of SEQ ID NO 29 is a signal peptide. In some aspects, the mature polypeptide is the amino acid sequence having SEQ ID NO 30. In some aspects, the mature polypeptide is amino acids 1 to 629 of SEQ ID NO 32 and amino acids −24 to −1 of SEQ ID NO 32 is a signal peptide. In some aspects, the mature polypeptide is the amino acid sequence having SEQ ID NO 33. In some aspects, the mature polypeptide is amino acids 1 to 629 of SEQ ID NO 35 and amino acids −24 to −1 of SEQ ID NO 35 is a signal peptide. In some aspects, the mature polypeptide is the amino acid sequence having SEQ ID NO 36. In some aspects, the mature polypeptide is amino acids 1 to 629 of SEQ ID NO 38 and amino acids −24 to −1 of SEQ ID NO 38 is a signal peptide. In some aspects, the mature polypeptide is the amino acid sequence having SEQ ID NO 39. In some aspects, the mature polypeptide is amino acids 1 to 605 of SEQ ID NO 41 and amino acids −19 to −1 of SEQ ID NO 41 is a signal peptide. In some aspects, the mature polypeptide is the amino acid sequence having SEQ ID NO 42. In some aspects, the mature polypeptide is amino acids 1 to 630 of SEQ ID NO 44 and amino acids −23 to −1 of SEQ ID NO 44 is a signal peptide. In some aspects, the mature polypeptide is the amino acid sequence having SEQ ID NO 45. In some aspects, the mature polypeptide is amino acids 1 to 678 of SEQ ID NO 47 and amino acids −21 to −1 of SEQ ID NO 47 is a signal peptide. In some aspects, the mature polypeptide is the amino acid sequence having SEQ ID NO 48. In some aspects, the mature polypeptide is amino acids 1 to 667 of SEQ ID NO 64 and amino acids −21 to −1 of SEQ ID NO 64 is a signal peptide. In some aspects, the mature polypeptide is the amino acid sequence having SEQ ID NO 65. In some aspects, the mature polypeptide is amino acids 1 to 632 of SEQ ID NO 67 and amino acids −21 to −1 of SEQ ID NO 67 is a signal peptide. In some aspects, the mature polypeptide is the amino acid sequence having SEQ ID NO 68. In some aspects, the mature polypeptide is amino acids 1 to 629 of SEQ ID NO 70 and amino acids −19 to −1 of SEQ ID NO 70 is a signal peptide. In some aspects, the mature polypeptide is the amino acid sequence having SEQ ID NO 71. In some aspects, the mature polypeptide is amino acids 1 to 647 of SEQ ID NO 70 and amino acids −22 to −1 of SEQ ID NO 73 is a signal peptide. In some aspects, the mature polypeptide is the amino acid sequence having SEQ ID NO 74. In some aspects, the mature polypeptide is amino acids 1 to 629 of SEQ ID NO 76 and amino acids −24 to −1 of SEQ ID NO 76 is a signal peptide. In some aspects, the mature polypeptide is the amino acid sequence having SEQ ID NO 77. In some aspects, the mature polypeptide is amino acids 1 to 629 of SEQ ID NO 79 and amino acids −24 to −1 of SEQ ID NO 79 is a signal peptide. In some aspects, the mature polypeptide is the amino acid sequence having SEQ ID NO 80. In some aspects, the mature polypeptide is amino acids 1 to 605 of SEQ ID NO 82 and amino acids −19 to −1 of SEQ ID NO 82 is a signal peptide. In some aspects, the mature polypeptide is the amino acid sequence having SEQ ID NO 83. In some aspects, the mature polypeptide is amino acids 1 to 662 of SEQ ID NO 85 and amino acids −29 to −1 of SEQ ID NO 85 is a signal peptide. In some aspects, the mature polypeptide is the amino acid sequence having SEQ ID NO 86. In some aspects, the mature polypeptide is amino acids 1 to 630 of SEQ ID NO 88 and amino acids −27 to −1 of SEQ ID NO 88 is a signal peptide. In some aspects, the mature polypeptide is the amino acid sequence having SEQ ID NO 89.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having activity. In one aspect, the mature polypeptide coding sequence is nucleotides 61 to 1995 of SEQ ID NO 1 and nucleotides 1 to 60 of SEQ ID NO 1 encodes a signal peptide. In one aspect, the mature polypeptide coding sequence is nucleotides 73 to 1959 of SEQ ID NO 4 and nucleotides 1 to 72 of SEQ ID NO 4 encodes a signal peptide. In one aspect, the mature polypeptide coding sequence is nucleotides 61 to 1995 of SEQ ID NO 7 and nucleotides 1 to 60 of SEQ ID NO 7 encodes a signal peptide. In one aspect, the mature polypeptide coding sequence is nucleotides 61 to 1998 of SEQ ID NO 10 and nucleotides 1 to 60 of SEQ ID NO 10 encodes a signal peptide. In one aspect, the mature polypeptide coding sequence is nucleotides 82 to 1995 of SEQ ID NO 13 and nucleotides 1 to 81 of SEQ ID NO 13 encodes a signal peptide. In one aspect, the mature polypeptide coding sequence is nucleotides 82 to 1995 of SEQ ID NO 16 and nucleotides 1 to 81 of SEQ ID NO 16 encodes a signal peptide. In one aspect, the mature polypeptide coding sequence is nucleotides 70 to 1887 of SEQ ID NO 19 and nucleotides 1 to 69 of SEQ ID NO 19 encodes a signal peptide. In one aspect, the mature polypeptide coding sequence is nucleotides 76 to 1995 of SEQ ID NO 22 and nucleotides 1 to 75 of SEQ ID NO 22 encodes a signal peptide. In one aspect, the mature polypeptide coding sequence is nucleotides 67 to 1206 of SEQ ID NO 25 and nucleotides 1 to 66 of SEQ ID NO 25 encodes a signal peptide. In one aspect, the mature polypeptide coding sequence is nucleotides 61 to 1995 of SEQ ID NO 28 and nucleotides 1 to 60 of SEQ ID NO 28 encodes a signal peptide. In one aspect, the mature polypeptide coding sequence is nucleotides 73 to 1959 of SEQ ID NO 31 and nucleotides 1 to 72 of SEQ ID NO 31 encodes a signal peptide. In one aspect, the mature polypeptide coding sequence is nucleotides 73 to 1959 of SEQ ID NO 34 and nucleotides 1 to 72 of SEQ ID NO 34 encodes a signal peptide. In one aspect, the mature polypeptide coding sequence is nucleotides 73 to 1959 of SEQ ID NO 37 and nucleotides 1 to 72 of SEQ ID NO 37 encodes a signal peptide. In one aspect, the mature polypeptide coding sequence is nucleotides 58 to 1872 of SEQ ID NO 40 and nucleotides 1 to 57 of SEQ ID NO 40 encodes a signal peptide. In one aspect, the mature polypeptide coding sequence is nucleotides 70 to 1959 of SEQ ID NO 43 and nucleotides 1 to 69 of SEQ ID NO 43 encodes a signal peptide. In one aspect, the mature polypeptide coding sequence is nucleotides 64 to 2097 of SEQ ID NO 46 and nucleotides 1 to 63 of SEQ ID NO 46 encodes a signal peptide. In one aspect, the mature polypeptide coding sequence is nucleotides 64 to 2064 of SEQ ID NO 63 and nucleotides 1 to 63 of SEQ ID NO 63 encodes a signal peptide. In one aspect, the mature polypeptide coding sequence is nucleotides 64 to 1959 of SEQ ID NO 66 and nucleotides 1 to 63 of SEQ ID NO 66 encodes a signal peptide. In one aspect, the mature polypeptide coding sequence is nucleotides 58 to 1944 of SEQ ID NO 69 and nucleotides 1 to 57 of SEQ ID NO 69 encodes a signal peptide.

In one aspect, the mature polypeptide coding sequence is nucleotides 67 to 2007 of SEQ ID NO 72 and nucleotides 1 to 66 of SEQ ID NO 72 encodes a signal peptide. In one aspect, the mature polypeptide coding sequence is nucleotides 73 to 1959 of SEQ ID NO 75 and nucleotides 1 to 72 of SEQ ID NO 75 encodes a signal peptide. In one aspect, the mature polypeptide coding sequence is nucleotides 73 to 1959 of SEQ ID NO 78 and nucleotides 1 to 72 of SEQ ID NO 78 encodes a signal peptide. In one aspect, the mature polypeptide coding sequence is nucleotides 58 to 1872 of SEQ ID NO 81 and nucleotides 1 to 57 of SEQ ID NO 81 encodes a signal peptide. In one aspect, the mature polypeptide coding sequence is nucleotides 88 to 2073 of SEQ ID NO 84 and nucleotides 1 to 87 of SEQ ID NO 84 encodes a signal peptide. In one aspect, the mature polypeptide coding sequence is nucleotides 82 to 1971 of SEQ ID NO 87 and nucleotides 1 to 81 of SEQ ID NO 87 encodes a signal peptide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Nomenclature: For purposes of the present invention, the nomenclature [E/Q] means that the amino acid at this position may be a glutamic acid (Glu, E) or a glutamine (Gln, Q). Likewise, the nomenclature [V/G/A/I] means that the amino acid at this position may be a valine (Val, V), glycine (Gly, G), alanine (Ala, A) or isoleucine (Ile, I), and so forth for other combinations as described herein. Unless otherwise limited further, the amino acid X is defined such that it may be any of the 20 natural amino acids.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

Variant: The term “variant” means a polypeptide having deacetylase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

### Assays and Detergent Composition

### Detergent Compositions

The below mentioned detergent composition may be used in combination with the enzyme of the invention.

Biotex Black (Liquid)

5-15% Anionic surfactants, <5% Nonionic surfactants, perfume, enzymes, DMDM and hydantoin.

Composition of Ariel Sensitive White & Color, Liquid Detergent Composition

Aqua, Alcohol Ethoxy Sulfate, Alcohol Ethoxylate, Amino Oxide, Citrid Acid, C12-18 topped palm kernel fatty acid, Protease, Glycosidase, Amylase, Ethanol, 1,2 Propanediol, Sodium Formate, Calcium Chloride, Sodium hydroxide, Silicone Emulsion, Trans-sulphated EHDQ (the ingredients are listed in descending order).

### Composition of WFK IEC-A Model Detergent (Powder)

Ingredients: Linear sodium alkyl benzene sulfonate 8,8%, Ethoxylated fatty alcohol C12-18 (7 EO) 4.7%, Sodium soap 3.2%, Anti foam DC2-42485 3.9%, Sodium aluminium silicate zeolite 4A28.3%, Sodium carbonate 11.6%, Sodium salt of a copolymer from acrylic and maleic acid (Sokalan CP5) 2.4%, Sodium silicate 3.0%, Carboxymethylcellulose 1.2%, Dequest 2066 2.8%, Optical whitener 0.2%, Sodium sulfate 6.5%, Protease 0.4%.

### Composition of Ariel Actilift (Liquid)

Ingredients: 5-15% Anionic surfactants; <5% Non-ionic surfactants, Phosphonates, Soap; Enzymes, Optical brighteners, Benzisothiazolinone, Methylisothiazolinone, Perfumes, Alpha-isomethyl ionone, Citronellol, Geraniol, Linalool.

### Composition of Ariel Actilift Colour & Style (Liquid)

Ingredients: 5-15% Anionic surfactants; <5% Non-ionic surfactants, Phosphonates, Soap; Enzymes, Perfumes, Benzisothiazolinone, Methylisothiazolinone, Alpha-isomethyl ionone, Butylphenyl methylpropional, Citronellol, Geraniol, Linalool.

### Composition of Persil Small & Mighty (Liquid)

Ingredients: 15-30% Anionic surfactants, Non-ionic surfacts, 5-15% Soap, <5% Polycarboxylates, Perfume, Phosphates, Optical Brighteners

Persil 2 in 1 with Comfort Passion Flower Powder

Sodium sulfate, Sodium carbonate, Sodium Dodecylbenzenesulfonate, Bentonite, Sodium Carbonate Peroxide, Sodium Silicate, Zeolite, Aqua, Citric acid, TAED, C12-15 Pareth-7, Stearic Acid, Parfum, Sodium Acrylic Acid/MA Copolymer, Cellulose Gum, Corn Starch Modified, Sodium chloride, Tetrasodium Etidronate, Calcium Sodium EDTMP, Disodium Anilinomorpholinotriazinyl-aminostilbenesulfonate, Sodium bicarbonate, Phenylpropyl Ethyl Methicone, Butylphenyl Methylpropional, Glyceryl Stearates, Calcium carbonate, Sodium Polyacrylate, Alpha-Isomethyl Ionone, Disodium Distyrylbiphenyl Disulfonate, Cellulose, Protease, Limonene, PEG-75, Titanium dioxide, Dextrin, Sucrose, Sodium Polyaryl Sulphonate, CI 12490, CI 45100, CI 42090, Sodium Thiosulfate, CI 61585.

### Persil Biological Powder

Sucrose, Sorbitol, Aluminum Silicate, Polyoxymethylene Melamine, Sodium Polyaryl Sulphonate, CI 61585, CI 45100, Lipase, Amylase, Xanthan gum, Hydroxypropyl methyl cellulose, CI 12490, Disodium Distyrylbiphenyl Disulfonate, Sodium Thiosulfate, CI 42090, Mannanase, CI 11680, Etidronic Acid, Tetrasodium EDTA.

### Persil Biological Tablets

Sodium carbonate, Sodium Carbonate Peroxide, Sodium bicarbonate, Zeolite, Aqua, Sodium Silicate, Sodium Lauryl Sulfate, Cellulose, TAED, Sodium Dodecylbenzenesulfonate, Hemicellulose, Lignin, Lauryl Glucoside, Sodium Acrylic Acid/MA Copolymer, Bentonite, Sodium chloride, Parfum, Tetrasodium Etidronate, Sodium sulfate, Sodium Polyacrylate, Dimethicone, Disodium Anilinomorpholinotriazinylaminostilbenesulfonate, Dodecylbenzene Sulfonic Acid, Trimethylsiloxysilicate, Calcium carbonate, Cellulose, PEG-75, Titanium dioxide, Dextrin, Protease, Corn Starch Modified, Sucrose, CI 12490, Sodium Polyaryl Sulphonate, Sodium Thiosulfate, Amylase, Kaolin,

### Persil Colour Care Biological Powder

Subtilisin, Imidazolidinone, Hexyl Cinnamal, Sucrose, Sorbitol, Aluminum Silicate, Polyoxymethylene Melamine, CI 61585, CI 45100, Lipase, Amylase, Xanthan gum, Hydroxypropyl methyl cellulose, CI 12490, Disodium Distyrylbiphenyl Disulfonate, Sodium Thiosulfate, CI 42090, Mannanase, CI 11680, Etidronic Acid, Tetrasodium EDTA.

### Persil Colour Care Biological Tablets

Sodium bicarbonate, Sodium carbonate, Zeolite, Aqua, Sodium Silicate, Sodium Lauryl Sulfate, Cellulose Gum, Sodium Dodecylbenzenesulfonate, Lauryl Glucoside, Sodium chloride, Sodium Acrylic Acid/MA Copolymer, Parfum, Sodium Thioglycolate, PVP, Sodium sulfate, Tetrasodium Etidronate, Sodium Polyacrylate, Dimethicone, Bentonite, Dodecylbenzene Sulfonic Acid, Trimethylsiloxysilicate, Calcium carbonate, Cellulose, PEG-75, Titanium dioxide, Dextrin, Protease, Corn Starch Modified, Sucrose, Sodium Thiosulfate, Amylase, CI 74160, Kaolin.

### Persil Dual Action Capsules Bio

MEA-Dodecylbenzenesulfonate, MEA-Hydrogenated Cocoate, C12-15 Pareth-7, Dipropylene Glycol, Aqua, Tetrasodium Etidronate, Polyvinyl Alcohol, Glycerin, Aziridine, homopolymer ethoxylated, Propylene glycol, Parfum, Sodium Diethylenetriamine Pentamethylene Phosphonate, Sorbitol, MEA-Sulfate, Ethanolamine, Subtilisin, Glycol, Butylphenyl Methylpropional, Boronic acid, (4-formylphenyl), Hexyl Cinnamal, Limonene, Linalool, Disodium Distyrylbiphenyl Disulfonate, Alpha-Isomethyl Ionone, Geraniol, Amylase, Polymeric Blue Colourant, Polymeric Yellow Colourant, Talc, Sodium chloride, Benzisothiazolinone, Mannanase, Denatonium Benzoate.

Persil 2 in 1 with Comfort Sunshiny Days Powder

Sodium sulfate, Sodium carbonate, Sodium Dodecylbenzenesulfonate, Bentonite, Sodium Carbonate Peroxide, Sodium Silicate, Zeolite, Aqua, Citric acid, TAED, C12-15 Pareth-7, Parfum, Stearic Acid, Sodium Acrylic Acid/MA Copolymer, Cellulose Gum, Corn Starch Modified, Sodium chloride, Tetrasodium Etidronate, Calcium Sodium EDTMP, Disodium Anilinomorpholinotriazinyl-aminostilbenesulfonate, Sodium bicarbonate, Phenylpropyl Ethyl Methicone, Butylphenyl Methylpropional, Glyceryl Stearates, Calcium carbonate, Sodium Polyacrylate, Geraniol, Disodium Distyrylbiphenyl Disulfonate, Cellulose, Protease, PEG-75, Titanium dioxide, Dextrin, Sucrose, Sodium Polyaryl Sulphonate, CI 12490, CI 45100, CI 42090, Sodium Thiosulfate, CI 61585.

Persil Small & Mighty 2 int with Comfort Sunshiny Days

Aqua, C12-15 Pareth-7, Sodium Dodecylbenzenesulfonate, Propylene glycol, Sodium Hydrogenated Cocoate, Triethanolamine, Glycerin, TEA-Hydrogenated Cocoate, Parfum, Sodium chloride, Polyquaternium-10, PVP, Polymeric Pink Colourant, Sodium sulfate, Disodium Distyrylbiphenyl Disulfonate, Butylphenyl Methylpropional, Styrene/Acrylates Copolymer, Hexyl Cinnamal, Citronellol, Eugenol, Polyvinyl Alcohol, Sodium acetate, Isopropyl alcohol, Polymeric Yellow Colourant, Sodium Lauryl Sulfate.

### Persil Small & Mighty Bio

Aqua, MEA-Dodecylbenzenesulfonate, Propylene glycol, Sodium Laureth Sulfate, C12-15 Pareth-7, TEA-Hydrogenated Cocoate, MEA-Citrate, Aziridine homopolymer ethoxylated, MEA-Etidronate, Triethanolamine, Parfum, Acrylates Copolymer, Sorbitol, MEA-Sulfate, Sodium Sulfite, Disodium Distyrylbiphenyl Disulfonate, Butylphenyl Methylpropional, Styrene/Acrylates Copolymer, Citronellol, Sodium sulfate, Peptides, salts, sugars from fermentation (process), Subtilisin, Glycerin, Boronic acid, (4-formylphenyl), Geraniol, Pectate Lyase, Amylase, Sodium Lauryl Sulfate, Mannanase, CI 42051.

### Persil Small & Mighty Capsules Biological

MEA-Dodecylbenzenesulfonate, MEA-Hydrogenated Cocoate, C12-15 Pareth-7, Dipropylene Glycol, Aqua, Glycerin, Polyvinyl Alcohol, Parfum, Aziridine homopolymer ethoxylated, Sodium Diethylenetriamine Pentamethylene Phosphonate, Propylene glycol, Sorbitol, MEA-Sulfate, Ethanolamine, Subtilisin, Glycol, Butylphenyl Methylpropional, Hexyl Cinnamal, Starch, Boronic acid, (4-formylphenyl), Limonene, Linalool, Disodium Distyrylbiphenyl Disulfonate, Alpha-Isomethyl lonone, Geraniol, Amylase, Talc, Polymeric Blue Colourant, Sodium chloride, Benzisothiazolinone, Denatonium Benzoate, Polymeric Yellow Colourant, Mannanase.

### Persil Small & Mighty Capsules Colour Care

MEA-Dodecylbenzenesulfonate, MEA-Hydrogenated Cocoate, C12-15 Pareth-7, Dipropylene Glycol, Aqua, Glycerin, Polyvinyl Alcohol, Parfum, Aziridine homopolymer ethoxylated, Sodium Diethylenetriamine Pentamethylene Phosphonate, Propylene glycol, MEA-Sulfate, Ethanolamine, PVP, Sorbitol, Butylphenyl Methylpropional, Subtilisin, Hexyl Cinnamal, Starch, Limonene, Linalool, Boronic acid, (4-formylphenyl), Alpha-Isomethyl lonone, Geraniol, Talc, Polymeric Blue Colourant, Denatonium Benzoate, Polymeric Yellow Colourant.

### Persil Small & Mighty Colour Care

Aqua, MEA-Dodecylbenzenesulfonate, Propylene glycol, Sodium Laureth Sulfate, C12-15 Pareth-7, TEA-Hydrogenated Cocoate, MEA-Citrate, Aziridine homopolymer ethoxylated, MEA-Etidronate, Triethanolamine, Parfum, Acrylates Copolymer, Sorbitol, MEA-Sulfate, Sodium Sulfite, Glycerin, Butylphenyl Methylpropional, Citronellol, Sodium sulfate, Peptides, salts, sugars from fermentation (process),Styrene/Acrylates Copolymer, Subtilisin, Boronic acid, (4-formylphenyl), Geraniol, Pectate Lyase, Amylase, Sodium Lauryl Sulfate, Mannanase, CI 61585, CI 45100.

### Composition of Fairy Non Bio (Liquid)

Ingredients: 15-30% Anionic Surfactants,5-15% Non-Ionic Surfactants, Soap, Benzisothiazolinone, Methylisothiazolinone, Perfumes

### Composition of Model Detergent T (Powder)

Ingredients: 11% LAS, 2% AS/AEOS, 2% soap, 3% AEO, 15.15% sodium carbonate, 3% sodium slilcate, 18.75% zeolite, 0.15% chelant, 2% sodium citrate, 1.65% AA/MA copolymer, 2.5% CMC and 0.5% SRP (all percentages are w/w).

### Composition of Model Detergent X (Powder)

Ingredients: 16.5% LAS, 15% zeolite, 12% sodium disilicate, 20% sodium carbonate, 1% sokalan, 35.5% sodium sulfate (all percentages are w/w).

### Composition of Ariel Actilift Colour & Style (Powder)

Ingredients: 15-30% Anionic surfactants, <5% Non-ionic surfactants, Phosphonates, Polycarboxylates, Zeolites; Enzymes, Perfumes, Hexyl cinnamal.

### Composition of Ariel Actilift (Powder)

Ingredients: 5-15% Anionic surfactants, Oxygen-based bleaching agents, <5% Non-ionic surfactants, Phosphonates, Polycarboxylates, Zeolites, Optical brightners, Enzymes, Perfumes, Butylphenyl Methylpropional, Coumarin, Hexyl Cinnamal

### Composition of Persil Megaperls (Powder)

Ingredients: 15-30% of the following: anionic surfactants, oxygen-based bleaching agent and zeolites, less than 5% of the following: non-ionic surfactants, phosphonates, polycarboxylates, soap, Further ingredients: Perfumes, Hexyl cinnamal, Benzyl salicylate, Linalool, optical brighteners, Enzymes and Citronellol.

### Gain Liquid, Original:

Ingredients: Water, Alcohol Ethoxysulfate, Diethylene Glycol, Alcohol Ethoxylate, Ethanolamine, Linear Alkyl Benzene Sulfonate, Sodium Fatty Acids, Polyethyleneimine Ethoxylate, Citric Acid, Borax, Sodium Cumene Sulfonate, Propylene Glycol, DTPA, Disodium Diaminostilbene Disulfonate, Dipropylethyl Tetramine, Sodium Hydroxide, Sodium Formate, Calcium Formate, Dimethicone, Amylase, Protease, Liquitint™, Hydrogenated Castor Oil, Fragrance

### Tide Liquid, Original:

Ingredients: Linear alkylbenzene sulfonate, propylene glycol, citric acid, sodium hydroxide, borax, ethanolamine, ethanol, alcohol sulfate, polyethyleneimine ethoxylate, sodium fatty acids, diquaternium ethoxysulfate, protease, diethylene glycol, laureth-9, alkyldimethylamine oxide, fragrance, amylase, disodium diaminostilbene disulfonate, DTPA, sodium formate, calcium formate, polyethylene glycol 4000, mannanase, Liquitint™ Blue, dimethicone.

### Liquid Tide, Free and Gentle:

Water, sodium alcoholethoxy sulfate, propylene glycol, borax, ethanol, linear alkylbenzene sulfonate sodium, salt, polyethyleneimine ethoxylate, diethylene glycol, trans sulfated & ethoxylated hexamethylene diamine, alcohol ethoxylate, linear alkylbenzene sulfonate, MEA salt, sodium formate, sodium alkyl sulfate, DTPA, amine oxide, calcium formate, disodium diaminostilbene, disulfonate, amylase, protease, dimethicone, benzisothiazolinone

### Tide Coldwater Liquid, Fresh Scent:

Water, alcoholethoxy sulfate, linear alkylbenzene sulfonate, diethylene glycol, propylene glycol, ethanolamine, citric acid, Borax, alcohol sulfate, sodium hydroxide, polyethyleneimine, ethoxylate, sodium fatty acids, ethanol, protease, Laureth-9, diquaternium ethoxysulfate, lauramine oxide, sodium cumene, sulfonate, fragrance, DTPA, amylase, disodium, diaminostilbene, disulfonate, sodium formate, disodium distyrylbiphenyl disulfonate, calcium formate, polyethylene glycol 4000, mannanase, pectinase, Liquitint™ Blue, dimethicone

### Tide TOTALCARE™ Liquid, Cool Cotton:

Water, alcoholethoxy sulfate, propylene glycol, sodium fatty acids, laurtrimonium chloride, ethanol, sodium hydroxide, sodium cumene sulfonate, citric acid, ethanolamine, diethylene glycol, silicone polyether, borax, fragrance, polyethyleneimine ethoxylate, protease, Laureth-9, DTPA, polyacrylamide quaternium chloride, disodium diaminostilbene disulfonate, sodium formate, Liquitint™ Orange, dipropylethyl tetraamine, dimethicone, cellulase,

### Liquid Tide Plus Bleach Alternative™, Vivid White and Bright, Original and Clean Breeze:

Water, sodium alcoholethoxy sulfate, sodium alkyl sulfate, MEA citrate, linear alkylbenzene sulfonate, MEA salt, propylene glycol, diethylene glycol, polyethyleneimine ethoxylate, ethanol, sodium fatty acids, ethanolamine, lauramine oxide, borax, Laureth-9, DTPA, sodium cumene sulfonate, sodium formate, calcium formate, linear alkylbenzene sulfonate, sodium salt, alcohol sulfate, sodium hydroxide, diquaternium ethoxysulfate, fragrance, amylase, protease, mannanase, pectinase, disodium diaminostilbene disulfonate, benzisothiazolinone, Liquitint™ Blue, dimethicone, dipropylethyl tetraamine.

### Liquid Tide HE, Original Scent:

Water, Sodium alcoholethoxy sulfate, MEA citrate, Sodium Alkyl Sulfate, alcohol ethoxylate, linear alkylbenzene sulfonate, MEA salt, sodium fatty acids, polyethyleneimine ethoxylate, diethylene glycol, propylene glycol, diquaternium ethoxysulfate, borax, polyethyleneimine, ethoxylate propoxylate, ethanol, sodium cumene sulfonate, fragrance, DTPA, disodium diaminostilbene disulfonate, Mannanase, cellulase, amylase, sodium formate, calcium formate, Lauramine oxide, Liquitint™ Blue, Dimethicone/polydimethyl silicone.

### Tide TOTALCARE HE Liquid, Renewing Rain:

Water, alcoholethoxy sulfate, linear alkylbenzene sulfonate, alcohol ethoxylate, citric acid, Ethanolamine, sodium fatty acids, diethylene glycol, propylene glycol, sodium hydroxide, borax, polyethyleneimine ethoxylate, silicone polyether, ethanol, protease, sodium cumene sulfonate, diquaternium ethoxysulfate, Laureth-9, fragrance, amylase, DTPA, disodium diaminostilbene disulfonate, disodium distyrylbiphenyl disulfonate, sodium formate, calcium formate, mannanase, Liquitint™ Orange, dimethicone, polyacrylamide quaternium chloride, cellulase, dipropylethyl tetraamine.

### Tide Liquid HE Free:

Water, alcoholethoxy sulfate, diethylene glycol, monoethanolamine citrate, sodium formate, propylene glycol, linear alkylbenzene sulfonates, ethanolamine, ethanol, polyethyleneimine ethoxylate, amylase, benzisothiazolin, borax, calcium formate, citric acid, diethylenetriamine pentaacetate sodium, dimethicone, diquaternium ethoxysulfate, disodium diaminostilbene disulfonate, Laureth-9, mannanase, protease, sodium cumene sulfonate, sodium fatty acids.

### Tide Coldwater HE Liquid, Fresh Scent:

Water, alcoholethoxy sulfate, MEA Citrate, alcohol sulfate, Alcohol ethoxylate, Linear alkylbenzene sulfonate MEA, sodium fatty acids, polyethyleneimine ethoxylate, diethylene glycol, propylene glycol, diquaternium ethoxysulfate, borax, polyethyleneimine ethoxylate propoxylate, ethanol, sodium cumene sulfonate, fragrance, DTPA, disodium diaminostilbene disulfonate, protease, mannanase, cellulase, amylase, sodium formate, calcium formate, lauramine oxide, Liquitint™ Blue, dimethicone.

### Tide for Coldwater HE Free Liquid:

Water, sodium alcoholethoxy sulfate, MEA Citrate, Linear alkylbenzene sulfonate: sodium salt, Alcohol ethoxylate, Linear alkylbenzene sulfonate: MEA salt, sodium fatty acids, polyethyleneimine ethoxylate, diethylene glycol, propylene glycol, diquaternium ethoxysulfate, Borax, protease, polyethyleneimine ethoxylate propoxylate, ethanol, sodium cumene sulfonate, Amylase, citric acid, DTPA, disodium diaminostilbene disulfonate, sodium formate, calcium formate, dimethicone.

### Tide Simply Clean & Fresh:

Water, alcohol ethoxylate sulfate, linear alkylbenzene sulfonate Sodium/Mea salts, propylene glycol, diethylene glycol, sodium formate, ethanol, borax, sodium fatty acids, fragrance, lauramine oxide, DTPA, Polyethylene amine ethoxylate, calcium formate, disodium diaminostilbene disulfonate, dimethicone, tetramine, Liquitint™ Blue.

### Tide Pods, Ocean Mist, Mystic Forest, Spring Meadow:

Linear alkylbenzene sulfonates, C12-16 Pareth-9, propylene glycol, alcoholethoxy sulfate, water, polyethyleneimine ethoxylate, glycerine, fatty acid salts, PEG-136 polyvinyl acetate, ethylene Diamine disuccinic salt, monoethanolamine citrate, sodium bisulfite, diethylenetriamine pentaacetate sodium, disodium distyrylbiphenyl disulfonate, calcium formate, mannanase, exyloglucanase, sodium formate, hydrogenated castor oil, natalase, dyes, termamyl, subtilisin, benzisothiazolin, perfume.

### Tide to Go:

Deionized water, Dipropylene Glycol Butyl Ether, Sodium Alkyl Sulfate, Hydrogen Peroxide, Ethanol, Magnesium Sulfate, Alkyl Dimethyl Amine Oxide, Citric Acid, Sodium Hydroxide, Trimethoxy Benzoic Acid, Fragrance.

### Tide Stain Release Liquid:

Water, Alkyl Ethoxylate, Linear Alkylbenzenesulfonate, Hydrogen Peroxide, Diquaternium Ethoxysulfate, Ethanolamine, Disodium Distyrylbiphenyl Disulfonate, tetrabutyl Ethylidinebisphenol, F&DC Yellow 3, Fragrance.

### Tide Stain Release Powder:

Sodium percarbonate, sodium sulfate, sodium carbonate, sodium aluminosilicate, nonanoyloxy benzene sulfonate, sodium polyacrylate, water, sodium alkylbenzenesulfonate, DTPA, polyethylene glycol, sodium palmitate, amylase, protease, modified starch, FD&C Blue 1, fragrance.

### Tide Stain Release, Pre Treater Spray:

Water, Alkyl Ethoxylate, MEA Borate, Linear Alkylbenzenesulfonate, Propylene Glycol, Diquaternium Ethoxysulfate, Calcium Chlorideenzyme, Protease, Ethanolamine, Benzoisothiazolinone, Amylase, Sodium Citrate, Sodium Hydroxide, Fragrance.

### Tide to Go Stain Eraser:

Water, Alkyl Amine Oxide, Dipropylene Glycol Phenyl Ether, Hydrogen Peroxide, Citric Acid, Ethylene Diamine Disuccinic Acid Sodium salt, Sodium Alkyl Sulfate, Fragrance.

Tide Boost with Oxi:

Sodium bicarbonate, sodium carbonate, sodium percarbonate, alcohol ethoxylate, sodium chloride, maleic/acrylic copolymer, nonanoyloxy benzene sulfonate, sodium sulfate, colorant, diethylenetriamine pentaacetate sodium salt, hydrated aluminosilicate (zeolite), polyethylene glycol, sodium alkylbenzene sulfonate, sodium palmitate, starch, water, fragrance.

### Tide Stain Release Boost Duo Pac:

Polyvinyl Alcoholpouch film, wherein there is packed a liquid part and a powder part:

Liquid Ingredients: Dipropylene Glycol, diquaternium Ethoxysulfate, Water, Glycerin, Liquitint™ Orange, Powder Ingredients: sodium percarbonate, nonanoyloxy benzene sulfonate, sodium carbonate, sodium sulfate, sodium aluminosilicate, sodium polyacrylate, sodium alkylbenzenesulfonate, maleic/acrylic copolymer, water, amylase, polyethylene glycol, sodium palmitate, modified starch, protease, glycerine, DTPA, fragrance.

### Tide Ultra Stain Release:

Water, sodium alcoholethoxy sulfate, linear alkyl benzene sulfonate, sodium/MEA salts, MEA citrate, propylene glycol, polyethyleneimine ethoxylate, ethanol, diethylene glycol, polyethyleneimine propoxyethoxylate, sodium fatty acids, protease, borax, sodium cumene sulfonate, DTPA, fragrance, amylase, disodium diaminostilbene disulfonate, calcium formate, sodium formate, gluconase, dimethicone, Liquitint™ Blue, mannanase.

Ultra Tide with a Touch of Downy® Powdered Detergent, April Fresh/Clean Breeze/April Essence:

Sodium Carbonate, Sodium Aluminosilicate, Sodium Sulfate, Linear Alkylbenzene Sulfonate, Bentonite, Water, Sodium Percarbonate, Sodium Polyacrylate, Silicate, Alkyl Sulfate, Nonanoyloxybenzenesulfonate, DTPA, Polyethylene Glycol 4000, Silicone, Ethoxylate, fragrance, Polyethylene Oxide, Palmitic Acid, Disodium Diaminostilbene Disulfonate, Protease, Liquitint™ Red, FD&C Blue 1, Cellulase.

Ultra Tide with a Touch of Downy Clean Breeze:

Water, sodium alcoholethoxy sulfate, MEA citrate, linear alkyl benzene sulfonate: sodium/MEA salts, propylene glycol, polyethyleneimine ethoxylate, ethanol, diethylene glycol, polyethyleneimine, propoxyethoxylate, diquaternium ethoxysulfate, alcohol sulfate, dimethicone, fragrance, borax, sodium fatty acids, DTPA, protease, sodium bisulfite, disodium diaminostilbene disulfonate, amylase, gluconase, castor oil, calcium formate, MEA, styrene acrylate copolymer, sodium formate, Liquitint™ Blue.

Ultra Tide with Downy Sun Blossom:

Water, sodium alcoholethoxy sulfate, MEA citrate, linear alkyl benzene sulfonate: sodium/MEA salts, propylene glycol, ethanol, diethylene glycol, polyethyleneimine propoxyethoxylate, polyethyleneimine ethoxylate, alcohol sulfate, dimethicone, fragrance, borax, sodium fatty acids, DTPA, protease, sodium bisulfite, disodium diaminostilbene disulfonate, amylase, castor oil, calcium formate, MEA, styrene acrylate copolymer, propanaminium propanamide, gluconase, sodium formate, Liquitint™ Blue.

Ultra Tide with Downy April Fresh/Sweet Dreams:

Water, sodium alcoholethoxy sulfate, MEA citrate, linear alkyl benzene sulfonate: sodium/MEA salts, propylene glycol, polyethyleneimine ethoxylate, ethanol, diethylene glycol, polyethyleneimin propoxyethoxylate, diquaternium ethoxysulfate, alcohol sulfate, dimethicone, fragrance, borax, sodium fatty acids, DTPA, protease, sodium bisulfite, disodium diaminostilbene disulfonate, amylase, gluconase, castor oil, calcium formate, MEA, styrene acrylate copolymer, propanaminium propanamide, sodium formate, Liquitint™ Blue.

### Ultra Tide Free Powdered Detergent:

Sodium Carbonate, Sodium Aluminosilicate, Alkyl Sulfate, Sodium Sulfate, Linear Alkylbenzene Sulfonate, Water, Sodium polyacrylate, Silicate, Ethoxylate, Sodium percarbonate, Polyethylene Glycol 4000, Protease, Disodium Diaminostilbene Disulfonate, Silicone, Cellulase.

### Ultra Tide Powdered Detergent, Clean Breeze/Spring Lavender/Mountain Spring:

Sodium Carbonate, Sodium Aluminosilicate, Sodium Sulfate, Linear Alkylbenzene Sulfonate, Alkyl Sulfate, Sodium Percarbonate, Water, Sodium Polyacrylate, Silicate, Nonanoyloxybenzenesulfonate, Ethoxylate, Polyethylene Glycol 4000, Fragrance, DTPA, Disodium Diaminostilbene Disulfonate, Palmitic Acid, Protease, Silicone, Cellulase.

Ultra Tide HE (high Efficiency) Pwdered Detergent, Clean Breeze:

Sodium Carbonate, Sodium Aluminosilicate, Sodium Sulfate, Linear Alkylbenzene Sulfonate, Water, Nonanoyloxybenzenesulfonate, Alkyl Sulfate, Sodium Polyacrylate, Silicate, Sodium Percarbonate, Ethoxylate, Polyethylene Glycol 4000, Fragrance, DTPA, Palmitic Acid, Disodium Diaminostilbene Disulfonate, Protease, Silicone, Cellulase.

### Ultra Tide Coldwater Powdered Detergent, Fresh Scent:

Sodium Carbonate, Sodium Aluminosilicate, Sodium Sulfate, Sodium Percarbonate, Alkyl Sulfate, Linear Alkylbenzene Sulfonate, Water, Nonanoyloxybenzenesulfonate, Sodium Polyacrylate, Silicate, Ethoxylate, Polyethylene Glycol 4000, DTPA, Fragrance, Natalase, Palmitic Acid, Protease, Disodium, Diaminostilbene Disulfonate, FD&C Blue 1, Silicone, Cellulase, Alkyl Ether Sulfate.

Ultra Tide with Bleach Powdered Detergent, Clean Breeze:

Sodium Carbonate, Sodium Aluminosilicate, Sodium Sulfate, Linear Alkylbenzene Sulfonate, Sodium Percarbonate, Nonanoyloxybenzenesulfonate, Alkyl Sulfate, Water, Silicate, Sodium Polyacrylate, Ethoxylate, Polyethylene Glycol 4000, Fragrance, DTPA, Palmitic Acid, Protease, Disodium Diaminostilbene Disulfonate, Silicone, FD&C Blue 1, Cellulase, Alkyl Ether Sulfate.

Ultra Tide with Febreeze Freshness ™ Powdered Detergent, Spring Renewal:

Sodium Carbonate, Sodium Aluminosilicate, Sodium Sulfate, Linear Alkylbenzene Sulfonate, Sodium Percarbonate, Alkyl Sulfate, Water, Sodium Polyacrylate, Silicate, Nonanoyloxybenzenesulfonate, Ethoxylate, Polyethylene Glycol 4000, DTPA, Fragrance, Cellulase, Protease, Disodium Diaminostilbene Disulfonate, Silicone, FD&C Blue 1.

Liquid Tide Plus with Febreeze Freshness—Sport HE Active Fresh:

Water, Sodium alcoholethoxy sulfate, MEA citrate, linear alkylbenzene sulfonate, sodium salt, linear alkylbenzene sulfonate: MEA salt, alcohol ethoxylate, sodium fatty acids, propylene glycol, diethylene glycol, polyethyleneimine ethoxylate propoxylate, diquaternium ethoxysulfate,

Ethanol, sodium cumene sulfonate, borax, fragrance, DTPA, Sodium bisulfate, disodium diaminostilbene disulfonate, Mannanase, cellulase, amylase, sodium formate, calcium formate,

Lauramine oxide, Liquitint™ Blue, Dimethicone/polydimethyl silicone.

### Tide Plus Febreeze Freshness Spring & Renewal:

Water, sodium alcoholethoxy sulfate, linear alkyl benzene sulfonate: sodium/MEA salts, MEA citrate, propylene glycol, polyethyleneimine ethoxylate, fragrance, ethanol, diethylene glycol, polyethyleneimine propoxyethoxylate, protease, alcohol sulfate, borax, sodium fatty acids, DTPA, disodium diaminostilbene disulfonate, MEA, mannanase, gluconase, sodium formate, dimethicone, Liquitint™ Blue, tetramine.

Liquid Tide Plus with Febreeze Freshness, Sport HE Victory Fresh:

Water, Sodium alcoholethoxy sulfate, MEA citrate, linear alkylbenzene sulfonate, sodium salt, linear alkylbenzene sulfonate: MEA salt, alcohol ethoxylate, sodium fatty acids, propylene glycol, diethylene glycol, polyethyleneimine ethoxylate propoxylate, diquaternium ethoxysulfate, ethanol, sodium cumene sulfonate, borax, fragrance, DTPA, Sodium bisulfate, disodium diaminostilbene disulfonate, Mannanase, cellulase, amylase, sodium formate, calcium formate,

Lauramine oxide, Liquitint™ Blue, Dimethicone/polydimethyl silicone.

### Tide Vivid White+Bright Powder, Original:

Sodium Carbonate, Sodium Aluminosilicate, Sodium Sulfate, Linear Alkylbenzene Sulfonate, Sodium Percarbonate, Nonanoyloxybenzenesulfonate, Alkyl Sulfate, Water, Silicate, Sodium Polyacrylate Ethoxylate, Polyethylene Glycol 4000, Fragrance, DTPA, Palmitic Acid, Protease, Disodium Diaminostilbene Disulfonate, Silicone, FD&C Blue 1, Cellulase, Alkyl Ether Sulfate.

### Model Detergents

Model detergent A wash liquor (100%) was prepared by dissolving 3.33 g/I of model detergent A containing 12% LAS, 11% AEO Biosoft N25-7 (NI), 7% AEOS (SLES), 6% MPG, 3% ethanol, 3% TEA (triethanolamine), 2.75% cocoa soap, 2.75% soya soap, 2% glycerol, 2% sodium hydroxide, 2% sodium citrate, 1% sodium formiate, 0.2% DTMPA and 0.2% PCA (all percentages are w/w (weight volume) in water with hardness 15 dH.

Triple-20 Nonionic Model Detergent (60% surfactant) was prepared by dissolving 3.33 g/I non-ionic detergent containing NaOH 0.87%, MPG (Monopropylenglycol) 6%, Glycerol 2%, Soap-soy 2.75%, Soap-coco 2.75%, PCA (Sokalon CP-5) 0.2%, AEO Biosoft N25-7(NI) 16%, Sodium formiate 1%, Sodium Citrate 2%, DTMPA 0.2%, Ethanol (96%) 3%, adjustment of pH with NaOH or Citric acid ass water to 100% (all percentages are w/w (weight volume) in water with hardness 15 dH.

Model detergent A (Example 7) 12% LAS, 11% AEO Biosoft N25-7 (NI), 5% AEOS (SLES), 6% MPG (monopropylene glycol), 3% ethanol, 3% TEA, 2.75% coco soap, 2.75% soya soap, 2% glycerol, 2% sodium hydroxide, 2% sodium citrate, 1% sodium formate, 0.2% DTMPA and 0.2% PCA (all percentages are w/w).

Model Detergent MC A medical cleaning model detergent (model detergent MC) was prepared containing 5% MPG (propylene glycol), 5% Pluronic PE 4300 (PO/EO block polymer; 70%/30%, approx. 1750 g/mol), 2% Plurafac LF 305 (fatty alcohol alkoxylate; C6-10+EO/PO), 1% MGDA (methyl glycine diacetic acid, 1% TEA (triethanolamine) (all percentages are w/w). The pH was adjusted to 8.7 with phosphoric acid.

### Wash Assays

### Mini Launder-O-Meter (MiniLOM) Model Wash System

MiniLOM is a modified mini wash system of the Launder-O-Meter (LOM), which is a medium scale model wash system that can be applied to test up to 20 different wash conditions simultaneously. A LOM is basically a large temperature controlled water bath with 20 closed metal beakers rotating inside it. Each beaker constitutes one small washing machine and during an experiment, each will contain a solution of a specific detergent/enzyme system to be tested along with the soiled and unsoiled fabrics it is tested on. Mechanical stress is achieved by the beakers being rotated in the water bath and by including metal balls in the beaker.

The LOM model wash system is mainly used in medium scale testing of detergents and enzymes at European wash conditions. In a LOM experiment, factors such as the ballast to soil ratio and the fabric to wash liquor ratio can be varied. Therefore, the LOM provides the link between small scale experiments, such as AMSA and mini-wash, and the more time consuming full scale experiments in front loader washing machines.

In miniLOM, washes are performed in 50 ml test tubes placed in Stuart rotator.

### Activity Assays

### Substrate: 1,6-β-(GIcNAc)4-SPh.

### N-Deacetylation.

a) PgaB (GHL13 enzymes of the invention) is buffer exchanged by ultrafiltration (using a 20 mL, 50 kDa MWCO filter) into 100 mM HEPES buffer (200 mM NaCI, pH 7.5). Final concentration of PgaB stock solution=74.8 μM.

b) To 120.3 μL PgaB (final reaction conc. 20 μM) is added 18 μL NiCl2 solution (stock conc. 2 mM, final reaction conc. 80 μM, in 100 mM HEPES, 200 mM NaCI, pH 7.5) and incubated at room temperature for 1 hour (gently shaking) before use.

c) To 225 μL of the 1,6-β-(GIcNAc)4-SPh solution in a MOPS/DMSO mixture (stock conc. 10 mM, final reaction conc. 5 mM) is added 86.7 μL HEPES buffer (100 mM HEPES, 200 mM NaCI, pH 7.5) followed by the 138.3 μL PgaB-NiCl2-solution (b). The reaction is left shaking (850 rpm) at 37 ° C. overnight. Then additional 60 μL buffer-exchanged PgaB+9 μL NiCl2 is added to the reaction (and 20 μL PgaB+3 μL NiCl2 to the control 1; and 7.7 μL buffer to control 2). After 8 hours, the reaction is left at 4° C. over the weekend. The degree of N-deacetylation is analyzed by OPA-assays. Before hydrolysis of the 1,6-β-(GIcNAc)4-SPh, 4.5 μL EDTA (stock conc. 25 mM, final conc. 250 μM) is added and left for 30 min while gently shaking at room temperature.

### OPA Assay (Detection of —NH2 Groups After N-Deacetylation).

a) Afresh o-phthaldialdehyde (OPA) mixture is prepared by adding 800 mg to 10 mL 95% EtOH, and mixing this with 1 L 0.5 M borate buffer (pH 9.0) containing 2 mL 2-mercaptoethanol.

b) To a 10 μL sample is added 100 μL OPA solution. The mixture is transferred to a 96-well plate and analyzed using a spectrophotometer (ex: 340 nm, em: 455 nm). Glucosamine solutions were used as standards for quantification of N-deacetylation of the 1,6-β-(GIcNAc)4-SPh. Labeling and quantification of the free NH2 groups after N-deacetylation by o-phthaldialdehyde (OPA) assay.

### EXAMPLES

### Example 1

### Cloning and Expression of PgaB Homologue (GHL13 Glycosyl Hydrolases) Polypeptides

### Strains

The DNA encoding the genes of the polypeptides shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 30, SEQ ID NO 33, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86 or SEQ ID NO 89 were isolated from bacterial strains and environmental bacterial communities isolated from soil samples collected in different countries (see table 1).

Chromosomal DNA from the different strains and bacterial communities was subjected to full genome sequencing using Illumina technology. The genome sequence was analyzed for protein sequences that had glycosyl hydrolase GHL13 domain. 14 genes and corresponding sequence were identified in the genomes. Chromosomal DNA from Halomonas zhanjiangensis DSM 21076 was purchased from Leibniz Institute DSMZ-German collection of microorganisms and cell cultures. The gene sequence of the polypeptide shown in SEQ ID 36 from Halomonas zhanjiangensis DSM 21076 and the polypeptide show in SEQ ID NO 27 from Acinetobacter bouvetii DSM 14964=CIP 107468 were found in the public database (Accession number AHGP: EFPXONKF; AHGN:EFNDB2FX for SEQ ID NO 36 and AHGP:EFP1T8X13, AHGN:EFNLG8DX for SEQ ID NO 27). The codon optimized synthetic DNA encoding the mature peptide sequence of the gene containing the GHL13 domain from Acinetobacter bouvetii (SEQ ID NO 25) DSM 14964=CIP 107468 was ordered from the company Geneart.

TABLE 1

| SEQ ID | donor | country of origin |
|---|---|---|
| SEQ ID 1 | Pseudomonas meridiana | Iceland |
| SEQ ID 4 | Halomonas sp-62262 | Denmark |
| SEQ ID 7 | Pseudomonas migulae | Denmark |
| SEQ ID 10 | Pseudomonas sp-62331 | Finland |
| SEQ ID 13 | Pseudomonas jessenii | Denmark |
| SEQ ID 16 | Pseudomonas koreensis | Denmark |
| SEQ ID 19 | Stenotrophomonas rhizophila | Denmark |
| SEQ ID 22 | Pseudomonas sp-62498 | China |
| SEQ ID 25 | Acinetobacter bouvetii DSM 14964 | Australia |
| SEQ ID 28 | Pseudomonas panacis | Sweden |
| SEQ ID 31 | Enviromental bacterial community L | Denmark |
| SEQ ID 34 | Halomonas zhanjiangensis DSM 21076 | China |
| SEQ ID 37 | Halomonas sp-63456 | United States |
| SEQ ID 40 | Luteibacter rhizovicinus | Sweden |
| SEQ ID 43 | Enviromental bacterial community R | China |
| SEQ ID 46 | Enviromental bacterial community H | United States |
| SEQ ID 65 | Vibrio proteolyticus | United States |
| SEQ ID 68 | Aquitalea magnusonii | United States |
| SEQ ID 71 | Halomonas ilicicola | Spain |
| SEQ ID 74 | Alkanindiges illinoisensis | United States |
| SEQ ID 77 | Halomonas sp | United States |
| SEQ ID 80 | Halomonas sp. | United States |
| SEQ ID 83 | Luteibacter sp. | Denmark |
| SEQ ID 86 | Variovorax boronicumulans | United States |
| SEQ ID 89 | Silvimonas terrae | United States |

### Cloning and Expression

The cloning and expression of the codon optimized synthetic gene encoding the mature peptide sequence of the gene containing GHL13 domain was inserted into a Bacillus expression vector as described in WO12/025577. Briefly, the DNA encoding the mature peptide of the gene was cloned in frame to a Bacillus clausii secretion signal (BcSP; with the following amino acid sequence: MKKPLGKIVASTALLISVAFSSSIASA (SEQ ID NO: 61). BcSP replaced the native secretion signal in the gene. Downstream of the BcSP sequence, an affinity tag sequence was introduced to ease the purification process (His-tag; with the following amino acid sequence: HHHHHHPR (SEQ ID NO: 62) The gene that was expressed therefore comprised the BcSP sequence followed by the His-tag sequence followed by the mature wild type GHL13 gene sequence. The final expression plasmid (BcSP-His-tag-GHL13) was transformed into a Bacillus subtilis expression host. The GHL13 BcSP-fusion gene was integrated by homologous recombination into the Bacillus subtilis host cell genome upon transformation. The gene construct was expressed under the control of a triple promoter system (as described in WO 99/43835). The gene coding for chloramphenicol acetyltransferase was used as maker (as described in (Diderichsen et al., 1993, Plasmid 30: 312-315)). Transformants were selected on LB media agar supplemented with 6 microgram of chloramphenicol per ml. One recombinant Bacillus subtilis clone containing the GHL13 expression construct was selected and was cultivated on a rotary shaking table in 500 ml baffled Erlenmeyer flasks each containing 100 ml yeast extract-based media. After 3-5 days' cultivation time at 30 ° C. to 37° C., the enzyme containing supernatant was harvested by centrifugation and the enzymes was purified by His-tag purification. The DNA encoding the mature peptide of the full-length GHL13 genes and corresponding truncations containing only the GHL13 domain was amplified from the genomic DNA of the corresponding bacterial strains by standard PCR techniques using specific primers containing an overhang to cloning vector. The amplified PCR fragments were consecutively cloned in frame to a Bacillus clausii secretion signal as described in above.

### Example 2

### His Tag Purification Method

The His-tagged GHL13 enzymes were purified by immobilized metal chromatography (IMAC) using Ni2+ as the metal ion on 5 mL HisTrap Excel columns (GE Healthcare Life Sciences). The purification took place at pH 7 and the bound protein was eluted with imidazole. The purity of the purified enzymes was checked by SDS-PAGE and the concentration of the enzyme determined by Absorbance 280 nm after a buffer exchange in 50 mM HEPES, 100 mM NaCI pH7.0

### Example 3

### MiniLom Deep-Cleaning Effects of the PgaB Homologues (GHL13 Glycosyl Hydrolases) in Liquid Model Detergent

Staphylococcus aureus 15981 (kind gift from Inigo Lasa (Valle, J., A. Toledo-Arana, C. Berasain, J. M. Ghigo, B. Amorena, J. R. Penades, and I. Lasa. 2003, Mol. Microbiol. 48:1075-1087) was used as model microorganism in the present example. S. aureus was restreaked on Tryptone Soya Agar (TSA) (pH 7.3) (CM0131; Oxoid Ltd, Basingstoke, UK) and incubated for 3 day at 37° C. A single colony was inoculated into 10 mL of TSB+1% glucose (24563; Roquette Freres) and the culture was incubated for 16 hours at 37° C. with shaking (200 rpm). After propagation, the S. aureus culture was diluted (1:100) in fresh TSB+1% glucose and 2 mL aliquots were added to the wells of 12-well polystyrene flat-bottom microplates (3512; Costar, Corning Incorporated, Corning, N.Y., USA), in which round swatches (diameter 2 cm) of sterile polyester (WFK30A, 100% white polyester pre-washed) had been placed. Sterile TSB+1% glucose was added to control wells. After 48 h at 37° C. (static incubation), the swatches were rinsed once with 15° dH water. Five rinsed swatches (sterile or with S. aureus biofilm) were placed in 50 mL test tubes and 10 mL of wash liquor (15° dH water with 0.2 g/L iron(III) oxide nanopowder (544884; Sigma-Aldrich) with 3.33 g/L liquid model A detergent) and 2 or 10 ppm enzyme was added to each tube. Washes without enzyme were included as controls. The test tubes were placed in a Stuart rotator and incubated for 1 hour at 37° C. at 20 rpm. The wash liquor was then removed, and the swatches were rinsed twice with 15° dH water and dried on filter paper over night.

The color difference (L) values were measured using a Handheld Minolta CR-300, and are displayed in table 2. Delta values (L_{(swatch washed with enzyme)}−L_{(swatch washed without enzyme)}) are also indicated.

The results show that the PgaB homologue polypeptides of the invention show deep-cleaning properties in model A liquid detergent.

TABLE 2
Deep-cleaning effects of the PgaB homologues (GHL13
glycosyl hydrolases) in model A detergent

| | | Enzyme | | ΔL |
|---|---|---|---|---|
| | | concentration | L | (Lwith enzyme − |
| Substrate | Enzyme | (ppm) | values | Lwithout enzyme) |
| Sterile | no enzyme | | 90.5 | |
| medium, | | | | |
| wfk30A | | | | |
| S. aureus | no enzyme | | 81.5 | |
| biofilm | | | | |
| swatches | | | | |
| S. aureus | SEQ ID NO 6 | 2.0 | 84.0 | 2.6 |
| biofilm | | | | |
| swatches | | | | |
| S. aureus | SEQ ID NO 6 | 10.0 | 86.1 | 4.7 |
| biofilm | | | | |
| swatches | | | | |
| S. aureus | SEQ ID NO 33 | 2.0 | 86.0 | 4.5 |
| biofilm | | | | |
| swatches | | | | |
| S. aureus | SEQ ID NO 33 | 10.0 | 88.4 | 7.0 |
| biofilm | | | | |
| swatches | | | | |
| S. aureus | SEQ ID NO 36 | 2.0 | 85.0 | 3.5 |
| biofilm | | | | |
| swatches | | | | |
| S. aureus | SEQ ID NO 36 | 10.0 | 87.8 | 6.3 |
| biofilm | | | | |
| swatches | | | | |
| S. aureus | SEQ ID NO 45 | 2.0 | 86.4 | 5.0 |
| biofilm | | | | |
| swatches | | | | |
| S. aureus | SEQ ID NO 45 | 10.0 | 87.8 | 6.4 |
| biofilm | | | | |
| swatches | | | | |

### Example 4

### MiniLom Deep-Cleaning Effects of the PgaB Homologues (GHL13 Glycosyl Hydrolases) in Liquid Model Detergent on PNAG Swatches

A crude extract of PNAG (poly-β(1-6)-N-acetylglucosamine) was prepared from Staphylococcus aureus (kind gift from Inigo Lasa (Valle, J., A. Toledo-Arana, C. Berasain, J. M. Ghigo, B. Amorena, J. R. Penades, and I. Lasa. 2003, Mol. Microbiol. 48:1075-1087) as follows; The strain was restreaked on Tryptone Soya Agar (TSA) (pH 7.3) (CM0131; Oxoid Ltd, Basingstoke, UK) and incubated for 1 day at 37° C. 500 mL of TSB+1% glucose (24563; Roquette Freres) was then inoculated, aliquoted into 50 ml conical centrifuge tubes (339652; Thermo Scientific Nunc) (33 ml in each), and incubated for 48 hours at 37° C. with shaking (200 rpm). The cells were subsequently pelleted by centrifugation (10 min, 6000 g, 25° C.), pooled and resuspended in a total of 4 ml 3M NaCI. The suspension was vortexed vigorously and incubated for 15 min at ambient temperature to extract the surface-associated PNAG. The cells were then re-pelleted (10 min, 5000 g, 25° C.) and the PNAG-containing supernatant was retrieved. The supernatant was sterile filtered twice (0.45 μm followed by 0.2 μm) and stored at −20° C. until further use (termed PNAG extract). 50 ul aliquots of the crude PNAG extract were spotted on sterile textile swatches (WFK20A) and incubated for 15 min at ambient temperature. The swatches (sterile or with PNAG) were placed in 50 mL test tubes and 10 mL of wash liquor (15° dH water with 0.2 g/L iron(III) oxide nano-powder (544884; Sigma-Aldrich) with 3.33 g/L liquid model A detergent) and 2 or 20 ppm enzyme(s) were added to each tube. Washes without enzyme were included as controls. The test tubes were placed in a Stuart rotator and incubated for 1 hour at 37° C. at 20 rpm. The wash liquor was then removed, and the swatches were rinsed twice with 15° dH water and dried on filter paper over night.

The Color difference (L) values were measured using a Macbeth Color-Eye 7000 (CE7000), and are displayed in table 3. Delta values (L_{with enzyme}−L_{without enzyme}) are also indicated.

TABLE 3
Deep-cleaning effects of the PgaB homologues (GHL13
glycosyl hydrolases) in model A detergent

| | | Enzyme | | ΔL |
|---|---|---|---|---|
| | | concentration | L | (L_{with enzyme} − |
| Substrate | Enzyme | (ppm) | values | L_{without enzyme}) |
| Sterile | no enzyme | 0.0 | 88.7 | |
| wfk30A | | | | |
| S. aureus | no enzyme | 0.0 | 73.9 | |
| PNAG | | | | |
| swatches | | | | |
| S. aureus | SEQ ID NO 6 | 2.0 | 76.3 | 2.4 |
| PNAG | | | | |
| swatches | | | | |
| S. aureus | SEQ ID NO 6 | 20.0 | 78.7 | 4.8 |
| PNAG | | | | |
| swatches | | | | |
| S. aureus | SEQ ID NO 33 | 2.0 | 78.5 | 4.6 |
| PNAG | | | | |
| swatches | | | | |
| S. aureus | SEQ ID NO 33 | 20.0 | 83.8 | 9.9 |
| PNAG | | | | |
| swatches | | | | |
| S. aureus | SEQ ID NO 36 | 2.0 | 77.7 | 3.7 |
| PNAG | | | | |
| swatches | | | | |
| S. aureus | SEQ ID NO 36 | 20.0 | 78.7 | 4.7 |
| PNAG | | | | |
| swatches | | | | |
| S. aureus | SEQ ID NO 45 | 2.0 | 76.1 | 2.1 |
| PNAG | | | | |
| swatches | | | | |
| S. aureus | SEQ ID NO 45 | 20.0 | 85.1 | 11.2 |
| PNAG | | | | |
| swatches | | | | |

### Example 5

### Biofilm Growth and Detachment Assay

Staphylococcus aureus was kindly provided by Inigo Lasa (Valle et al., Mol Microbiol.2003 May; 48 (4):1075-87). The strain was grown on trypticase soy agar (TSA) at 37° C. overnight. Next day, a single colony was transferred to 15 ml tripticase soy broth (TSB) and incubated 5 hours at 37° C. under shaking. The culture was diluted 1:100 in TSB+1% glucose and 100 μL of the bacterial suspension was transferred to each well of a 96-well microtiter plates (Thermo Scientific, Nunclon Delta Surface, cat #167008) and incubated 24 hours at 37° C. without shaking. Supernatant was aspirated and wells were washed with 100 μL of 0.9% sodium chloride and filled with 100 μL of either hard water or 6.6 gr/L non ionic detergent or 3.3 gr/L model A detergent containing 0 (control) or 80, 40, 20, 10, 5, 2.5, 1.25, 0.62, 0.31, 0.16, 0.08, 0.04 μg/mL of enzyme. In some studies, the starting concentration was 40 μg/mL instead of 80 μg/mL. After incubation at 37° C. for 1 hour, wells were washed with water and stained for 15 min with 100 μL of 0.095% crystal violet solution (SIGMA V5265). Wells were then rinsed twice with 100 μL water, dried and the plates were scanned. The lowest concentration of each enzyme that could detach the biofilm of the S. aureus organisms after 1 hour incubation, in the presence and absence of detergent was determined (see Table 4). All enzymes were assayed per duplicate in two to six independent tests. The average of minimal concentration for biofilm detachment was calculated for each of the enzymes and listed in the table below.

TABLE 4
Minimal concentration of enzyme that can detach the visible
formation of S. aureus after 1 hour incubation in either
hard water, non-ionic detergent or model A detergent.

| | Minimal | Minimal | |
|---|---|---|---|
| | concentration | concentration | Minimal |
| | for biofilm | for biofilm | concentration |
| | detachment | detachment | for biofilm |
| | in Hard | in non- | detachment in |
| | water | ionic detergent | Model A |
| | (μg/mL) | (μg/mL) | (μg/mL) |
| SEQ ID NO 3 | 3.75 | 10 | >40-80 |
| SEQ ID NO 6 | 1.21 | 10.83 | 35 |
| SEQ ID NO 49 | 1.25 | 11.25 | >40-80 |
| SEQ ID NO 9 | 5 | 22.5 | >40-80 |
| SEQ ID NO 50 | 0.23 | 3.13 | >40-80 |
| SEQ ID NO 12 | 40 | >80 | >80 |
| SEQ ID NO 51 | 1.88 | 12.5 | >40-80 |
| SEQ ID NO 15 | 3.13 | 22.5 | >40-80 |
| SEQ ID NO 52 | 0.39 | 3.13 | >40-80 |
| SEQ ID NO 30 | 3.75 | 12.5 | >40-80 |
| SEQ ID NO 53 | 0.47 | 1.88 | >40-80 |
| SEQ ID NO 18 | 2.5 | 10 | >40-80 |
| SEQ ID NO 54 | 1.88 | 7.5 | >40-80 |
| SEQ ID NO 21 | 20 | >80 | >80 |
| SEQ ID NO 24 | 1.88 | 7.5 | >40-80 |
| SEQ ID NO 55 | 1.56 | 3.75 | >40-80 |
| SEQ ID NO 27 | 0.37 | 1.48 | >40-80 |
| SEQ ID NO 33 | 0.26 | 3.33 | 8.33 |
| SEQ ID NO 36 | 0.39 | 1.56 | 3.75 |
| SEQ ID NO 56 | 2.58 | 5.31 | 50 |
| SEQ ID NO 39 | 2.5 | 40 | >40 |
| SEQ ID NO 57 | 3.75 | 40 | >40 |
| SEQ ID NO 58 | 2.5 | 20 | >40 |
| SEQ ID NO 45 | 0.83 | 4.17 | 30 |
| SEQ ID NO 48 | 3.13 | 11.25 | >80 |
| SEQ ID NO 42 | 5.63 | 21.25 | >80 |

### Example 6

### Construction of Clades and Phylogenetic Trees

The polypeptides in the invention includes a GHL13 domain. A phylogenetic tree was constructed, of polypeptide sequences containing a GHL13 domain, as defined in PFAM (PF14883, Pfam version 31.0 Finn (2016). Nucleic Acids Research, Database Issue 44: D279-D285). The phylogenetic tree was constructed from a multiple alignment of mature polypeptide sequences containing at least one GHL13 domain. The sequences were aligned using the MUSCLE algorithm version 3.8.31 (Edgar, 2004. Nucleic Acids Research 32(5): 1792-1797), and the trees were constructed using FastTree version 2.1.8 (Price et al., 2010, PIoS one 5(3)) and visualized using iTOL (Letunic & Bork, 2007. Bioinformatics 23(1): 127-128). The polypeptide of the GHL13 domain may comprise one or more of several motifs. One example is [Y/W]PX[D/N]F(SEQ ID NO 59) corresponding to positions 601 to 605 in H. zhanjiangensis (SEQ ID NO 36). Another motif which may be comprised by the polypeptides of the invention is [M/E/Y/F]AM[P/G] (SEQ ID NO:60), situated in positions corresponding to positions 532 to 535 in SEQ ID NO 36. The polypeptides containing a GHL13 domain can be separated into distinct sub-clusters. The sub-clusters are defined by one or more short sequence motifs, as well as containing a GHL13 domain as defined in PFAM (PF14883, Pfam version 31.0 Finn (2016). Nucleic Acids Research, Database Issue 44: D279-D285). we denoted one cluster comprising the motif [Y/W]PX[D/N]F (SEQ ID NO 59) as the YPPDF clade. Another motif characteristic of this domain is [M/E/Y/F]AM[P/G] (SEQ ID NO 60). All polypeptide sequences containing a GHL13 domain as well as the motifs will be denoted as belonging to the YPPDF clade.

### Generation of YPDDF Clade

The YPDDF clade comprises GHL13 polypeptides of bacterial origin, having PNAG-hydrolyzing activity. A phylogenetic tree was constructed, of polypeptide sequences containing a GHL13 domain, as defined above. The phylogenetic tree was constructed from a multiple alignment of mature polypeptide sequences containing at least one GHL13 domain. The sequences were aligned using the MUSCLE algorithm version 3.8.31 (Edgar, 2004. Nucleic Acids Research 32(5): 1792-1797), and the tree was constructed using FastTree version 2.1.8 (Price et al., 2010, PIoS one 5(3)) and visualized using iTOL (Letunic & Bork, 2007. Bioinformatics 23(1): 127-128). The polypeptides in GHL13 can be separated into sub-clusters, where we denoted one YPPDF. A characteristic motif for this group is the motif [Y/W]PX[D/N]F (SEQ ID NO 59) corresponding to amino acid 601 to 605 in SEQ ID NO 36. Another motif characteristic of this domain is [W/E/Y/F]AM[P/G] (SEQ ID NO 60), corresponding to amino acid 532 to 535 in SEQ ID NO 36. The YPDDF clade may also comprise polypeptides including a CE4 domain with the motif example WPY, corresponding to positions 211 to 213 of SEQ ID NO 36. The WPY motif is located within the CE4 domain.

### Generation of Phylogenetic Trees

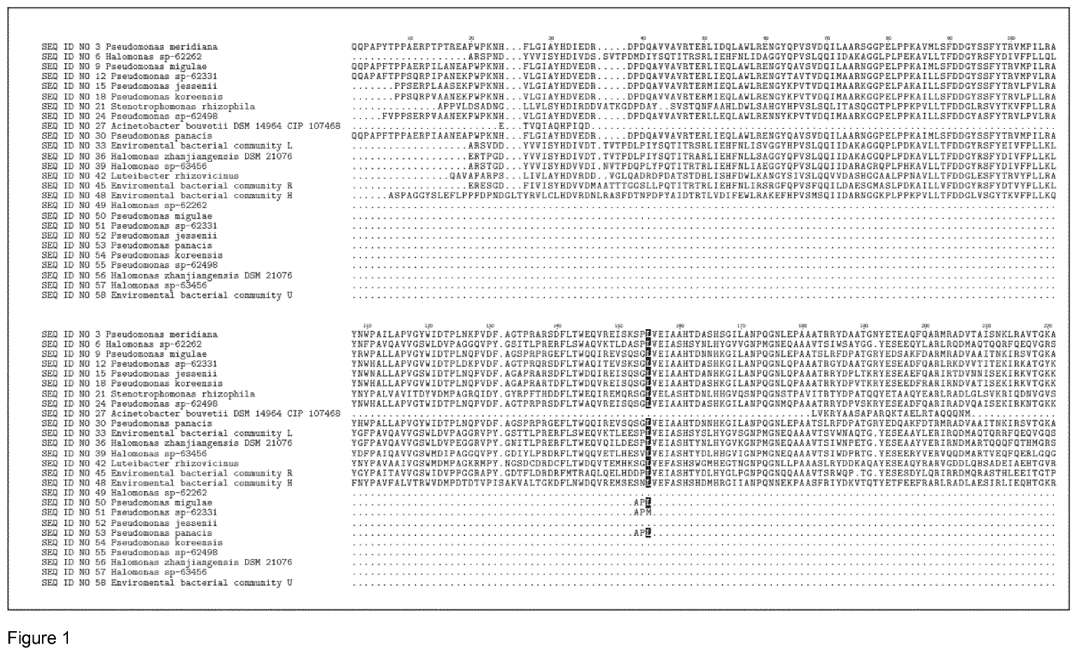

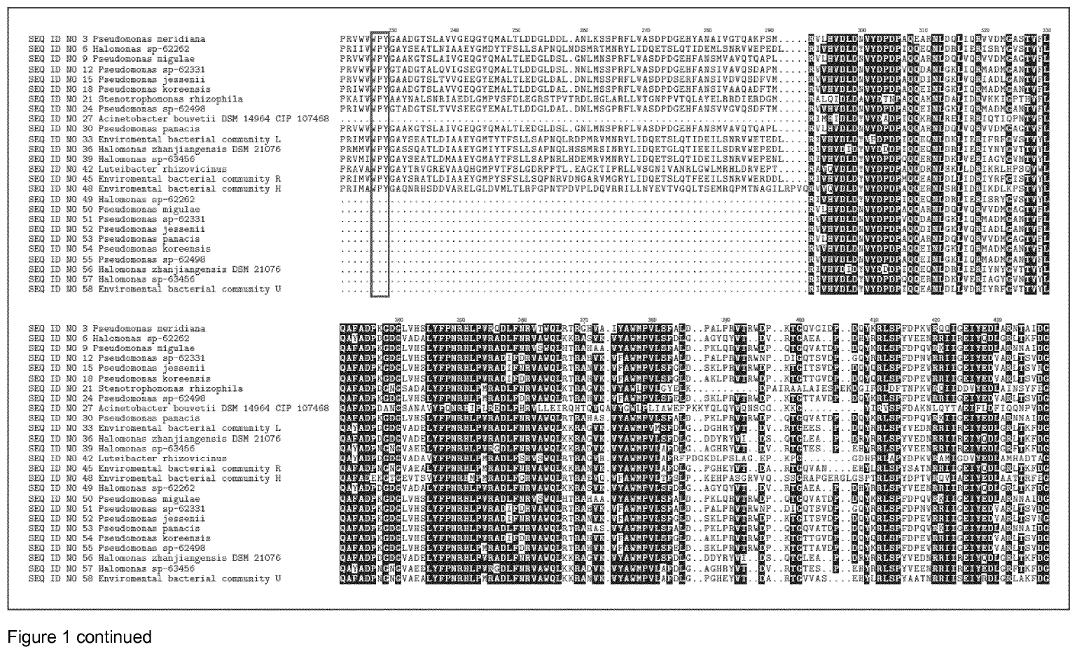

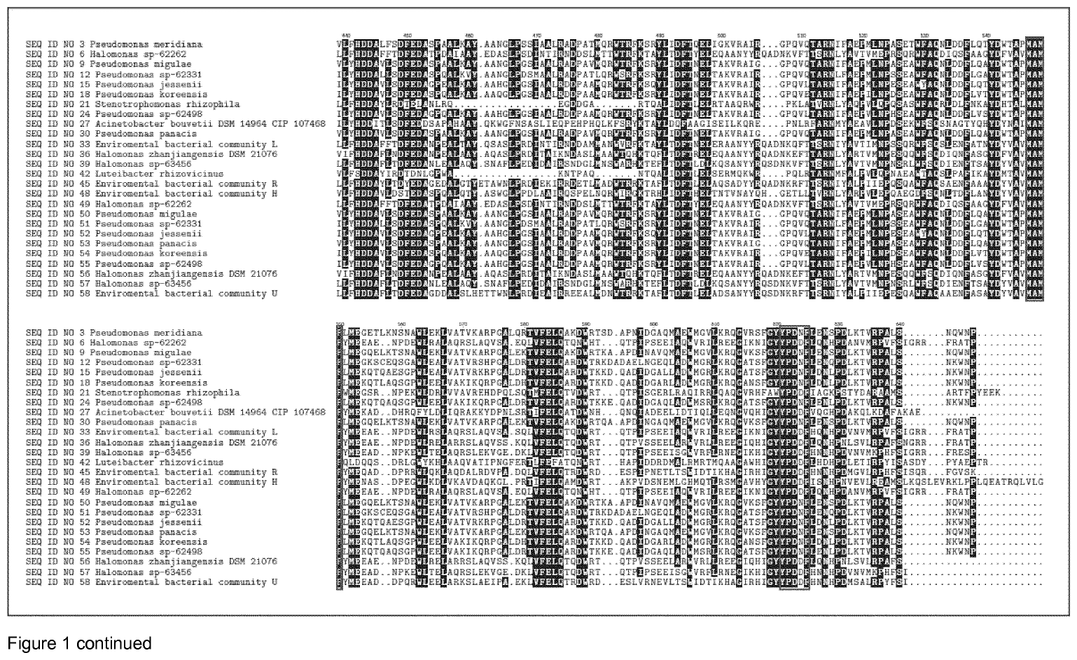

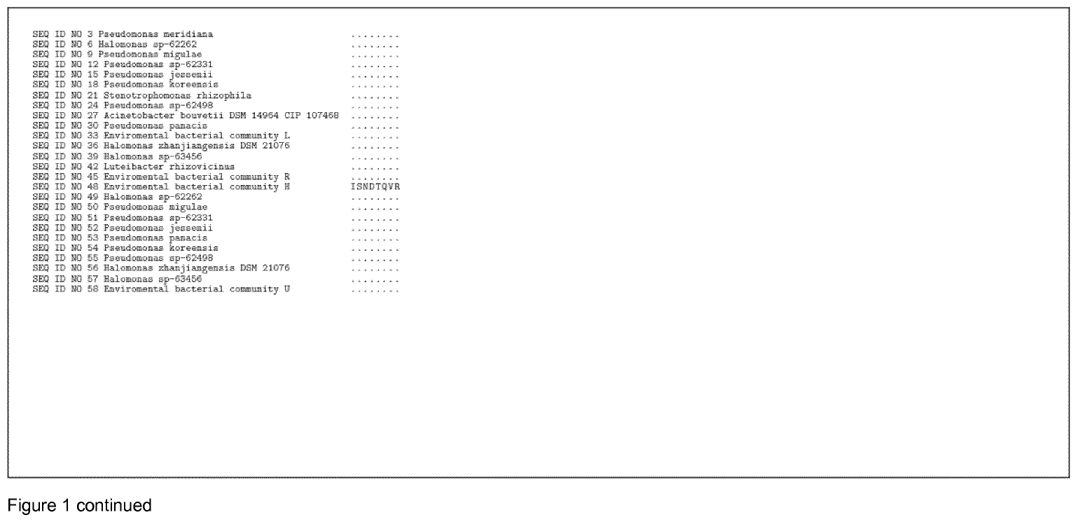

A phylogenetic tree was constructed, of polypeptide sequences containing a GHL13 domain, as defined above. The phylogenetic tree was constructed from a multiple alignment of mature polypeptide sequences containing at least one GHL13 domain. The sequences were aligned using the MUSCLE algorithm version 3.8.31 (Edgar, 2004. Nucleic Acids Research 32(5): 1792-1797), and the tree was constructed using FastTree version 2.1.8 (Price et al., 2010, PIoS one 5(3)) and visualized using iTOL (Letunic & Bork, 2007, Bioinformatics 23(1): 127-128). An alignment of the polypeptides of the invention is shown in FIG. 1, with the [Y/W]PX[D/N]F, [M/E/Y/F]AM[P/G], and WPY motifs highlighted.

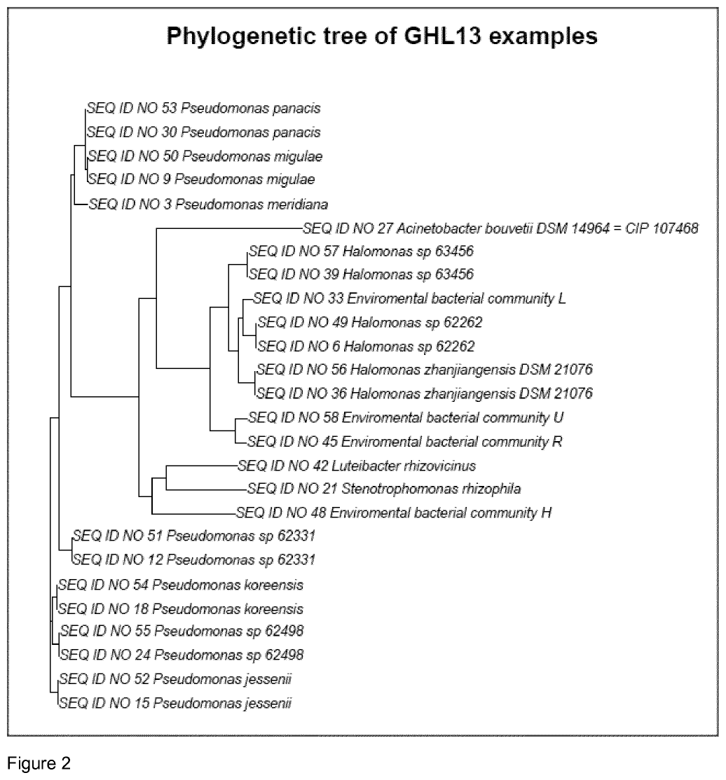

A phylogenetic tree of the polypeptides of the invention is shown in FIG. 2.

### Example 7

### Deep-Cleaning in Different Liquid Model Detergents on EPS Swatches

A crude extract of PNAG (poly-6(1-6)-N-acetylglucosamine) was prepared from Staphylococcus aureus 15981 as described above. A crude EPS (extracellular polymeric substances) extract was prepared from PNAG-producing Pseudomonas fluorescens as follows: P. fluorescens was restreaked on TSA and incubated for 1 day at 20° C. The strain was inoculated in TSB and incubated O/N at 20° C. After propagation, the culture was diluted (1:100) in M63 supplemented medium (15 mM (NH₄)₂SO₄, 100 mM KH₂PO₄, 1.8 μM FeSO₄, 1 mM MgSO₄.7H2O, 0.4% (w/v) glycerol, 0.2% (w/v) Casamino acids and 0.0001% (w/v) Thiamine), added to a Corning® CelIBIND® 225 cm² Angled Neck Cell Culture Flasks with Vent Cap (400 ml per flask) and incubated statically for 3 days at 20° C. The biofilm culture was subsequently pelleted by centrifugation (10 min, 8000 g, 25° C.), and the cells were resuspended in 3M NaCI (4 ml per flask) and incubated for 30 min at 30° C. to extract the surface-associated EPS. The EPS-containing supernatant obtained after centrifugation (10 min, 5000 g, 25° C.) was stored at −20° C. until further use. 50 ul aliquots of the PNAG extracts were spotted on sterile textile swatches (WFK20A) and incubated for 15 min at ambient temperature. 50 ul aliquots of 3M NaCI were spotted on control swatches. The swatches (sterile or with PNAG) were placed in 50 mL test tubes and 10 mL of wash liquor (15° dH water with 0.2 g/L iron(III) oxide nano-powder (544884; Sigma-Aldrich) with 3.33 g/L liquid model A or NI detergent) and enzyme was added. Washes without enzyme were included as controls. The test tubes were placed in a Stuart rotator and incubated for 1 hour at 37° C. at 20 rpm. The wash liquor was then removed, and the swatches were rinsed twice with 15° dH water and dried on filter paper over night. The remission (REM^{460 nm}) values were measured using a Macbeth Color-Eye 7000 (CE7000), and are displayed in table 4 and 5. Wash performance (WP), i.e. delta values (REM^{460 nm}_{(swatch washed with enzyme)}−REM^{460 nm}_{(swatch washed without enzyme)}) are also indicated.

TABLE 4

| | | | Model NI | | Model A | |
|---|---|---|---|---|---|---|
| | | conc. | Average | WP | Average | WP |
| Swatch | Enzyme | (μg/ml) | REM^{460 nm} | (ΔREM^{460 nm}) | REM^{460 nm} | (ΔREM^{460 nm}) |
| wfk20A, no EPS | No | 0 | 65.8 | | 55.5 | |
| | enzyme | | | | | |
| wfk20A, | No | 0 | 35.5 | | 24.2 | |
| P. fluorescens EPS | enzyme | | | | | |
| wfk20A, | SEQ ID | 0.2 | 38.8 | 3.3 | 28.2 | 3.9 |
| P. fluorescens EPS | NO 36 | | | | | |
| wfk20A, | SEQ ID | 2 | 65.5 | 30.0 | 39.3 | 15.1 |
| P. fluorescens EPS | NO 36 | | | | | |
| wfk20A, | SEQ ID | 10 | 71.7 | 36.1 | 48.3 | 24.0 |
| P. fluorescens EPS | NO 36 | | | | | |
| wfk20A, | SEQ ID | 2 | 42.3 | 6.8 | 25.9 | 1.6 |
| P. fluorescens EPS | NO 89 | | | | | |

TABLE 5

| | | | Model NI | | Model A | |
|---|---|---|---|---|---|---|
| | | conc. | Average | WP | Average | WP |
| Swatch | Enzyme | (μg/ml) | REM^{460 nm} | (ΔREM^{460 nm}) | REM^{460 nm} | (ΔREM^{460 nm}) |
| wfk20A, no EPS | No | 0 | 63.2 | | 54.6 | |
| | enzyme | | | | | |
| wfk20A, | No | 0 | 37.8 | | 40.0 | |
| S. aureus EPS | enzyme | | | | | |
| wfk20A, | SEQ ID | 0.2 | 54.8 | 17.0 | 52.2 | 12.2 |
| S. aureus EPS | NO 36 | | | | | |
| wfk20A, | SEQ ID | 2 | 65.7 | 27.9 | 66.5 | 26.4 |
| S. aureus EPS | NO 36 | | | | | |
| wfk20A, | SEQ ID | 10 | 69.2 | 31.3 | 69.6 | 29.5 |
| S. aureus EPS | NO 36 | | | | | |
| wfk20A, | SEQ ID | 2 | 60.4 | 22.6 | 46.0 | 6.0 |
| S. aureus EPS | NO 89 | | | | | |
| wfk20A, | SEQ ID | 10 | 65.7 | 27.8 | 48.2 | 8.1 |
| S. aureus EPS | NO 89 | | | | | |

### Example 8

### Endoscope Cleaning in Liquid Model Detergent

Endoscope biofilms were established using S. aureus (Valle et al., Mol Microbiol.2003 May; 48 (4):1075-87) as follows: The strain was inoculated into 10 mL of TSB and incubated for 16 hours at 37° C. with shaking (200 rpm). After propagation, the culture was diluted (1:100) in fresh TSB+1% glucose (24563; Roquette Freres) and 2 mL aliquots were added to the wells of 24-well polystyrene flat-bottom microplates (144530; Thermo Fisher Scientific) containing sterile pieces (1 cm) of endoscope tubing (4.7 mm diameter, FluoroelastomerNiton®, USP Class VI, Endoscopy Development Company, LLC). Sterile medium was added to control wells. After 24 h at 37° C. (static incubation), the endoscope pieces were rinsed with water (5° dH), and treated with 2 ml of a model cleaning solution (5 g/L Model detergent MC in 5° dH water) containing no enzyme or 20 μg/mL enzyme for 1 hour at 37° C. under static conditions. The endoscope pieces were then rinsed with 5° dH water and stained with 0.095% crystal violet (SIGMA V5265) for 15 min. Following staining, the endoscope pieces were rinsed twice, blotted on absorbent paper and the remaining dye was dissolved using 95% ethanol. 200 μl aliquots of the suspensions were transferred to a 96-well microtiter plate and the absorbance was measured at 595 nm. The results are displayed in table 6 as percentage of remaining biofilm after enzymatic treatment as compared to the control (endoscope biofilm cleaned without enzyme).

TABLE 6
Endoscope cleaning properties in medical cleaning model detergent

| | Enzyme dosage | Remaining biofilm |
|---|---|---|
| Enzyme | (μg/ml) | (% of untreated control) |
| no enzyme | 0 | 100.0 |
| SEQ ID NO 45 | 20 | 0.4 |

The results show that the polypeptides of the invention have endoscope cleaning properties i.e. disrupt and/or remove the biofilm or components of the biofilm tested when compared to samples comprising no enzyme.

1. A composition comprising at least 0.01 mg of active polypeptide per gram of composition, wherein the polypeptide comprises a GHL13 domain and at least one cleaning component.

2. The composition according to claim 1, wherein the polypeptide further comprises a CE4 domain.

3. A composition according to claim 1, wherein the polypeptide is of the YPDDF clade, comprising one or more of the motif(s) [YW]PX[D/N]F (SEQ ID NO 59), [M/E/Y/F]AM[P/G] (SEQ ID NO 60) or WPY.

4. The composition of claim 1, wherein the polypeptide has at least 60% sequence identity to the mature polypeptide shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36, SEQ ID NO 39, SEQ ID NO 42, SEQ ID NO 45, SEQ ID NO 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86 or SEQ ID NO 89.

5. The composition of claim 1, wherein the polypeptide
(a) comprising or consisting of SEQ ID NO: 3 or the mature polypeptide of SEQ ID NO: 2;
(b) comprising or consisting of SEQ ID NO: 6 or the mature polypeptide of SEQ ID NO: 5;
(c) comprising or consisting of SEQ ID NO: 9 or the mature polypeptide of SEQ ID NO: 8;
(d) comprising or consisting of SEQ ID NO: 12 or the mature polypeptide of SEQ ID NO: 11;
(e) comprising or consisting of SEQ ID NO: 15 or the mature polypeptide of SEQ ID NO: 14;
(f) comprising or consisting of SEQ ID NO: 18 or the mature polypeptide of SEQ ID NO: 17;
(g) comprising or consisting of SEQ ID NO: 21 or the mature polypeptide of SEQ ID NO: 20;
(h) comprising or consisting of SEQ ID NO: 24 or the mature polypeptide of SEQ ID NO: 23;
(i) comprising or consisting of SEQ ID NO: 27 or the mature polypeptide of SEQ ID NO: 26;
(j) comprising or consisting of SEQ ID NO: 30 or the mature polypeptide of SEQ ID NO: 29;
(k) comprising or consisting of SEQ ID NO: 33 or the mature polypeptide of SEQ ID NO: 32;
(l) comprising or consisting of SEQ ID NO: 36 or the mature polypeptide of SEQ ID NO: 35;
(m) comprising or consisting of SEQ ID NO: 39 or the mature polypeptide of SEQ ID NO: 38;
(n) comprising or consisting of SEQ ID NO: 42 or the mature polypeptide of SEQ ID NO: 41;
(o) comprising or consisting of SEQ ID NO: 45 or the mature polypeptide of SEQ ID NO: 44;
(p) comprising or consisting of SEQ ID NO: 48 or the mature polypeptide of SEQ ID NO: 47;
(q) comprising or consisting of SEQ ID NO: 65 or the mature polypeptide of SEQ ID NO: 64;
(r) comprising or consisting of SEQ ID NO: 68 or the mature polypeptide of SEQ ID NO: 67;
(s) comprising or consisting of SEQ ID NO: 71 or the mature polypeptide of SEQ ID NO: 70;
(t) comprising or consisting of SEQ ID NO: 74 or the mature polypeptide of SEQ ID NO: 73;
(u) comprising or consisting of SEQ ID NO: 77 or the mature polypeptide of SEQ ID NO: 76;
(v) comprising or consisting of SEQ ID NO: 80 or the mature polypeptide of SEQ ID NO: 79;
(x) comprising or consisting of SEQ ID NO: 83 or the mature polypeptide of SEQ ID NO: 82;
(y) comprising or consisting of SEQ ID NO: 86 or the mature polypeptide of SEQ ID NO: 85; and
(z) comprising or consisting of SEQ ID NO: 89 or the mature polypeptide of SEQ ID NO: 88.

6. The composition of claim 1, wherein the composition is a cleaning composition such as a laundry or dish wash composition.

7. The composition according to claim 6, wherein the cleaning component is selected from the group consisting of,
a) at least one builder,
b) at least one surfactant, and
c) at least one bleach component.

8. A polypeptide having hydrolytic and/or deacetyl activity, wherein the polypeptide is of the YPDDF clade, comprising one or more of the motif(s) [Y/W]PX[D/N]F (SEQ ID NO 59), [M/E/Y/F]AM[P/G] (SEQ ID NO 60) or WPY and wherein the polypeptide is selected from the group consisting of:
(a) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 3;
(b) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 6;
(c) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 9;
(d) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 12;
(e) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 15;
(f) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 18;
(g) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 21;
(h) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 24;
(i) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO 27;
(j) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 30;
(k) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 33;
(l) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 36;
(m) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 39;
(n) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 42;
(o) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 45;
(p) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 48;
(q) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 65;
(r) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 68;
(s) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 71;
(t) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 74;
(u) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 77;
(v) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 80;
(x) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 83;
(y) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 86;
(z) a polypeptide having at least 60%, at least 65%, at least 70%, at least 75%, at least 80%, at least 85%, at least 90% at least 95%, at least 96%, at least 97%, at least 98%, at least 99% or 100% sequence identity to the polypeptide of SEQ ID NO: 89;
(aa) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO 65, SEQ ID NO 68, SEQ ID NO 71, SEQ ID NO 74, SEQ ID NO 77, SEQ ID NO 80, SEQ ID NO 83, SEQ ID NO 86 and SEQ ID NO 89, wherein the variant has hydrolytic and/or deacetylase activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions;
(bb) a polypeptide comprising the polypeptide of (a) to (aa) and a N-terminal and/or C-terminal His-tag and/or HQ-tag;
(cc) a polypeptide comprising the polypeptide of (a) to (aa) and a N-terminal and/or C-terminal extension of between 1 and 10 amino acids; and
(dd) a fragment of the polypeptide of (a) to (aa) having hydrolytic and/or deacetylase activity and having at least 90% of the length of the mature polypeptide.

9. The polypeptide according to claim 8, wherein the polypeptide has at least 80% sequence identity to the polypeptide shown in SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45,SEQ ID NO: 48, SEQ ID NO 65, SEQ ID NO 68, SEQ ID NO 71, SEQ ID NO 74, SEQ ID NO 77. SEQ ID NO 80, SEQ ID NO 83, SEQ ID NO 86 and SEQ ID NO 89.

10. The polypeptide of claim 8, which is encoded by a polynucleotide, wherein the polynucleotide has at least 85% sequence identity to the mature polypeptide coding sequence of SEQ ID NO 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43SEQ ID NO: 46, SEQ ID NO 63, SEQ ID NO 66, SEQ ID NO 69, SEQ ID NO 72, SEQ ID NO 75, SEQ ID NO 78, SEQ ID NO 81, SEQ ID NO 84 or SEQ ID NO 87.

11. A method for laundering an item comprising the steps of:
a. exposing an item to a wash liquor comprising the polypeptide according to claim 8;
b. completing at least one wash cycle; and
c. optionally rinsing the item,
wherein the item is a textile.

12. (canceled)

13. (canceled)

14. (canceled)

15. (canceled)
